(19) United States
(12) Patent Application Publication
(10) Pub. No.: US 2022/0307078 A1
(43) Pub. Date: Sep. 29, 2022

(54) SYSTEMS AND METHODS FOR DETECTING GENETIC VARIATION IN NUCLEIC ACIDS

(71) Applicant: ZEPTO LIFE TECHNOLOGY, LLC, St. Paul, MN (US)

(72) Inventors: Matthew NELSON, Lino Lakes, MN (US); Yi-Hsuan SU, Maplewood, MN (US); Jung Min SONG, Roseville, MN (US); Vicci L. KORMAN, Minneapolis, MN (US); Todd Michael KLEIN, Wayzata, MN (US); Wei WANG, St. Paul, MN (US); Ian STUYVENBERG, Minneapolis, MN (US); Gemma Roselle MENDONSA, Edina, MN (US)

(21) Appl. No.: 17/641,718

(22) PCT Filed: Jan. 22, 2020
(86) PCT No.: PCT/US2020/014570

§ 371 Date: Mar. 9, 2022

Related U.S. Application Data
(60) Provisional application No. 62/897,561, filed on Sep. 9, 2019.
(60) Provisional application No. 62/958,510, filed on Jan. 8, 2020.

(51) Int. Cl. C12Q 1/6844; C12Q 1/6827; C12Q 1/6886

(52) U.S. Cl. CPC C12Q 1/6844; C12Q 1/6827; C12Q 1/6886; C12Q 2600/112

(57) ABSTRACT

Methods of detecting one or more genetic variants, including allelic variants, in one or more query samples include providing one or more sensors that each include capture nucleic acids disposed on a functionalized surface of one or more giant magnetoresistance (GMR) sensors. The methods detect the presence of one or more analytes in one or more query samples by measuring magnetoresistance change of the one or more GMR sensors based on determining magnetoresistance before and after passing magnetic particles over the one or more sensors.

### CROSS-REFERENCE TO RELATED APPLICATIONS

The present application is a U.S. National stage entry of International Application No. PCT/US2020/014570, filed on Jan. 22, 2020, which claims priority to U.S. Provisional Patent Application No. 62/897,561, filed Sep. 9, 2019, and U.S. Provisional Patent Application No. 62/958,510, filed Jan. 8, 2020, the contents of which are hereby incorporated by reference in their entireties.

### INTRODUCTION

The present disclosure is generally related to, inter alia, systems and methods for sensing and identifying analytes, such as nucleic acid and nucleic acid variation, in one or more samples. The present disclosure also relates to analyte- and nucleic acid-sensing devices, such as microfluidic devices and Giant Magneto-Resistance (GMR) sensors and methods of detection based on such microfluidic devices and Giant Magneto-Resistance (GMR) sensors.

Genetic information of living organisms (e.g., animals, plants, microorganisms, viruses) is encoded in deoxyribonucleic acid (DNA) or ribonucleic acid (RNA). Genetic information is a succession of nucleotides or modified nucleotides representing the primary structure of nucleic acids. The nucleic acid content (e.g., DNA) of an organism is often referred to as a genome. In most humans, the complete genome typically contains about 30,000 genes located on twenty-three pairs of chromosomes. Most genes encode a specific protein, which after expression via transcription and translation fulfills one or more biochemical functions within a living cell.

Many medical conditions are caused by, or its risk of occurrence is influenced by, one or more genetic variations within a genome. Some genetic variations may predispose an individual to, or cause, any of a number of diseases such as, for example, diabetes, arteriosclerosis, obesity, various autoimmune diseases and cancer (e.g., colorectal, breast, ovarian, lung). Such genetic variations can take the form of an addition, substitution, insertion or deletion of one or more nucleotides within a genome.

Genetic variation and/or genetic polymorphism also exists among and between different organisms, including closely related organisms. Such organisms may be classified and/or distinguished as belonging to the same, similar, or different taxonomic group, such as a given domain, kingdom, phylum, class, order, family, genus, or species. It is often desirable to identify, detect, and or distinguish between closely related organisms, such as pathogenic organisms, that are otherwise closely related, such as by belonging to same or similar families, genuses, or species, from one or more samples obtained from subject or from the environment.

Genetic variations can be identified by analysis of nucleic acids. Nucleic acids of a genome can be analyzed by various methods including, for example, methods that involve massively parallel sequencing, microarray analysis, and multiplex ligation probe amplification. Such method can be costly, time consuming and may require substantial computer processing, which is problematic when it is desired to quickly and accurately detect the presence or absence of known genetic variation (e.g., a single nucleotide mutation) in a genome of a subject suspected of having a disease or condition associated with the genetic variation.

GMR sensors enable development of multiplex assays and multiplex detection schemes, for example for, performing multiplex assays for detecting more than one analyte in the same query sample or in difference query samples, with high sensitivity and low cost in a compact system, and therefore have the potential to provide a platform suitable for a wide variety of applications. Reliable analyte sensing remains a challenge. The present disclosure provides exemplary solutions.

Devices and methods presented herein offer significant advances and improvements to current nucleic acid analysis techniques. Such advances and improvements described herein can help expedite screening for genetic variations in a sample by methods that are low cost and highly sensitive.

The present disclosure is generally related to a microfluidic device and uses thereof to detect analytes and/or genetic variation, in one or more samples. The devices and methods presented herein also utilize magnetic sensors. The devices and methods presented herein also utilize, in some embodiments, DNA binding proteins and magnetic sensors. In some embodiments, the present disclosure relates to a microfluidic device comprising a Giant MagnetoResistance (GMR) sensor.

### SUMMARY

In some aspects, presented herein is a method of detecting the presence of a first genetic variant in a target nucleic acid comprising (a) contacting the target nucleic acid with (i) a first primer, (ii) a second primer comprising a first member of a binding pair, (iii) a polymerase and (iv) a blocking oligonucleotide, wherein the blocking oligonucleotide comprises a sequence complementary to a second genetic variant of the target nucleic acid, and the first and second primers are configured for amplification of the target nucleic acid; (b) amplifying the target nucleic acid thereby providing amplicons of the target nucleic acid; (c) contacting the amplicons with a capture nucleic acid, wherein the capture nucleic acid comprises a sequence complementary to the first genetic variant of the target sequence, thereby providing captured amplicons comprising the first member of the binding pair; (d) contacting the captured amplicons with a detectable label comprising a second member of the binding pair; and (e) detecting a presence, absence, amount, or change thereof, of the detectable label. In some embodiments, the method comprising detecting the presence or absence of a cancer in a subject according to the presence or absence of the first genetic variant in a target nucleic acid. In some embodiments, a method comprises administering a suitable treatment to a subject when a first genetic variant is detected. In some embodiments, the capture nucleic acid is attached to a surface of a sensor. In some embodiments, the detecting of (e) comprises detecting the presence, absence, amount, or change thereof, of the detectable label at the surface of the sensor. In some embodiments, the detecting of (e) comprises a dynamic detection process. In some embodiments, the dynamic detection process comprises increasing the stringency of hybridization conditions at the surface of the sensor. In some embodiments, the dynamic detection process comprises increasing a temperature at or near the sensor, or at the surface of the sensor, during the detecting of (e). In some embodiments, the dynamic detection process comprises changing a salt or cation concentration at or near the sensor, or at the surface of the sensor, during the detecting of (e). In some embodiments, the dynamic detection process comprises flowing a fluid across the surface of the sensor during the detecting of (e). In some embodiments, the detecting of (e) comprises detecting binding of one or more amplicons that bind to the capture nucleic acid. In some embodiments, the detecting of (e) comprises detecting a change in an amount of amplicons that are bound to the surface of the sensor. In some embodiments, the detectable label comprises a magnetic particle and the second member of the binding pair. In some embodiments, the second member of the binding pair comprises streptavidin. In some embodiments, the first binding pair comprises biotin. In some embodiments, the genetic variant comprises an allelic genetic variant. In some embodiments, the genetic variant comprises a variant that distinguishes the presence of one organism from another in the sample.

In some embodiments, the sensor comprises a magnetic sensor, the detectable label comprises a magnetic particle, and the detecting of (e) comprises detecting a presence, absence, or amount of magnetic particles at or near the surface of the magnetic sensor. In some embodiments, the detecting of (e) comprises detecting a change in magnetoresistance at the surface of a sensor. In some embodiments, the detecting of the change in magnetoresistance of (e) comprises increasing the temperature on or near the surface of the sensor by at least 5° C. or a period of time while detecting the magnetoresistance, or changes thereof, at the surface of the sensor prior to, during and/or after increasing the temperature. In some embodiments, the detecting of the change in magnetoresistance of (e) comprises increasing the temperature on or near the surface of the sensor by at least 20° C. or a period of time while detecting the magnetoresistance, or changes thereof, at the surface of the sensor prior to, during and/or after increasing the temperature. In some embodiments, the presence of the first genetic variant in the target nucleic acid is determined according to the change of magnetoresistance detected in (e). In some embodiments, the detecting of the change in magnetoresistance of (e) comprises decreasing the sodium ion concentration by at least 50% while detecting the magnetoresistance, or changes thereof, at the surface of the sensor prior to, during and/or after changing the sodium concentration. In some embodiments, a detection of a change in magnetoresistance distinguishes between the presence of the first genetic variant, and the presence of a second genetic variant, or any other genetic variant or a mixture of genetic variants that bind to the capture nucleic acid. In some embodiments, the first genetic variant, second genetic variant, and any other genetic variant each comprises an allelic variant. In some embodiments, the first genetic variant, second genetic variant, and any other genetic variant each comprises a variant that distinguishes the presence of one organism from another organism in the sample.

In some embodiments, the first primer is attached to a substrate or surface, for example a surface of an amplification chamber. In some embodiments, the first primer comprises a free 5′-hydroxy group.

In some embodiments, the blocking oligonucleotide comprises a melting temperature (Tm) of at least 75° C., at least 80° C. or at least 85° C. In some embodiments, the blocking oligonucleotide has a length in a range from 9 to 30 oligonucleotides. In some embodiments, the blocking oligonucleotide has a length in a range from 9 to 20 oligonucleotides. In some embodiments, the blocking oligonucleotide comprises one or more locked nucleotides. In some embodiments, the blocking oligonucleotide comprises at least 3 locked nucleotides.

In some embodiments, the blocking oligonucleotide, when hybridized to a second allelic variant of the target nucleic acid, substantially prevents amplification of the target nucleic acid comprising said second variant.

In some embodiments, a first primer comprises a 5′-phosphoylate nucleotide.

In some embodiments, after (b), the amplicons are contacted with a 5′-3′ exonuclease.

In some embodiments, the capture nucleic acid has a length in a range from 9 to 30 oligonucleotides. In some embodiments, the capture nucleic acid comprises a melting temperature of at least 50° C., at least 55° C. or at least 65° C. In some embodiments, the capture nucleic acid comprises one or more locked nucleotides. In some embodiments, the capture nucleic acid comprises at least 3 locked nucleotides.

In some embodiments, the first member of the binding pair comprises biotin and the second member of the binding pair comprises streptavidin.

In some embodiments, the amplifying of (b) comprises a polymerase chain reaction. In some embodiments, the amplifying of (b) comprises at least 40, or at least 50 cycles a polymerase chain reaction.

In some embodiments, the method is conducted on a sample obtained from a subject, wherein the sample comprises the target nucleic acid. In some embodiments, the sample is obtained from a pregnant female.

In some embodiments the sample comprises, or is suspected of comprising, at least one genetic variant comprising an organism. In some embodiments the sample comprises, or is suspected of comprising, at least one organism comprising a genetic. In some embodiment

In some embodiments a method comprises distinguishing at least one genetic variant from another genetic variant. In some embodiments a method comprises distinguishing at least one genetic variant from another genetic variant, thereby detecting and/or distinguishing one organism in a sample comprising, or suspected of comprising, a plurality of organisms.

In some embodiments, the presence of a genetic variant in a target nucleic acid is determined according to a change of magnetoresistance. In some embodiments, the presence of a first genetic variant in a target nucleic acid is determined according to a change of magnetoresistance. In some embodiments, the presence of at least one genetic variant in a target nucleic acid is determined according to a change of magnetoresistance. In some embodiments, the presence of at least one genetic variant in a sample containing, or suspected of containing, at least one genetic variant. In some embodiments, a first genetic variant, at least one genetic variant, or a plurality of genetic variants comprises an allelic variant, an at least one allelic variant, and/or a plurality of allelic variants.

In some embodiments, provided are methods of detecting at least one genetic variant comprising at least one target nucleic acid in a sample comprising, or suspected of comprising, the at least least one genetic variant, the method comprising: providing the sample; contacting the sample with (i) a plurality of different first primers and (ii) a plurality of different second primers, wherein each second primer comprises a first member of a binding pair, and (iii) a polymerase; amplifying the at least one genetic variant thereby providing amplicons of the at least one genetic variant; (c) contacting the amplicons with at plurality of different capture nucleic acids, wherein each different capture nucleic acid comprises a sequence complementary to at a different genetic variant of a class of genetic variants, thereby providing distinguishable captured amplicons comprising the first member of the binding pair; (d) contacting the distinguishable captured amplicons with a first detectable label comprising a second member of the binding pair; and (e) detecting a presence, absence, amount, or change thereof, of the first detectable label. In some embodiments, the detecting of (e) comprises detecting the presence, absence, amount, or change thereof, of the first detectable label at the surface of the sensor. In some embodiments, the detecting of (e) comprises a dynamic detection process. In some embodiments, the dynamic detection process comprises increasing a temperature at or near the sensor, or at the surface of the sensor, during the detecting of (e). In some embodiments, the the dynamic detection process comprises changing a salt or cation concentration at or near the sensor, or at the surface of the sensor, during the detecting of (e). In some embodiments, the dynamic detection process comprises flowing a fluid across the surface of the sensor during the detecting of (e). In some embodiments, the detecting (e) comprises detecting binding of one or more of the distinguishable amplicons that bind to the capture nucleic acid, thereby distinguishing one genetic variant from another genetic variant. In some embodiments, the detecting of (e) comprises detecting a change in an amount of distinguishable amplicons that are bound to the surface of the sensor.

In some embodiments, the sensor comprises a magnetic sensor, the first detectable label comprises a magnetic particle, and the detecting of (e) comprises detecting a presence, absence, amount, or change of magnetoresistance at or near the surface of the magnetic sensor. In some embodiments, the detecting of (e) comprises detecting a change in magnetoresistance at the surface of the sensor. In some embodiments, the presence of the at least one genetic variant in the target nucleic acid is determined according to a change of magnetoresistance detected in (e). In some embodiments, the detecting of (e) comprises distinguishing the presence, absence, or amount of the at least one genetic variant at the surface of the sensor compared to a presence, absence, or amount of another genetic variant at the surface of the sensor. In some embodiments, the detecting of (e) comprises distinguishing the presence, absence, or amount of at least one genetic variant at the surface of the sensor compared to a presence, absence or amount of another nucleic acid at the surface of the sensor. In some embodiments, the first member of the binding pair comprises biotin and the second member of the binding pair comprises streptavidin. In some embodiments, the method is conducted on a sample obtained from a subject, wherein the sample comprises, or is suspected of comprising, the least one genetic variant. In some embodiments, the sample comprises cell free DNA.

In some embodiments, prior to (a) the sample is contacted with a microfluid channel, wherein the microfluidic channel is operably and/or fluidically connected to the sensor. In some embodiments, prior to (a) the sample is contacted with a membrane configured to reversibly and/or non-specifically bind to nucleic acids in the sample, thereby providing bound nucleic acids, wherein the membrane is operably and/or fluidically connected to the microfluidic channel and to the sensor. In some embodiments, the amplifying is performed inside of an amplification chamber that is operably and/or fluidically connected to a microfluidic channel, to the sensor and optionally to a membrane. In some embodiments, prior to (a), the method comprises (i) contacting the sample with (i) a cell lysis solution, (ii) a membrane, (iii) optionally a wash solution, and (iv) an elution buffer, wherein after the contacting of (iv) bound nucleic acids are released from the membrane. In some embodiments, the nucleic acids in the sample are transported through the microfluidic channel to the membrane, transported through the microfluidic channel from the membrane to the amplification chamber, and transported through the microfluidic channel from the amplification chamber to the surface of the sensor.

In some embodiments, the sensor comprises a giant magnetomagnetoresistance (GMR) sensor.

In some embodiments, the at least one genetic variant comprises a single nucleotide polymorphism (SNP). In some embodiments, the at least one genetic variant comprises at least two single nucleotide polymorphisms (SNP).

In some embodiments, the at least one genetic variant comprises a single nucleotide mutation. In some embodiments, the at least one genetic variant comprises at least two single nucleotide mutations.

In some embodiments, the at least one genetic variant comprises a single nucleotide deletion or insertion. In some embodiments, the at least one genetic variant comprises at least two single nucleotide deletions or insertions.

In some embodiments, the captured amplicons are in fluid contact with a buffer and prior to, or during, the detecting of (e), a concentration of positively charged cations in the buffer is decreased by at least 50%. In some embodiments, the positively charged cations comprise sodium, potassium, calcium or magnesium.

In some embodiments, a sensitivity of detection of the at least one genetic variant is less than 15 copies per mL of the sample.

In some embodiments, the method detects the presence of the at least one genetic variant at a concentration as low as 0.01% of the target sequence in a sample.

In some embodiments, the method detects the presence of the at least one genetic variant at a concentration as low as 1% of the target sequence in the sample.

In some embodiments, the method is performed in a mircrofluidic device.

In some embodiments are provided methods of detecting the presence of a first genetic variant in a target nucleic acid comprising: (a) contacting the target nucleic acid with (i) a first primer, (ii) a second primer, (iii) a polymerase and (iv) a blocking oligonucleotide, wherein the blocking oligonucleotide comprises a sequence complementary to a second genetic variant of the target nucleic acid, and the first and second primers are configured for amplification of the target nucleic acid, and amplifying the target nucleic acid thereby providing amplicons of the target nucleic acid, wherein the amplifying is performed within an amplification chamber of a microfluidic device; (b) contacting the amplicons with a plurality of capture nucleic acids thereby providing captured amplicons, wherein (i) the capture nucleic acids are attached to a surface of a magnetic sensor, (ii) the contacting of (b) comprises transporting the amplicons through a first microfluidic channel to the magnetic sensor, (iii) the first microfluidic channel and the magnetic sensor are disposed within the microfluidic device, and (iv) each of the capture nucleic acids comprises a sequence complementary to the first genetic variant of the target nucleic acid; (c) contacting the captured amplicons with a plurality of magnetic particles, wherein the magnetic particles a disposed within a first chamber of the microfluidic device, and the contacting of (c) comprises transporting the magnetic particles through a second microfluidic channel from the first chamber to the sensor; (d) washing the sensor with a wash solution, wherein the wash solution is disposed within a second chamber of the microfluidic device, and the washing comprises transporting the wash solution through a third microfluidic channel from the second chamber to the sensor; and (e) detecting a presence, absence, amount, of one or more of the magnetic particles that are associated with the surface of the sensor, wherein the detecting is performed prior to, during and/or after (d).

In some embodiments are provided methods of detecting at least one genetic variant comprising at least one target nucleic acid in a sample comprising, or suspected of comprising, the at least least one genetic variant, the method comprising: providing the sample; contacting the sample with (i) a plurality of different first primers and (ii) a plurality of different second primers, wherein each second primer comprises a first member of a binding pair, and (iii) a polymerase; amplifying the at least one genetic variant thereby providing amplicons of the at least one genetic variant, wherein the amplifying is performed within an amplification chamber of a microfluidic device; (b) contacting the amplicons with a plurality of different capture nucleic acids, wherein each different capture nucleic acid comprises a sequence complementary to at a different genetic variant of a class of genetic variants, thereby providing distinguishable captured amplicons, wherein (i) the capture nucleic acids are attached to a surface of a magnetic sensor, (ii) the contacting of (b) comprises transporting the distinguishable captured amplicons through a first microfluidic channel to the magnetic sensor, (iii) the first microfluidic channel and the magnetic sensor are disposed within the microfluidic device, and (iv) each of the capture nucleic acids comprises a sequence complementary to the at least one genetic variant of the target nucleic acid; (c) contacting the distinguishable captured amplicons with a plurality of magnetic particles, wherein the magnetic particles a disposed within a first chamber of the microfluidic device, and the contacting of (c) comprises transporting the magnetic particles through a second microfluidic channel from the first chamber to the sensor; (d) washing the sensor with a wash solution, wherein the wash solution is disposed within a second chamber of the microfluidic device, and the washing comprises transporting the wash solution through a third microfluidic channel from the second chamber to the sensor; and (e) detecting a presence, absence, amount, of one or more of the magnetic particles that are associated with the surface of the sensor, wherein the detecting is performed prior to, during and/or after (d).

In some embodiments are provided methods of detecting the presence of at least two different genetic variants in at least two different target nucleic acids in a multiplex detection scheme, the method comprising: (a) providing spacially disposed giant magnetoresistance (GMR) sensors, wherein at least two of the GMR sensors comprise at least two different capture nucleic acids disposed on a functionalized surface of the at least two (GMR) sensors, wherein each of the different capture nucleic acids is complimentary to one of the at least two genetic variants; (b) contacting each of the at least two different target nucleic acids with (i) a first primer, (ii) a second primer comprising a first member of a binding pair, (iii) a polymerase and (iv) a blocking oligonucleotide, wherein the blocking oligonucleotide comprises a sequence complementary to a genetic variant of the target nucleic acid, and the first and second primers are configured for amplification of the at least two target nucleic acids, and amplifying the at least two target target nucleic acids, thereby providing amplicons of the at least two target nucleic acids, (c) contacting the amplicons with a plurality of different capture nucleic acids, wherein each different capture nucleic acid comprises a sequence complementary to at a different genetic variant of a class of genetic variants, thereby providing distinguishable captured amplicons comprising the first member of the binding pair; (d) contacting the distinguishable captured amplicons with a plurality of first detectable labels comprising magnetic particles and a second member of the binding pair; and (e) detecting the presence, absence amount or change thereof, of the first detectable label.

In some embodiments are provided methods of any detecting at least one genetic variant comprising at least one target nucleic acid in a sample comprising, or suspected of comprising, the at least least one genetic variant in a multiplex detection scheme, the method comprising: providing spacially disposed giant magnetoresistance (GMR) sensors, wherein at least two of the GMR sensors comprise at least two different capture nucleic acids disposed on a functionalized surface of the at least two (GMR) sensors, wherein each of the different capture nucleic acids is complimentary to one of the at least two genetic variants; providing the sample; contacting the sample with (i) a plurality of different first primers and (ii) a plurality of different second primers, wherein each second primer comprises a first member of a binding pair, and (iii) a polymerase; amplifying the at least one genetic variant thereby providing amplicons of the at least one genetic variant; (c) contacting the amplicons with a plurality of different capture nucleic acids, wherein each different capture nucleic acid comprises a sequence complementary to at a different genetic variant of a class of genetic variants, thereby providing distinguishable captured amplicons comprising the first member of the binding pair; (d) contacting the distinguishable captured amplicons with a plurality of first detectable labels comprising magnetic particles and a second member of the binding pair; and (e) detecting a presence, absence, amount, or change thereof, of the first detectable label.

In some embodiments, a method described herein comprises detecting and/or distinguishing between a nucleic acid (e.g., a target nucleic acid) that comprises a genetic variation, also referred to interchangeably throughout as a genetic variant. In some embodiments, a method described herein comprises detecting and/or distinguishing between a nucleic acid (e.g., a target nucleic acid) that comprises a genetic variation comprising one or more nucleotide deletions, duplications, additions, insertions, substitutions, mutations, repeats, genetic homologues, genetic orthologs, and/or polymorphisms.

In some embodiments, a method described herein comprises detecting and/or distinguishing between one or more genetic variants comprising one or more allelic variants. In some embodiments, a method described herein comprises detecting and/or distinguishing between one or more allelic variants comprising one or more polymorphisms present in different members of the same species. In some embodiments, such allelic variants result in expression of proteins with similar but slightly different functional characteristics, which predispose subjects to, or result in, certain disease states or conditions.

In some embodiments, a method described herein comprises detecting the presence or absence of, and/or distinguishing between one or more allelic variants comprising a mutation in an oncogene. In some embodiments, a method described herein comprises detecting the presence or absence of, and/or distinguishing between one or more allelic variants comprising a mutation in gene that, predisposes and/or gives rise to, a cancer in a subject.

In some embodiments, a method described herein comprises detecting the presence or absence of, and/or distinguishing between one or more allelic variants comprising a mutation in an EGFR gene. In some embodiments, a method described herein comprises detecting the presence or absence of, and/or distinguishing between one or more allelic variants comprising a mutation in an EGFR gene comprising a c.2573T>G (T becomes a G) substitution in exon 21 of EGFR.

In some embodiments, a method described herein comprises detecting the presence or absence of, and/or distinguishing between one or more allelic variants comprising a mutation in KRAS gene. In some embodiments, a method described herein comprises detecting the presence or absence of, and/or distinguishing between one or more allelic variants comprising a G to a T or a G to an A at position 35 of the KRAS gene (i.e., the codon of the KRAS gene that codes for amino acid 12 and gives rise to the G12D and G12V mutation, respectively. In some embodiments, a method described herein comprises detecting the presence or absence of, and/or distinguishing between one or more allelic variants comprising a polymorphism or mutation that produces at least one of a G12D, G12V, G13D, G12C, G12A, G12S, G12R, or G13C amino acid mutation.

In some embodiments, a method described herein comprises detecting the presence or absence of, and/or distinguishing between one or more allelic variants comprising a mutation in a KRAS gene comprising employing at least one of the following primers and blocking oligonucleotides in the method:

Forward primer:
(SEQ ID NO: 7)
/5Biosg/ATTGTTGGATCATATTCGTCCAC
Reverse primer:
(SEQ ID NO: 8)
/5Phos/AGGCCTGCTGAAAATGACTG
Blocking oligonucleotide:
(SEQ ID NO: 9)
5′-C+T+G+G+T+G+G+C+G+T+A-3′,
where “+” indicates locked nucleic acid.

In some embodiments, a method described herein comprises detecting the presence or absence of, and/or distinguishing between one or more allelic variants comprising a mutation in a KRAS gene comprising employing the following primers and blocking oligonucleotides in the method:

Forward primer:
(SEQ ID NO: 7)
/5Biosg/ATTGTTGGATCATATTCGTCCAC
Reverse primer:
(SEQ ID NO: 8)
/5Phos/AGGCCTGCTGAAAATGACTG
Blocking oligonucleotide:
(SEQ ID NO: 9)
5′-C+T+G+G+T+G+G+C+G+T+A-3′,
where “+” indicates locked nucleic acid.

In some embodiments, a method described herein comprises detecting the presence or absence of, and/or distinguishing between one or more allelic variants comprising a mutation in a KRAS gene comprising employing at least one of the following capture nucleic acids:

KRAS G12D Probe:
(SEQ ID NO: 10)
/5AmMC6/AAAAAAAAAAGTTGGAG+CTG+ATG+GCGTAG,
KRAS G12V probe:
(SEQ ID NO: 11)
/5AmMC6/AAAAAAAAAAGTTGGAG+CTG+TT+GGC+GTAG
KRAS G12C probe:
(SEQ ID NO: 12)
/5AmMC6/AAAAAAAAAAGTTGGAG+CT+TGT+GGC+GTAG
KRAS G12A probe:
(SEQ ID NO: 13)
/5AmMC6/AAAAAAAAAAGTTGGAGCTG+CTGGCGTAG
KRAS G12S probe:
(SEQ ID NO: 14)
/5AmMC6/AAAAAAAAAAGTTGGAG+CT+AGT+GGC+GTAG

In some embodiments, a method described herein comprises detecting the presence or absence of, and/or distinguishing between one or more allelic variants comprising a mutation in a KRAS gene comprising employing the following capture nucleic acids:

KRAS G12D Probe:
(SEQ ID NO: 10)
/5AmMC6/AAAAAAAAAAGTTGGAG+CTG+ATG+GCGTAG,
KRAS G12V probe:
(SEQ ID NO: 11)
/5AmMC6/AAAAAAAAAAGTTGGAG+CTG+TT+GGC+GTAG
KRAS G12C probe:
(SEQ ID NO: 12)
/5AmMC6/AAAAAAAAAAGTTGGAG+CT+TGT+GGC+GTAG
KRAS G12A probe:
(SEQ ID NO: 13)
/5AmMC6/AAAAAAAAAAGTTGGAGCTG+CTGGCGTAG
KRAS G12S probe:
(SEQ ID NO: 14)
/5AmMC6/AAAAAAAAAAGTTGGAG+CT+AGT+GGC+GTAG

In some embodiments, a method described herein comprises detecting the presence or absence of, and/or distinguishing between one or more allelic variants comprising a mutation in a KRAS gene comprising employing the following capture nucleic acids the following primers and blocking oligonucleotides and capture nucleic acids in the method:

Forward primer:
(SEQ ID NO: 7)
/5Biosg/ATTGTTGGATCATATTCGTCCAC
Reverse primer:
(SEQ ID NO: 8)
/5Phos/AGGCCTGCTGAAAATGACTG
Blocking oligonucleotide:
(SEQ ID NO: 9)
5′-C+T+G+G+T+G+G+C+G+T+A-3′,
where “+” indicates locked nucleic acid
Capture nucleic acids:
KRAS G12D Probe:
(SEQ ID NO: 10)
/5AmMC6/AAAAAAAAAAGTTGGAG+CTG+ATG+GCGTAG,
KRAS G12V probe:
(SEQ ID NO: 11)
/5AmMC6/AAAAAAAAAAGTTGGAG+CTG+TT+GGC+GTAG
KRAS G12C probe:
(SEQ ID NO: 12)
/5AmMC6/AAAAAAAAAAGTTGGAG+CT+TGT+GGC+GTAG
KRAS G12A probe:
(SEQ ID NO: 13)
/5AmMC6/AAAAAAAAAAGTTGGAGCTG+CTGGCGTAG
KRAS G12S probe:
(SEQ ID NO: 14)
/5AmMC6/AAAAAAAAAAGTTGGAG+CT+AGT+GGC+GTAG

In some embodiments, a method described herein comprises detecting and/or distinguishing between one or more homologues or orthologs present in different organisms. In some embodiments, a method described herein comprises detecting and/or distinguishing between one or more homologues or orthologs present in different organisms based on the detection of one or more such genetic variants in one or more samples. In some embodiments, such organisms comprise pathogenic organisms.

In some embodiments, a method described herein comprises providing or employing a plurality of primers or sets of primers, capture nucleic acids, and/or detectable labels is employed in order to distinguish between one or more organisms present in, or suspected of being present in, one or more samples. In some embodiments, such organisms comprise pathogenic organisms.

In some embodiments, a method described herein comprises providing or employing a plurality of primers or sets of primers, capture nucleic acids, and/or detectable labels is employed in order to distinguish between organisms that belong to or may otherwise be classified into groups, such as phylogenetic and/or taxonomic groups. In such embodiments, a plurality of primers or sets of primers, capture nucleic acids, and/or detectable labels is provided or employed in order to distinguish between organisms that belong to or may otherwise be classified into groups, such as phylogenetic and/or taxonomic groups. In some embodiments, a plurality of primers or sets of primers, capture nucleic acids, and/or detectable labels is provided or employed in order to distinguish between organisms that belong to the same or similar taxonomic groups, such as the same or a similar order, the same or a similar family, the same or a similar genus, the same or a similar subgenus, or the same or a similar species. In some embodiments, such organisms comprise pathogenic organisms.

In some embodiments, a method described herein comprises providing or employing a plurality of primers or sets of primers, capture nucleic acids, and/or detectable labels is employed in order to distinguish between organisms that may be classified into groups on the bases of one or more distinguishable features or traits that allows for distinguishing between at least one such organism from other organisms in a sample. In some embodiments, such organisms comprise pathogenic organisms.

In some embodiments, a method described herein comprises providing or employing a plurality of primers or sets of primers, capture nucleic acids, and/or detectable labels is employed in order to distinguish between bacterial organisms, fungal organisms, protozoan organisms, plant organisms, animal organisms in one or more samples. In some embodiments, such organisms comprise pathogenic organisms.

In some embodiments, a method described herein comprises providing or employing a plurality of primers or sets of primers, capture nucleic acids, and/or detectable labels is employed in order to distinguish between fungal organisms belonging to one or more of the following groups:
1. Candida auris, Candida albicans, Candida tropicalis, Candida parapsilosis, Candida glabrata, Candida krusei, Candida haemulonis
2. Aspergillus fumigatus, Aspergillus flavus, Aspergillus niger, Aspergillus terreus
3. Cryptococcus neoformans, Cryptococcus gattii
4. Coccidioides immitis, Coccidioides posadasii
5. Fusarium solani, Fusarium oxysporum, Fusarium verticillioidis, and Fusarium moniliforme
6. Pneumocystis jirovecii
7. Blastomyces dermatitidis
8. Histoplasma capsulatum
9. Rhizopus oryzae, Rhizopus microspores
10. Candida auris

In some embodiments, a method described herein comprises providing or employing a plurality of primers comprising at least one of the following primers is employed in order to distinguish between one or more organisms present in, or suspected of being present in, one or more samples:

Reverse Primer:
(SEQ ID NO: 17)
/5Phos/GGAGTGATTTGTCTGCTTAATTGC
Forward Primer:
(SEQ ID NO: 18)
5Biosg/GGCTTGAGCCGATAGTCCC;
or
Forward Primer:
(SEQ ID NO: 33
5Biosg/CATCGGCTTGAGCCGATAGTC
Forward Primer:
(SEQ ID NO: 19)
5Biosg/GCCTCAAACTTCCATCGACTTC
Reverse Primer:
(SEQ ID NO: 20)
/5Phos/CGATAACGAACGAGACCTTAACC
Reverse Primer:
(SEQ ID NO: 21)
/5Phos/CAGGTCTGTGATGCCCTTAG
Forward Primer:
(SEQ ID NO: 22)
5Biosg/CAATGCTCTATCCCCAGCAC

In some embodiments, a method described herein comprises providing or employing a plurality of primers selected from the group consisting of the following primers is employed in order to distinguish between one or more organisms present in, or suspected of being present in, one or more samples:

Reverse Primer:
(SEQ ID NO: 17)
/5Phos/GGAGTGATTTGTCTGCTTAATTGC
Forward Primer:
(SEQ ID NO: 18)
5Biosg/GGCTTGAGCCGATAGTCCC;
or
Forward Primer:
(SEQ ID NO: 33
5Biosg/CATCGGCTTGAGCCGATAGTC
Forward Primer:
(SEQ ID NO: 19)
5Biosg/GCCTCAAACTTCCATCGACTTC
Reverse Primer:
(SEQ ID NO: 20)
/5Phos/CGATAACGAACGAGACCTTAACC
Reverse Primer:
(SEQ ID NO: 21)
/5Phos/CAGGTCTGTGATGCCCTTAG
Forward Primer:
(SEQ ID NO: 22)
5Biosg/CAATGCTCTATCCCCAGCAC

In some embodiments, a method described herein comprises providing or employing a plurality of capture nucleic acids comprising at least one of the following capture nucleic acids is employed in order to distinguish between one or more organisms present in, or suspected of being present in, one or more samples:

(SEQ ID NO: 23)
/5AmMC6/AAAAAAAAAAGTGCTGCCAGCGCGCCTCTTG
(SEQ ID NO: 24)
/5AmMC6/AAAAAAAAAACCGACCC+ACGT+TTG+TGG
(SEQ ID NO: 25)
/5AmMC6/AAAAAAAAAACGAtCCCGCGT+CTG+CG
(SEQ ID NO: 26)
/5AmMC6ANANANANAACGAGACCT+CG+GCCCTTAA
(SEQ ID NO: 27)
/5AmMC6/AAAAAAAAAACACTGACG+GA+GCCAGC
(SEQ ID NO: 28)
/5AmMC6/AAAAAAAAAAGAGTCTTA+CC+GC+CTTGGC
(SEQ ID NO: 29)
/5AmMC6/AAAAAAAAAAGCCAGC+AA+GT+T+CATTTCC
(SEQ ID NO: 30)
/5AmMC6/AAAAAAAAAAGTACT+TC+C+TT+GGCCGAAAG
(SEQ ID NO: 31)
/5AmMC6/AAAAAAAAAACACT+GA+TG+AA+G+TCAGCG
(SEQ ID NO: 32)
/5AmMC6/AAAAAAAAAAGTACATCA+CCTTGG+CCG

In some embodiments, a method described herein comprises providing or employing a plurality of capture nucleic acids selected from the group consisting of the following capture nucleic acids is employed in order to distinguish between one or more organisms present in, or suspected of being present in, one or more samples:

(SEQ ID NO: 23)
/5AmMC6/AAAAAAAAAAGTGCTGCCAGCGCGCCTCTTG
(SEQ ID NO: 24)
/5AmMC6/AAAAAAAAAACCGACCC+ACGT+TTG+TGG
(SEQ ID NO: 25)
/5AmMC6/AAAAAAAAAACGA+CCCGCGT+CTG+CG
(SEQ ID NO: 26)
/5AmMC6/AAAAAAAAAACGAGACCT+CG+GCCCTTAA
(SEQ ID NO: 27)
/5AmMC6/AAAAAAAAAACACTGACG+GA+GCCAGC
(SEQ ID NO: 28)
/5AmMC6/AAAAAAAAAAGAGTCTTA+CC+GC+CTTGGC
(SEQ ID NO: 29)
/5AmMC6/AAAAAAAAAAGCCAGC+AA+GT+T+CATTTCC
(SEQ ID NO: 30)
/5AmMC6/AAAAAAAAAAGTACT+TC+C+TT+GGCCGAAAG
(SEQ ID NO: 31)
/5AmMC6/AAAAAAAAAACACT+GA+TG+AA+G+TCAGCG
(SEQ ID NO: 32)
/5AmMC6/AAAAAAAAAAGTACATCA+CCTTGG+CCG

In some embodiments, a method described herein comprises providing or employing primers or primer sets that are configured to amplify target nucleic acids that are shared by such one or more organisms but have one or more nucleotide differences between such one or more organisms, and thus may serve as target nucleic acids which may be used to distinguish between such one or more organisms in accordance with the methods and devices disclosed herein and throughout. In some embodiments, such organisms comprise pathogenic organisms.

In some embodiments, a method described herein comprises providing or employing one or more target nucleic acids that are configured to capture one or more amplified target nucleic acids (also referred interchangeable throughout as amplicons and/or distinguishable amplicons) that are shared by such one or more organisms but have one or more nucleotide differences between such one or more organisms, and thus may serve as target nucleic acids which may be used to distinguish between such one or more organisms in accordance with the methods and devices disclosed herein and throughout.

In some embodiments, a method described herein comprises employing plurality of primers or sets of primers, capture nucleic acids, and/or detectable labels is employed in order to distinguish between pathogenic organisms that are present, or are suspected of being present, in a sample.

In some embodiments, the sample is obtained from a biological source (living or dead). In some embodiments, the sample is obtained from a subject, such as a mammalian subject, such as a human subject. In some embodiments the sample is obtained from a patient. In some embodiments, the sample is obtained from an environmental source. In some embodiments, the sample is obtained from an environmental source, such as a water source, such as an ocean, lake, river, stream, swamp, lagoon, marsh, tidal pool, swimming pool, tributary, wastewater facility, wastewater reservoir, water reservoir, potable water reservoir, water treatment facility, and/or the like. In some embodiments, the sample is obtained from the environment, such as soil, dirt, sludges, slimes, scums, composts and the like.

In some embodiments, a method described herein further comprises amplifying a detection signal measured by performing a detecting step, comprising, prior to performing the detecting step: contacting the captured amplicons with a second detectable label comprising magnetic particles and the second member of the binding pair, wherein the first detectable label associates with the second detectable label through an interaction between the first and second binding pairs of the first and second detectable labels; thereby amplifying the detection signal that is measured upon performing the detecting step.

In some embodiments, a first genetic variant and a second genetic variant each comprise an allelic variant. In some embodiments, an at least one genetic variant comprises an allelic variant. In some embodiments, at least two genetic variants comprise allelic variants. In some embodiments each genetic variant that is detected distinguishes the presence of one organism from another organism in the sample.

In some aspects, embodiments herein relate to methods of detecting the presence of a first genetic variant in a target nucleic acid, in a query sample comprising: (a) providing a sensor and a capture nucleic acid, wherein the capture nucleic acid comprises a sequence complementary to the first genetic variant of the target sequence, wherein the capture nucleic acid is capable of being attached to a functionalized surface of a giant magnetoresistance (GMR) sensor; (b) contacting the target nucleic acid with (i) a first primer, (ii) a second primer comprising a first member of a binding pair, (iii) a polymerase and (iv) a blocking oligonucleotide, wherein the blocking oligonucleotide comprises a sequence complementary to a second genetic variant of the target nucleic acid, and the first and second primers are configured for amplification of the target nucleic acid; (c) amplifying the target nucleic acid thereby providing amplicons of the target nucleic acid; (d) contacting the amplicons with the capture nucleic acid, thereby providing captured amplicons comprising the first member of the binding pair; (e) contacting the captured amplicons with a detectable label comprising a magnetic particle and a second member of the binding pair; (f) passing the captured amplicons contacted with detectable label of step (e) over the GMR sensor; and (g) detecting a presence, absence, amount, or change thereof, of the detectable label. In some embodiments, the method comprises attaching the capture nucleic acid on the surface of the sensor prior to performing one or more of steps (b) through (e). In some embodiments, the method comprises detecting the presence or absence of a cancer in a subject according to the presence or absence of the first genetic variant in a target nucleic acid. In some embodiments, a method comprises administering a suitable treatment to a subject when a first genetic variant is detected. In some embodiments, the detecting step (f) comprises a dynamic detection process. In some embodiments, the dynamic detection process comprises increasing the stringency of hybridization conditions at the surface of the sensor. In some embodiments, the second member of the binding pair comprises streptavidin. In some embodiments, the first binding pair comprises biotin. In some embodiments, the first genetic variant, second genetic variant, and any other genetic variant each comprises an allelic variant. In some embodiments, the first genetic variant, second genetic variant, and any other genetic variant each comprises a variant that distinguishes the presence of one organism from another organism in the sample.

In some aspects, embodiments herein relate to methods of amplifying a signal at the surface of a sensor for detecting the presence, absence, amount, or change thereof, of a first genetic variant in a target nucleic acid in a query sample comprising: (a) providing a sensor and a capture nucleic acid, wherein the capture nucleic acid comprises a sequence complementary to the first genetic variant of the target sequence, wherein the capture nucleic acid is capable of being attached to a functionalized surface of a giant magnetoresistance (GMR) sensor; (b) contacting the target nucleic acid with (i) a first primer, (ii) a second primer comprising a first member of a binding pair, (iii) a polymerase and (iv) a blocking oligonucleotide, wherein the blocking oligonucleotide comprises a sequence complementary to a second genetic variant of the target nucleic acid, and the first and second primers are configured for amplification of the target nucleic acid; (c) amplifying the target nucleic acid thereby providing amplicons of the target nucleic acid; (d) contacting the amplicons with the capture nucleic acid, thereby providing captured amplicons comprising the first member of the binding pair; (e) contacting the captured amplicons with a first plurality of magnetic particles comprising a second member of the binding pair; (f) passing the captured amplicons contacted plurality of magnetic particles of step (e) over the GMR sensor; (g) passing a second plurality of magnetic particles comprising the second member of the binding pair over the sensor after step, wherein the first member of the binding pair of the second plurality of magnetic particles binds to the second member of the binding pair of the first plurality of particles; and (h) detecting a presence, absence, amount, or change thereof, of the first and second plurality of magnetic particles, thereby amplifying the signal at the surface of the sensor. In some embodiments, the method comprises attaching the capture nucleic acid on the surface of the sensor prior to performing one or more of steps (b) through (e). In some embodiments, such methods further comprise passing one or more subsequent pluralities of magnetic particles comprising the first member of the binding pair, and one or more subsequent pluralities of magnetic nanoparticles comprising the second member of the binding pair, over the GMR sensor. In some embodiments, the binding pair comprises streptavidin and biotin. In some embodiments, the first member of the binding pair comprises streptavidin. In some embodiments, the second member of the binding pair comprises biotin. In some embodiments, the method comprises detecting the presence or absence of a cancer in a subject according to the presence or absence of the first genetic variant in a target nucleic acid. In some embodiments, a method comprises administering a suitable treatment to a subject when a first genetic variant is detected. In some embodiments, the detecting step (f) comprises a dynamic detection process. In some embodiments, the dynamic detection process comprises increasing the stringency of hybridization conditions at the surface of the sensor. In some embodiments, the first genetic variant, second genetic variant, and any other genetic variant each comprises an allelic variant. In some embodiments, the first genetic variant, second genetic variant, and any other genetic variant each comprises a variant that distinguishes the presence of one organism from another organism in the sample.

In some aspects, embodiments herein relate to methods amplifying a detection signal for detecting the presence of a first genetic variant in a target nucleic acid in a query sample comprising: (a) providing a sensor comprising a first biomolecule disposed on a functionalized surface of a giant magnetoresistance (GMR) sensor, the first biomolecule comprising a conditional binding site for a second biomolecule comprising a binding site for a magnetic particle; (b) passing the query sample over the sensor; (c) passing the second biomolecule over the sensor; (d) passing a plurality of magnetic particles comprising a first member of a binding pair over the sensor after passing the query sample over the sensor, then passing a plurality of magnetic particles comprising a second member of the binding pair over the sensor; and (e) detecting the presence of the analyte in the query sample by measuring magnetoresistance change of the GMR sensor based on determining magnetoresistance before and after passing magnetic particles over the sensor, wherein determining magnetoresistance change of the GMR sensor comprises using at least one reference resistor to perform phase-sensitive solution of magnetoresistance change of the GMR sensor; thereby amplifying the detection signal.

In some aspects, embodiments herein relate to methods of amplifying a detection signal for detecting the presence of an analyte in a query sample comprising: (a) providing a sensor comprising a first biomolecule disposed on a functionalized surface of a giant magnetoresistance (GMR) sensor, the biomolecule comprising a binding site for a magnetic particle when the analyte is present;(b) passing the query sample over the sensor; (c) passing a plurality of magnetic particles comprising a first member of a binding pair over the sensor after passing the query sample over the sensor, then passing a plurality of magnetic particles comprising a second member of the binding pair over the sensor; and (e) detecting the presence of the analyte in the query sample by measuring magnetoresistance change of the GMR sensor based on determining magnetoresistance before and after passing magnetic particles over the sensor, wherein determining magnetoresistance change of the GMR sensor comprises using at least one reference resistor to perform phase-sensitive solution of magnetoresistance change of the GMR sensor; thereby amplifying the detection signal.

In some aspects the first member of the binding pair comprises streptavidin and the second member of the binding pair comprises biotin.

In some aspects the first member first member of the binding pair comprises biotin and the second member of the binding pair comprises streptavidin.

In some aspects the first member the magnetoresistance change of the GMR sensor comprises an amplified magnetoresistance change.

In some embodiments are provided methods of detecting the presence of one or more genetic variants in one or more query samples in a multiplex detection scheme, the method comprising: providing spacially disposed giant magnetoresistance (GMR) sensors, wherein at least two of the GMR sensors comprise at least two different capture nucleic acids, wherein each capture nucleic acid comprises a sequence complimentary to that can genetic variants disposed on a functionalized surface of the at least two (GMR) sensors, (a) passing the one or more query samples over the sensors, thereby allowing cleavage and removal of the cleavable portion with the associated receptor from at least two biomolecules if at least one of the one or more analytes is present; (b) passing magnetic particles over the sensors after passing the one or more query samples over the sensors; and (c) detecting the presence of at least one of the one or more analytes in the one or more query samples by measuring magnetoresistance change of at least one of the at least two GMR sensors based on determining magnetoresistance before and after passing magnetic particles over the sensors.

In embodiments are provided methods of detecting the presence of one or more analytes in one or more query samples comprising in a multiplex detection scheme: (a) providing at least two spacially disposed giant magnetoresistance (GMR) sensors, wherein at least two of the GMR sensors comprise at least two different genetic variants disposed on a functionalized surface of the at least two (GMR) sensors, each different biomolecule comprising: an antigenic portion that binds an antibody at an antigen binding site, the antibody further comprising a portion separate from the antigen binding site configured to bind a magnetic nanoparticle; (b) passing a mixture of the one or more query samples and the antibody over the sensors, wherein the antigen binding site of the antibody binds the one or more analytes if present in the one or more query samples, thereby preventing binding of the antibody to the antigenic portion of at least one of the at least two biomolecules; (c) passing magnetic particles over the sensors after passing the mixture over the sensors; and (d) detecting the presence of the analyte in the query sample by measuring a magnetoresistance change of at least one of the at least two GMR sensors based on determining magnetoresistance before and after passing magnetic particles over the sensors.

In embodiments are provided methods methods of detecting the presence of one or more analytes in one or more query samples comprising in a multiplex detection scheme comprising: (a) providing at least two spacially disposed giant magnetoresistance (GMR) sensors, wherein at least two of the GMR sensors comprise at least two different biomolecules disposed on a functionalized surface of a GMR sensor, each different biomolecule comprising: a binding region configured to bind one of at least two different detection proteins, the at least two different detection proteins also being capable of binding one of the one or more analytes; wherein when one of the at least two different detection proteins binds one of the analytes, it prevents binding of said one of the at least two detection proteins to the binding region of the biomolecule; (b) passing the at least two different detection proteins over the sensors; (c) passing the one or more query samples over the sensors; (d) passing at least one reporter protein over the sensors after passing the one or more query samples over the sensor, the at least one reporter protein capable of binding the at least two detections proteins and the at least one reporter protein configured to bind to magnetic particles; (e) passing magnetic particles over the sensors after passing the at least one reporter protein over the sensors; and (f) detecting the presence of one or more analyte by measuring magnetoresistance change of the at least two GMR sensors based on determining magnetoresistance before and after passing magnetic particles over the sensors.

In some embodiments, the least two spacially disposed GMR sensors are disposed in the channel of a GMR sensor chip, wherein the GMR sensor chip comprises at least one channel. In some embodiments, the least two spacially disposed GMR sensors are disposed in the channel of a GMR sensor chip, wherein the GMR sensor chip comprises a plurality of channels. In some embodiments the least two spacially disposed GMR sensors are each disposed different channels of a GMR sensor chip, wherein the GMR sensor chip comprises a plurality of channels.

In some embodiments, passing magnetic particles over the sensors comprises passing a plurality of magnetic particles comprising a first member of a binding pair over the sensor after passing the reporter protein over the sensor, and subsequently passing a plurality of magnetic particles comprising a second member of the binding pair over the sensor, and wherein magnetoresistance change of the GMR sensors comprises an amplified magnetoresistance change of the GMR sensors.

In some embodiments, some or all of the steps of a method described herein are conducted in a microfluidic device comprising the sensor, and a plurality of valves, chambers, microfluidic channels and ports that are configured to direct flow of the sample, the magnetic particles and optionally, one or more wash buffers, over the surface of the sensor.

In some embodiments, the methods disclosed herein are performed in a microfluidic device.

In some embodiments, a method described herein is performed in a microfluidic device described herein, wherein the device comprises one or more microfluidic channels that are operably and/or fluidically connected to an amplification chamber and a magnetic sensor.

In some embodiments, provided herein is a microfluidics device comprising one or more microfluidic channels that are operably and/or fluidically connected to an amplification chamber and a sensor.

In some embodiments, provided herein is a microfluidics device for carrying out a method described herein, wherein the microfluidics device comprises: (a) a microfluidic channel; (b) a first chamber comprising a membrane; (c) an amplification chamber; (d) 3 or more miniature solenoid valves; and (d) a sensor comprising a surface comprising a plurality of capture nucleic acids; wherein the microfluidic channel is operably connected and/or fluidically connected with the first chamber, the amplification chamber, the 3 or more valves and the sensor. The microfluidic device of claim 47, wherein the sensor is a magnetic sensor.

In some embodiments, a micorofluidics device provided herein comprises a sample port, and one or more wash chambers comprising a wash buffer, wherein the sample port and one or more wash chambers are operably and/or fluidically connected to the microfluidic channel and the first chamber. The mircrofluidic device of any one of claims 47 to 49, further comprising a second chamber comprising magnetic particles, wherein the second chamber is operably and/or fluidically connected to the microfluidic channel and to the magnetic sensor. In some embodiments, the magnetic sensor is housed in a third chamber. In some embodiments, the mircrofluidic device further comprises one or more waste collection chambers, wherein the one or more waste collection chambers are operably and/or fluidically connected to the microfluidic channel. In some embodiments, the mircrofluidic device further comprises a first heat source operably connected to the amplification chamber. In some embodiments, the mircrofluidic device further comprises a cooling source operably connected to the amplification chamber. In some embodiments, the mircrofluidic device further comprises a second heat source operably connected to the magnetic magnetoresistance sensor and/or to the third chamber. In some embodiments, the microfluidics channel is operably connected to one or more diaphragm pumps or vacuum pumps. In some embodiments, the mircrofluidic device comprises one or more electrical contact pads that are operably connected to the three or more valves. In some embodiments, the microfluidic device comprises a memory chip. In some embodiments, the microfluidic device has a length of 3 to 10 cm long, a width of 1 to 5 cm, and a thickness of 0.1 to 0.5 cm. In some embodiments, the microfluidic device comprises or consists of a self contained cartridge or card comprising lyophilized amplification reagents, and lyophilized magnetic beads.

In some embodiments, the microfluidic device is configured for integration with a controller and or computer. For example, in some embodiments, the microfluidic device is in the form of a removable card or cartridge.

In other aspects, embodiments relate to the systems configured to carry out the foregoing methods.

Other aspects, features, and advantages of the present disclosure will become apparent from the following detailed description, the accompanying drawings, and the appended claims.

### BRIEF DESCRIPTION OF THE DRAWINGS

Various embodiments of the present disclosure will be described herein below with reference to the Figures wherein:

FIG. 1 is a perspective view of an exemplary cartridge reader unit used in a system in accordance with an embodiment of the present disclosure.

FIG. 2A is a perspective view of an exemplary cartridge assembly used in the system, in accordance with an embodiment of the present disclosure.

FIG. 2B is an exploded view of the cartridge assembly of FIG. 2A, in accordance with an embodiment herein.

FIG. 2C is a schematic drawing of the cartridge assembly of FIG. 2A, in accordance with an embodiment herein.

FIG. 2D shows a cross section of the cartridge assembly of FIG. 2A, illustrating a connection interface between a sample processing card and a sensing and communication substrate thereof.

FIG. 3 is a schematic diagram of the system in accordance with an embodiment of the present disclosure.

FIG. 4 shows steps of a method for performing analyte detection in a sample when using features of the herein disclosed system of FIG. 3, in accordance with an embodiment.

FIG. 5A shows a serpentine channel comprising a plurality of GMR sensors, in accordance with an embodiment.

FIG. 5B shows an arrangement of a plurality of channels on a substrate for GMR sensing, in accordance with an embodiment.

FIG. 6A shows a cross-section of a linear length of channel with GMR sensors disposed therein, in accordance with an embodiment.

FIG. 6B shows a cross-section of a linear length of channel having circular channel expansions where GMR sensors reside, in accordance with an embodiment.

FIG. 6C shows a cross-section of a linear length of channel having square channel expansions where GMR sensors reside, in accordance with an embodiment.

FIG. 6D shows a cross-section of a linear length of channel having triangular channel expansions where GMR sensors reside, in accordance with an embodiment.

FIG. 6E shows a section of a serpentine channel with GMR sensors disposed therein, in accordance with an embodiment.

FIG. 6F shows a section of a serpentine channel with GMR sensors disposed circular channel expansions, in accordance with an embodiment.

FIG. 6G shows a section of a channel having a bifurcation and with GMR sensors disposed therein, in accordance with an embodiment.

FIG. 7 shows a cross-section of a linear length of channel having circular channel expansions where differing GMR sensors reside, in accordance with an embodiment.

FIG. 8A shows a GMR sensor chip having a plurality of channels with GMR sensors incorporated at circular expansions and the connectivity of the GMR sensors to contact pads via wiring, in accordance with an embodiment.

FIG. 8B shows an expansion of the area around the GMR sensors in the circular channel expansions showing the wiring network, in accordance with an embodiment.

FIG. 8C shows the structure of a switch, in accordance with an embodiment.

FIG. 9 shows a cross-section representation of a circular channel expansion and the GMR residing therein along with attachment to a contact pad via a wire, in accordance with an embodiment.

FIG. 10A shows a cross-section representation of a channel with no expansion and the GMR residing therein along with a biosurface layer disposed over the GMR sensor, in accordance with an embodiment.

FIG. 10B shows the basic structure and operating principle of GMR sensors, in accordance with an embodiment.

FIG. 11A shows a structure state diagram of a subtractive GMR sensing process, in accordance with an embodiment.

FIG. 11B shows a process flow diagram for the GMR sensing process of FIG. 11A.

FIG. 12A shows a structure state diagram of an additive GMR sensing process, in accordance with an embodiment.

FIG. 12B shows a process flow diagram for the GMR sensing process of FIG. 12A.

FIG. 13A shows another structure state diagram of an additive GMR sensing process, in accordance with an embodiment.

FIG. 13B shows a process flow diagram for the GMR sensing process of FIG. 13A.

FIG. 13C shows an alternative flow diagram for the GMR sensing process of FIG. 13A.

FIG. 14A shows a structure state diagram of an additive GMR sensing process in which an analyte modifies a molecule bound to a biosurface, in accordance with an embodiment.

FIG. 14B shows a process flow diagram for the GMR sensing process of FIG. 14A.

FIG. 15A shows an alternative structure state diagram of an additive GMR sensing process in which an analyte modifies a molecule bound to a biosurface, in accordance with an embodiment.

FIG. 15B shows a process flow diagram for the GMR sensing process of FIG. 15A.

FIG. 16A shows a structure state diagram of an additive GMR sensing process employing an exemplary “sandwich” antibody process.

FIG. 16B shows a process flow diagram for the GMR sensing process of FIG. 16A.

FIG. 17A shows a plot of data generated with a GMR sensor for detecting D-dimer cardiac biomarker: solid line is a positive control; dashed line is a sample run; line indicated with “+” is a negative control.

FIG. 17B shows a calibration curve for D-dimer using a GMR sensor for detecting D-dimer cardiac biomarker.

FIG. 17C shows a graph of data generated with a GMR sensor for detecting troponin cardiac biomarker.

FIG. 18 shows an example of amplification of a GMR signal, in accordance with an embodiment of the present teaching.

FIG. 19 shows a schematic overview of an exemplary method described herein. In some embodiments, a sample is introduced into a first chamber (104) comprising a membrane, which is configured to reversibly binds to nucleic acids. Any cells that are present can be lysed in the sample chamber 100 by introduction of a cell lysis solution (lysis buffer). Nucleic acids that are bound to the membrane may be washed, eluted and transported via a microfluidic channel to an amplification chamber 208. Various reagents for amplification can be introduced into the amplification chamber 208, such as primers, dNTPs, blocking oligonucleotides, polymerase and salts. Such reagents may be present in the amplification chamber prior to introduction of the target nucleic acids. The amplification chamber may be subjected to thermal cycling such that PCR may be conducted. Heating and cooling components may be present on the microfluidics device. Amplicons may be transported though a microfluidic channel to a second chamber 204 comprising an exonuclease and/or directed to a sensor 300 (e.g., a GMR sensor) comprising a capture nucleic acid. Optionally, an exonuclease may be introduced into the amplification chamber (e.g., after amplicons are generated), into an adjacent chamber, or into a chamber housing the sensor. Particles (e.g., magnetic beads) that are housed in a storage chamber 230 can be introduced into a chamber comprising the sensor. In some embodiments, a sensor and or an amplification chamber are operably connected to one or more heating and/or cooling sources. Magnetoresistance and/or changes in magnetoresistance can be detected on the sensor 300.

FIG. 20 shows an example of an amplification process using a first primer comprising a 5′-phosphate and a second primer comprising a biotin moiety. The 5′phosphate of the first primer allows degradation of the amplicon strands that comprise the 5′phosphate by a 5′-3′ exonuclease. The presence of a blocking oligonucleotide comprising locked nucleotides is configured to hybridize to nucleic acids that do not have a variant/mutation of interest. The blocking oligonucleotide is configured to anneal to a non-mutated template thereby forming a double stranded duplex having a high melting temperature. The high melting temperature of the duplex formed by the blocking oligonucleotide substantially prevents amplification of templates that do not have the mutation of interest.

FIG. 21 illustrates that a 5′-3′ exonuclease specifically degrades amplicons having a 5′-phosphate group. Amplicons that comprise a biotin group and/or which lack a free 5′-hydroxyl are not digested by the exonuclease.

FIG. 22 illustrates capture of biotinylated amplicons on the surface of a senor bearing a capture nucleic acid. In some embodiments, the capture nucleic acid comprises locked nucleotides. The capture nucleic acid is configured to anneal specifically to a region of the biotinylated amplicons having the genetic variation (e.g., mutation) of interest. The presence of the locked nucleotides in the capture nucleic acid improves specificity of the hybridization. Magnetic beads/particles (MNP) comprising streptavidin (S) bind to the biotin (B) on the captured amplicons.

FIG. 23 shows a process where heat is applied to the magnetic sensor surface while magnetoresistance at the surface of the sensor is detected and/or measured. Amplicons that non-specifically hybridize to the capture nucleic acids will be released from the sensor surface at a lower temperature than amplicons having an exact complementary sequence to the capture nucleic acids. Changes in magnetoresistance detected at higher temperatures are more indicative of the presence of a specific mutation of interest in a target nucleic acid.

FIG. 24 shows an exemplary workflow diagram for a detection method described herein that takes place on a microfluidic device comprising one or more microfluidic channels 105. Valves 120 (e.g., V1-V14), in some embodiments, are miniature piloting solenoid valves (e.g., Lee valves) that can be controlled off-card, each independently. The microfluid channels can be operably connected to one or more diaphragm pumps or syringes pumps that, in cooperation with valves 120, can control and direct sample flow through the device. Each valve and pump can operate independently or together.

FIG. 25 shows a front view of an exemplary microfluidic device contained on a cartridge 600 designed to integrate with a computer/controller and one or more pumps. Cartridge 600 can implement the workflow described herein and as illustrated in FIG. 24.

FIG. 26 shows a rear view of cartridge 600.

FIG. 27 shows how different capture nucleic acids can be used to detect L858R DNA (i.e., a c.2573T>G mutation) of the EGFR gene in a nucleic acid sample, where each capture nucleic acid can be differentiated according to its melting temp. The data shown in FIG. 27 is an overlay of 6 different experimental runs using a dynamic detection process. The capture nucleic acid of SEQ ID NO:6 (/5AmMC6/AAAAAAAAAAAAAAAGTTTGG+CC+CGCCC+AAA) is shown in purple. Four other capture nucleic acids were also tested, each configured to hybridize to the same target sequence comprising the T>G mutation, and each having a different melting temperature. The red line showing the highest signal is generated from a biotinylated probe attached directly to the sensor surface. The yellow line represents a negative control where the capture nucleic acid does not bind to DNA in the sample. Magnetoresistance at the sensor surface (signal) is measured over a period of about 1400 seconds (x axis). The “signal” shown on the y-axis is unitless in and of itself. The signal (y-axis) is calculated by dividing the magnetoresistance at the sensor at any one time by a base magnetoresistance, resulting in the signal. Magnetic beads comprising streptavidin are added to the GMR sensor at about 600 seconds. The beads bind to biotinylated amplicons that are captured at the sensor surface or to the control probe (red). The binding of the magnetic beads results in a sharp increase in signal at the sensor surface at about 620 seconds. Next, the temperature at the sensor is slowly increased from 45° C. (at about 620 seconds) to 85° C. (at about 1400 seconds) by increasing the temperature of the buffer flowing over the surface of the sensor. The captured amplicons start to denature and leave the sensor surface as the temperature increases. Capture nucleic acids with higher melting temperatures denature at higher temperatures and can be distinguished from the other capture nucleic acids. In this experiment, the melting temperature (shown at the top of the figure) of each capture nucleic acid was determined empirically at a point where the peak signal (y-axis, at about 625 seconds) decreases by 50%.

FIG. 28 shows dynamic detection of biotinylated amplicons comprising only wild type target sequence of EGFR (i.e., no mutated amplicons are present). The experiment utilizes the capture nucleic acid of SEQ ID NO:6 (/5AmMC6/AAAAAAAAAAAAAAAGTTTGG+CC+CGCCC+AAA, which contains a mismatch nucleotide with the wild-type target sequence. The experiment demonstrates that binding of the wild-type amplicons to the capture nucleic acid (false positive) can be differentiated from binding of the mutated target sequence (true positive, data not shown) by increasing stringency of the hybridization conditions at the sensor surface. In this experiment, streptavidin labeled magnetic beads were contacted with the captured biotinylated amplicons at about 175 seconds resulting in a strong signal peak at the sensor surface at about 180 seconds. The sodium ion concentration of the buffer flowing across the sensor surface was quickly changed from about 50 mM sodium to 10 mM sodium, reaching 10 mM at about 210 seconds. Because the system is dynamic and the sensor is always flushed with constantly moving buffer, denatured amplicons that are attached to magnetic beads get washed away. Accordingly, this initial increase in stringency resulted in about a 75% drop in signal (e.g., see signal at 300 sec.). The temperature of the buffer flowing across the sensor surface was then slowly increased from about 45° C. at about 300 seconds to about 85° C. at about 1000 seconds. This increase in temperature denatured the remaining hybridized amplicons demonstrating a melting temperature for the false positive hybridization at 52° C. (10 mM Na), calculated at about 550 seconds, which is a melting temperature about 15° C. lower than the melting temperature of the capture nucleic acid with a mutated target sequence (true signal, not shown). The change in sodium ion concentration alone did not affect the melting temperature of the capture nucleic acid to the mutated target DNA (i.e., 67° C., data not shown). This experiment demonstrates that a false positive signal (i.e., a mismatched amplicon) can be differentiated from a true positive signal (i.e., a perfect matched amplicon) by increasing the stringency of hybridization conditions at the sensor surface (e.g., by dynamically lowering sodium ion concentration and increasing temperature).

FIG. 29A & 29B show the results of a dynamic detection process using a microfluidic device described herein comprising a GMR sensor. This experiment was conducted on a sample obtained from a healthy subject having no cancer (FIG. 29A) where the sample cfDNA only contained wild-type target sequence of the EGFR gene. The sequence at the top of FIG. 29A shows an alignment of the mismatch between the capture nucleic acid and amplicons derived from the subjects wild-type DNA, which was detected with as a low signal (blue line, blue filled circles) as stringency conditions were increased at the sensor surface over time. Accordingly, the data of FIG. 29A (blue line) shows an absence of the c.2573T>G mutation in the EGFR gene of the subject and therefore an absence of cancer in this subject. The experiment was repeated on a subject known to have cancer as a results of a c.2573T>G mutation in the EGFR gene (FIG. 29B). The sequence alignment at the top of FIG. 29B shows a perfect between the capture nucleic acid and amplicons derived from the subjects mutated DNA, which was detected with as a high signal (blue line, blue filled circles) as stringency conditions were increased at the sensor surface over time. Accordingly, FIG. 29B shows positive detection of a c.2573T>G mutation in the EGFR gene of the patient and therefore confirms the presence of cancer in this subject. The patient of FIG. 29B is heterozygous for the c.2573T>G mutation and this cfDNA sample comprised mismatched wild-type target DNA and mutated target sequence at a ratio of 99:1. Accordingly, this assay is sensitive enough to detect small amounts of mutated target sequence among copious amounts of wild-type sequence.

FIG. 30 show the results multiple replicates of a dynamic detection process using a microfluidic device described herein comprising a GMR sensor from samples obtained from patients showing detection of a KRAS G12D mutation with G12D mutation as low as 0.1%. This data also demonstrates that the same blocker and primers can be used to detect multiple different mutations within a single region.

FIG. 31 show the results of a dynamic detection process using a microfluidic device described herein comprising a GMR sensor from cell-free DNA samples showing detection of different mutations within a gene.

FIG. 32 shows the results of a dynamic detection process using a microfluidic device described herein comprising GMR sensors from fungal DNA samples showing detection and identification of fungi from patient samples.

### DETAILED DESCRIPTION

Presented herein are microfluidic devices comprising a sensor that can be used to detect a genetic variation in sample (e.g., a plasma sample) comprising nucleic acids. Such microfluidic devices comprise, for example sensors, such as magnetic sensors. In some embodiments, such microfluidic devices comprise Giant MagnetoResistance (GMR) sensors.

The devices and methods described herein can accurately detect a single point mutation in cell-free DNA found in as little as 1 ml of plasma, in some embodiments. The devices presented herein enable rapid, non-invasive, and highly sensitive detection of cancers and other disorders that are caused by, or that are associated with, genetic variations.

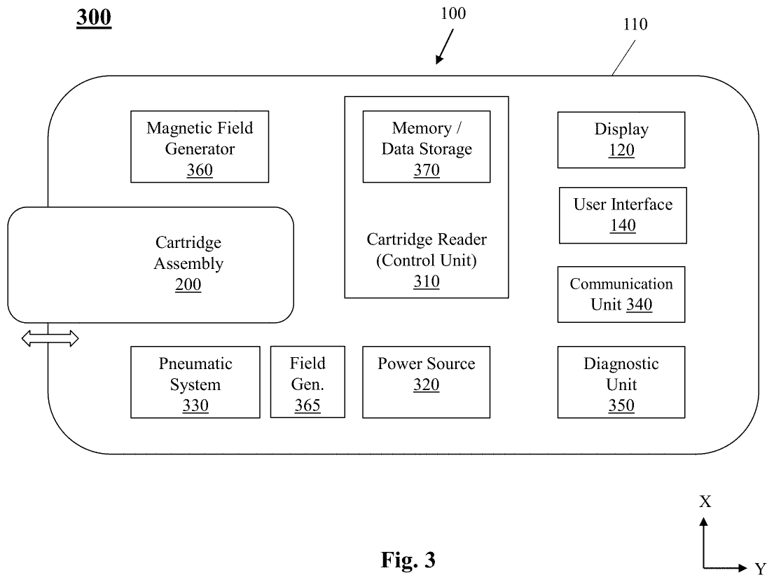

As evident by the drawings and below description, this disclosure relates to a sample handling system (or “system” as noted throughout this disclosure) which may be used for detecting presence of an analyte (or analytes) in a sample. In an embodiment, this system, depicted as system 300 in FIG. 3, may include (1) a sample handling system or “cartridge assembly” that includes sample preparation microfluidic channel(s) and at least one sensing device (or sensor) for sensing biomarkers in a test sample, and (2) a data processing and display device or “cartridge reader unit” that includes a processor or controller for processing any sensed data of the sensing device of the cartridge assembly and a display for displaying a detection event. Together these two components make up the system. In an embodiment, these components may include variable features including, without limitation, one or more reagent cartridges, a cartridge for waste, and a flow control system which may be, for example, a pneumatic flow controller.

Generally, the process for preparing a sample in the cartridge assembly, in order for detection of analytes, biomarkers, etc. to happen by the assembly and output via the cartridge reader unit, is as-follows: A raw patient sample is loaded onto a card, optionally filtered via a filter membrane, after which a negative pressure generated by off-card pneumatics filters the sample into a separated test sample (e.g., plasma). This separated test sample is quantitated on-card through channel geometry. The sample is prepared on card by interaction with mixing materials (e.g., reagent(s) (which may be dry or wet), buffer and/or wash buffer, beads and/or beads solution, etc.) from a mixing material source (e.g., blister pack, storage chamber, cartridge, well, etc.) prior to flow over the sensor/sensing device. The sample preparation channels may be designed so that any number of channels may be stacked vertically in a card, allowing multiple patient samples to be used. The same goes for sensing microfluidic devices, which may also be stacked vertically. A sample preparation card, which is part of the cartridge assembly, includes one or more structures providing functionalities selected from filtering, heating, cooling, mixing, diluting, adding reagent, chromatographic separation and combinations thereof; and a means for moving a sample throughout the sample preparation card. Further description regarding these features is provided later below.

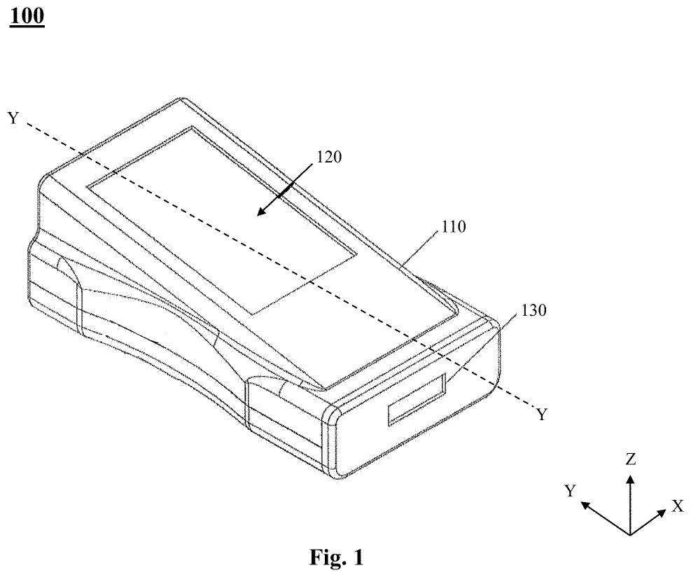

FIG. 1 shows an example of a cartridge reader unit 100, used in system 300 (see FIG. 3) in accordance with an embodiment. The cartridge reader unit 100 may be configured to be compact and/or small enough to be a hand-held, mobile instrument, for example. The cartridge reader unit 100 includes a body or housing 110 that has a display 120 and a cartridge receiver 130 for receiving a cartridge assembly. The housing 110 may have an ergonomic design to allow greater comfort if the reader unit 100 is held in an operator's hand. The shape and design of the housing 110 is not intended to be limited, however.

The cartridge reader unit 100 may include an interface 140 and a display 120 for prompting a user to input and/or connect the cartridge assembly 200 with the unit and/or sample, for example. In accordance with an embodiment, in combination with the disclosed cartridge assembly 200, the system 300 may process, detect, analyze, and generate a report of the results, e.g., regarding multiple detected biomarkers in a test sample, e.g., five cardiac biomarkers, using sensor (GMR) technology, and further display the biomarker results, as part of one process.

The display 120 may be configured to display information to an operator or a user, for example. The display 120 may be provided in the form of an integrated display screen or touch screen (e.g., with haptics or tactile feedback), e.g., an LCD screen or LED screen or any other flat panel display, provided on the housing 110, and (optionally) provides an input surface that may be designed for acting as end user interface (UI) 140 that an operator may use to input commands and/or settings to the unit 100, e.g., via touching a finger to the display 120 itself. The size of the display 120 may vary. More specifically, in one embodiment, the display 120 may be configured to display a control panel with keys, buttons, menus, and/or keyboard functions thereon for inputting commands and/or settings for the system 300 as part of the end user interface. In an embodiment, the control panel includes function keys, start and stop buttons, return or enter buttons, and settings buttons. Additionally, and/or alternatively, although not shown in FIG. 1, the cartridge reader 100 may include, in an embodiment, any number of physical input devices, including, but not limited to, buttons and a keyboard. In another embodiment, the cartridge reader 100 may be configured to receive input via another device, e.g., via a direct or wired connection (e.g., using a plug and cord to connect to a computer (PC or CPU) or a processor) or via wireless connection. In yet another embodiment, display 120 may be to an integrated screen, or may be to an external display system, or may be to both. Via the display control unit 120, the test results (e.g., from a cartridge reader 310, described with reference to FIG. 3, for example) may be displayed on the integrated or external display. In still yet another embodiment, the user interface 140 may be provided separate from the display 120. For example, if a touch screen UI is not used for display 120, other input devices may be utilized as user interface 140 (e.g., remote, keyboard, mouse, buttons, joystick, etc.) and may be associated with the cartridge reader 100 and/or system 300. Accordingly, it should be understood that the devices and/or methods used for input into the cartridge reader 100 are not intended to be limiting. All functions of the cartridge reader 100 and/or system 300 may, in one embodiment, be managed via the display 120 and/or input device(s), including, but not limited to: starting a method of processing (e.g., via a start button), selecting and/or altering settings for an assay and/or cartridge assembly 200, selecting and/or settings related to pneumatics, confirming any prompts for input, viewing steps in a method of processing a test sample, and/or viewing (e.g., via display 120 and/or user interface 140) test results and values calculated by the GMR sensor and control unit/cartridge reader. The display 120 may visually show information related to analyte detection in a sample. The display 120 may be configured to display generated test results from the control unit/cartridge reader. In an embodiment, real-time feedback regarding test results that have been determined/processed by the cartridge reader unit/controller (by receiving measurements from the sensing device, the measurements being determined as a result of the detected analytes or biomarkers), may be displayed on the display 120.

Optionally, a speaker (not shown) may also be provided as part of the cartridge reader unit 100 for providing an audio output. Any number of sounds may be output, including, but not limited to speech and/or alarms. The cartridge reader unit 100 may also or alternatively optionally include any number of connectors, e.g., a LAN connector and USB connector, and/or other input/output devices associated therewith. The LAN connector and/or USB connector may be used to connect input devices and/or output devices to the cartridge reader unit 100, including removable storage or a drive or another system.

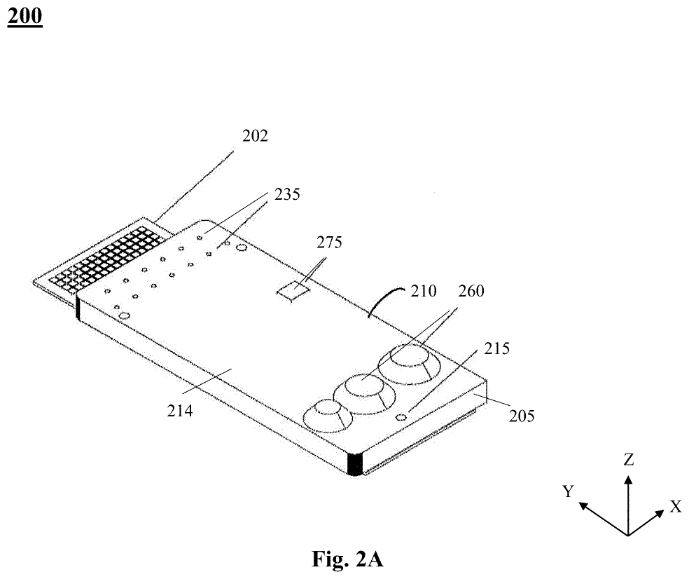

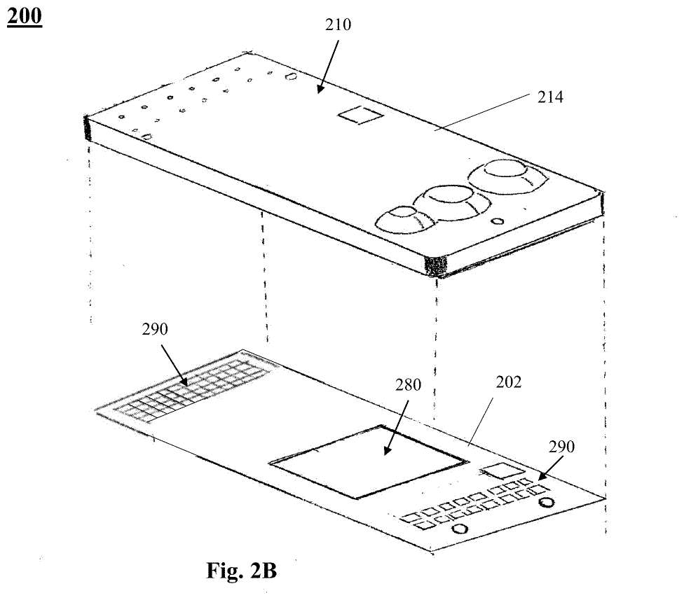

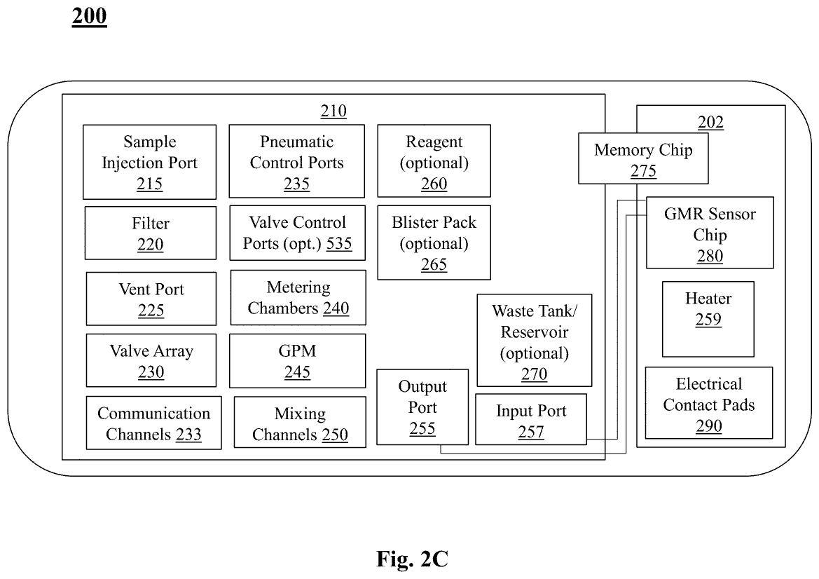

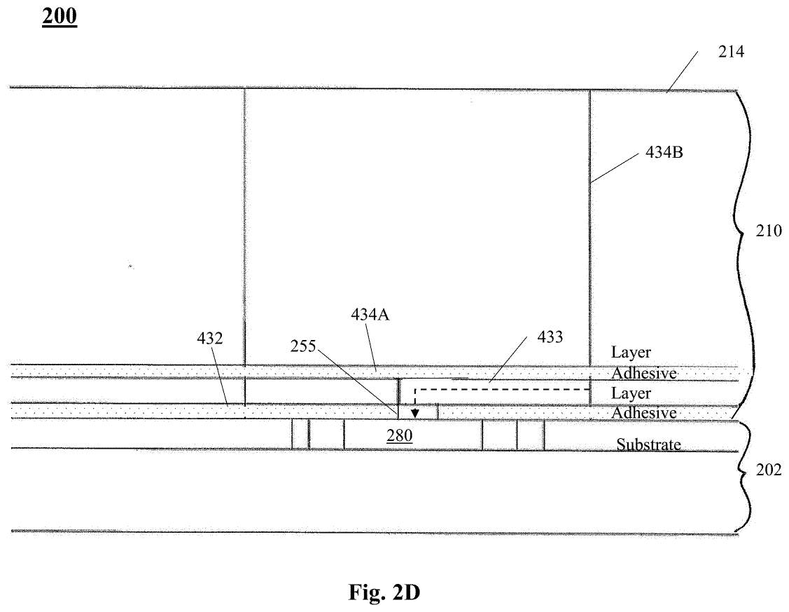

In accordance with an embodiment, the cartridge receiver 130 may be an opening (such as shown in FIG. 1) within the housing 110 in which a cartridge assembly (e.g., cartridge assembly 200 of FIG. 2) may be inserted. In another embodiment, the cartridge receiver 130 may include a tray that is configured to receive a cartridge assembly therein. Such a tray may move relative to the housing 110, e.g., out of and into an opening therein, and to thereby receive the cartridge assembly 200 and move the cartridge assembly into (and out of) the housing 110. In one embodiment, the tray may be a spring-loaded tray that is configured to releasably lock with respect to the housing 110. Additional details associated with the cartridge reader unit 100 are described later with respect to FIG. 3.

As previously noted, cartridge assembly 200 may be designed for insertion into the cartridge reader unit 100, such that a sample (e.g., blood, urine) may be prepared, processed, and analyzed. FIGS. 2A-2C illustrate an exemplary embodiment of a cartridge assembly 200 in accordance with embodiments herein. Some general features associated with the disclosed cartridge assembly 200 are described with reference to these figures. However, as described in greater detail later, several different types of cartridge cards and thus cartridge assemblies may be utilized with the cartridge reader unit 100 and thus provided as part of system 300. In embodiments, the sampling handling system or cartridge assembly 200 may take the form of disposable assemblies for conducting individual tests. That is, as will be further understood by the description herein, depending on a type of sample and/or analytes being tested, a different cartridge card configuration(s) and/or cartridge assembly(ies) may be utilized. FIG. 2A shows a top, angled view of a cartridge assembly 200, in accordance with an embodiment herein. The cartridge assembly 200 includes a sample processing card 210 and a sensing and communication substrate 202 (see also FIG. 2B). Generally, the sample processing card 210 is configured to receive the sample (e.g., via a sample port such as injection port, also described below) and, once inserted into the cartridge reader unit 100, process the sample and direct flow of the sample to produce a prepared sample. Card 210 may also store waste from a sample and/or fluid used for preparing the test sample in an internal waste chamber(s) (not shown in FIG. 2A, but further described below). Memory chip 275 may be read and/or written to and is used to store information relative to the cartridge application, sensor calibration, and sample processing required, for example. In an embodiment, the memory chip 275 is configured to store a pneumatic system protocol that includes steps and settings for selectively applying pressure to the card 210 of the cartridge assembly 200, and thus implementing a method for preparation of sample for delivery to a sensor (e.g., GMR sensor chip 280). The memory chip may be used to mistake-proof each cartridge assembly 200 inserted into the unit 100, as it includes the automation recipe for each assay. The memory chip 275 also contain traceability to the manufacturing of each card 210 and/or cartridge assembly 200. The sensing and communication substrate 202 may be configured to establish and maintain communication with the cartridge reader unit 100, as well as receive, process, and sense features of the prepared sample. The substrate 202 establishes communication with a controller in the cartridge reader unit 100 such that analyte(s) may be detected in a prepared sample. The sample processing card 210 and the sensing and communication substrate 202 (see, e.g., FIG. 2B) are assembled or combined together to form the cartridge assembly 200. In an embodiment, adhesive material (see, e.g., FIG. 2D) may optionally be used to adhere the card 210 and substrate 202 to one another. In an embodiment, the substrate 202 may be a laminated layer applied to the sample processing card 210. In one embodiment, the substrate 202 may be designed as a flexible circuit that is laminated to sample processing card 210. In another embodiment, the sample processing card 210 may be fabricated from a ceramic material, with the circuit, sensor (sensor chip 280) and fluid channels integrated thereon. Alternatively, the card 210 and substrate 202 may be mechanically aligned and connected together. In one embodiment, a portion of the substrate 202 may extend from an edge or an end of the card 210, such as shown in FIG. 2A. In another embodiment, such as shown in FIG. 2B, the substrate 202 may be aligned and/or sized such that it has similar or smaller edges than the card 210.

FIG. 2C schematically illustrates features of the cartridge assembly 200, in accordance with an embodiment. As shown, some of the features may be provided on the sample processing card 210, while other may be associated with the substrate 202. Generally, to receive a test sample (e.g., blood, urine) (within a body of the card), the cartridge assembly 200 includes a sample injection port 215, which may be provided on a top of the card 210. Also optionally provided as part of the card 210 are filter 220 (also referred to herein as a filtration membrane), vent port 225, valve array 230 (or valve array zone 230), and pneumatic control ports 235. Communication channels 233 are provided within the card 210 to fluidly connect such features of the card 210. Pneumatic control ports 235 are part of a pneumatic interface on the cartridge assembly 200 for selectively applying pressurized fluid (air) to the communication channels 233 of the card, for directing flow of fluids (air, liquids, test sample, etc.) therein and/or valve array 230. Optionally, the card 210 may include distinct valve control ports 535 connected to designated communication channels 233 for controlling the valves in the valve array 230. The card 210 may also have one or more metering chambers 240, gas permeable membranes 245, and mixing channels 250 that are fluidly connected via communication channels 233. Metering chamber(s) are designed to receive at least the test sample (either directly or filtered) therein via communication channels 233. Generally, a sample may be injected into the cartridge assembly 200 through port 215 and processed by means of filtering with filter (e.g., filter 220), metering in metering chamber(s) 240, mixing in mixing channel(s) 250, heating and/or cooling (optional), and directing and changing the flow rate via communication channels 233, pneumatic control ports 235, and valve array 230. For example, flow of the fluid may be controlled using internal micro fluidic channels (also generally referred to as communication channels 233 throughout this disclosure) and valves via a connection of a pneumatic system (e.g., system 330 in the cartridge reader unit 100, as shown in FIG. 3) and a pneumatic interface e.g., on the card 210 that has pneumatic control ports 235 or a similar connection section. Optional heating of the test sample and/or mixing materials/fluids within the card 210 may be implemented, in accordance with an embodiment, via a heater 259 which may be in the form of a wire trace provided on a top side of a PCB/substrate 202 with a thermistor. Optional cooling of the test sample and/or mixing materials/fluids within the card 210 may be implemented, in accordance with an embodiment, via a TEC module integrated in the cartridge assembly 200 (e.g., on the substrate 202), or, in another embodiment, via a module integrated inside of the cartridge reader unit 100. For example, if the cooling module is provided in the unit 100, it may be pressed against the cartridge assembly 200 should cooling be required. Processing may also optionally include introduction of reagents via optional reagent sections 260 (and/or blister packs) on the card 210 and/or via reagent cartridges in the housing 110 the cartridge reader unit 100. Reagents may be released or mixed as required by the process for that sample and the cartridge assembly 200 being analyzed. Further, optional blister packs 265 may be provided on the card 210 to introduce materials such as reagents, eluants, wash buffers, magnetic nanoparticles, bead solution, or other buffers to the sample via communication channels 233 during processing. One or more internal waste chambers (also referred to herein as waste tanks for waste reservoirs) 270 may also be optionally provided on the card 210 to store waste from the sample and reagents. An output port 255—also referred to as a sensor delivery port, or input port to the sensor—is provided to output a prepared sample from the card 210 to a GMR sensor chip 280, as discussed below, for detecting analytes in the test sample. The output port 255 may be fluidly connected to a metering chamber for delivering the test sample and one or more mixing materials to the sensor. Accordingly, the sensor may be configured to receive the test sample and the one or more mixing materials via the at least one output port 255. In embodiments, an input port 257—also referred to as a waste delivery port, or output port from the sensor—is provided to output any fluid or sample from the GMR sensor chip 280 to a waste chamber 270. Waste chamber(s) 270 may be fluidly connected to other features of the card 210 (including, for example, metering chamber(s) 240, an input port 257, or both) via communication channels 233.

The cartridge assembly 200 has the ability to store, read, and/or write data on a memory chip 275, which may be associated with the card 210 or the substrate 202. As noted previously, the memory chip 275 may be used to store information related and/or relative to the cartridge application, sensor calibration, and required sample processing (within the sample processing card), as well as receive additional information based on a prepared and processed sample. The memory chip 275 may be positioned on the sample processing card 210 or on the substrate 200.

As previously noted, a magnetoresistance sensor may be utilized, in accordance with embodiments herein, to determine analytes (such as biomarkers) within a test sample using the herein disclosed system. While the description and Figures note use of a particular type of magnetoresistance sensor, i.e., a giant magnetoresistance (GMR) sensor, it should be understood that this disclosure is not limited to a GMR sensor platform. In accordance with some embodiments, the sensor may be an anisotropic magnetoresistive (AMR) sensor and/or magnetic tunnel junction (MTJ) sensors, for example. In embodiments, other types of magnetoresistive sensor technologies may be utilized. Nonetheless, for explanatory purposes only, the description and Figures reference use of a GMR sensor as a magnetoresistive sensor.

The substrate 202 of cartridge assembly 200 may be or include an electronic interface and/or a circuit interface such as a PCB (printed circuit board) that may have a giant magnetoresistance (GMR) sensor chip 280 and electrical contact pads 290 (or electrical contact portions) associated therewith. Other components may also be provided on the substrate 202. The GMR sensor chip 280 is attached at least to the substrate 202, in accordance with an embodiment. The GMR sensor chip 280 may be placed on and attached to the substrate 202 using adhesive, for example. In an embodiment, a liquid adhesive or a tape adhesive may be used between the GMR sensor 280 and the PCB substrate 202. Such a design may require a bond to the PCB at the bottom and a bond to the processing card at the top, for example. Alternatively, other approaches for attaching the GMR sensor chip 280 to the substrate 202 include, but are not limited to: friction fitting the GMR sensor to the PCB, and connecting a top of the GMR sensor chip 280 directly to the sample processing card 210 (e.g., in particular when the substrate 202 is provided in the form of a flexible circuit that is laminated (to the back) of sample processing card 210. The GMR sensor chip 280 may be designed to receive a prepared sample from the output port 255 of the sample processing card 210. Accordingly, placement of the GMR sensor chip 280 on the substrate may be changed or altered based on a position of the output port 255 on card 210 (thus, the illustration shown in FIG. 2B is not intended to be limiting)—or vice versa. In an embodiment, the GMR sensor chip 280 is positioned on a first side of the substrate 202 (e.g., a top side that faces an underside of the card 210, as shown in FIG. 2B), e.g., so as to receive the prepared sample from an output port that outputs on an underside of the card 210, and the contact pads 290 are positioned on an opposite, second side of the substrate (e.g., on a bottom side or underside of the substrate 202, such that the contact pads 290 are exposed on a bottom side of the cartridge assembly 200 when fully assembled for insertion into the cartridge reader unit 100). The GMR sensor chip 280 may include its own associated contact pads (e.g., metal strips or pins) that are electrically connected via electronic connections on the PCB/substrate 202 to the electrical contact pads 290 provided on the underside thereof. Accordingly, when the cartridge assembly 200 is inserted into the cartridge reader 100, the electrical contact pads 290 are configured to act as an electronic interface and establish an electrical connection and thus electrically connect with electronics (e.g., cartridge reader 310) in the cartridge reader unit 100. Thus, any sensors in the sensor chip 280 are connected to the electronics in the cartridge reader unit 100 through the electrical contact pads 290 and contact pads of the GMR sensor chip 280.

FIG. 2D shows a view of an exemplary cross section of a mating or connection interface of card 210 and substrate 202. More specifically, FIG. 2D illustrates an interface, in accordance with one embodiment, between an output port 255 on the card 210 and GMR sensor chip 280 of the substrate 202. For example, shown is a PCB substrate 202 positioned below and adjacent to a card 210 according to any of the herein disclosed embodiments. The substrate 202 may be attached to bottom surface of the card 210. The card 210 has a channel feature, labeled here as microfluidic channel 433 (which is one of many communication channels within the card 210), in at least one layer thereof, designed to direct a test sample that is processed within the card 210 to an output port 255 directed to GMR sensor 280. Optionally, adhesive material may be provided between layers of the card 210, e.g., adhesive 434A may be provided between a layer in the card that has reagent ports 434B and a layer with the channel 433. The substrate 202 includes a GMR sensor chip 280 that is positioned adjacent to the channel 433 and output port 255 of the card 210.

Magnetic field (from a magnetic coil 365 that is different than magnetic field generator 360, described below with reference to FIG. 3) may be used to excite the nanoparticle magnetic particles located near sensors.

GMR sensors have sensitivities that exceed those of anisotropic magnetoresistance (AMR) or Hall sensors. This characteristic enables detection of stray fields from magnetic materials at nanometer scales. For example, stray fields from magnetic nanoparticles that bound on sensor surface will alter the magnetization in the magnetic layers, and thus change the magnetoresistance of the GMR sensor. Accordingly, changes in the number of magnetic nanoparticles bound to the GMR sensor per unit area can be reflected in changes of the magnetoresistance value of the GMR sensor.

For such reasons, the sensor utilized in cartridge assembly 200, in accordance with the embodiments described herein, is a GMR sensor chip 280.

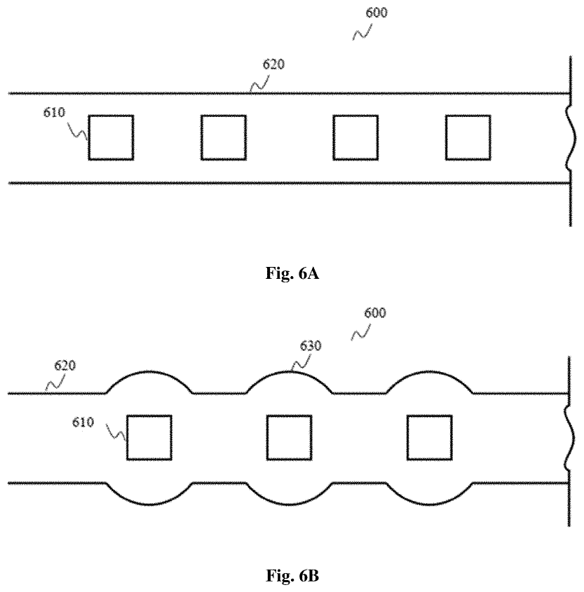

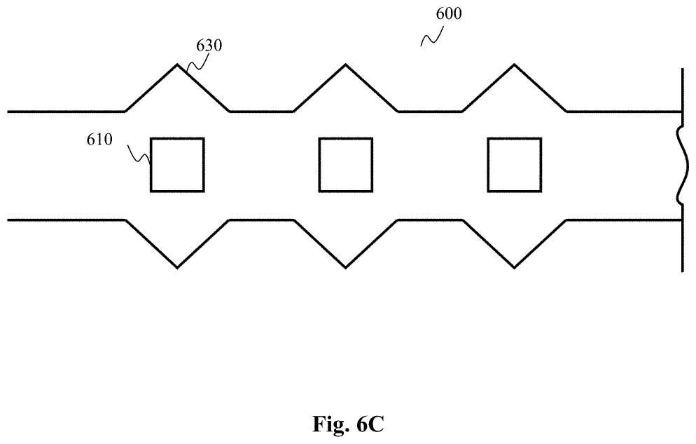

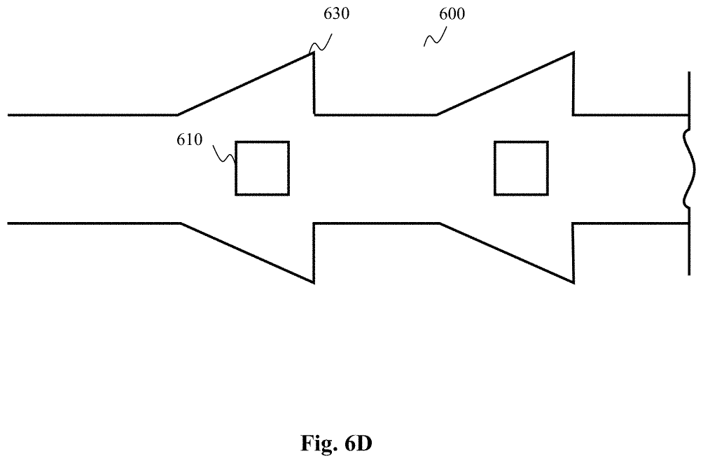

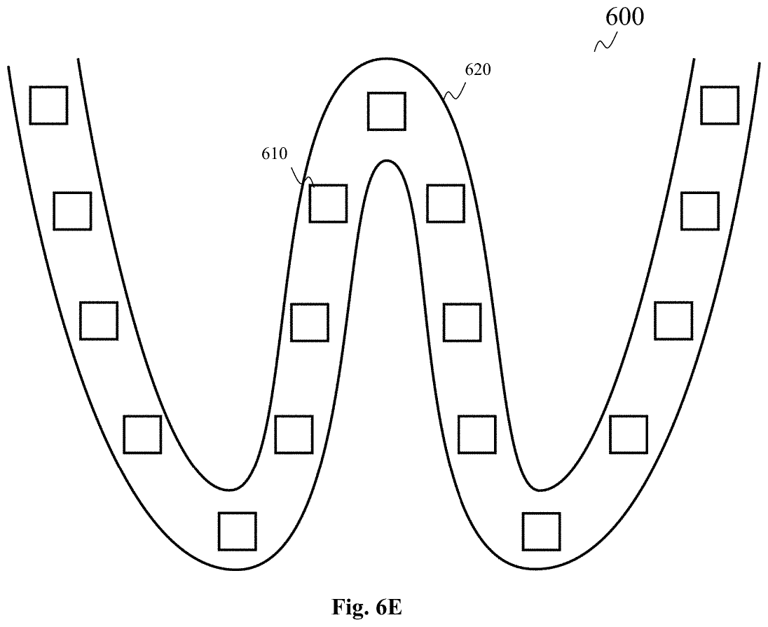

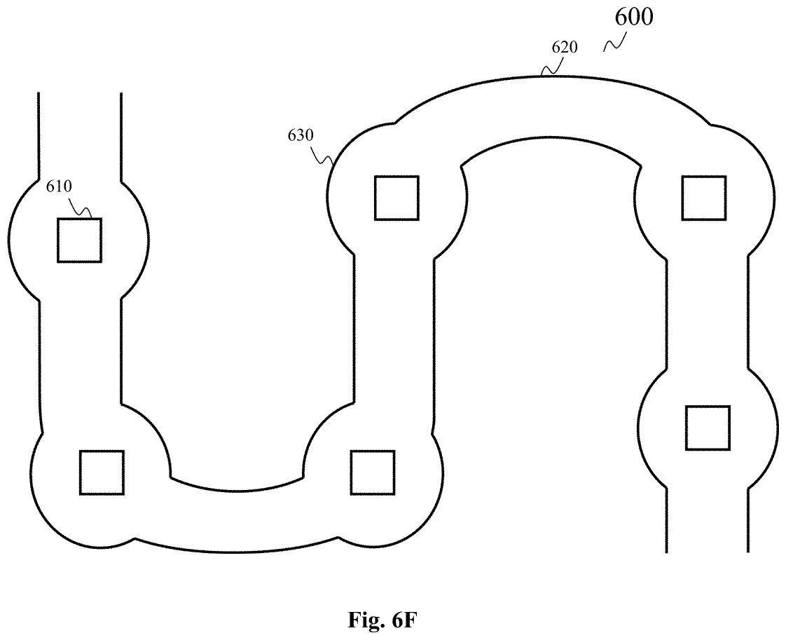

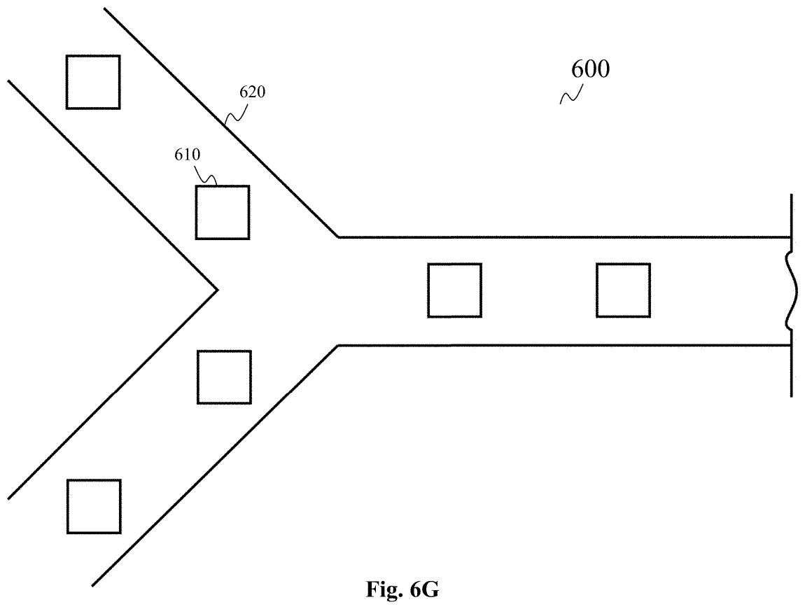

Referring now to FIG. 3, an overview of features provided in the system are shown. In particular, some additional features of the cartridge reader unit 100 are schematically shown to further describe how the cartridge reader unit 100 and cartridge assembly 200 are configured to work together to provide the system 300 for detecting analyte(s) in a sample. As depicted, the cartridge assembly 200 may be inserted into the housing 110 of the cartridge reader unit 100. Generally, the housing 110 of the cartridge reader unit 100 may further include or contain a processor or control unit 310, also called a “controller” and/or a “cartridge reader” 310 herein throughout, a power source 320, a pneumatic system 330, a communications unit 340, a (optional) diagnostic unit 350, a magnetic field generator 360, and a memory 370 (or data storage), along with its user interface 140 and/or display 120. Optionally, a reagent opener (e.g., puncture system 533 in FIG. 6), e.g., for opening a reagent source on an inserted cartridge assembly or for introducing reagent into the cartridge assembly (e.g., if the reagent is not contained in the assembly in a particular reagent section), may also be provided as part of the cartridge reader unit 100. Once a cartridge assembly 200 is inserted into the housing 110 of the cartridge reader unit 100, and the electrical and pneumatics system(s) are connected, and the cartridge memory chip 275 may be read from the cartridge assembly 200 (e.g., read by cartridge reader 310/control unit, or PCB assembly, in the unit 100) to determine the pneumatic system protocol that includes steps and settings for selectively applying pressure to the card 210 of the cartridge assembly 200, and thus implementing a method for preparation of sample for delivery to a sensor (e.g., GMR sensor chip 280), and thus the sample placed in the assembly 200 may be prepped, processed, and analyzed. The control unit or cartridge reader 310 may control inputs and outputs required for automation of the process for detecting the analyte(s) in a sample. The cartridge reader 310 may be a real-time controller that is configured to control, among other things, the giant magnetoresistance (GMR) sensor chip 280 and/or memory chip 275 associated with the cartridge assembly 200 and the pneumatic system 330 within the housing 110, as well as the controls from user interface, driving the magnetic field generator 360, and receiving and/or sending signals from/to sensor chip and/or memory associated with the cartridge assembly 200, for example. In an embodiment, the cartridge reader 310 is provided in the form of a PCB (printed circuit board) which may include additional chips, memory, devices, therein. The cartridge reader 310 may be configured to communicate with and/or control an internal memory unit, a system operation initializer, a signal preparing unit, a signal preparing unit, a signal processing unit, and/or data storage (none of which are shown in the Figures), for example. The cartridge reader 310 may also be configured to send and receive signals with respect to the communications unit 340 such that network connectivity and telemetry (e.g., with a cloud server) may be established, and non-volatile recipes may be implemented, for example. Generally, the communications unit 340 allows the cartridge reader unit 100 to transmit and receive data using wireless or wired technology. Power can be supplied to the cartridge reader unit 100 via power source 320 in the form of an internal battery or in the form of a connector that receives power via an external source that is connected thereto (e.g., via a cord and a plug). The pneumatic system 330 is used to process and prepare a sample (e.g., blood, urine) placed into the cartridge assembly 200 by means of moving and directing fluids inside and along the sample processing card 210 (e.g., via pneumatic connection 235, through its channels and connecting to direct elastomeric valves). The pneumatic system 330 may be a system and/or device for moving fluid, which could use, for example, plungers and/or pistons in contact with fluids (further described later below). The magnetic field generator 360 may be an external magnetic coil or other field generating device that is mounted in the unit 100 or integrated in some fashion with one or more of the chips (e.g., sensor chip 280) provided on the cartridge assembly 200 or provided on the circuit board of the cartridge reader unit 100. The magnetic field generator 360 is used to stimulate magnetic nanoparticles near the GMR sensor chip 280 while reading the signal. In accordance with embodiments, a second magnetic field generator 365, which may be a coil or other field generating device, may be provided as part of the cartridge reader unit 100 and in the housing 110. For example, in accordance with an embodiment, the second magnetic field generator 365 may be separate and distinct from magnetic field generator 360. This second magnetic field generator 365 may be configured to generate a non-uniform magnetic field such that it may apply such a magnetic field to a part (e.g., top, bottom, sides) of the sample processing card 210 during preparation and processing of a sample, e.g., when moving mixing material(s), such as a buffer and/or magnetic beads from a mixing material source, and test sample within the card. In an embodiment, the second magnetic field generator 365 is provided on an opposite end or side of the cartridge reader unit (e.g., located in a top of the housing 110 of unit 100), i.e. away from the magnetic field generator 360, which is used for GMR sensing. In one embodiment, the second magnetic field generator 365 is provided on an opposite end of the cartridge reader unit as compared to the magnetic field generator 360 (e.g., second magnetic field generator is located in a top of the housing 110 of unit 100 and magnetic field generator 360 is provided at a bottom end of the unit 100 (e.g., near cartridge receiver 130)). In an embodiment, the total magnetic field for sensing biomarkers/analytes includes an applied field from magnetic field generator 360 (either external or integrated with the sensor chip) along with any disturbance from magnetic nanoparticles near the GMR sensor chip 280. The reagent opener is optionally used to introduce reagents during the sample processing and reading of the GMR sensor chip 280 (e.g., if the reagent is not contained in the card in a particular reagent section). As described previously, the user interface 140/display 120 allows an operator to input information, control the process, provide system feedback, and display (via an output display screen, which may be a touch screen) the test results.

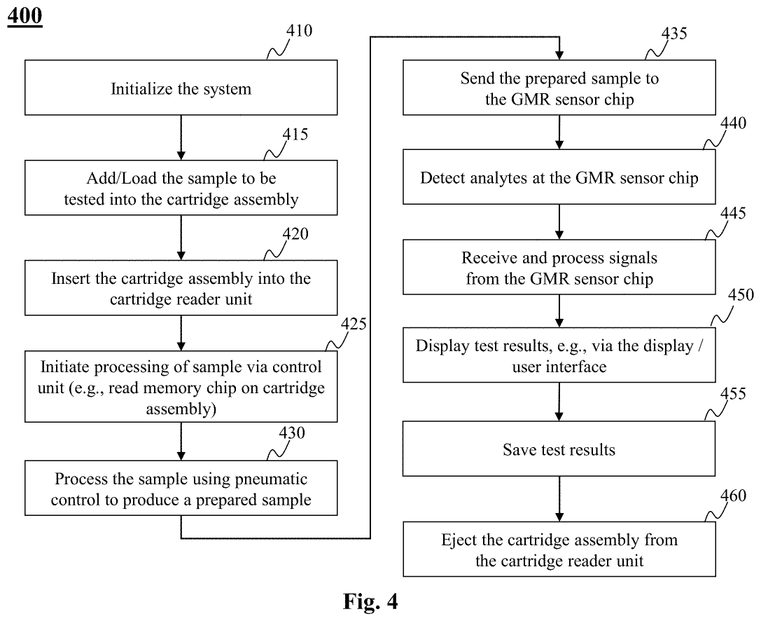

FIG. 4 shows general steps of a method 400 for performing analyte detection in a sample using the herein disclosed system 300. At step 410, the system is initialized. For example, initialization of the system may include: applying power to the system 300 (including cartridge reader unit 100), determining configuration information for the system, reading computations, determining that features (e.g., magnetic coil and carrier signals) are online and ready, etc. At step 415, a whole test sample is added or loaded into the cartridge assembly 200 (e.g., sample is injected into the injection port 215, as shown in FIG. 2C). The order of steps 410 and 415 may be changed; i.e., the addition of the whole test sample to the assembly 200 may be before or after the system is initialized. At step 420, the cartridge assembly 200 is inserted into the cartridge reader unit 100. Optionally, as part of method 400, user instruction may be input to the cartridge reader unit 100 and/or system 300 via the user interface/display 120. Then, at step 425, the processing of sample is initiated via the control unit 310. This initiation may include, for example, receiving input via an operator or user through the user interface/display 120 and/or a system that is connected to the reader unit 100. In another embodiment, processing may be initiated automatically via insertion of the cartridge assembly 200 into the cartridge reader unit 100 and detecting presence of the cartridge assembly 200 therein (e.g., via electrical connection between electrical contact pads 290 on the assembly 200 with the control unit 310, and automatically reading instructions from memory chip 275). The sample is processed at step 425 using pneumatic control instructions (e.g., obtained from memory chip 275) in order to produce a prepared sample. As generally described above (and further later below), the processing of the sample may be dependent upon the type of sample and/or the type of cartridge assembly 200 inserted into the reader unit 100. In some cases, the processing may include a number of steps, including mixing, introduction of buffers or reagents, etc., before the sample is prepared. Once the sample is prepared, the prepared sample is sent (e.g., through channels in the card 210 and to output port 255, via pneumatic control through pneumatic system 330 and control unit 310) to the GMR sensor chip 280. At step 440, analytes in the prepared sample are detected at the GMR sensor chip 280. Then, at step 445, signals from the GMR sensor chip 280 are received and processed, e.g., via cartridge reader 310 (control unit; which may include one or more processors, for example). Once the signals are processed, test results may be displayed at 450, e.g., via the display 120/user interface. At 455, test results are saved. For example, test results may be saved in a cloud server and/or memory chip 275 on board the cartridge assembly 200. In embodiments, any fluids or sample may be directed from the GMR sensor chip 280 through an input port 257 to waste chamber 270. Thereafter, once all tests are preformed and read by the sensing device/GMR sensor chip 280, the cartridge assembly 200 may be ejected from the cartridge reader unit 100. In accordance with an embodiment, this may be automatically performed, e.g., mechanics within the housing 110 of the cartridge reader unit 100 may push the assembly 200 out of the housing 110, or performed manually (by way of a button or force) by the operator, for example.

In an embodiment, the system 300 described herein may utilize a pneumatic control system as disclosed in International Patent Application No. PCT/US2019/043720, entitled “SYSTEM AND METHOD FOR GMR-BASED DETECTION OF BIOMARKERS” (Attorney Docket No. 026462-0504846) and filed on the same day, which is hereby incorporated by reference herein in its entirety.

In an embodiment, the system 300 described herein may utilize a cartridge assembly (e.g., for sample preparation and delivery to the sensor(s)) as disclosed in International Patent Application No. PCT/US2019/043753, entitled “SYSTEM AND METHOD FOR SAMPLE PREPARATION IN GMR-BASED DETECTION OF BIOMARKERS” (Attorney Docket No. 026462-0504847) and filed on the same day, which is hereby incorporated by reference herein in its entirety.

In an embodiment, the system 300 described herein may sense, detect, and/or measure analytes at the GMR sensor as disclosed in International Patent Application No. PCT/US2019/043766, entitled “SYSTEM AND METHOD FOR SENSING ANALYTES IN GMR-BASED DETECTION OF BIOMARKERS (Attorney Docket No. 026462-0504848) and filed on the same day, which is hereby incorporated by reference herein in its entirety.

In an embodiment, the system 300 described herein may process signals at the GMR sensor as disclosed in International Patent Application No. PCT/US2019/043791, entitled “SYSTEM AND METHOD FOR PROCESSING ANALYTE SIGNALS IN GMR-BASED DETECTION OF BIOMARKERS (Attorney Docket No. 026462-0504850) and filed on the same day, which is hereby incorporated by reference herein in its entirety. For example, as noted above, at step 445, signals from the GMR sensor chip 280 are received and processed, e.g., via cartridge reader 310. In an embodiment, cartridge reader 310 is configured to perform the function of processing results from the GMR sensor chip 280 using a sample preparation control part having a memory reader unit and a sample preparation control unit (e.g., used to receive signals indicating that a cartridge assembly 200 has been inserted into the cartridge reader unit 100, read information stored in the memory chip 275, and generate pneumatic control signals and send them to the pneumatic system 330) and a signal processing part adapted to control electrical elements, prepare and collect signals, and process, display, store, and/or relay detection results to external systems, including processing measurements signals to obtain test results of the analyte detection, as described in detail in the −0504850 application. Additional features relating to the cartridge reader 310 and signal processor of the unit 100 are provided in greater detail later in this disclosure.

It should be understood that, with regards to FIGS. 1 and 2A-2D, the features shown are representative schematics of a cartridge reader unit 100 and cartridge assembly 200 that are part of the herein disclosed system 300 for detecting the analyte(s) in a sample. Accordingly, the illustrations are explanatory only and not intended to be limiting.

Turning back to the features of the sample processing card 210 and cartridge assembly 200 as previously discussed with reference to FIG. 2C, the arrangement, placement, inclusion, and number of features provided on a sample processing card 210 in the cartridge assembly 200 may be based on the test sample being analyzed and/or the test being performed (e.g., detection of biomarkers, detection of metal, etc.), for example. Further, the card 210 may be arranged, in some embodiments, such that there are zones on the card, and/or such that features are provided in different layers (however, such layers do not need to be distinct layers with a body thereof; rather, layered relative to one another at a depth or height (in the Z-direction)). In accordance with embodiments herein, the sample processing card 210 may be formed using parts that are laser cut to form inlets, channels, valve areas, etc. and sandwiched and connected/sealed together. In other embodiments, one or more layers of the sample processing card may be laser cut, laminated, molded, etc. or formed from a combination of processes. The method of forming the sample processing card 210 is not intended to be limiting. For illustrative purposes herein, some of the Figures include a depiction of layers to show positioning of parts of the sample processing card 210 relative to one another (e.g., positioning within the card relative to other features that are placed above and/or below). Such illustrations are provided to show exemplary depths or placement of the features (channels, valves, etc.) within a body of the sample processing card 210, without being limiting.

Generally, each card 210 has body 214 extending in a longitudinal direction along a longitudinal centerline A-A (provided in the Y-direction) when viewed overhead or from the top. In an embodiment, each card 210 may have dimensions defined by a length extending in the longitudinal direction (i.e., along or relative to centerline A-A), a width extend laterally to the length (e.g., in the X-direction), and a height (or depth or thickness) in the Z-direction, or vertical direction. In a non-limiting embodiment, the body 214 of the card 210 may be of a substantially rectangular configuration. In one embodiment, the cartridge receiver 130 (and/or any related tray) in the cartridge reader unit 100 is sized to accommodate the dimensions of the sample processing card 210, such that the card 210 may be inserted into the housing of the unit 100.

The illustrated structural features shown in the Figures of this disclosure are not intended to be limiting. For example, the numbers of sets, valves, metering chambers, membranes, mixing channels, and/or ports are not intended to be limited with regards to those shown. In some embodiments, more channels may be provided. In some embodiments, less channels may be provided. The number of valves is also not intended to be limiting.

Although the cartridge assembly 200 and sample processing card 210 may be described herein as being used with a reagent and a patient or medical blood sample, it should be noted that the herein disclosed cartridge assembly 200 is not limited to use with blood or solely in medical practices. Other fluids that may be separable and combined with a reagent or reactionary material may be employed in the herein disclosed cartridge for assaying. Other samples may derive from saliva, urine, fecal samples, epithelial swabs, ocular fluids, biopsies (both solid and liquid) such as from the mouth, water samples, such as from municipal drinking water, tap water, sewage waste, ocean water, lake water, and the like.

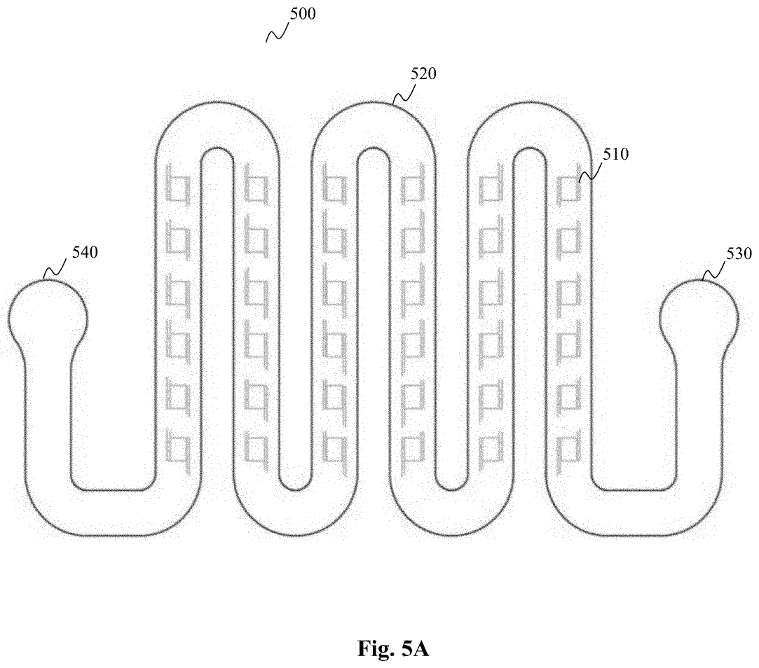

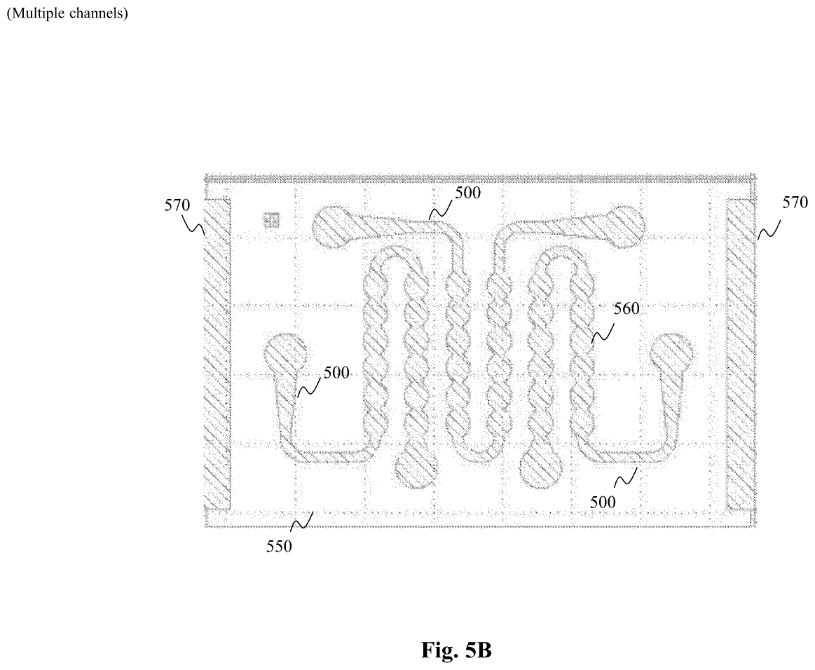

A sensing microfluidic device comprises one or more microfluidic channels and a plurality of sensor pads disposed within the one or more microfluidic channels. Referring now to FIG. 5A there is shown an exemplary channel 500 in accordance with some embodiments. Channel 500 is shown as serpentine in structure, but it need not be so limited in geometry. Channel 500 comprises a plurality of GMR sensors 510 disposed within the channel body 520. GMR sensors 510 may be all identically configured to detect a single analyte, the redundancy allowing for enhanced detection. GMR sensors 510 may also be all configured differently to detect a myriad of analytes or a combination of differently configured sensors with some redundancies. Channel 500 further comprises a channel entrance 530 where any samples, reagents, bead suspensions, or the like enter channel body 520. Flow through channel body 520 may be mediated under positive pressure at channel entrance 530 or under vacuum applied at channel exit 540.

FIG. 5B shows a plurality of channels 500 disposed within base 550. Each channel 500 features channel expansions 560 which is an expanded area surrounding each GMR sensor 510 (FIG. 5A; not shown in FIG. 5B for clarity). Without being bound by theory, it is postulated that channel expansions 560 provide a means for better mixing of materials as they pass over the GMR sensors. At the periphery of base 550 are disposed a pair of contact pads 570 which serve as an electrical conduit between the GMR sensors located in channel expansions 560 and the rest of the circuitry. GMR sensors 510 are electronically linked via wiring (not shown) to contact pads 570.

FIG. 6A shows a cross-section of a channel 600 comprising a plurality of GMR sensors 610 in a channel body 620 having a straight configuration. In such embodiments, the flow direction of materials can be from either direction. In other embodiments, as indicated in FIG. 6B, channel 600 can comprise a similar plurality of GMR sensors 610 incorporated within channel body 620 at channel expansions 630 that are shaped roughly circular or oval. In still further embodiments, as indicated in FIG. 6C, channel 600 can have GMR sensors 610 disposed in channel expansions 630 that are roughly square or rectangular. Although not shown such square or rectangular channel expansions can also be disposed so that the sides, rather than the points of the square or rectangle are part of channel expansion 630 rather than the vertices. Other configures of channel expansions 1030 are possible, including that shown in FIG. 6D where channel 600 has GMR sensors 610 disposed in triangular (or trapezoidal)-shaped. Channel expansions 630 can have any geometry and can be selected for desired flow and mixing properties, as well as residence times over GMR sensors 610.

As indicated in FIG. 6D, channel 600 may have a channel body 620 that is serpentine in shape, with GMR sensors 610 disposed along the length of the serpentine path. In some embodiments, such serpentine structures may allow for more sensors to packed into a small area compared to a linear channel 600. As shown FIG. 6F, channel 600 can incorporate both a body 620 that is serpentine in structure as well as having channel expansions 630 wherein GMR sensors 610 reside. Further optional structural features of channel 1000 are shown in FIG. 6G which shows channel 600 with GMR sensors disposed therein and which has a channel body 620 that incorporates a bifurcation. In some such embodiments, the flow direction can be modulated in either direction, depending on the exact application. For example, when flowing to the left in the drawing, materials can be split into two different pathways. This may represent, for example, the use of different GMR sensors 610 along the two bifurcation arms. The width of channel body 620 can vary before and after the bifurcation and can be selected for specific flow characteristics.

In some embodiments, referring as non-limiting examples to FIGS. 6A, 6B, 6C, 6D, 6F, and 6G, multiplex detection schemes are provided, for example, for performing multiplex assays for detecting more than one analyte in the same query sample or in difference query samples, may be achieved by spatially disposing different GMR sensors 610 within channel 620, wherein each different GMR sensor 610 is configured with differential tagging and/or coating such that each differentially tagged and/or coated GMR sensor 610 interacts with different molecules, such as different capture nucleic acids, different probes, different primers, different captured amplicons, different distinguishable captured amplicons, and/or the like described herein and throughout, thereby allowing for the detection of different analytes in the same sample, or different analytes in different samples, to be detected. In some embodiments, each differentially tagged and/or coated GMR sensor 610 interacts with different capture nucleic acids. In some embodiments, each differentially tagged and/or coated GMR sensor 610 interacts with different captured amplicons. In some embodiments, each differentially tagged and/or coated GMR sensor 610 interacts with different distinguishable captured amplicons.

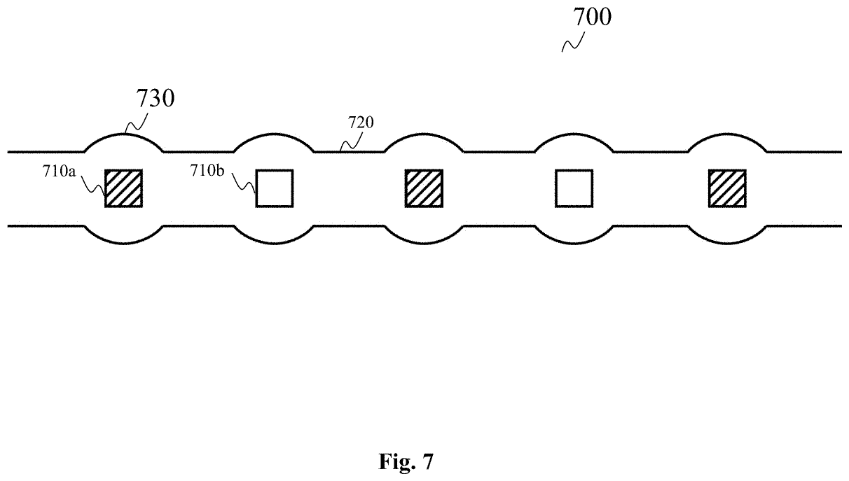

Referring now to FIG. 7, there is shown channel 700 which incorporates within channel body 720, channel expansions 730 in which different GMR sensors 710a and 710b are disposed. Although FIG. 7 shows different GMR sensors 710a and 710b alternating, it need not follow this pattern. For example, all of one type of GMR sensors 710a may be clustered together adjacent to each other and likewise all of the other type of GMR sensors 710b may be clustered together. In some embodiments, such alternating GMR sensors 710a and 710b Referring back to FIG. 6G, different sensors may also appear along the separated lines of a bifurcation.

In some embodiments, referring as a non-limiting example to FIG. 7, multiplex detection schemes are provided, for example, for performing multiplex assays for detecting more than one analyte in the same query sample or in difference query samples, may be achieved by spatially disposing different GMR sensors 710a and 710b within channel 720, wherein each different GMR sensor 710a and 710b is configured with differential tagging and/or coating such that each differentially tagged and/or coated GMR sensor 710a and 710b interacts with different a molecule, such as different capture nucleic acids, different probes, different primers, different captured amplicons, different distinguishable captured amplicons, and/or the like described herein and throughout, thereby allowing for the detection of different analytes in the same sample, or different analytes in different samples, to be detected. In some embodiments, each differentially tagged and/or coated GMR sensor 710a and 710b interacts with different capture nucleic acids. In some embodiments, each differentially tagged and/or coated GMR sensor 710a and 710b interacts with different captured amplicons. In some embodiments, each differentially tagged and/or coated GMR sensor 710a and 710b interacts with different distinguishable captured amplicons.

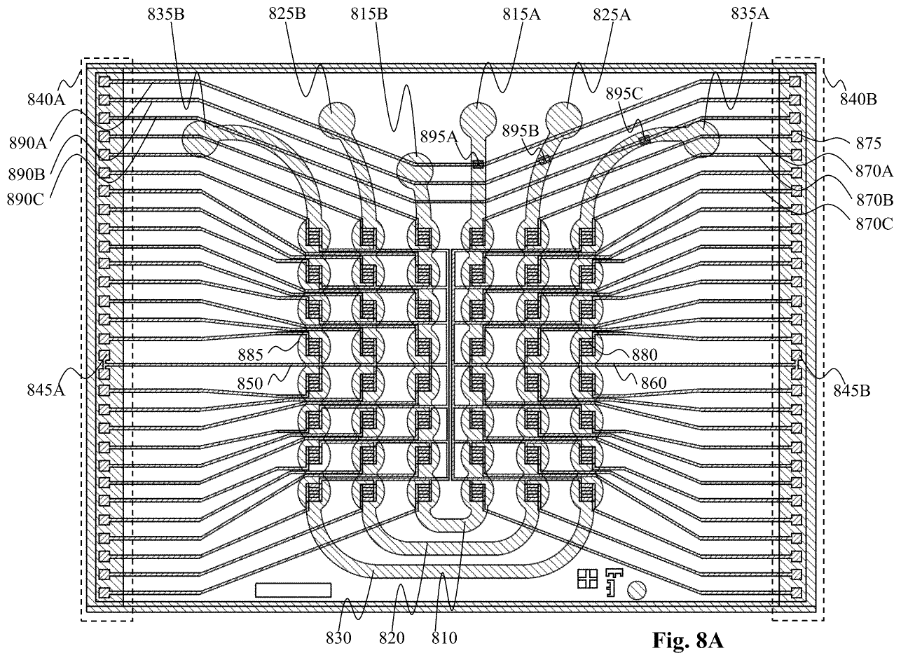

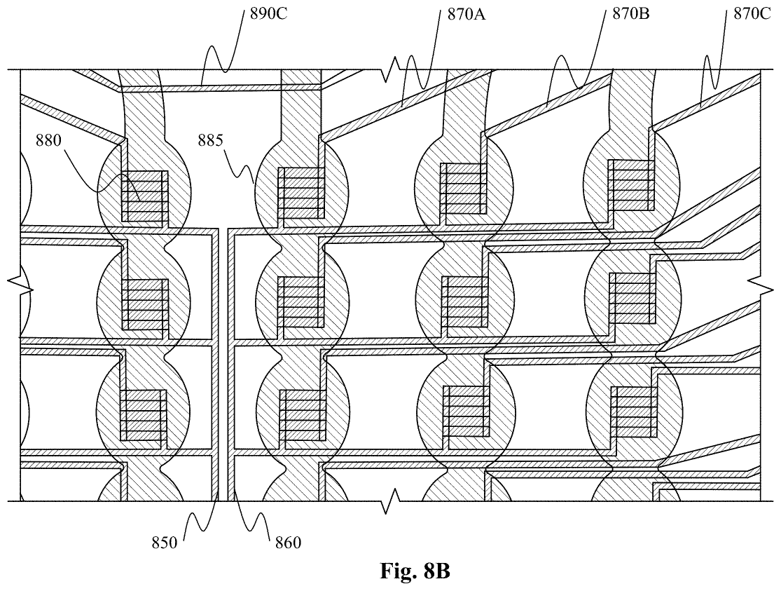

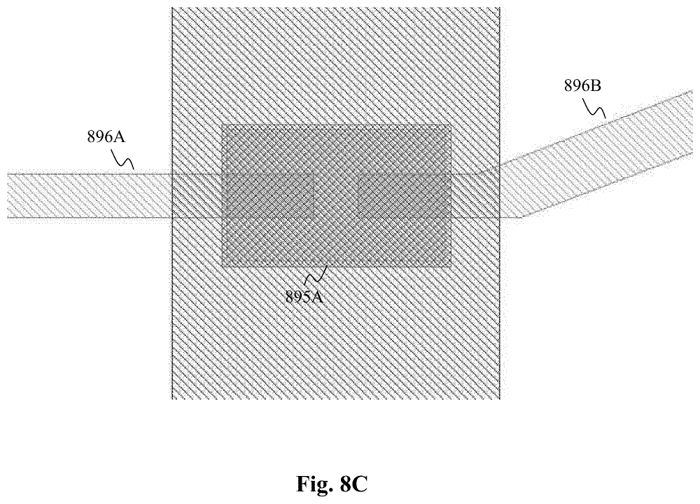

FIGS. 8A, 8B and 8C schematically illustrate the structure of a GMR sensor chip 280 which can be mounted on the cartridge assembly 200 according to an embodiment of the present disclosure. As shown in FIG. 8A, the GMR sensor chip 280 includes: at least one of channels 810, 820 and 830 arranged approximately in the center of the chip; a plurality of GMR sensors 880 disposed within the channels; electric contact pads 840A, 840B arranged on two opposing ends of the GMR sensor chip; and metal wires 850, 860, 870A, 870B, 870C, 890A, 890B, 890C coupled to the electric contact pads 840A, 840B.

The channels 810, 820 and 830 each can have a serpentine shape to allow for more sensors to be packed inside. A plurality of channel expansions 885 can be arranged along the channels to receive the plurality of GMR sensors. Fluid to be tested flows into and out of the channels 810, 820, 830 via channel entrances 815A, 825A, 835A and channel exits 815B, 825B, 835B, respectively. Although FIG. 8A shows that the GMR sensors 880 are arranged in an 8×6 sensor array, with 16 sensors received in each of three channels 810, 820, 830, other combinations can be used to satisfy the specific needs of the analyte to be sensed.

In some embodiments, referring as non-limiting examples to FIGS. 8A and 8B, multiplex detection schemes, for example, for performing multiplex assays for detecting more than one analyte in the same query sample or in difference query samples, may be achieved by spatially disposing one or more different GMR sensors 880, or one or more different sets of GMR sensors 880, within one or more of channels 810, 820, and 830, wherein each different GMR sensor 880 or each different set of GMR sensors 880 is configured with differential tagging and/or coating such that each differentially tagged and/or coated GMR sensor 880 or sets of GMR sensor 880 set interacts with different molecules, such as different capture nucleic acids, different probes, different primers, different captured amplicons, different distinguishable captured amplicons, and/or the like described herein and throughout, thereby allowing for the detection of different analytes in the same sample, or different analytes in different samples, to be detected. In some embodiments, each differentially tagged and/or coated GMR sensor 880 interacts with different capture nucleic acids. In some embodiments, each differentially tagged and/or coated GMR sensor 880 interacts with different captured amplicons. In some embodiments, each differentially tagged and/or coated GMR sensor 880 interacts with different distinguishable captured amplicons. In some embodiments, a different analyte for the same query sample or from different query samples is detected from each channel 810, 820, and/or 830.

The electric contact pads 840A, 840B comprise a plurality of electric contact pins. The metal wires 850, 860, 870A, 870B, 870C connect the GMR sensors to corresponding electric contact pins 845A, 845B, 875. The electric contact pads 840A, 840B are in turn connected to the electrical contact pads 290 provided on the cartridge assembly 200. When the cartridge assembly 200 is inserted to the cartridge reader 310, electric connection is formed between the GMR sensor chip 280 and the cartridge reader 310 to enable sending of measurement signals from the GMR sensors to the cartridge reader 310.

FIG. 8B shows more details of the GMR sensors. For example, each GMR sensor can be comprised of five GMR strips which are connected in parallel. At one end, each GMR sensor is connected by one of two main metal wires (i.e., either wire 850 or 860) to one of two common pins (i.e., either pin 845A or 845B). The other ends of the GMR sensors are connected by separate metal wires 870A, 870B, 870C to distinct pins 875 on the electric contact pads 840A or 840B.

FIG. 8A also shows fluid detection metal wires 890A, 890B, 890C which are arranged in the proximity of the channel entrances and/or exits, each corresponding to one of the channels. The fluid detection function is carried out by switches 895A, 895B, 895C arranged in the respective fluid detection metal wires. FIG. 8C shows the structure of the switch 895A in detail. In response to recognition that conductive fluid (for example, plasma) flows over it, the switch 895A can couple the wire 896A on one side to the wire 896B on the other side, generating a fluid detection signal.

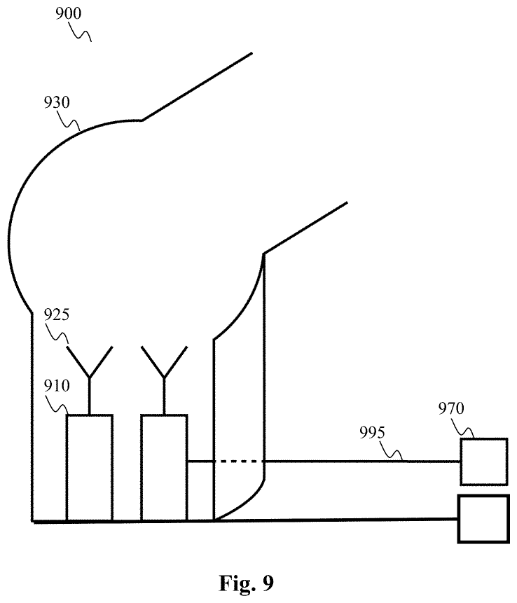

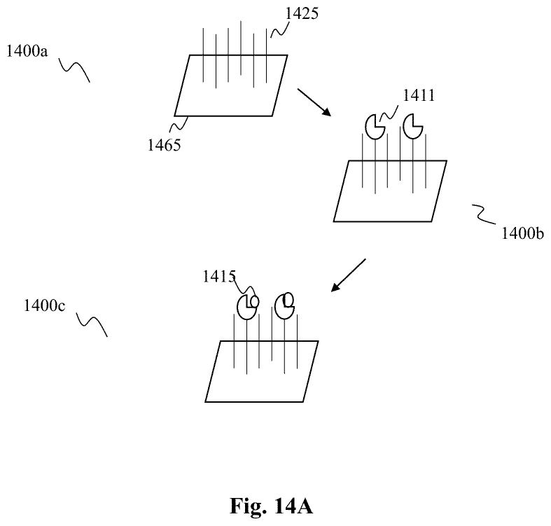

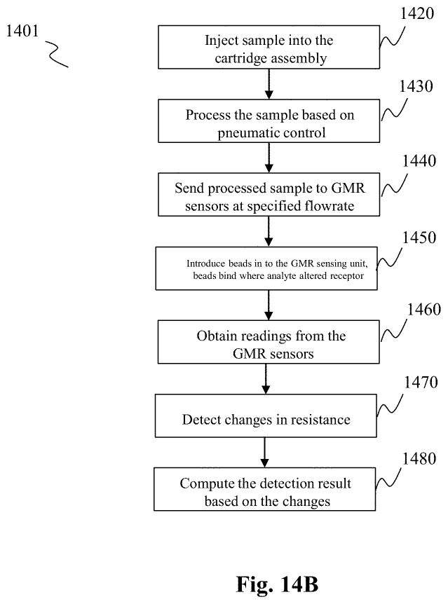

The structure and wiring of the GMR sensor chip shown in FIGS. 8A-C are only exemplary in nature, it will be apparent to those skilled in the art that other structures and wirings are feasible to achieve the same or similar functions. Referring now to FIG. 9, there is shown a cross-sectional view of channel 900 at a channel expansion 930. Disposed within channel expansion 930 is GMR sensor 910 on which is immobilized one or more biomolecules 925. Immobilization of biomolecule 925 to GMR sensor 910 is via conventional surface chemistry (shown in some further detail in FIG. 14). Biomolecule 925 may be a peptide or protein, DNA, RNA, oligosaccharide, hormone, antibody, glycoprotein or the like, depending on the nature of the specific assay being conducted. Each GMR sensor 910 is connected by wire 995 to a contact pad 970 located outside of channel 900. In some embodiments, wire 995 is connect to GMR sensor 910 at the bottom of the sensor.

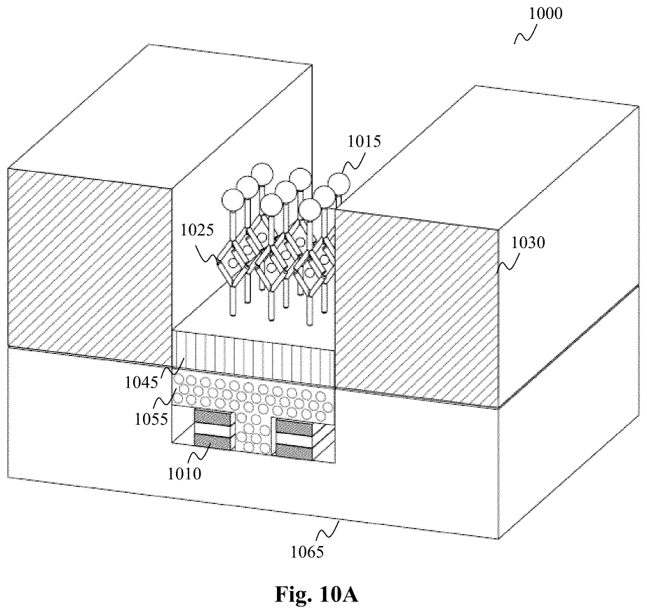

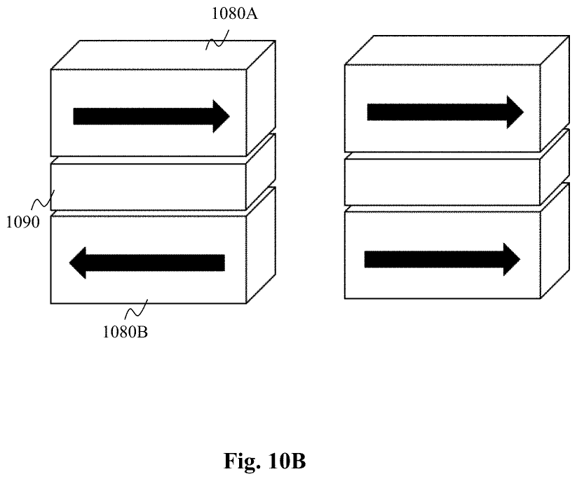

Referring now to FIG. 10A, there is shown a more detailed cross-sectional view of a channel 1000 having a channel body 1030 lacking a channel expansion at the location of a GMR sensor 1010. Biomolecule 1025 is immobilized with respect to the sensor via attachment to a biosurface 1045. Such immobilization chemistry is known in the art. See, for example, Cha et al. “Immobilization of oriented protein molecules on poly(ethylene glycol)-coated Si(111),” Proteomics 4:1965-1976, (2004); Zellander et al. “Characterization of Pore Structure in Biologically Functional Poly(2-hydroxyethyl methacrylate)-Poly(ethylene glycol) Diacrylate (PHEMA-PEGDA),” PLOS ONE 9(5):e96709, (2014).

In some embodiments, biosurface 1045 comprises polymer composition comprising at least two hydrophilic polymers crosslinked with a crosslinking reagent. Such polymer compositions comprising at least two hydrophilic polymers and a crosslinking reagent, comprising such polymer compositions, polymer compositions and/or biosurfaces further comprising a biomolecule, such as a nucleic acid, a protein, and antibody, and the like, and methods of crosslinking and/or preparing such polymer compositions and/or biosurfaces are described in U.S. Provisional Patent Application No. 62/958,510, entitled “POLYMER COMPOSITIONS AND BIOSURFACES COMPRISING THEM ON SENSORS,” filed on Jan. 8, 2020 (Attorney Docket No. 026462-0506342), which is hereby incorporated by reference in its entirety.

In some embodiments, biosurface 1045 comprises a polymer composition comprising a PEG polymer crosslinked with PHEMA.

In some embodiments, the crosslinking reagent is represented by Formula (I):

(I)
PA-L-PA

wherein each PA is a photo- or metal-activated or activated group, and L is a linking group. In some embodiments, each PA is independently selected from a photo-activated group or a metal-activated group, and L is a linking group. In some embodiments, each PA is the same and in other embodiments each PA is different. In some embodiments, each PA independently comprises an azide (—N₃), a diazo (—N₂) group, an aryl azide, an acyl azide, an azidoformate, a sulfonyl azide, a phosphoryl azide, a diazoalkane, a diazoketone, a diazoacetate, a diazirine, an aliphatic azo, an aryl ketone, benzophenone, acetophenone, anthroquinone, and anthrone. In some embodiments, each PA independently comprises an azide (—N₃), or a diazo (—N₂) group. In some embodiments, such polymer compositions do not comprise a block co-polymer of a PEG polymer and a PHEMA polymer.

In some embodiments PA is photo- or metal-activated to form a nitrene intermediate capable of C—H and/or O—H insertion. See, for example, “Photogenerated reactive intermediates and their properties,” Chapter 2 in Laboratory Techniques in Biochemistry and Molecular Biology, Elsevier Press, 12:8-24 (1983). In some embodiments, PA is metal activated to form a carbene or carbenoid intermediate capable of C—H and/or O—H insertion. See, for example, Doyle et al. “Catalytic Carbene Insertion into C—H Bonds,” Chem. Rev. 2:704-724 (2010).

In some embodiments, each PA is an azide (—N₃) moiety and photoactivation generates nitrene intermediates capable of C—H and/or O—H insertion thereby mediating crosslinking of at least to hydrophilic polymers, such as PEG and PHEMA polymers. In some embodiments, each PA is a diazo (—N₂) and metal catalyzed decomposition reaction forms a carbene or carbenoid intermediate capable of C—H and/or O—H insertion thereby mediating crosslinking of PEG and PHEMA polymers. Both azide and diazo preparations are well known in the art, and in the case of azide are readily prepared by S_{N}² displacement reaction of azide anion, N₃⁻ with an appropriate organic moiety possessing a leaving group.

In some embodiments, L comprises at least one Y and one or more X, wherein: (a) each at least one Y is independently selected from the group consisting of: an optionally substituted divalent alkylene; an optionally substituted arylene; and optionally substituted divalent heteroaromatic ring moiety; having from 1 to 20 atoms; an alkylene, —(CR₂)ₚ—, wherein p is an integer from 1 to 10, 1 to 6, or 1 to 4, and wherein R₂ is independently selected from the group consisting of H and lower alkyl, C₁-C₅ alkyl, and C₁-C₃ alkyl; and/or a divalent heteroaromatic ring having from 4 to 20 carbon atoms and contains at least one heteroatom selected from the group consisting of O, N, and S; and (b) each X is independently selected from the group consisting of alkylene, —NR₁—, —O—, —S—, —S—S—, —CO—NR₁—, —NR₁—CO—, —CO—O—, —O—CO—, —CO—, and a bond, wherein R₁ is independently selected from the group consisting of H and lower alkyl.

In some embodiments, the crosslinking reagent is represented by Formula (II):

(II)
PA-Y₁-X₁-X₂-X₃-X₄-X₅-X₆-X₇-X₈-X₉-Y₂-PA

wherein each PA is a photo-activated group or a metal-activated group, and Y₁-X₁-X₂-X₃-X₄-X₅-X₆-X₇-X₈-X₉-Y₂ is a linking group. In some embodiments, each PA independently comprises an azide (—N₃), a diazo (—N₂) group, an aryl azide, an acyl azide, an azidoformate, a sulfonyl azide, a phosphoryl azide, a diazoalkane, a diazoketone, a diazoacetate, a diazirine, an aliphatic azo, an aryl ketone, benzophenone, acetophenone, anthroquinone, and anthrone. In some embodiments, each PA independently comprises an azide (—N₃), or a diazo (—N₂) group. In some embodiments, Y₁-X₁-X₂-X₃-X₄-X₅-X₆-X₇-X₈-X₉-Y₂ is a linking group. In some embodiments, each of X₁, X₂, X₃, X₄, X₅, X₆, X₇, X₈, and X₉ is independently selected from the group consisting of alkylene, —NR₁—, —O—, —S—, —S—S—, —CO—NR₁—, —NR₁—CO—, —CO—O—, —O—CO—, —CO—, and a bond, wherein R₁ is independently selected from the group consisting of H and lower alkyl. In some embodiments, each of Y₁ and Y₂ are each, independently, selected from the group consisting of: an optionally substituted divalent alkylene; an optionally substituted arylene; and optionally substituted divalent heteroaromatic ring moiety; having from 1 to 20 atoms; an alkylene, —(CR₂)ₚ—, wherein p is an integer from 1 to 10, 1 to 6, or 1 to 4, and wherein R₂ is independently selected from the group consisting of H and lower alkyl, C₁-C₅ alkyl, and C₁-C₃ alkyl; and/or a divalent heteroaromatic ring having from 4 to 20 carbon atoms and contains at least one heteroatom selected from the group consisting of O, N, and S.

The term “alkyl,” as used herein, alone or in combination, refers to a straight-chain or branched-chain alkyl radical containing from 2 to 20 carbon atoms. In some embodiments, the alkyl may comprise from 2 to 10 carbon atoms. In further embodiments, the alkyl group may comprise from 2 to 6 carbon atoms. Alkyl groups may be optionally substituted as defined herein below. Examples of alkyl group (given as radicals) include, without limitation methyl, ethyl, n-propyl, isopropyl, n-butyl, isobutyl, sec-butyl, tert-butyl, pentyl, iso-amyl, hexyl, octyl, nonyl and the like.

The term “alkenyl,” as used herein, alone or in combination, refers to a straight-chain or branched-chain hydrocarbon radical having one or more double bonds and containing from 2 to 20 carbon atoms. In some embodiments, the alkenyl group may comprise from 2 to 6 carbon atoms.

The term “alkenylene” refers to a carbon-carbon double bond system attached at two or more positions such as ethenylene [(—CH═CH—), (—C::C—)]. Examples of suitable alkenyl radicals include propenyl, 2-methylpropenyl, 1,4-butadienyl and the like.

The term “alkynyl,” as used herein, alone or in combination, refers to a straight-chain or branched chain hydrocarbon radical having one or more triple bonds and containing from 4 to 20 carbon atoms. In certain embodiments, said alkynyl comprises from 4 to 6 carbon atoms. Examples of alkynyl groups include butyn-1-yl, butyn-2-yl, pentyn-1-yl, 3-methylbutyn-1-yl, hexyn-2-yl, and the like.

The term “aryl,” as used herein, alone or in combination, means a carbocyclic aromatic system containing one, two or three rings wherein such rings may be attached together in a pendent manner or may be fused. In some embodiments, “Aryl” groups include groups having one or more 5- or 6-member aromatic rings. Aryl groups contain no heteroatoms in the aryl rings. Aryl groups are optionally substituted with one or more non-hydrogen substituents.

The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylene,” refers to a divalent aromatic radical which consists of the elements carbon and hydrogen. The divalent aromatic radical may include only one benzene ring, or a plurality of benzene rings as in diphenyl, naphthyl, oranthracyl.

The term “aralkyl,” as used herein, alone or in combination, refers to an aryl group attached to the parent molecular moiety through an alkyl group.

The term “heteroaryl” and “heteroaromatic rings”, as used herein, refer to and include groups having one or more aromatic rings in which at least one ring contains a heteroatom (a non-carbon ring atom). Heteroaryl groups include those having one or two heteroaromatic rings carrying 1, 2 or 3 heteroatoms. Heteroaryl groups can contain 5-20, 5-12 or 5-10 ring atoms. Heteroaryl groups include those having one aromatic ring contains a heteroatom and one aromatic ring containing carbon ring atoms. Heteroaryl groups include those having one or more 5- or 6-member aromatic heteroaromatic rings and one or more 6-member carbon aromatic rings. Heteroaromatic rings can include one or more N, O, or S atoms in the ring. Heteroaromatic rings can include those with one, two or three N, those with one or two O, and those with one or two S, or combinations of one or two or three N, O or S. Specific heteroaryl groups include furyl, pyridinyl, pyrazinyl, pyrimidinyl, quinolinyl, and purinyl groups.

The term “lower alkyl” refers to, for example, C₁-C₉ alkyl, C₁-C₈ alkyl, C₁-C₇ alkyl, C₁-C₆ alkyl, C₁-C₆ alkyl, C₁-C₅ alkyl, C₁-C₄ alkyl, C₁-C₃ alkyl, or C₁-C₂ alkyl.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed.

Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.” The term “lower,” as used herein, alone or in combination, means containing from 1 to and including 6 carbon atoms.

In some embodiments, the crosslinking reagent comprises bis[2-(4-azidosalicylamido)ethyl]disulfide or dithiobis(phenylazide).

In some embodiments, L in Formula (I) can be any organic fragment that will support the presence of each PA moiety. It can be a simple C₂-C₂₀ hydrocarbon chain that is straight chained or branched. Such hydrocarbons can include fluorinated variants with any degree of fluorine substitution. In some embodiments, LG can include aromatic hydrocarbons including, without limitation, benzene, naphthalene, biphenyl, binaphthyl, or combinations of aromatic structures with C₂-C₂₀ hydrocarbon chains. Thus, in some embodiments, LG can be alkyl, aryl, or aralkyl in structure. In some embodiments, alkyl linking groups may have one or more carbons in their chains substituted with oxygen (0), or an amine (NR), where R is H or C₁-C₆ alkyl.

In accordance with the foregoing embodiments, a crosslinked PEG-PHEMA structure may be given by Formula (III):

PEG-A-L-A-PHEMA

wherein PEG is the polyethylene glycol moiety, each A is an attachment atom from the catalytic reaction of azide or diazo, i.e., CH₂ or NH, and LG is the linking group as described above.

In some embodiments, each A in Formula (I), Formula (II), and/or Formula (III) represents an attachment atom derived from the decomposition reaction of an azide (—N₃), a diazo (—N₂) group, an aryl azide, an acyl azide, an azidoformate, a sulfonyl azide, a phosphoryl azide, a diazoalkane, a diazoketone, a diazoacetate, a diazirine, an aliphatic azo, an aryl ketone, benzophenone, acetophenone, anthroquinone, or an anthrone.

In some embodiments, a polymer composition, such as a PEG-PHEMA composition that may be employed to functionalize the surface of a sensor, such as a GMR sensor, can be prepared by mixing a PEG solution comprising, for example, N-hydroxysuccinimide (NHS)-PEG-NHS (MW 600) dissolved in a suitable solvent (e.g., isopropyl alcohol, acetone or methanol, and/or water), a PHEMA solution comprising polyhydroxyethyl methacrylate (MW 20,000) dissolved in a suitable solvent (e.g., isopropyl alcohol, acetone or methanol, and/or water), and an optional crosslinker. The resulting solution can be coated on a sensor surface using a suitable coating process (e.g., micro-printing, dip coating, spin coating or aerosol coating). After coating a surface with the PEG-PHEMA solution, the surface can be cured using UV light followed by washing with a suitable solvent, such as isopropyl alcohol and/car water. In some embodiments a surface of a sensor is covalently attached to one or more nucleic acids. In some embodiments, the coated surface can be used to bind with primary amines (e.g., to attach a protein, and antibody, an antigen-binding portion of an antibody, and the like). A PEG-PHEMA coating can protect a sensor surface against corrosion. In some embodiments, a surface of a sensor comprises a surface described in International Patent Application No. PCT/US2019/043766.

In FIG. 10A, a magnetic bead-bound entity 1015 is configured to interact with biomolecule 1025 or an analyte of interest, such as in a sandwich complex of antibody-analyte-magnetic bead-bound antibody. Below biosurface 1045 is a further insulating layer 1055. Insulating layer 1055 may be in direct contact with GMR sensors 1010 and may comprise, for example, a metal oxide layer. Biosurface layer 1045 is in direct contact with insulating layer 1045. A base 1065 serves as the scaffold for each component above it, the GMR sensors 1010, insulating layer 1055, and biosurface layer 1045. In some embodiments, base 1065 is made from silicon wafer.

FIG. 10B schematically illustrates the basic structure and principle of GMR sensors. A typical GMR sensor consists of a metallic multi-layered structure with a non-magnetic conductive interlayer 1090 sandwiched between two magnetic layers 1080A and 1080B. The non-magnetic conductive interlayer 1490 is often a thin copper film. The magnetic layers 1080A and 1080B can be made of ferromagnetic alloy material.

The electrical resistance of the metallic multi-layered structure changes depending on the relative magnetization direction of the magnetic layers 1080A and 1080B. Parallel magnetization (as shown in the right half of FIG. 10B) results in lower resistance, while anti-parallel magnetization (as shown in the left half of FIG. 10B) results in higher resistance. The magnetization direction can be controlled by a magnetic field applied externally. As a result, the metallic multi-layered structure displays a change in its electrical resistance as a function of the external magnetic field.

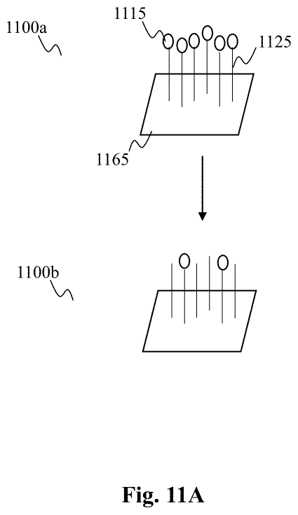

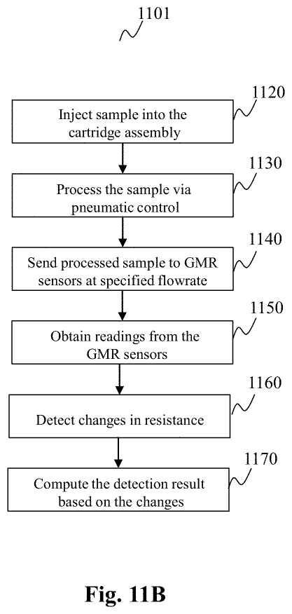

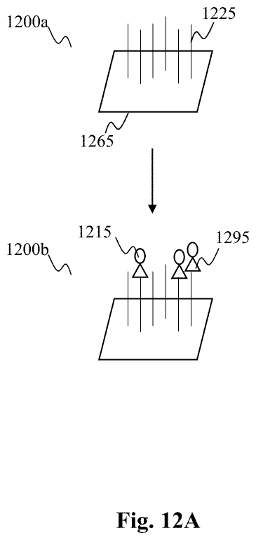

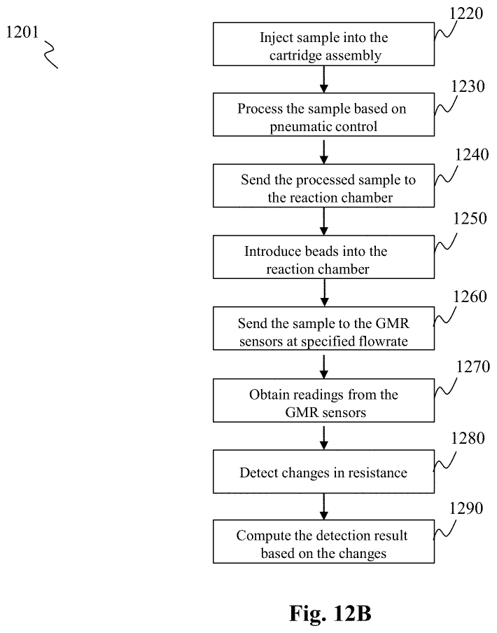

Referring now to FIGS. 11A and 12A, there are shown two exemplary basic modes by which GMR sensors operate in accordance with various assay applications described herein. In the first mode, exemplified in FIG. 11A, magnetic beads 1115 are loaded proximal to a GMR sensor (see FIG. 11A, 1010) via biosurface 1165 at the start of the assay. During the assay the presence of a query analyte results in magnetic beads 1115 being displaced from biosurface 1165 (and thus, displaced away from the GMR sensor); this mode is the so-called subtractive mode because magnetic beads are being taken away from the proximity of the sensor surface. The second main mode operation, typified in FIG. 12A, is the additive mode. In such assays, there is a net addition of magnetic beads 1215 in the vicinity of the GMR sensor (see FIG. 10A, 1010) when a query analyte is present. Either mode, subtractive or additive, relies on the changed state in the number of beads (1115, 1215) proximal to the sensor surface thereby altering the magnetoresistance in the GMR sensor system. The change in magnetoresistance is measured and query analyte concentrations can be determined quantitatively.

Referring back to FIG. 11A, there is shown a sensor structure diagram illustrating the sensor structures throughout an exemplary subtractive process. At the start of the process the system is in state 1100a in which the GMR sensor has disposed on its biosurface 1165 a plurality of molecules (typically biomolecules) 1125 with associated magnetic beads 1115. The volume above biosurface 1165 may begin dry or with a solvent present. When dry, the detection process may include a solvent priming step with, for example, a buffer solution. After introduction of analyte, the system takes the form of state 1100b in which some of magnetic beads 1115 have been removed from the molecules 1125 in proportion to the concentration of analyte. The change in states 1100a and 1100b provide a measurable change in magnetoresistance that allows quantitation of the analyte of interest. In some embodiments, the analyte may simply displace beads directly from molecules 1125. In other embodiments, the analyte may chemically react with molecules 1125 to cleave a portion of the molecule attached to beads 1115, thereby releasing beads 1115 along with the cleaved portion of molecule 1125.

In embodiments, biosurface 1165 comprises a polymer. The specific polymer may be chosen to facilitate covalent attachment of molecules 1125 to biosurface 1165. In other embodiments, molecules 1125 may be associated with biosurface 1165 via electrostatic interactions. Polymer coatings may be selected for or modified to use conventional linking chemistries for covalently anchoring biomolecules, for example. Linking chemistries include any chemical moieties comprising an organic functional group handle including, without limitation, amines, alcohols, carboxylic acids, and thiol groups. Covalent attachment chemistry includes, without limitation, the formation of esters, amides, thioesters, and imines (which can be subsequently subjected to reduction, i.e., reductive amination). Biosurface 1165 may include surface modifiers, such as surfactants, including without limitation, anionic surfactants, cationic surfactants, and zwitterionic surfactants.

Molecules 1125 can include any number of receptor/ligand entities which can be attached to biosurface 1165. In some embodiments molecules 1125 include any of a variety of biomolecules. Biomolecules include DNA, RNA, and proteins that contains free amine groups can be covalently immobilized on GMR sensor surface with functional NHS groups. For the immunoassays, primary antibody (mouse monoclonal IgG) specific to analyte is attached onto GMR surface. All primary antibodies have multiple free amine groups and most proteins have lysine and/or alpha-amino groups. As long as lysine free primary amines are present, antibodies will be covalently immobilized on GMR sensor. To immobilize antibody on sensors surface, 1.2 nL of primary antibody (1 mg/mL in PBS buffer) are injected onto sensors surface using a printer system (sciFLEXARRAYER, Scienion, Germany). All printed surfaces are incubated overnight at 4° C. under a relative humidity of ˜85%. The surfaces will be washed three times with blocking buffer (50 mM ethanolamine in Tris buffer), and are further blocked with the same buffer for 30 min.

In embodiments, magnetic beads 1115 may be nanoparticulate, including spheroidal nanoparticles. In some embodiments, such nanoparticles have effective diameters in a range from about 1 to about 1000 nanometers (nm), 1 nm to about 500 nm, about 5 nm to about 1000 nm, about 10 nm to about 1000 nm, about 5 nm to about 500 nm, about 5 nm to about 400 nm, about 5 nm to about 300 nm, about 5 nm to about 300 nm, about 5 nm to about 200 nm, about 5 nm to about 100 nm, about 2 to about 50 nm, about 5 to about 20 nm, or about 5 to about 10 nm, and/or ranges in between. In some embodiments such nanoparticles may have effective diameters in a range from about 2 to about 50 nm, or about 5 to about 20 nm, or about 5 to about 10 nm. In embodiments, magnetic beads 1115 may be coated to facilitate covalent attachment to molecules 1125. In other embodiments magnetic beads 1115 may be coated to facilitate electrostatic association with molecules 1125. Magnetic beads 1115 may be differentially tagged and/or coated to facilitate multiplex detection schemes, for example, for performing multiplex assays for detecting more than one analyte in the same query sample or in difference query samples. In such embodiments, the differential tagging and/or coating is configured such that the different beads interact with different molecules disposed on different GMR sensors or on a single sensor in which different molecules are spatially organized to create addressable signals.

In some embodiments, referring as a non-limiting example to FIG. 5A multiplex detection schemes, for example, for performing multiplex assays for detecting more than one analyte in the same query sample or in difference query samples, may be achieved by spatially disposing different GMR sensors 510 within serpentine channel 540, wherein each different GMR sensor 510 is configured to with differential tagging and/or coating such that each differentially tagged and/or coated GMR sensor 510 interacts with different molecules, such as different capture nucleic acids, different probes, different primers, different captured amplicons, different distinguishable captured amplicons, and/or the like described herein and throughout, thereby allowing for the detection of different analytes in the same sample, or different analytes in different samples, to be detected.

In some embodiments, referring as a non-limiting example to FIG. 5B, multiplex detection schemes for example, for performing multiplex assays detecting more than one analyte in the same query sample or in difference query samples, may be achieved by spatially disposing GMR sensors tagged and/or coated with one tag or coating within one of channel 500, and disposing one or more different GMR sensors tagged and/or coated with a different tag or coating in a different channels 500, such GMR sensors in the one channel 500 interact with different molecules, such as different capture nucleic acids, different probes, different primers, different captured amplicons, different distinguishable captured amplicons, and/or the like described herein and throughout, than GMR sensors in the one or more different channels 500, thereby allowing for different samples to be flowed though different channels 500 and thereby allowing for either the same analyte to be measures from different samples or for different analytes to be measured from different samples.

Referring back to FIG. 11B, shown is a process flow 1101 associated with the sensor structure scheme of FIG. 11A. The process commences at 1120 by injecting a sample into a cartridge assembly. The sample may then undergo processing at step 1130 through any necessary steps such as filtration, dilution, and/or chemical modification. The sequencing of these pre-process steps will depend on the nature of the sample and query analyte to be detected. Movement through the system may be controlled pneumatically. Step 1140 involves sending the processed sample to the GMR sensor at a target specified flow rate. Such flow rate may be selected to reflect the kinetics of the chemistry on the GMR sensor surface. Step 1150 provides obtaining readings from the GMR sensors that reflect changes in the concentration of magnetic beads at the surface of the GMR sensor. These readings allow detecting changes in magnetoresistance at step 1160. Finally, step 1170 provides computing the detect result based on the changes in magnetoresistance.

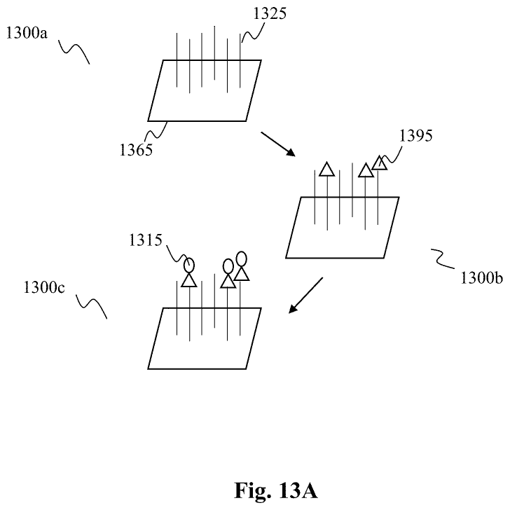

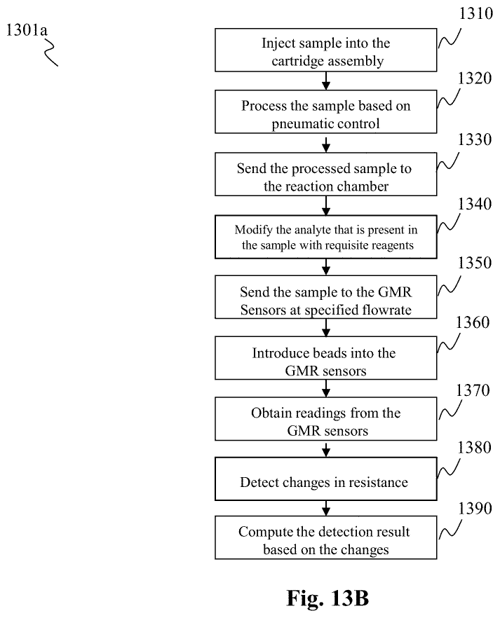

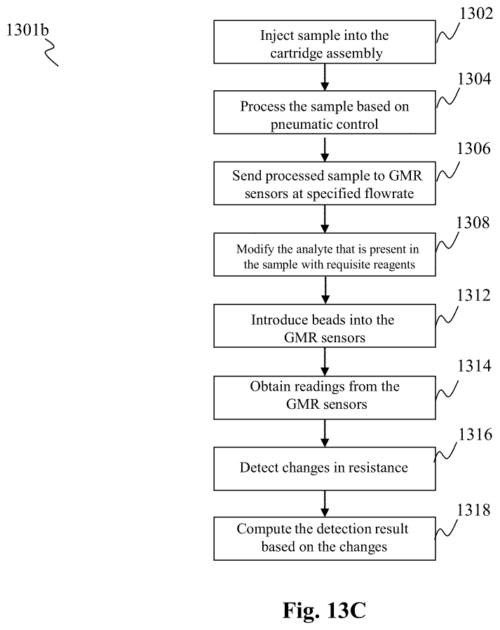

Referring now to FIG. 12A, there is shown a sensor structure diagram illustrating the sensor structures throughout an exemplary additive process. At the start of the process the system is in state 1200a in which the GMR sensor has disposed on its biosurface 1265 a plurality of molecules (typically biomolecules) 1225. The plurality of molecules 1225 is selected to bind a query analyte 1290, as indicated in second state 1200b. Query analyte 1295 is configured to bind magnetic beads 1215. In some embodiments, query analyte 1295 is associated with the bead prior to passing over biosurface 1265. For example, this may take place during pre-processing of the sample being tested. (In other embodiments, query analyte 1295 may pass over the biosurface first, then query analyte 1295 may be modified with magnetic beads 1215 after the analyte is bound to biosurface 1265, as described below with reference to FIG. 13A). In some embodiments, a given query analyte 1295 may require chemical modification prior to binding magnetic particles 1215. In some embodiments, magnetic beads 1215 may be modified to interact with query analyte 1295. The ability to quantitate analyte is provided by changes in measured magnetoresistance from state 1200a, where no magnetic beads 1215 are present, to state 1200b, where magnetic beads 1215 are associated with biosurface 1265.

FIG. 12B shows an exemplary process flow 1201 associated with the sensor structure scheme of FIG. 12A. The process commences at 1220 by injecting a sample into a cartridge assembly. The sample may then undergo processing at step 1230 through any necessary steps such as filtration, dilution, and/or chemical modification. The sequencing of these pre-process steps will depend on the nature of the sample and query analyte to be detected. Movement through the system may be controlled pneumatically. Step 1240 involves sending the processed sample to a reaction chamber and then in step 1250 beads are introduced into the reaction chamber to modify the query analyte. As described above, such modification may be performed directly on the sensor surface rather than in the reaction chamber. In step 1260, the modified sample is sent to the GMR sensors at a target flow rate. Such flow rate may be selected to reflect the kinetics of the chemistry on the GMR sensor surface. Step 1270 provides obtaining readings from the GMR sensors that reflect changes in the concentration of magnetic beads at the surface of the GMR sensor. These readings allow detecting changes in magnetoresistance at step 1280. Finally, step 1290 provides computing the detect result based on the changes in magnetoresistance.

Referring now to FIG. 13A, there is shown a sensor structure diagram illustrating the sensor structures states 1300a-c throughout an exemplary additive process. At the start of the process the system is in state 1300a in which the GMR sensor has disposed on its biosurface 1365 a plurality of molecules (typically biomolecules) 1325. The plurality of molecules 1325 is selected to bind a query analyte 1395, as indicated in second state 1300b. Query analyte 1395 is configured to bind magnetic beads 1315, as indicated in state 1300c. In some embodiments, a given query analyte 1395 may require chemical modification prior to binding magnetic particles 1315. In other embodiments, query analyte 1395 may bind magnetic nanoparticles 1315 without chemical modification. In some embodiments, magnetic beads 1315 are coated or otherwise modified to interact with query analyte 1395. The ability to quantitate query analyte 1395 is provided by changes in measured magnetoresistance from state 1300a, where no magnetic beads 1315 are present, to state 1300c, where magnetic beads 1315 are associated with biosurface 1365.

FIG. 13B shows an exemplary process flow 1301a associated with the sensor structure scheme of FIG. 13A. The process commences at 1310 by injecting a sample into a cartridge assembly. The sample may then undergo processing at step 1320 through any necessary steps such as filtration, dilution, and/or the like. The sequencing of these pre-process steps will depend on the nature of the sample and query analyte to be detected. At 1330, the process sample is sent to a reaction chamber. Movement through the system may be controlled pneumatically. Step 1340 involves modifying the analyte present in the sample chamber with reagents to allow it to interact with magnetic particles. At step 1350, the modified sample is sent to the GMR sensors at a target flow rate. Such flow rate may be selected to reflect the kinetics of the chemistry on the GMR sensor surface. Next, step 1360 introduces beads into the GMR sensors, which can now interact with the modified analyte. In some embodiments, the beads may be modified as well, such as with a coating or some other linking molecule that will enable interaction with the modified analyte. Step 1370 provides obtaining readings from the GMR sensors that reflect changes in the concentration of magnetic beads at the surface of the GMR sensor. These readings allow detecting changes in magnetoresistance at step 1380. Finally, step 1390 provides computing the detect result based on the changes in magnetoresistance.

FIG. 13C shows an alternative exemplary process flow 1301b associated with the sensor structure scheme of FIG. 13A. The process commences at 1302 by injecting a sample into a cartridge assembly. The sample may then undergo processing at step 1304 through any necessary steps such as filtration, dilution, and/or the like. The sequencing of these pre-process steps will depend on the nature of the sample and query analyte to be detected. Movement through the system may be controlled pneumatically. At step 1306, the modified sample is sent to the GMR sensors at a target flow rate. Such flow rate may be selected to reflect the kinetics of the chemistry on the GMR sensor surface. Step 1308 involves modifying the analyte present in the sample with reagents to allow it to interact with magnetic particles. Next, step 1312 introduces beads into the GMR sensors, which can now interact with the modified analyte. In some embodiments, the beads may be modified as well, such as with a coating or some other linking molecule that will enable interaction with the modified analyte. Step 1314 provides obtaining readings from the GMR sensors that reflect changes in the concentration of magnetic beads at the surface of the GMR sensor. These readings allow detecting changes in magnetoresistance at step 1316. Finally, step 1318 provides computing the detect result based on the changes in magnetoresistance.

Referring now to FIG. 14A, there is shown a sensor structure diagram illustrating the sensor structures states 1400a-c throughout an exemplary additive process. At the start of the process the system is in state 1400a in which the GMR sensor has disposed on its biosurface 1465 a plurality of molecules (typically biomolecules) 1425. The plurality of molecules 1425 is selected to interact (chemically react) with a query analyte. Such interaction modifies molecules 1425 (in proportion to analyte concentration) to provide modified molecules 1411, as indicated in second state 1400b. Modified molecules 1411 are configured to bind magnetic beads 1415, as indicated in state 1300c. In some embodiments, modified molecules 1411 may require further chemical modification prior to binding magnetic particles 1415. In other embodiments, modified molecules 1411 may bind magnetic nanoparticles 1415 without chemical modification. In some embodiments, magnetic beads 1415 are coated or otherwise modified to interact with modified molecules 1411. The ability to quantitate query analyte is provided by changes in measured magnetoresistance from state 1400a, where no magnetic beads 1415 are present, to state 1400c, where magnetic beads 1415 are associated with biosurface 1465 via modified molecules 1411. Note, in the overall process, the query analyte is merely serving as a reagent to chemically modify the plurality of molecules 1425 and does not otherwise remain a part of the process once it has performed this function.

FIG. 14B shows an exemplary process flow 1401 associated with the sensor structure scheme of FIG. 14A. The process commences at 1420 by injecting a sample into a cartridge assembly. The sample may then undergo processing at step 1430 through any necessary steps such as filtration, dilution, and/or the like. The sequencing of these pre-process steps will depend on the nature of the sample and query analyte to be detected. Movement through the system may be controlled pneumatically. At 1440, the process sample is sent to GMR sensors at a specified flowrate. Such flow rate may be selected to reflect the kinetics of the chemistry on the GMR sensor surface. Next, step 1450 introduces beads into the GMR sensors, which can now interact with the modified molecules on the biosurface. In some embodiments, the beads may be modified as well, such as with a coating or some other linking molecule that will enable interaction with the modified molecules on the biosurface. Step 1460 provides obtaining readings from the GMR sensors that reflect changes in the concentration of magnetic beads at the surface of the GMR sensor. These readings allow detecting changes in magnetoresistance at step 1470. Finally, step 1480 provides computing the detect result based on the changes in magnetoresistance.

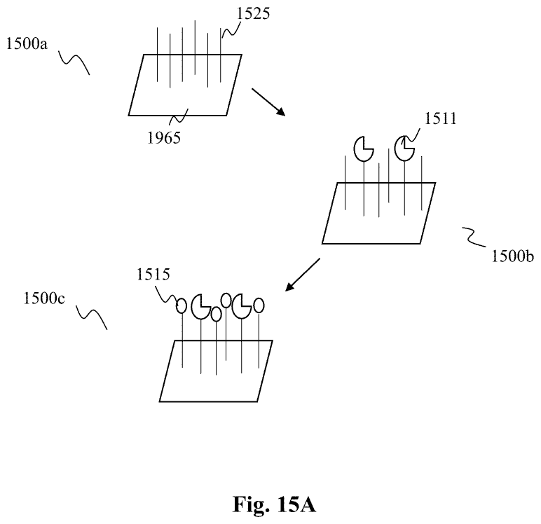

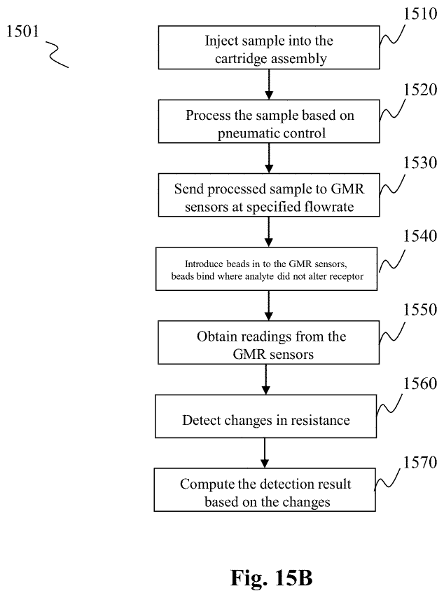

Referring now to FIG. 15A, there is shown a sensor structure diagram illustrating the sensor structures states 1500a-c throughout an exemplary additive process. At the start of the process the system is in state 1500a in which the GMR sensor has disposed on its biosurface 1565 a plurality of molecules (typically biomolecules) 1525. The plurality of molecules 1525 is selected to interact (chemically react) with a query analyte. Such interaction modifies molecules 1525 (in proportion to analyte concentration) to provide modified molecules 1511, as indicated in second state 1500b. Modified molecules 1511 are configured to prevent binding of magnetic beads 1515, as indicated in state 1500c, in which magnetic beads only bind to molecules 1525 that were not modified by the analyte. In some embodiments, magnetic beads 1515 are coated or otherwise modified to interact with molecules 1525. The ability to quantitate query analyte is provided by changes in measured magnetoresistance from state 1500a, where no magnetic beads 1515 are present, to state 1500c, where magnetic beads 1515 are associated with biosurface 1565 via molecules 1525. Note, in the overall process, the query analyte is merely serving as a reagent to chemically modify the plurality of molecules 1525 and does not otherwise remain a part of the process once it has performed this function.

FIG. 15B shows an exemplary process flow 1501 associated with the sensor structure scheme of FIG. 15A. The process commences at 1510 by injecting a sample into a cartridge assembly. The sample may then undergo processing at step 1520 through any necessary steps such as filtration, dilution, and/or the like. The sequencing of these pre-process steps will depend on the nature of the sample and query analyte to be detected. Movement through the system may be controlled pneumatically. At step 1530, the processed sample is sent to GMR sensors at a specified flowrate. Such flow rate may be selected to reflect the kinetics of the chemistry on the GMR sensor surface. Next, step 1540 introduces beads into the GMR sensors, which can now interact with the unmodified molecules on the biosurface. In some embodiments, the beads may be modified, such as with a coating or some other linking molecule that will enable interaction with the unmodified molecules. Step 1550 provides obtaining readings from the GMR sensors that reflect changes in the concentration of magnetic beads at the surface of the GMR sensor. These readings allow detecting changes in magnetoresistance at step 1560. Finally, step 1570 provides computing the detect result based on the changes in magnetoresistance.

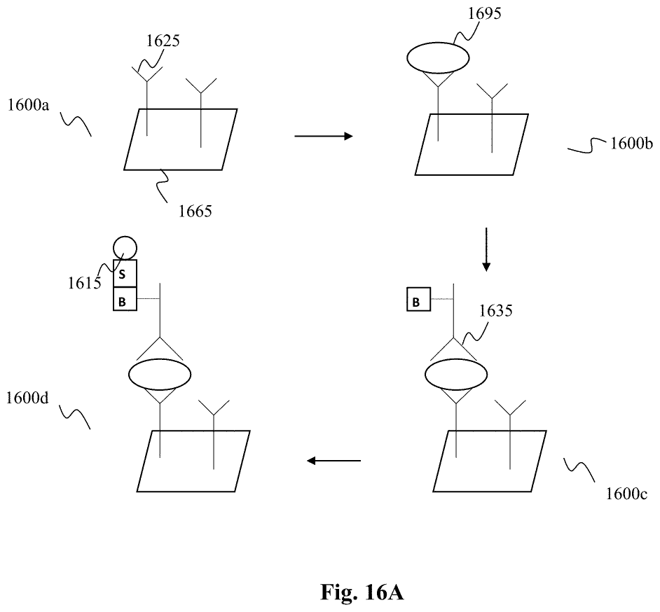

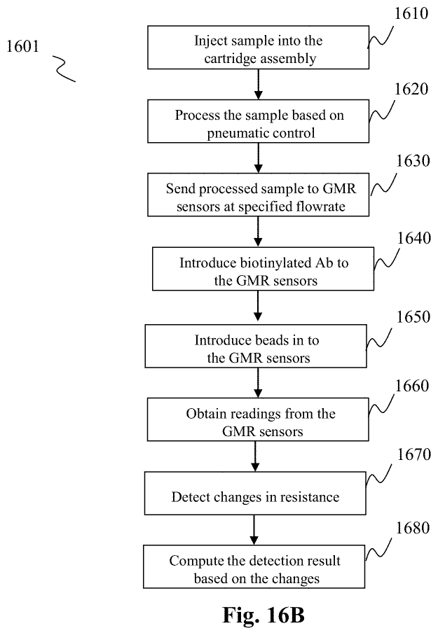

Referring now to FIG. 16A, there is shown a sensor structure diagram illustrating the sensor structure states 1600a-d throughout an exemplary additive process that employs a sandwich antibody strategy for detection of analyte 1695 (state 1600b). At the start of the process the system is in state 1600a in which the GMR sensor has disposed on its biosurface 1665 a plurality of antibodies 1625. Analyte 1695 is then passed over biosurface 1665, allowing binding of analyte 1695 to antibody 1625, as indicated in state 1600b. Analyte 1695 is then modified by binding to a second antibody 1635 to which a covalently linked biotin moiety (B) is provided, as indicated in state 1600c. Magnetic beads 1615 modified with streptavidin (S) are then added, thereby allowing the strong biotin-streptavidin association to provide state 1600d. In some embodiments, streptavidin is provided as a coating on magnetic beads 1615.

FIG. 16B shows an exemplary process flow 1601 associated with the sensor structure scheme of FIG. 16A. The process commences at 1610 by injecting a sample into a cartridge assembly. The sample may then undergo processing at step 1620 through any necessary steps such as filtration, dilution, and/or the like. The sequencing of these pre-process steps will depend on the nature of the sample and query analyte to be detected. Movement through the system may be controlled pneumatically. At step 1630, the processed sample is sent to GMR sensors at a specified flowrate. Such flow rate may be selected to reflect the kinetics of the chemistry on the GMR sensor surface between biosurface-bound antibody and the analyte. Next, step 1640 introduces biotinylated antibody (Ab) to the GMR sensors. This creates the “sandwich” structure of the analyte between two antibodies. At step 1650 streptavidin coated beads are introduced into the GMR sensors, which can now interact with the biotin-bound antibody. Step 1660 provides obtaining readings from the GMR sensors that reflect changes in the concentration of magnetic beads at the surface of the GMR sensor. These readings allow detecting changes in magnetoresistance at step 1670. Finally, step 1680 provides computing the detect result based on the changes in magnetoresistance.

In some embodiments a microfluidic device described herein comprises one or more membranes. A membrane of a microfluidic device binds non-specifically and reversibly to nucleic acids. Any suitable membrane can be used for a microfluidic device or method described herein. Non-limiting examples of membranes include silica, glass fibers, Celite, modified glass and ion-exchange membranes. In some embodiments, a membrane is a porous membrane.

In some embodiments, is a microfluidic device configured to detect a genetic variation in a target nucleic acid that is present in a sample obtained from a subject. In some embodiments, the device comprises one or more components shown in FIGS. 1-15 and 24-26. In some embodiments, a device comprises a configuration, or a variation thereof, shown in FIGS. 1-15 and 24-26. In some embodiments, a device comprises one or more microfluidic channels that are operably and/or fluidically connected to each of the components of the device.

Components or parts that are “fluidically connected” are components and parts of a device that are in contact with and/or can be contacted with (e.g., by opening or closing a valve) a liquid or fluid disposed within the device. A well of a 96-well plate is not considered to be fluidically connected to another well in a 96-well plate. Similarly, an Eppendorf tube is not fluidically connected to another Eppendorf tube even when fluid can be transferred from one tube to another. The term “operably connected” means that the particular components or parts of the device can communicate, are attached, or are connected, respectively, in such a way that they cooperate to achieve their intended function or functions. An operable “connection” may be direct, indirect, physical, or remote.

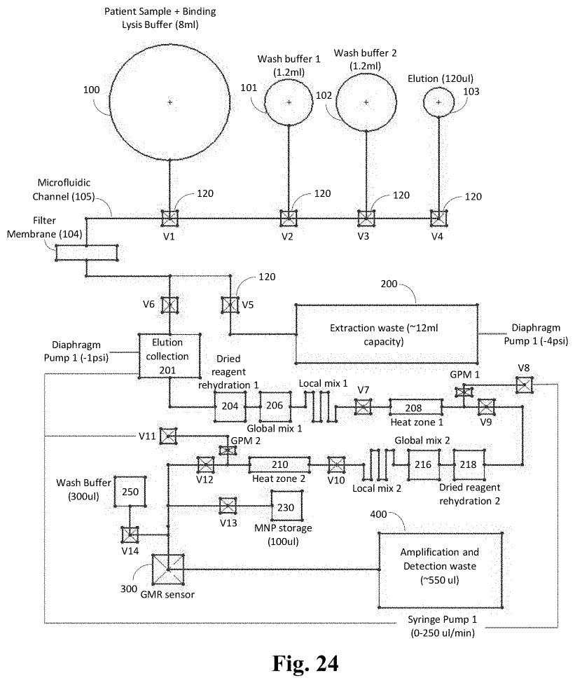

In some embodiments, and turning now to FIGS. 24, 25 and 26, a microfluidic device comprises one or more components selected from a microfluidic channel (e.g., 105), a chamber, a membrane (e.g., 104), an amplification chamber (e.g., 208), a valve 120, a sensor (e.g., 300, e.g., a magnetic sensor), lyophilized reagents, solubilized reagents, a heating source, a cooling source, a pump, a port (e.g., a flow control port 602 or a sample loading port 605). In some embodiments, some or all of the components of the device are operably and/or fluidically connected (e.g., by associated microfluidic channels and valves). In some embodiments, a device comprises one or more chambers selected from a sample chamber (e.g., 100), wash chamber (e.g., 101, 102, 250), collection chambers (e.g., 201), waste collection chambers (e.g., 200, 400), mixing chambers (e.g., 206, 216), reagents chambers (e.g., 204, 218) or magnetic particle chamber (e.g., 230).

In some embodiments, a microfluidic device comprises one or more microfluidic channels (e.g., 105). A microfluidic channel may comprise a suitable geometry in cross-section non-limiting examples of which include circular, oval, rectangular, triangular, the like or combinations thereof. A microfluidic channel may comprise a suitable structure non-liming examples of which include straight, curved, serpentine, and/or elevated, and may include one or more junctions that fluidically connect one or more microfluidic channels and associated components of a microfluidic device described herein. In some embodiments, a microfluidic channel as an average, mean or absolute inside diameter of about 10 nanometers to 1000 micrometers, 50 nanometers to 500 micrometers, 100 nanometers to 500 micrometers, or 100 nanometers to 100 micrometers. In some embodiments, one or more of a valve (120), chamber (100-103, 200, 201, 204, 206, 208, 210, 216, 218, 230, 250), membrane 104, and/or sensor 300 are disposed within a channel body of a microfluidic channel. In some embodiments, a membrane 104 and/or a sensor 300 are disposed within a chamber that is operably and/or fluidically connected to one ore more microfluidic channels. In some embodiments, a microfluidic channel comprises a sample port for introduction of a sample, or one or more reagents, into a microfluidic device.

In some embodiments, a microfluidic device comprises a sample chamber and a sensor that are operably and/or fluidically connected by one or more microfluidic channels and valves such that a direction of flow of a fluid disposed within the device is generally in a direction from the sample chamber toward the sensor. Accordingly, for reference, a first component that is proximal to second component, is a first component that is upstream of the second component with reference to the direction of flow of fluid toward the sensor. Similarly, a first component that is distal to a second component is a first component that is downstream of the second component with reference to the direction of flow of fluid toward the sensor.

In some embodiments, a chamber is a sample chamber. In some embodiments, a sample chamber comprises a sample or is configured to contain a sample. In some embodiments, a sample chamber comprises one or more reagents. In some embodiments, a sample chamber comprises a cell lysis solution which may comprise one or more of a detergent, a salt, a buffer, a chaotropic agent and an alcohol. In some embodiments, a cell lysis solution can be introduced into a sample chamber from another chamber or by introduction through a sample loading port.

In some embodiments, a chamber is a wash chamber. A wash chamber is configured to contain a suitable wash solution. In some embodiments, a wash solution is disposed within a wash chamber (e.g., 101, 102, 250). A wash solution is often configured to wash nucleic acids that are non-covalently or covalently bound to a membrane (e.g., a silica membrane) or a surface (e.g., a surface of a sensor). A wash chamber may comprise any suitable wash solution. In some embodiments, a wash solution comprises one or more of a buffer (e.g., Tris or HEPES), an alcohol, a detergent, a chelating agent, a salt and/or a chaotropic agent.

In some embodiments, a chamber is an elution chamber. An elution chamber is configured to contain a suitable elution solution. An elution solution is configured to remove nucleic acids from a membrane, where the nucleic acids are reversibly and non-covalently bound to the membrane. In some embodiments, an elution solution is disposed within an elution chamber. In some embodiments, an elution solution comprises a buffer (e.g., Tris).

In some embodiments, a sample chamber (e.g., 100), one or more wash chambers (e.g., 101, 102) and/or an elution chamber (e.g., 103) are operably and/or fluidically connected, in parallel to a microfluid channel (e.g., 105), where the microfluid channel comprises one or more valves (e.g., see FIG. 24; V1, V2, V3 and V4) operably connected to the one or more chambers. In some embodiments, each of the one or more chambers (e.g., sample chamber 100, wash chambers (101, 102), elution chamber 103) are located proximal to a membrane (e.g., 104), where each of the chambers are operably and/or fluidically connected to the membrane. In some embodiments, a membrane is housed within a membrane chamber. In some embodiments, a membrane is disposed within a microfluidic channel. In some embodiments, a membrane is in-line with a microfluidic channel, such that a fluid disposed within the device flows through the membrane. In some embodiments, a membrane (e.g., 104) is operably and/or fluidically connected to an amplification chamber located distal to (i.e., downstream of) the membrane.

In some embodiments, a microfluidic device comprises a sample port configured for introduction of a sample into the device. In certain embodiments, a sample port is operably connected and/or fluidically connected to one or more chambers. In some embodiments, a device comprises a sample port 605 and a sample chamber 100, where the sample port is proximal to the sample chamber. In some embodiments, a sample port 605 is configured for introduction of a sample into the sample chamber 100. In some embodiments, a sample port is located proximal to a sample chamber. In some embodiments, a sample port is a sample injection port.

In some embodiments, a device comprises a waste chamber (e.g., 200) configured for collection of fluid and wash solutions that have contacted a membrane (e.g., 104). In some embodiments, a waste chamber is operably and/or fluidically connected to a membrane (e.g., 104). A waste chamber (e.g., 200) can be located downstream of a membrane (e.g., 104), and or downstream of a sample chamber and or wash chamber such that excess fluid and wash buffers can be diverted into the waste chamber (e.g., by opening proximal valve V5, FIG. 24) after contacting the membrane. In some embodiments, a waste chamber (e.g., 200) is operably connected to a pump (e.g., a diaphragm or syringe style pump) capable of inducing a negative pressure that can divert fluid flow from the membrane (e.g., 104) into the waste chamber (e.g., 200) when valve V5 is open.

In some embodiments, a device comprises an elution collection chamber (e.g., 201) that is operably and/or fluidically connected to a membrane (e.g., 104) and an amplification chamber (e.g., 208) wherein the elution collection chamber is distal to (i.e., downstream of) the membrane. In some embodiments, the elution collection chamber is proximal to the amplification chamber. An elution collection chamber is configured to temporarily collect nucleic acids that are eluted from membrane 104. Nucleic acids that are disposed within an elution chamber can subsequently be diverted to an amplification chamber. In some embodiments, a device comprises a reagent chamber (e.g., 204) and/or a mixing chamber (e.g., 206) that are operably and/or fluidically connected to a proximal membrane (e.g., 104) and/or a proximal elution chamber (e.g., 201). In some embodiments, a reagent chamber (e.g., 204) and/or a mixing chamber (e.g., 206) are operably and/or fluidically connected to an amplification chamber (e.g., 104). In some embodiments, a reagent chamber and a mixing chamber are located adjacent to each other, where the mixing chamber is downstream and distal to the reagent chamber. In some embodiments, a reagent chamber and mixing chamber are located between a membrane and an amplification chamber.

In certain embodiments, reagents are disposed within a reagent chamber (e.g., 204, 218). Reagents disposed within a reagent chamber may be dried and or lyophilized. In some embodiments, reagents disposed within a reagent chamber are solubilized or dispersed in a liquid. In certain embodiments, dried or lyophilized reagents located within a reagent chamber are substantially solubilized when contacted with a fluid (e.g., eluted nucleic acids) when a fluid enters the reagent chamber. A downstream mixing chamber (e.g., 206) often aids in the solubilization process. In some embodiments, solubilization is aided by a downstream or distal microfluidic channel arranged in a serpentine configuration (e.g., see “Local mix 1” and “Local mix 2” in FIG. 24). Accordingly, in some embodiments, a mixing chamber (e.g., 206, 216) and/or a serpentine channel are located distal to a reagent chamber (e.g., 204, 218). In some embodiments, a reagent chamber (e.g., 204, 218) comprises one or more reagents, non-limiting examples of which include amplification primers, one or more blocking oligonucleotides, one or more polymerases (e.g., a heat stable polymerase), an exonuclease (e.g., a 5′-3′ exonuclease), dNTPs, salts, buffers, detergents, the like and combinations thereof. In some embodiments, a reagent chamber located proximal to an amplification chamber comprises a polymerase. In some embodiments, a reagent chamber located distal to an amplification chamber comprises an exonuclease.

In some embodiments, a device comprises an amplification chamber. An amplification chamber is configured to perform an amplification process (e.g., polymerase chain reaction (PCR)). In some embodiments, an amplification chamber is located distal to (i.e., downstream of) a sample chamber and/or a membrane, and proximal to a sensor. In some embodiments, an amplification chamber is operably connected to a heating source and/or cooling source. In certain embodiments, an amplification chamber comprises a heating source and/or a cooling source. Any suitable heating or cooling source can be used in a device described herein. In some embodiments, a heating or cooling source is located proximal to an amplification chamber, such that a temperature of a fluid entering into an amplification chamber can be regulated and/or adjusted. In some embodiments, an amplification chamber comprises one or more amplification reagents (e.g., dried reagents), non-limiting examples of which include primers, blocking oligonucleotides, salts, buffers, a polymerase, a detergent, dNTPs, the like and combinations thereof. In some embodiments, an amplification chamber comprises a surface disposed within the amplification chamber, where the surface is operably and/or fluidically connected to one or more components of the device. In some embodiments, a surface of an amplification chamber comprises one or more primers or blocking oligonucleotides that are covalently attached to the surface of the chamber. For example, in some embodiments a first primer is attached to the surface of the amplification chamber and a second primer comprising a member of a binding pair is not attached to the surface of the chamber, such that amplicons derived from the first primer remain in the chamber. In some embodiments, an amplification chamber (e.g., 208) is operably and/or fluidically connected to a distal reagent chamber (e.g., 218).

In some embodiments, a microfluidic device comprises a sensor (e.g., 300, e.g., a magnetic sensor). Any suitable sensor can be used for a device or method described herein, non-limiting examples of which include a camera (e.g., digital camera, a coupled-charge device (CCD) camera), a light sensing diode, a photocell, mass spectrometer, a fluorescence microscope, a confocal laser scanning microscope, laser scanning cytometer, a magnetic sensor (e.g., a giant magnetoresistance (GMR) sensor), the like and combinations thereof. In some embodiments a sensor is a magnetic sensor. In some embodiments a magnetic sensor is a magnetoresistance sensor. In some embodiments a magnetic sensor is a giant magnetoresistance (GMR) sensor. In some embodiments a magnetic sensor is an anisotropic magnetoresistance (AMR) sensor and/or a magnetic tunnel junction (MTJ) sensors. In some embodiments, a magnetic sensor detects magnetoresistance, current and/or voltage potential, or changes thereof. In some embodiments, a magnetic sensor detects magnetoresistance, current and/or voltage potential, or changes thereof on the surface of the sensor. In some embodiments, a magnetic sensor detects magnetoresistance, current and/or voltage potential, or changes thereof over a period of time non-limiting examples of which include 1 nanosecond to 1 hour, 1 second to 60 minutes, 1 second to 10 minutes, 1 second to 1000 seconds or intervening periods thereof. In some embodiments, a magnetic sensor detects the presence, absence or amount of magnetic particles that are bound to (e.g., indirectly bound to) or associated with a surface of the magnetic sensor according to a magnetoresistance, current and/or voltage potential, or changes thereof, that are detected by the magnetic sensor. In some embodiments, a magnetic sensor detects the presence, absence or amount of a genetic variation present in a sample according to a presence, absence or amount of magnetic particles that are bound to (e.g., indirectly bound to) or associated with a surface of the magnetic sensor. Accordingly, in some embodiments, a magnetic sensor detects the presence, absence or amount of a genetic variation present in a sample according to a magnetoresistance, current and/or voltage potential, or changes thereof, that are detected or measured at the surface of the magnetic sensor.

In some embodiments, a sensor comprises a capture nucleic acid. In some embodiments, a capture nucleic acid is attached (e.g., covalently) to a surface of a sensor using a suitable chemistry, non-limiting examples of which include a chemistry described in Cha et al. (2004) “Immobilization of oriented protein molecules on poly(ethylene glycol)-coated Si(111)” Proteomics 4:1965-1976 and Zellander et al. (2014) “Characterization of Pore Structure in Biologically Functional Poly(2-hydroxyethyl methacrylate)-Poly(ethylene glycol) Diacrylate (PHEMA-PEGDA),” PLOS ONE 9(5):e96709.

In some embodiments a sensor is located distal to an amplification chamber. In some embodiments, a sensor comprises a surface disposed on the sensor. In some embodiments, one or more capture nucleic acids are attached (e.g., covalently) to the surface of a sensor. In some embodiments a device comprises two or more sensors, each comprising a surface comprising a different capture nucleic acid. In some embodiments, a surface of a sensor comprises addressable locations, each comprising a different capture nucleic acid. In some embodiments, a sensor is disposed within a microfluidic channel. In some embodiments, a sensor is disposed within a chamber that is operably and/or fluidically connected to other components of the device. In some embodiments, a device comprises a heating and/or cooling source. In some embodiment, a sensor is operably connected to a heating and/or cooling source (e.g., 210) that is configured to regulate, maintain, increase and/or decrease the temperature of fluid that contacts a sensor. In some embodiments, a device comprises a heating and/or cooling source located proximal to a sensor.

In some embodiments, a device comprises a particle chamber (e.g., a magnetic particle (MNP) chamber (e.g., 230)) located proximal to (upstream of) a sensor. A particle chamber often comprises particles, where the particle are often attached to a member of a binding pair (e.g., streptavidin). Particles housed within a particle chamber can be lyophilized or dispersed within a fluid. In some embodiments, a particle chamber is operably and/or fluidically connected to a valve (e.g., V13) that when open, disperses particles into a microfluidic channel that is upstream or proximal to a sensor, such the particles proceed to contact and/or flow over the sensor.

In some embodiments, a device comprises a magnetic particle (MNP) chamber (e.g., 230) located proximal to a magnetic sensor. A MNP chamber often comprises magnetic particles. In some embodiments, magnetic particles in an MNP chamber are attached to a member of a binding pair (e.g., streptavidin). Magnetic particles housed within an MNP chamber can be lyophilized or dispersed within a fluid. In some embodiments, an MNP chamber is operably and/or fluidically connected to a valve (e.g., V13) that when open, disperses magnetic particles into a microfluidic channel that is upstream or proximal to a magnetic sensor, such the magnetic particles proceed to contact and/or flow over the magnetic sensor.

In some embodiments, one or more wash chambers (e.g., 250) are located proximal to a sensor where each wash chamber (e.g., 250) comprises a wash buffer. In some embodiments, the wash buffer comprises one or more positively charged ions (e.g., Mg⁺⁺, Ca⁺⁺, Na⁺, K⁺, the like or combinations thereof). In some embodiments, a wash chamber comprises one or more reagents selected from a salt, a buffer, a detergent, an alcohol, the like and combinations thereof.

In some embodiments, a device comprises one or more waste chambers (e.g., 400) located distal to a sensor.

In certain embodiments, a microfluidic device is disposed on a card or cartridge. Accordingly, in some embodiments, a microfluidic device, or a card or cartridge comprising a microfluidic device described herein has a length of 3 to 10 cm, a width of 1 to 10 cm, and a thickness of 0.1 to 1 cm.

In some embodiments, a microfluidic device comprises a printed circuit board (PSB) 502. In some embodiments a PSB comprises one or more electrical pad connections (e.g., 500). In some embodiments the one or more electrical pad connections of a PSB are operably (e.g., electronically) connected to one or more valves (e.g., 120), a sensor and/or one or more pumps of a microfluid device. In some embodiments a PSB comprises one or more components non-limiting examples of which include a sample chamber (e.g., 100), a membrane (e.g., 104), valves (e.g., 120), amplification chambers (e.g., 208), sensors, waste chambers (e.g., 200, 400), wash chambers (e.g., 101, 102, 250), control ports (e.g., 602), magnetic particle storage chamber (230), heat zones (e.g., 208, 210), mixing chambers (e.g., 206, 216), reagent chambers (e.g., 204, 218), microfluidic channel(s) (e.g., 105), the like or combinations thereof, wherein one or more, or all of the components are operably and/or fluidically connected to each other by one or more microfluid channels and/or associated valves.

In some embodiments, a microfluidic device is disposed on a cartridge or card (e.g., 600) that comprises a PSB, and one or more components selected from a sample chamber 100, membrane 104, valves 120, amplification chambers 208, sensors, waste chambers (e.g., 200, 400), wash chambers (e.g., 101, 102, 250), control ports (602), magnetic particle storage chamber (230), heat zones (e.g., 208, 210), mixing chambers (206, 216), reagent chambers (e.g., 204, 218) and microfluidic channel(s) (105), wherein one or more, or all of the components are operably and/or fluidically connected to each other by the microfluid channel(s) and/or associated valves. In some embodiments, a cartridge 600 is configured for insertion or attachment to a controller, memory and/or computer. In some embodiments a controller comprises pumps (e.g., diaphragm or syringe type pumps) that operably connect to one or more flow control ports 602 located on a cartridge.

In some embodiments, a microfluidic device, PSB or cartridge described herein comprises one or more components, subcomponents or parts described in International Patent Application No. PCT/US2019/043720, entitled “SYSTEM AND METHOD FOR GMR-BASED DETECTION OF BIOMARKERS” (Attorney Docket No. 026462-0504846) filed Jul. 26, 2019, International Patent Application No. PCT/US2019/043753, entitled “SYSTEM AND METHOD FOR SAMPLE PREPARATION IN GMR-BASED DETECTION OF BIOMARKERS” (Attorney Docket No. 026462-0504847) filed Jul. 26, 2019, International Patent Application No. PCT/US2019/043766, entitled “SYSTEM AND METHOD FOR SENSING ANALYTES IN GMR-BASED DETECTION OF BIOMARKERS” (Attorney Docket No. 026462-0504848) filed Jul. 26, 2019 or, International Patent Application No. PCT/US2019/043791, entitled “SYSTEM AND METHOD FOR PROCESSING ANALYTE SIGNALS IN GMR-BASED DETECTION OF BIOMARKERS” (Attorney Docket No. 026462-0504850) filed Jul. 26, 2019, all of which are hereby incorporated by reference herein in their entirety. In some embodiments, a method described herein utilizes one or more components, subcomponents or parts described in International Patent Application No. PCT/US2019/043720, PCT/US2019/043753, PCT/US2019/043766, or PCT/US2019/043791. In some embodiments, a microfluidic device described herein comprises a magnetic sensor and/or magnetic sensor assembly described in International Patent Application No. PCT/US2019/043720, PCT/US2019/043753, PCT/US2019/043766, or PCT/US2019/043791.

In some embodiments, any one chamber (e.g., 00-103, 200, 201, 204, 206, 208, 210, 216, 218, 230, 250) and/or a chamber housing a membrane or a chamber housing a sensor comprises a volume independently selected from 1 μl to 20 ml, 1 μl to 15 ml, 1 μl to 5 ml, 1 μl to 1 ml, 1 μl to 500 μl, 1 μl to 100 μl, and intermediate volumes thereof. In some embodiments, a chamber housing a membrane comprises a volume of 10 μl to 500 μl. In some embodiments, a chamber housing a sensor comprises a volume of 100 μl to 1000 μl.

The following is a non-limiting list of applications of analyte sensing that may be accomplished, in accordance with the principles detailed herein.

(1) Blood or other biological or environmental samples samples can include analytes, such as nucleic acid, such as DNA, RNA, and the like, that can be measured by employing the microfluidic devices, GMR devices, and genetic variation detection assays disclosed herein and throughout. Exemplary, non-limiting disease states associated with such analytes analytes that may be detected are summarized in Table 1 below.

TABLE 1

| Diseases | Analytes |
|---|---|
| Cardiac | Apolipoprotein A1, Apolipoprotein B, CK-MB, hsCRP, Cystatin |
| | C, D-Dimer, GDF-15, Myoglobin, NT-proBNP, BNP, Troponin I, |
| | Troponin T; genetic and/or allelic variants of the above. |
| Cancer | AFP, CA 125, CA 15-3, CA 19-9, CA 72-4, CEA, Cyfra 21-1, |
| | hCG plus beta, HE4, NSE, proGRP, PSA free, PSA total, SCC, S- |
| | 100, Thyreoglobulin (TG II), Thyreoglobulin confirmatory, b2- |
| | Microglobulin, KRAS, EGFR; genetic and/or allelic variants of the above. |
| Drugs of Abuse | Acetaminophen/Paracetamol (APAP), Amphetamines (AMP), |
| | Methamphetamines (mAMP), Barbiturates (BAR), |
| | Benzodiazepines (BZO), Cocaine (COC), Methadone (MTD), |
| | Opiates (OPI), Phencyclidine (PCP), THC, and Tricyclic Antidepressants (TCA). |
| Infectious | Anti-HAV, Anti-HAV IgM, Anti-HBc, Anti-HBc IgM, Anti-Hbe, |
| | HBeAg, Anti-HBs, HBsAg, HBsAg confirmatory, HBsAg |
| | quantitative, Anti-HCV, Chagas4, CMV IgG, CMV IgG Avidity, |
| | CMV IgM, HIV combi PT, HIV-Ag, HIV-Ag confirmatory, HSV- |
| | 1 IgG, HSV-2 IgG, HTLV-I/II, Rubella IgG, Rubella IgM, |
| | Syphilis, Toxo IgG, Toxo IgG Avidity, Toxo IgM, TPLA |
| | (Syphilis); genetic and/or allelic variants of the above. |
| Inflammation | Anti-CCP, ASLO, C3c, Ceruloplasmin, CRP, Haptoglobin, IgA, |
| | IgE, IgG, IgM, Immunglobulin A CSF, Immunglobulin M CSF, |
| | Interleukin 6, Kappa light chains, Kappa light chains free, Lambda |
| | light chains, Lambda light chains free, Prealbumin, Procalcitonin, |
| | Rheumatoid factor, a1-Acid Glycoprotein, a1-Antitrypsin, SSA; |
| | genetic and/or allelic variants of the above. |
| Pathogenic | Candida auris, Candida albicans, Candida tropicalis, Candida |
| Organisms | parapsilosis, Candida glabrata, Candida krusei, Candida |
| | haemulonis, Aspergillus fumigatus, Aspergillus flavus, Aspergillus |
| | niger, Aspergillus terreus, Cryptococcus neoformans, |
| | Cryptococcus gattii, Coccidioides immitis, Coccidioides posadasii, |
| | Fusarium solani, Fusarium oxysporum, Fusarium verticillioidis, |
| | Fusarium moniliforme, Pneumocystis jirovecii, Blastomyces |
| | dermatitidis, Histoplasma capsulatum, Rhizopus oryzae, Rhizopus |
| | microspores, Candida auris; genetic and/or allelic variants of the above. |

(2) GMR systems described herein may be use in urine analyte detection. Any protein, nucleic acid, such as DNA, RNA, and the like, metal or other substance in urine can be measured and/or detected by the GMR devices described herein. Urine associated biomarkers include, without limitation, preeclampsia, human chorionic gonadotropin (hCG), kidney injury molecule-1 (KIM-1), neutrophil gelatinase-associated lipocalin (NGAL), interleukin (IL)-18, and fatty-acid binding proteins (FABPs), nuclear matrix protein 22 (NMP22), BLCA-4, and epidermal growth factor receptor (EGFR), etc. Drugs and/or their major urinary metabolites include Acetaminophen/Paracetamol (APAP), Amphetamines (AMP), Methamphetamines (mAMP), Barbiturates (BAR), Benzodiazepines (BZO), Cocaine (COC), Methadone (MTD), Opiates (OPI), Phencyclidine (PCP), THC, and Tricyclic Antidepressants (TCA), etc.

(3) GMR systems described herein may be use in saliva analyte detection. Any protein, DNA, metal or other substance in saliva or mouth epithelium can be measured and/or detected by the GMR devices described herein. Exemplary biomarkers include, without limitation, matrix metalloproteinases (i.e., MMP1, MMP3, MMP9), cytokines (i.e., interleukin-6, interleukin-8, vascular endothelial growth factor A (VEGF-A), tumor necrosis factor (TNF), transferrins, and fibroblast growth factors, myeloid-related protein 14 (MRP14), profilin, cluster of differentiation 59 (CD59), catalase, and Mac-2-binding protein (M2BP), etc. Drugs include Amphetamines (AMP), Barbiturates (BAR), Benzodiazepines (BZO), Buprenorphine (BUP), Cocaine (COC), Cotinine (COT), Fentanyl (FYL), K2/Spice (K2), Ketamine (KET), Methamphetamine (MET), Methadone (MTD), Opiates (OPI), Oxycodone (OXY), Phencyclidine (PCP), Marijuana (THC), and Tramadol (TML).

(4) GMR systems described herein may be use in ocular fluid analyte detection. Any protein, DNA, metal or other substance in ocular fluid can be measured and/or detected by the GMR devices described herein. Ocular fluid biomarkers include, without limitation α-enolase, α-1 acid glycoprotein 1, S100 A8/calgranulin A, S100 A9/calgranulin B, S100 A4 and S100 A11 (calgizzarin), prolactin-inducible protein (PIP), lipocalin-1 (LCN-1), lactoferrin and lysozyme, b-amyloid 1-40, Neutrophil defensins NP-1 and NP-2, etc, can be measured in accordance with the assays and devices disclosed herein.

(5) Embodiments disclosed herein may employ a liquid biopsy as a sample for query analytes, such as biomarkers. In some such embodiments, there may be provided methods for identifying cancer in patients' blood. Methods described herein may be used to detect “rare” mutations in DNA found in the blood. DNA from cancer cells frequently enter the blood stream, however most of the blood borne DNA (>99%) will be from healthy cells. The methods disclosed herein provide for detecting these “rare” mutations and verifying the results. Methods disclosed herein provide for a multistep process to be captured in a single assay using a GMR detection platform.

In some such embodiments, there may be provided methods for detecting and/or distinguishing between one or more organisms present, or suspected of being present, in one or more samples. Methods disclosed herein may be used to detect and/or distinguish one ore more pathogenic organisms by employing nucleic acid probes that are designed to distinguish between the one or more pathogenic organisms in accordance with the assays and devices disclosed herein. Exemplary, non-limiting organisms which may be detected and or distinguished from one or more samples using the assays and devices disclosed herein include, for example, Candida auris, Candida albicans, Candida tropicalis, Candida parapsilosis, Candida glabrata, Candida krusei, Candida haemulonis, Aspergillus fumigatus, Aspergillus flavus, Aspergillus niger, Aspergillus terreus, Cryptococcus neoformans, Cryptococcus gattii, Coccidioides immitis, Coccidioides posadasii, Fusarium solani, Fusarium oxysporum, Fusarium verticillioidis, Fusarium moniliforme, Pneumocystis jirovecii, Blastomyces dermatitidis, Histoplasma capsulatum, Rhizopus oryzae, Rhizopus microspores, and Candida auris.

Methods disclosed herein comprise extracting nucleic acid, such as DNA, RNA, and/or the like, from blood, saliva, semen, or other biological sample, or from an environmental sample, which in accordance with embodiments herein, are automated in a cartridge which can perform the requisite extract and purification of DNA from the sample. In some embodiments, a silica membrane is employed as part of the extraction process, but methods herein are not so limited. After extraction and purification, the methods provide for selectively amplifying the query biomarker of interest. In some embodiments, methods for amplifying just the cancer DNA involves the use of locked nucleic acids to act as a blocker to prevent normal DNA from being amplified. Other selective amplification methods are known in the art. The next step in the methods is detecting whether the cancer DNA biomarker of interest is present in the patient sample. In some embodiments, this is achieved using exonuclease to convert double-stranded DNA (dsDNA) to single-stranded DNA (ssDNA). Other ways to convert dsDNA to ssDNA are known in the art. The methods continue with capturing the ssDNA by using a complimentary segment of DNA printed on the biosurface. In some embodiments, the ssDNA has a biotin attached to the end, and this biotin captures a streptavidin tagged magnetic bead. In some embodiments, methods include verifying whether the ssDNA (from the patient) is perfectly complimentary to the printed probe (synthetic segment of DNA). Verification can be accomplished using heat to denature the binding between two pieces of DNA. Imperfect binding will denature (or separate) at a lower temperature, than the perfect binding. This allows for verification of the signal, determining if the signal is caused by a true-positive or a false-positive. By using this verification step one can achieve a higher level of accuracy in diagnosing patients. There are other methods besides heating to denature DNA are known in the art.

Provided herein are methods and compositions for analyzing nucleic acids. In some embodiments, nucleic acid fragments in a mixture of nucleic acid fragments are analyzed. Nucleic acid may be isolated from any type of suitable biological specimen or sample (e.g., a test sample). In some embodiments, a sample comprises nucleic acids. A sample or test sample can be any specimen that is isolated or obtained from a subject (e.g., a mammal, a human). Non-limiting examples of specimens include fluid or tissue from a subject, including, without limitation, blood, amniotic fluid, cerebrospinal fluid, spinal fluid, lavage fluid (e.g., bronchoalveolar, gastric, peritoneal, ductal, ear, arthroscopic), a biopsy sample, urine, feces, sputum, saliva, nasal mucous, prostate fluid, lavage, semen, lymphatic fluid, bile, tears, sweat, breast milk, the like or combination thereof In some embodiments, a biological sample is blood, or a blood product (e.g., plasma or serum). Nucleic acid may be derived from one or more samples or sources.

In some embodiments, a sample is contacted with one or more suitable cell lysis reagents. Lysis reagents are often configured to lyse whole cells, and/or separate nucleic acids from contaminants (e.g., proteins, carbohydrates and fatty acids). Non-limiting examples of cell lysis reagents include detergents, hypotonic solutions, high salt solutions, alkaline solutions, organic solvents (e.g., phenol, chloroform), chaotropic salts, enzymes, the like, or combination thereof. Any suitable lysis procedure can be utilized for a method described herein.

The term “nucleic acid” refers deoxyribonucleic acid (DNA, e.g., complementary DNA (cDNA), genomic DNA (gDNA) and the like) and/or ribonucleic acid (RNA, e.g., mRNA, short inhibitory RNA (siRNA)), DNA or RNA analogs (e.g., containing base analogs, sugar analogs and/or a non-native backbone and the like), RNA/DNA hybrids and polyamide nucleic acids (PNAs), the like and combinations thereof. Nucleic acids can be single- or double-stranded. In some embodiments, a nucleic acid is a primer. In some embodiments, a nucleic acid is a target nucleic acid. A target nucleic acid is often a nucleic acid of interest.

Nucleic acid may be provided for conducting methods described herein without processing of a sample containing the nucleic acid, in certain embodiments. In some embodiments, nucleic acid is provided for conducting methods described herein after processing of a sample containing the nucleic acid. For example, a nucleic acid can be extracted, isolated, purified, partially purified or amplified from a sample prior to, during or after a method described herein.

In some embodiments, a nucleic acid is amplified by a process comprising nucleic acid amplification wherein one or both strands of a nucleic acid are enzymatically replicated such that copies or complimentary copies of a nucleic acid strand are generated. Copies of a nucleic acid that are generated by an amplification process are often referred to as amplicons. A nucleic acid amplification process can linearly or exponentially generates amplicons having the same or substantially the same nucleotide sequence as a template or target nucleic acid, or segment thereof. A nucleic acid may be amplified by a suitable nucleic acid amplification process non-limiting examples of which include polymerase chain reaction (PCR), nested (n) PCR, quantitative (q) PCR, real-time PCR, reverse transcription (RT) PCR, isothermal amplification (e.g., loop mediated isothermal amplification (LAMP)), quantitative nucleic acid sequence-based amplification (QT-NASBA), the like, variations thereof, and combinations thereof. In some embodiments, an amplification process comprises a polymerase chain reaction. In some embodiments, an amplification process comprises an isothermal amplification process.

In some embodiments, a nucleic acid amplification process comprises the use of one or more primers (e.g., a short oligonucleotide that can hybridize specifically to a nucleic acid template or target). A hybridized primer can often be extended by a polymerase during a nucleic acid amplification process). In some embodiments, a sample comprising nucleic acids is contacted with one or more primers. In some embodiments, a nucleic acid is contacted with one or more primers. A primer can be attached to a solid substrate or may be free in solution.

In some embodiments a nucleic acid or primer, comprises one or more distinguishable identifiers. Any suitable distinguishable identifier and/or detectable identifier can be used for a composition or method described herein. In certain embodiments a distinguishable identifier can be directly or indirectly associated with (e.g., bound to) a nucleic acid. For example, a distinguishable identifier can be covalently or non-covalently bound to a nucleic acid. In some embodiments a distinguishable identifier is attached to a member of binding pair that is covalently or non-covalently bound to a nucleic acid. In some embodiments a distinguishable identifier is reversibly associated with a nucleic acid. In certain embodiments a distinguishable identifier that is reversibly associated with a nucleic acid can be removed from a nucleic acid using a suitable method (e.g., by increasing salt concentration, denaturing, washing, adding a suitable solvent and/or by heating).

In some embodiments a distinguishable identifier is a label. In some embodiments a nucleic acid comprises a detectable label, non-limiting examples of which include a radiolabel (e.g., an isotope), a metallic label, a fluorescent label, a chromophore, a chemiluminescent label, an electrochemiluminescent label (e.g., Origen™), a phosphorescent label, a quencher (e.g., a fluorophore quencher), a fluorescence resonance energy transfer (FRET) pair (e.g., donor and acceptor), a dye, a protein (e.g., an enzyme (e.g., alkaline phosphatase and horseradish peroxidase), an enzyme substrate, a small molecule, a mass tag, quantum dots, the like or combinations thereof. Any suitable fluorophore can be used as a label. A light emitting label can be detected and/or quantitated by a variety of suitable methods such as, for example, by a photocell, digital camera, flow cytometry, gel electrophoresis, exposure to film, mass spectrometry, cytofluorimetric analysis, fluorescence microscopy, confocal laser scanning microscopy, laser scanning cytometry, the like and combinations thereof.

In some embodiments a distinguishable identifier is a barcode. In some embodiments a nucleic acid comprises a nucleic acid barcode (e.g., indexing nucleotides, sequence tags or “barcode” nucleotides). In certain embodiments a nucleic acid barcode comprises a distinguishable sequence of nucleotides usable as an identifier to allow unambiguous identification of one or more nucleic acids (e.g., a subset of nucleic acids) within a sample, method or assay. In certain embodiments a nucleic acid barcode is specific and/or unique to a certain sample, sample source, a particular nucleic acid genus or nucleic acid species, chromosome or gene, for example.

In some embodiments a nucleic acid or primer comprises one or more binding pairs. In some embodiments a nucleic acid or primer comprises one or more members of a binding pair. In some embodiments a binding pair comprises at least two members (e.g., molecules) that bind non-covalently and specifically to each other. Members of a binding pair often bind reversibly to each other, for example where the association of two members of a binding pair can be dissociated by a suitable method. Any suitable binding pair, or members thereof, can be utilized for a composition or method described herein. Non-limiting examples of a binding pair includes antibody/antigen, antibody/antibody receptor, antibody/protein A or protein G, hapten/anti-hapten, sulfhydryl/maleimide, sulfhydryl/haloacetyl derivative, amine/isotriocyanate, amine/succinimidyl ester, amine/sulfonyl halides, biotin/avidin, biotin/streptavidin, folic acid/folate binding protein, receptor/ligand, vitamin B 12/intrinsic factor, analogues thereof, derivatives thereof, binding portions thereof, the like or combinations thereof. Non-limiting examples of a member of a binding pair include an antibody or antibody fragment, antibody receptor, an antigen, hapten, a peptide, protein, a fatty acid, a glyceryl moiety (e.g., a lipid), a phosphoryl moiety, a glycosyl moiety, a ubiquitin moiety, lectin, aptamer, receptor, ligand, metal ion, avidin, neutravidin, biotin, B12, intrinsic factor, analogues thereof, derivatives thereof, binding portions thereof, the like or combinations thereof. In some embodiments, a nucleic acid or primer comprises biotin. In some embodiments, a nucleic acid or primer is covalently attached to biotin.

In some embodiments a nucleic acid or primer is attached non-covalently or covalently to a suitable solid substrate. In some embodiments, a capture oligonucleotide and/or a member of a binding pair is attached to a solid substrate. A capture oligonucleotide is often a nucleic acid configured to hybridize specifically to a target nucleic acid. In some embodiments a capture nucleic acid is a primer that is attached to a solid substrate. Non-limiting examples of a solid substrate include surfaces provided by microarrays and particles such as beads (e.g., paramagnetic beads, magnetic beads, microbeads, nanobeads), microparticles, and nanoparticles. Solid substrates also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semi-conductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, a solid substrate is coated using passive or chemically-derivatized coatings with any number of materials, including polymers, such as dextrans, acrylamides, gelatins or agarose. Beads and/or particles may be free or in connection with one another (e.g., sintered). In some embodiments, a solid substrate refers to a collection of particles. In some embodiments, particles comprise an agent that confers a paramagnetic property to the particles. In some embodiments a first solid substrate (e.g., a plurality of magnetic particles) is non-covalently and/or reversibly attached to a second solid substrate (e.g., a surface). In some embodiments, a second substrate or surface can be magnetized electronically such that magnetic particles are reversibly attached to the second substrate when the surface is magnetized, and the magnetic particles can be released when the second substrate is demagnetized or where the magnetic polarity of the second substrate is changed.

In some embodiments, a nucleic acid is a capture nucleic acid, such as a capture oligonucleotide. In some embodiments, a capture nucleic acid is a nucleic acid that is attached covalently or non-covalently to a solid substrate. A capture oligonucleotide typically comprises a nucleotide sequence capable of hybridizing or annealing specifically to a nucleic acid of interest (e.g. target nucleic acid) or a portion thereof. In some embodiments, a capture nucleic acid comprises a nucleic acid sequence that is substantially complimentary to a target nucleic acid, or portion thereof. In some embodiments, a capture oligonucleotide is a primer that is attached to a solid substrate. A capture oligonucleotide may be naturally occurring or synthetic and may be DNA or RNA based. Capture oligonucleotides can allow for specific separation of, for example, a target nucleic acid from other nucleic acids or contaminants in a sample.

In some embodiments, a method described herein comprises contacting a plurality of nucleic acids (e.g., nucleic acids in a sample) with at least one primer comprising a member of a binding pair. In some embodiments, a member of a binding pair comprise biotin. In some embodiments, the plurality of nucleic acids is contacted with a first primer and a second primer, where one of the first or second primers comprise biotin. In some embodiments, a plurality of nucleic acids comprises a target nucleic acid (e.g., a target RNA or DNA molecule). A target nucleic acid is often a nucleic acid of interested (e.g., a gene, a transcript or portion thereof). In some embodiments, a target nucleic comprises RNA. In some embodiments a target nucleic acid is amplified by a nucleic acid amplification process. In some embodiments, the nucleic amplification process comprises contacting a sample, nucleic acids of a sample and/or a target nucleic acid with a first primer, a second primer that is biotinylated and a polymerase under suitable conditions that promote nucleic acid amplification (e.g., conditions conducive to PCR or isothermal amplification). In some embodiments, a nucleic acid amplification process results in the production of amplicons. In some embodiments, amplicons comprise DNA amplicons, RNA amplicons, or a combination thereof. In some embodiments, amplicons comprise biotinylated DNA amplicons, RNA amplicons, or a combination thereof. In some embodiments, amplicons comprising RNA and biotinylated DNA (e.g., RNA/DNA duplexes) are contacted with a nuclease (e.g., an RNA exonuclease). In some embodiments, DNA amplicons are non-covalently attached to a solid substrate comprising a capture oligonucleotide, where the DNA amplicons, or a portion thereof, hybridize specifically to the capture oligonucleotide. In some embodiments, biotinylated amplicons are contacted with, and/or are attached to magnetic beads comprising streptavidin, or a variant thereof.

In some embodiments, the methods further comprise calculating a concentration of analyte in the query sample based on the magnetoresistance change of the GMR sensor.

In one or more of the preceding embodiments, methods include performing a buffer wash over the sensor prior to passing the query sample over the sensor.

In one or more of the preceding embodiments, methods include performing a buffer wash over the sensor after passing the query sample over the sensor but before passing the magnetic particles over the sensor.

In one or more of the preceding embodiments, methods include performing a buffer wash over the sensor after passing the magnetic particles over the sensor.

In one or more of the preceding embodiments, the query sample is water.

In one or more of the preceding embodiments, the query sample is derived from the blood of a subject.

In one or more of the preceding embodiments, methods include determining magnetoresistance change of the GMR sensor comprises using at least one reference resistor to perform phase-sensitive solution of magnetoresistance change of the GMR sensor.

In one or more of the preceding embodiments, a plurality of biomolecules are attached on the surface of the sensor in a density of about 1×10⁹ to about 5×10¹⁰ biomolecules per/mm².

In one or more of the preceding embodiments, a sensitivity limit of detection is in a range from about 1 nanomolar to about 10 nanomolar in the analyte.

In one or more of the preceding embodiments, passing the query sample over the detector comprises a flow rate of the query sample over the sensor at a rate of about 1 microL/min to about 20 microL/min.

In one or more of the preceding embodiments, at least one the first primer, second primer, blocking oligonucleotide, polymerase, capture nucleic acid, and query sample are mixed prior to passing them over the sensor.

In one or more of the preceding embodiments, at least one the first primer, second primer, blocking oligonucleotide, polymerase, and query sample is passed over the sensor after the capture nucleic acid is attached to the surface of the sensor.

In one or more of the preceding embodiments, the magnetic particles comprise streptavidin-linked particles.

In some embodiments, there are provided methods of detecting the presence of an analyte, such as a genetic variant, in a query sample comprising providing a sensor comprising a first biomolecule disposed on a functionalized surface of a giant magnetoresistance (GMR) sensor, the first biomolecule comprising a conditional binding site for a second biomolecule comprising a binding site for a magnetic particle, passing the query sample over the sensor, passing the second biomolecule over the sensor, passing magnetic particles over the sensor after passing the query sample over the sensor, and detecting the presence of the analyte in the query sample by measuring magnetoresistance change of the GMR sensor based on determining magnetoresistance before and after passing magnetic particles over the sensor, wherein determining magnetoresistance change of the GMR sensor comprises using at least one reference resistor to perform phase-sensitive solution of magnetoresistance change of the GMR sensor.

In some embodiments, there are provided methods of detecting the presence of an analyte, such as a genetic variant, in a query sample comprising providing a sensor comprising a first biomolecule disposed on a functionalized surface of a giant magnetoresistance (GMR) sensor, the first biomolecule comprising a conditional binding site for a second biomolecule comprising a binding site for a magnetic particle, passing the query sample over the sensor, passing the second biomolecule over the sensor, passing a plurality of magnetic nanoparticles comprising a first member of a binding pair over the sensor after passing the second biomolecule over the sensor, then passing a plurality of magnetic nanoparticles comprising a second member of the binding pair over the sensor and detecting the presence of the analyte by measuring an amplified magnetoresistance change of the GMR sensor based on determining magnetoresistance before and after passing magnetic particles over the GMR sensor. In some embodiments, such methods further comprise passing a second plurality of magnetic nanoparticles comprising the first member of a binding pair over the sensor after passing the plurality of magnetic nanoparticles comprising the second member of the binding pair over the sensor. In some embodiments, such methods further comprise passing a second plurality of magnetic nanoparticles comprising the second member of the binding pair over the sensor after passing the second plurality of magnetic nanoparticles comprising first second member of the binding pair over the GMR sensor. In some embodiments, such methods further comprise passing one or more subsequent pluralities of magnetic nanoparticles comprising the first member of the binding pair, and one or more subsequent pluralities of magnetic nanoparticles comprising the second member of the binding pair, over the GMR sensor. In some embodiments, the binding pair comprises streptavidin and biotin. In some embodiments, the first member of the binding pair comprises streptavidin. In some embodiments, the second member of the binding pair comprises biotin.

In one or more of the preceding embodiments, the presence of the analyte prevents the binding of the second biomolecule.

In one or more of the preceding embodiments, the presence of the analyte enables the binding of the second molecule to the first biomolecule.

In some embodiments, there are provided methods of detecting the presence of an analyte in a query sample comprising providing a sensor comprising a first biomolecule disposed on a functionalized surface of a giant magnetoresistance (GMR) sensor, the biomolecule comprising a binding site for a magnetic particle when the analyte is present, passing the query sample over the sensor, passing magnetic particles over the sensor after passing the query sample over the sensor, and detecting the presence of the analyte in the query sample by measuring magnetoresistance change of the GMR sensor based on determining magnetoresistance before and after passing magnetic particles over the sensor, wherein determining magnetoresistance change of the GMR sensor comprises using at least one reference resistor to perform phase-sensitive solution of magnetoresistance change of the GMR sensor.

In one or more of the preceding embodiments, methods may further comprise calculating a concentration of analyte in the query sample based on the magnetoresistance change of the GMR sensor.

In one or more of the preceding embodiments, the biomolecule comprises a nucleic acid, such as a target nucleic acid.

In some embodiments, there are provided systems configured to carry out the methods disclosed herein comprising, the system comprising a sample processing subsystem, a sensor subsystem comprising a microfluidics network comprising a GMR sensor having disposed on a functionalized surface of the sensor a biomolecule, a plurality of wires connected to a plurality of contact pads to carry a signal to a processor, a processor, and a pneumatic control subsystem for moving samples, reagents, and solvents throughout the sample processing subsystem and the sensor subsystem.

In one or more of the preceding embodiments, methods may further comprise performing a buffer wash over the sensor prior to passing the query sample over the sensor.

In one or more of the preceding embodiments, methods may further comprise performing a buffer wash over the sensor after passing the query sample over the sensor but before passing the magnetic particles over the sensor.

In one or more of the preceding embodiments, methods may further comprise performing a buffer wash over the sensor after passing the magnetic particles over the sensor.

In one or more of the preceding embodiments, the surface of the GMR sensor is functionalized by a crosslinked polymer composition comprising at least two hydrophilic polymers, such as PEG-PHEMA polymer.

In some embodiments a polymer composition comprising at least two hydrophilic polymers and a crosslinking reagent is employed to functionalize the surface of the GMR sensor.

In some embodiments the polymer composition comprises a PEG polymer, a PHEMA polymer, and a crosslinking reagent.

In one or more of the preceding embodiments, the polymer is coated with a surfactant.

In one or more of the preceding embodiments, the surfactant is cetyl trimethylammonium bromide.

In one or more of the preceding embodiments, sensors may further comprise a plurality of wires connected to a plurality of contact pads configured to carry an electronic signal from the sensor to a processor.

In one or more of the preceding embodiments, the microfluidics system is pneumatically controlled.

In one or more of the preceding embodiments, the cartridge further comprises one or more hardware chips to control flowrate throughout the microfluidics system.

In one or more of the preceding embodiments, the sensor is configured to be in electronic communication with a plurality of contact pins to carry an electronic signal from the sensor to a processor.

In one or more of the preceding embodiments, the magnetic particles comprise streptavidin-linked nanoparticles.

### Subjects

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation. A subject can be a subject having, or suspected of having a disease or condition characterized or caused by the presence in the subject of one or more organisms, such as pathogenic organisms.

### Samples

Provided herein are methods and compositions for analyzing a sample. In some embodiments, a sample is a liquid sample. In some embodiments a liquid sample is an aqueous sample. A liquid sample may comprise, in some embodiments, fine particulate matter suspended in a liquid. Solid samples (such as soil or tissues) can be washed or extracted with a liquid to obtain a liquid sample suitable for conducting a method described herein.

A sample can be obtained from any suitable environmental source or from a suitable subject. A sample isolated from an environmental source is sometimes referred to as an environmental sample, non-limiting examples of which include liquid samples obtained from a lake, stream, river, ocean, well, run-off, tap water, bottled water, purified or treated water, waste water, irrigation water, ice, snow, dirt, soil, waste, the like, and combinations thereof. In some embodiments, a sample is isolated, obtained or extracted from a product of manufacture, non-limiting examples of which include recycled materials, polymers, plastics, pesticides, wood, textiles, fabric, synthetic fibers, clothes, food, beverages, rubber, detergents, oils, fuels, the like, or combinations thereof.

In some embodiments, a sample is a biological sample, for example a sample obtained from a living organism or a subject. A sample can be isolated or obtained directly or indirectly from a subject or part thereof. In some embodiments, a sample is obtained indirectly from an individual or medical professional who then provides the sample for analysis. A sample can be any specimen that is isolated or obtained from a subject or part thereof. A sample can be any specimen that is isolated or obtained from multiple subjects. Non-limiting examples of biological samples include blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. In some embodiments, a sample is a cell free sample. In some embodiments, a liquid sample is obtained from cells or tissues using a suitable method. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof In some embodiments, a sample is filtered to remove insoluble matter or debris to obtain a liquid sample suitable for analysis by a method described herein.

In some embodiments, a sample is a fluid or liquid sample (e.g., blood or plasma) obtained from a subject. A sample may comprise cells or tissues that are normal, healthy, diseased (e.g., infected), and/or cancerous (e.g., cancer cells). A sample obtained from a subject may comprise cells or cellular material (e.g., nucleic acids) of multiple organisms (e.g., virus nucleic acid, fetal nucleic acid, bacterial nucleic acid, fungal nucleic acid, parasite nucleic acid, and the like).

In some embodiments a sample has a pH in a range of 4 to 10, 6 to 10, 7 to 10 or about 6 to 8.5. In some embodiments, a pH of a sample is adjusted to a pH in a range of 4 to 10, 6 to 10, 7 to 10 or about 6 to 8.5, or to prior to contacting the sample with a sensor.

In some embodiments, a sample comprises nucleic acid, or fragments thereof. A sample can comprise nucleic acids obtained from one or more subjects. In some embodiments a sample comprises nucleic acid obtained from a single subject. In some embodiments, a sample comprises a mixture of nucleic acids. A mixture of nucleic acids can comprise two or more nucleic acid species having different nucleotide sequences (e.g., different allelic sequences), different fragment lengths, different origins (e.g., genomic origins, cell or tissue origins, cancer or non-cancer origin, different subjects), the like, or combinations thereof.

### Nucleic Acids and Genes

The terms “nucleic acid” refers to one or more nucleic acids (e.g., a set or subset of nucleic acids), non-limiting examples of which include DNA (e.g., cDNA, genomic DNA (gDNA), cell-free DNA, mitochondrial DNA, microbial DNA, the like or combinations thereof), RNA (e.g., message RNA (mRNA), short inhibitory RNA (siRNA), ribosomal RNA (rRNA), tRNA, microRNA, nucleic acids comprising DNA or RNA analogs (e.g., containing base analogs, sugar analogs and/or a non-native backbone and the like), RNA/DNA hybrids and polyamide nucleic acids (PNAs), locked nucleic acids (LNAs), the like or combinations thereof, all of which can be single- or double-stranded, and unless otherwise limited, can encompass known analogs of natural nucleotides that can function in a similar manner as naturally occurring nucleotides. In some embodiments nucleic acid refers to genomic DNA. A nucleic acid can be of any length of, for example, 2 or more, 3 or more, 4 or more, 5 or more, 10 or more, 50 or more or 100 or more contiguous nucleotides. A nucleic acid typically comprises a specific 5′ to 3′ order of nucleotides known in the art as a sequence (e.g., a nucleic acid sequence, e.g., a sequence).

In some embodiments, a nucleic acid is a native nucleic acid (e.g., a naturally occurring nucleic obtained from a sample or subject). In some embodiments, a nucleic acid is synthesized, copied or altered (e.g., by a technician, scientist or one of skill in the art). In some embodiments, a nucleic acid is an amplicon (e.g., an amplification product) that is derived from an amplification reaction (e.g., PCR or a non-thermal or displacement amplification reaction). Amplicons can be single or double stranded and typically represent an exact copy or complementary copy of a nucleic acid template that was subjected to an amplification reaction. Oligonucleotides are relatively short nucleic acids. In some embodiments, a nucleic acid is an oligonucleotide. In some embodiments, an oligonucleotide is a single stranded nucleic acid having a length of about 4 to 150, 4 to 100, 5 to 50, or 5 to about 35 nucleic acids in length, or intermediate lengths thereof. In certain embodiments, oligonucleotides are primers. Primers are often configured to hybridize to a selected complementary nucleic acid and are configured to be extended by a polymerase after hybridizing. A “primer pair” refers to two primers configured to amplify a target nucleic acid.

A target nucleic acid is a nucleic acid that is subjected to analysis by a method described herein. Any nucleic acid of interest can be a target nucleic acid. In some embodiments a target nucleic acid is a nucleic acid suspected of having a genetic variation. In some embodiments a target nucleic acid comprises a gene or a portion thereof (e.g., a gene of interest). In some embodiments a target nucleic acid has a length of about 20 to about 100,000 nucleotides, about 20 to about 500 nucleotides, about 20 to about 400 nucleotides, about 20 to about 300 nucleotides, about 20 to about 200 nucleotides, about 20 to about 100 nucleotides, or about 20 to about 50 nucleotides.

In some embodiments a target nucleic acid comprises a gene of interest, or a portion thereof. In certain embodiments a gene of interest comprises, or is suspected of having, a genetic variation associated with a disease, condition or disorder. In certain embodiments a gene of interest comprises, or is suspected of having a genetic variation associated with a subjects predisposed to a disease, condition or disorder. A gene of interest may comprise exons, introns, 5′ flanking regions, 3′ flanking regions, plus strands and/or minus strands of a gene.

Locked Nucleic Acids

In some embodiments a nucleic acid (e.g., a blocking oligonucleotide, capture nucleic acid or primer) is a locked nucleic acid. In some embodiments, a locked nucleic acid comprises one or more modified nucleotide monomers termed locked nucleotides. Locked nucleotides are modified nucleotide bases that when present in a hybridized nucleic acid, increase the melting temperature of the hybridized duplex compared to the melting temperature of the same duplex that consists of only naturally occurring nucleotide bases. Non-limiting examples of locked nucleic acids include traditional locked nucleic acids (i.e., LNAs, e.g., bicyclic nucleic acids), bridged nucleic acids (BNAs, e.g., constrained nucleic acids), nucleic acids comprising CS-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, non-limiting examples of locked nucleotides include modified RNA nucleotides comprising a modified ribose moiety with an extra bridge connecting the 2′ oxygen and 4′ carbon, BNA monomers that comprise a five-membered, six-membered or even a seven-membered bridged structure (e.g., BNA monomers that include 2′,4′-BNANC[NH], 2′,4′-BNANC[NMe], and 2′,4′-BNANC[NBn]) and the like. Any suitable locked nucleotide (e.g., modified nucleotide) that increase the melting temperature of a hybridized nucleic acid duplex can be used to make a locked nucleic acid for use herein. In some embodiments, a locked nucleic acid is one disclosed in U.S. Patent Application No. 2003/0144231, which is incorporated herein by reference. In some embodiments, a locked nucleic acid comprises one or more locked nucleotides described in U.S. Patent Application No. 2003/0144231. Non-base modifiers can also be incorporated into a locked nucleic acid to increase Tm (or binding affinity), non-limiting examples of which include a minor grove binder (MGB), spermine, G-clamp, a Uaq anthraquinone cap, the like or combinations thereof. More than one type of Tm-enhancing modification can be employed in a locked nucleic acid (e.g., a blocking oligonucleotide or capture nucleic acid), such as a combination of locked nucleotide monomers and a terminal MGB group. Many methods of increasing the Tm of complementary nucleic acids are known to those of skill in the art and the use of all such modifications is considered within the scope of the inventions herein.

In some embodiments, a locked nucleic acid (e.g., a blocking oligonucleotide or capture nucleic acid) comprises at least 1, at least 2, at least 3, at least 4, at least 5, at least 6, at least 7, at least 8, at least 9, or at least 10 locked nucleotides. in some embodiments, a locked nucleic acid comprises 1 to 20, 1 to 10 or 1 to 5 locked nucleotides. In some embodiments, all of the nucleotides of a locked nucleic acid are locked nucleotides. In some embodiments, a locked nucleic acid comprises a length of at least 5 nucleotides. In some embodiments, a locked nucleic acid comprises a length a 5 to 100, 5 to 30 or 5 to 20 nucleotides, or intermediate ranges thereof, in some embodiments, a locked nucleic acid, when hybridized to a target nucleic acid, has a melting temperature of at least 50° C., at least 52° C., at least 55° C., at least 60° C., at least 65° C., at least 70° C., at least 75° C., or at least 80° C. in some embodiments, a locked nucleic acid, when hybridized to a target nucleic acid, has a melting temperature between about 40° C. and about 80° C., about 45° C. and about 80° C., about 50° C. and about 80° C., about 55° C. and about 80° C., about 60° C. and about 80° C. or between about 65° C. and about 80° C.

### Blocking Oligonucleotides

In some embodiments, a device or method comprises the use of a blocking oligonucleotide. In some embodiments, a blocking oligonucleotide is a locked nucleic acid. In some embodiments, a blocking oligonucleotide comprises at least 1, at least 2, at least 3, at least 4, at least 5, at least 6, at least 7, at least 8, at least 9, or at least 10 locked nucleotides. in some embodiments, a blocking oligonucleotide comprises 1 to 20, 1 to 10 or 1 to 5 locked nucleotides. In some embodiments, all of the nucleotides of a blocking oligonucleotide are locked nucleotides. In some embodiments, a blocking oligonucleotide comprises a length of at least 5 nucleotides. In some embodiments, a blocking oligonucleotide comprises a length of 5 to 100, 5 to 30 or 5 to 20 nucleotides, or intermediate ranges thereof. In some embodiments, a blocking oligonucleotide, when hybridized to a target nucleic acid, has a melting temperature of at least 50° C., at least 52° C., at least 55° C., at least 60° C., at least 65° C., at least 70° C., at least 75° C., or at least 80° C. In some embodiments, a blocking oligonucleotide, when hybridized to a target nucleic acid, has a melting temperature between about 40° C. and about 80° C., about 45° C. and about 80° C., about 50° C., and about 80° C., about 55° C. and about 80° C., about 60° C. and about 80° C., or between about 65° C. and about 80° C.

In certain embodiments, a blocking oligonucleotide is configured to hybridize to a target nucleic acid that does not comprise a genetic variation of interest. In some embodiments, a blocking oligonucleotide that is configured to hybridize to a target nucleic acid that does not comprise a genetic variation of interest, is an oligonucleotide comprising one or more locked nucleotides and a nucleic acid sequence that at least 98%, at least 99% or 100% identical to the compliment sequence of a nucleic acid (e.g., a target nucleic acid), or portion thereof, that does not include a genetic variation of interest (e.g., SNP or mutation of interest). A blocking oligonucleotide is often configured to substantially block amplification of a specific nucleic acid that may be present in an amplification reaction. In some embodiments, a blocking oligonucleotide is configured to substantially block amplification of a target nucleic acid that may be present in an amplification reaction, where the target nucleic acid does not include a genetic variation of interest. For example, a blocking oligonucleotide is often configured to form a hybridized duplex with a target nucleic acid (e.g., a target nucleic acid that does not contain a genetic variation of interest) wherein the duplex has a high melting temperature relative to the primers used in an amplification reaction.

### Primers

In some embodiments, a method or process comprises the use of one or more primers. In some embodiments, a nucleic acid amplification process comprises the use of one or more primers (e.g., a short oligonucleotide that can hybridize specifically to a nucleic acid template or target). A hybridized primer can often be extended by a polymerase during a nucleic acid amplification process). In some embodiments, a sample comprising nucleic acids is contacted with one or more primers. In some embodiments, nucleic acids (e.g., a target nucleic acid) is contacted with one or more primers. A primer can be attached to a solid substrate or may be free in solution. Any suitable primers can be used for a method described herein.

### Capture Nucleic Acids

In some embodiments a nucleic acid or primer is attached non-covalently or covalently to a suitable solid substrate. In certain embodiment, a capture nucleic acid is a nucleic acid or oligonucleotide that is attached non-covalently or covalently to a solid substrate. A capture oligonucleotide is often a nucleic acid configured to hybridize specifically to a target nucleic acid, or portion thereof. In some embodiments a capture nucleic acid is a primer that is attached to a solid substrate. In some embodiments, a capture nucleic acid comprises a nucleic acid sequence that is substantially complimentary, or exactly complementary to a target nucleic acid, or portion thereof. In some embodiments, a capture nucleic acid comprises a nucleic acid sequence that is at least 90%, at least 95%, at least 98%, or at least 99% identical to the complement or reverse compliment of a target nucleic acid, or portion thereof. In some embodiments, a capture nucleic acid comprises a nucleic acid sequence that is 100% identical to the complement or reverse compliment of a target nucleic acid, or portion thereof. A capture oligonucleotide may be naturally occurring or synthetic and may be DNA and/or RNA based, In some embodiments, a capture nucleic acid is a locked nucleic acid comprising one or more locked nucleotides. In some embodiments, a capture nucleic acid comprises at least 1, at least 2, at least 3, at least 4, at least 5, at least 6, at least 7, at least 8, at least 9, or at least 10 locked nucleic acids. In some embodiments, a capture nucleic acid comprises 1 to 20, 1 to 10 or 1 to 5 locked nucleotides, some embodiments, a capture nucleic acid, when hybridized to a target nucleic acid, has a melting temperature of at least 50° C., at least 52° C. at least 55° C., at least 60° C. at least 65° C. at least 70° C. at least 75° C., or at least 80° C. In some embodiments, a capture nucleic acid, when hybridized to a target nucleic acid, has a melting temperature between about 40° C. and about 80° C., about 45° C. and about 80° C., about 50° C. and about 80° C., about 55° C. and about 80° C., about 60° C. and about 80° C., or between about 65° C. and about 80° C.

### Detectable/Particles/Binding Pairs

In some embodiments, a method or process described herein comprises a use of one or more, or a plurality of detectable labels. In some embodiments, the one or more detectable labels comprise one or more magnetic particles. In some embodiments a primer, a probe, a blocking oligonucleotide, and/or a capture nucleic acid surface comprises one or more magnetic particles. In some embodiments a member of a binding pair comprises one or more magnetic particles. In some embodiments, a magnetic particle comprises a member of a binding pair. In some embodiments, a magnetic particle comprises a first member of a binding pair. In some embodiments, a magnetic particle comprises a second member of a binding pair. In some embodiments, a first magnetic particle comprises a first member of a binding pair. In some embodiments, a second magnetic particle comprises a second member of a binding pair. In sonic embodiments, a first plurality of magnetic particles comprises magnetic particles, wherein each member of the first plurality comprises a first member of a binding pair. In some embodiments, a second plurality of magnetic particles comprises magnetic particles, wherein each member of the second plurality comprise a second member of a binding pair.

In some embodiments, a magnetic particle, or each member of a first or a second plurality of magnetic particles, comprises a member of a binding pair comprising streptavidin. In some embodiments, a magnetic particle, or each member of a first or a second plurality of magnetic particles, comprises a member of a binding pair comprising biotin. In some embodiments, a magnetic particle, or each member of a first or a second plurality of magnetic particles, comprises a member of a binding pair comprising biotin. In some embodiments, a magnetic particle, or each member of a first plurality of magnetic particles, comprises a member of a binding pair comprising biotin. In some embodiments, a magnetic particle, or each member of a first plurality of magnetic particles, comprises a member of a binding pair comprising streptavidin. In some embodiments, a magnetic particle, or each member of a second plurality of magnetic particles, comprises a member of a binding pair comprising biotin. In some embodiments, a magnetic particle, or each member of a second plurality of magnetic particles, comprises a member of a binding pair comprising streptavidin.

A suitable magnetic particle can be used for a composition, device or method described herein. Non-limiting examples of magnetic particles include paramagnetic beads, magnetic beads, magnetic nanoparticles, heavy metallic microbeads, metallic nanobeads, heavy metallic microparticles, heavy metallic nanoparticles, the like or combinations thereof. In some embodiments, a magnetic particle comprises an average or absolute diameter of about 1 to about 1000 nanometers (nm), 1 nm to about 500 nm, about 5 nm to about 1000 nm, about 10 nm to about 1000 nm, about 5 nm to about 500 nm, about 5 nm to about 400 nm, about 5 nm to about 300 nm, about 5 nm to about 300 nm, about 5 nm to about 200 nm, about 5 nm to about 100 nm, about 2 to about 50 nm, about 5 to about 20 nm, or about 5 to about 10 nm, and/or ranges in between. In some embodiments, a magnetic particle is coated to facilitate covalent attachment to a member of a binding pair. In other embodiments a magnetic particle is coated to facilitate electrostatic association with molecules. In some embodiments magnetic particles comprises different shapes, sizes and/or diameters to facilitate different amounts of magnetism. In some embodiments, magnetic particles are substantially uniform (e.g., all are substantially the same; e.g., same size, same diameter, same shape and/or same magnetic properties) to facilitate more accurate detections and/or quantitation at the surface of the magnetic sensor. In some embodiments, magnetic beads comprise the same or different members of a binding pair to allow multiplex detection of multiple different analytes in the same query sample or in different query samples. In some embodiments, such analytes in the same sample or in different samples comprise one or more heavy metals. In some embodiments, the presence, absence and/or number of magnetic particles can be detected and/or quantitated by a suitable magnetic sensor. In some embodiments, a magnetic sensor comprises a surface.

In some embodiments a substrate, a particle (e.g., a magnetic particle), a bead, a protein, an antibody, a capture nucleic acid, or a surface, comprises one or more members of a binding pair. In some embodiments a capture nucleic acid comprises one or more members of a binding pair. In certain embodiments, a first member of a binding pair can bind, and/or binds to, a second member of a binding pair. In certain embodiments, a first member of a binding pair is configured to bind specifically to a second member of a binding pair. In some embodiments a binding pair comprises at least two members (e.g., molecules) that bind non-covalently and specifically to each other. Members of a binding pair often bind reversibly to each other, for example where the association of two members of a binding pair can be dissociated by a suitable method. Any suitable binding pair, or members thereof, can be utilized for a composition or method described herein. Non-limiting examples of a binding pair (e.g., first member/second member) include antibody/antigen, antibody/antibody receptor, antibody/protein A or protein G, antibody/GST, hapten/anti-hapten, sulfhydryl/maleimide, suflhydryl/haloacetyl derivative, amine/isotriocyanate, amine/succinimidyl ester, amine/sulfonyl halides, biotin/avidin, biotin/streptavidin, folic acid/folate binding protein, receptor/ligand, GST/GT, vitamin B12/intrinsic factor, analogues thereof, derivatives thereof, binding portions thereof, the like or combinations thereof. Non-limiting examples of a member of a binding pair include an antibody or antibody fragment, antibody receptor, an antigen, hapten, a peptide, protein, a fatty acid, a glyceryl moiety (e.g., a lipid), a phosphoryl moiety, a glycosl moiety, a ubiquitin moiety, lectin, aptamer, receptor, ligand, heavy metal ion, avidin, neutravidin, streptavidin, biotin, B12, intrinsic factor, analogues thereof, derivatives thereof, binding portions thereof, the like or combinations thereof.

In some embodiments, a nucleic acid or primer is covalently attached to member of a binding pair. In some embodiments, member of a binding pair is attached covalently to a primer. In some embodiments, a member of a binding pair is attached (e.g., covalently) to the free 5′hydroxyl of a primer. In some embodiments, a nucleic acid or primer comprises biotin. In some embodiments, biotin is attached covalently to a primer. In some embodiments, biotin is attached (e.g., covalently) to the free 5′hydroxyl of a primer.

In some embodiments, a method or process described herein comprises a use of one or more, or a plurality of magnetic particles. In some embodiments, a composition or device described herein comprises one or more magnetic particles. In some embodiments a nucleic acid, a substrate, a protein, an antibody, a secondary reagent, a bead, a surface, and/or an MPR comprises one or more magnetic particles. In some embodiments a member of a binding pair comprises one or more a magnetic particle. Ire some embodiments, a magnetic particle is attached to a member of a binding pair. In some embodiments, a magnetic particle comprises streptavidin, or a variant thereof. In certain embodiments a magnetic particle is directly or indirectly attached to (e.g., bound to, e,g., covalently or non-covalently) a nucleic acid, a substrate, an antibody, a secondary reagent, a bead, a surface, a member of a binding pair, and/or an MPR, or the like.

### Surfaces

In some embodiments. a sensor comprises a surface. In some embodiments, a surface of a sensor comprises one or more oligonucleotides or capture nucleic acids. A surface of a sensor may comprise a suitable material, non-limiting examples of which include glass, modified or functionalized glass (e.g., controlled-pore glass (CPG)), quartz, mica, polyformaldehyde, cellulose, cellulose acetate, ceramics, metals, metalloids, semi-conductive materials, plastic (including acrylics, polystyrene, copolymers of styrene or other materials, polybutylene, polyurethanes, TEFLON™, polyethylene, polypropylene, polyamide, polyester, polyvinylidenedifluoride (PVDF), and the like), resins, silica or silica-based materials including silicon, silica gel, and modified silicon, Sephadex®, SEPHAROSE®, carbon, metals (e.g., steel, gold, silver, aluminum, silicon and copper), conducting polymers (including polymers such as polypyrole and polyindole); micro or nanostructured surfaces, nanotube, nanowire, or nanoparticulate decorated surfaces; or porous surfaces or gels such as methacrylates, acrylamides, sugar polymers, cellulose, silicates, or other fibrous or stranded polymers. In some embodiments, a surface is functionalized using passive or chemically-derivatized coatings with any number of materials, including polymers, such as dextrans, acrylamides, gelatins or agarose. In some embodiments a surface of a sensor is non-covalently and/or reversibly attached to an oligonucleotide or capture nucleic acid. In some embodiments a surface of a sensor is covalently attached to an oligonucleotide or capture nucleic acid.

In some embodiments, a surface of a sensor comprises and/or is coated with a polymer composition comprising at least two hydrophilic polymers and a crosslinking reagent. In some embodiments, such polymer compositions, biosurfaces comprising such polymer compositions, and methods of functionalizing sensor surfaces with such polymer compositions in accordance with the methods and devices disclosed herein and throughout are described in, for example, U.S. Provisional Patent Application No. 62/958,510, entitled “POLYMER COMPOSITIONS AND BIOSURFACES COMPRISING THEM ON SENSORS,” filed on Jan. 8, 2020 (Attorney Docket No. 026462-0506342), which is hereby incorporated by reference in its entirety.

In some embodiments, a surface of a sensor comprises and/or is coated with a polymer composition comprising a crosslinked PEG-PHEMA polymer. A PEG-PHEMA polymer surface can be prepared by mixing a PEG solution comprising N-Hydroxysuccinimide (NHS)-PEG-NHS (MW 600) dissolved in a suitable solvent (e.g., isopropyl alcohol, acetone or methanol, and/or water), a PHEMA solution comprising polyhydroxyethyl methacrylate (MW 20,000) dissolved in a suitable solvent (e.g., isopropyl alcohol, acetone or methanol, and/or water), and an optional crosslinker. The resulting solution can be coated on a sensor surface using a suitable coating process (e.g., micro-printing, dip coating, spin coating or aerosol coating). After coating a surface with the PEG-PHEMA solution, the surface can be cured using UV light followed by washing with a suitable solvent, such as isopropyl alcohol and/or water. In some embodiments a surface of a sensor is covalently attached to one or more nucleic acids. In some embodiments, the coated surface can be used to bind with primary amines (e.g., to attach a protein). A PEG-PHEMA coating can protect a sensor surface against corrosion. In some embodiments, a surface of a sensor comprises a surface described in International Patent Application No. PCT/US2019/043766.

### Genetic Variations/Genetic Variants

In some embodiments, a a plurality of primers or sets of primers, capture nucleic acids, and/or detectable labels is employed in order to distinguish between pathogenic organisms that are present, or are suspected of being present, in a sample. In some embodiments, the sample is obtained from a biological source (living or dead). In some embodiments, the sample is obtained from a subject, such as a mammalian subject, such as a human subject. In some embodiments the sample is obtained from a patient. In some embodiments, the sample is obtained from an environmental source. In some embodiments, the sample is obtained from an environmental source, such as a water source, such as an ocean, lake, river, stream, swamp, lagoon, marsh, tidal pool, swimming pool, tributary, wastewater facility, wastewater reservoir, water reservoir, potable water reservoir, water treatment facility, and/or the like. In some embodiments, the sample is obtained from the environment, such as soil, dirt, sludges, slimes, scums, composts and the like.

In some embodiments, a nucleic acid (e.g., a target nucleic acid) comprises a genetic variation, also referred to interchangeably throughout as a genetic variant, non-limiting examples of which include one or more nucleotide deletions, duplications, additions, insertions, substitutions, mutations, repeats, genetic homologues, genetic orthologs, and/or polymorphisms.

In some embodiments, one or more genetic variants comprise one or more allelic variants. In some embodiments, allelic variants, comprise polymorphisms present in different members of the same species. In some embodiments, allelic variants result in expression of proteins with similar but slightly different functional characteristics, which predispose subjects to, or result in, certain disease states or conditions.

In some embodiments, genetic variants as used herein and throughout may comprise a homologues or orthologs present in different organisms that may be employed in accordance with the methods and devices disclosed herein in order to distinguish between the presence of one or more organisms from other organisms based on the detection of one or more such genetic variants in one or more samples. In some embodiments, such organisms comprise pathogenic organisms.

In some embodiments, a plurality of primers or sets of primers, capture nucleic acids, and/or detectable labels is employed in order to distinguish between such one or more organisms present in, or suspected of being present in, one or more samples. In some embodiments, such organisms comprise pathogenic organisms.

In some embodiments, a plurality of primers or sets of primers, capture nucleic acids, and/or detectable labels is employed in order to distinguish between organisms that belong to or may otherwise be classified into groups, such as phylogenetic and/or taxonomic groups. In some embodiments, a plurality of primers or sets of primers, capture nucleic acids, and/or detectable labels is employed in order to distinguish between organisms that belong to or may otherwise be classified into groups, such as phylogenetic and/or taxonomic groups. In some embodiments, In some embodiments, a plurality of primers or sets of primers, capture nucleic acids, and/or detectable labels is employed in order to distinguish between organisms that belong to the same or similar taxonomic groups, such as the same or a similar order, the same or a similar family, the same or a similar genus, the same or a similar subgenus, or the same or a similar species. In some embodiments, such organisms comprise pathogenic organisms.

In some embodiments, a plurality of primers or sets of primers, capture nucleic acids, and/or detectable labels is employed in order to distinguish between organisms that may be classified into groups on the bases of one or more distinguishable features or traits that allows for distinguishing between at least one such organism from other organisms in a sample in accordance with the methods and devices disclosed herein and throughout. In some embodiments, such organisms comprise pathogenic organisms.

In some embodiments, a plurality of primers or sets of primers, capture nucleic acids, and/or detectable labels is employed in order to distinguish between bacterial organisms, fungal organisms, protozoan organisms, plant organisms, animal organisms in one or more samples. In some embodiments, such organisms comprise pathogenic organisms.

In some embodiments a plurality of primers or sets of primers, capture nucleic acids, and/or detectable labels is employed in order to distinguish between fungal organisms belonging to one or more of the following groups:
1. Candida auris, Candida albicans, Candida tropicalis, Candida parapsilosis, Candida glabrata, Candida krusei, Candida haemulonis
2. Aspergillus fumigatus, Aspergillus flavus, Aspergillus niger, Aspergillus terreus
3. Cryptococcus neoformans, Cryptococcus gattii
4. Coccidioides immitis, Coccidioides posadasii
5. Fusarium solani, Fusarium oxysporum, Fusarium verticillioidis, and Fusarium moniliforme
6. Pneumocystis jirovecii
7. Blastomyces dermatitidis
8. Histoplasma capsulatum
9. Rhizopus oryzae, Rhizopus microspores
10. Candida auris

In some embodiments a plurality of primers comprising at least one of the following primers is employed in order to distinguish between one or more organisms present in, or suspected of being present in, one or more samples:

Reverse Primer:
(SEQ ID NO: 17)
/5Phos/GGAGTGATTTGTCTGCTTAATTGC
Forward Primer:
(SEQ ID NO: 18)
5Biosg/GGCTTGAGCCGATAGTCCC;
or
Forward Primer:
(SEQ ID NO: 33
5Biosg/CATCGGCTTGAGCCGATAGTC
Forward Primer:
(SEQ ID NO: 19)
5Biosg/GCCTCAAACTTCCATCGACTTC
Reverse Primer:
(SEQ ID NO: 20)
/5Phos/CGATAACGAACGAGACCTTAACC
Reverse Primer:
(SEQ ID NO: 21)
/5Phos/CAGGTCTGTGATGCCCTTAG
Forward Primer:
(SEQ ID NO: 22)
5Biosg/CAATGCTCTATCCCCAGCAC

In some embodiments a plurality of primers selected from the group consisting of the following primers is employed in order to distinguish between one or more organisms present in, or suspected of being present in, one or more samples:

Reverse Primer:
(SEQ ID NO: 17)
/5Phos/GGAGTGATTTGTCTGCTTAATTGC
Forward Primer:
(SEQ ID NO: 18)
5Biosg/GGCTTGAGCCGATAGTCCC;
or
Forward Primer:
(SEQ ID NO: 33
5Biosg/CATCGGCTTGAGCCGATAGTC
Forward Primer:
(SEQ ID NO: 19)
5Biosg/GCCTCAAACTTCCATCGACTTC
Reverse Primer:
(SEQ ID NO: 20)
/5Phos/CGATAACGAACGAGACCTTAACC
Reverse Primer:
(SEQ ID NO: 21)
/5Phos/CAGGTCTGTGATGCCCTTAG
Forward Primer:
(SEQ ID NO: 22)
5Biosg/CAATGCTCTATCCCCAGCAC

In some embodiments a plurality of capture nucleic acids comprising at least one of the following capture nucleic acids is employed in order to distinguish between one or more organisms present in, or suspected of being present in, one or more samples:

(SEQ ID NO: 23)
/5AmMC6/AAAAAAAAAAGTGCTGCCAGCGCGCCTCTTG
(SEQ ID NO: 24)
/5AmMC6/AAAAAAAAAACCGACCC+ACGT+TTG+TGG
(SEQ ID NO: 25)
/5AmMC6/AAAAAAAAAACGA+CCCGCGT+CTG+CG
(SEQ ID NO: 26)
/5AmMC6/AAAAAAAAAACGAGACCT+CG+GCCCTTAA
(SEQ ID NO: 27)
/5AmMC6/AAAAAAAAAACACTGACG+GA+GCCAGC
(SEQ ID NO: 28)
/5AmMC6/AAAAAAAAAAGAGTCTTA+CC+GC+CTTGGC
(SEQ ID NO: 29)
/5AmMC6/AAAAAAAAAAGCCAGC+AA+GT+T+CATTTCC
(SEQ ID NO: 30)
/5AmMC6/AAAAAAAAAAGTACT+TC+C+TT+GGCCGAAAG
(SEQ ID NO: 31)
/5AmMC6/AAAAAAAAAACACT+GA+TG+AA+G+TCAGCG
(SEQ ID NO: 32)
/5AmMC6/AAAAAAAAAAGTACATCA+CCTTGG+CCG

In some embodiments a plurality of capture nucleic acids selected from the group consisting of the following capture nucleic acids is employed in order to distinguish between one or more organisms present in, or suspected of being present in, one or more samples:

(SEQ ID NO: 23)
/5AmMC6/AAAAAAAAAAGTGCTGCCAGCGCGCCTCTTG
(SEQ ID NO: 24)
/5AmMC6/AAAAAAAAAACCGACCC+ACGT+TTG+TGG
(SEQ ID NO: 25)
/5AmMC6/AAAAAAAAAACGA+CCCGCGT+CTG+CG
(SEQ ID NO: 26)
/5AmMC6/AAAAAAAAAACGAGACCT+CG+GCCCTTAA
(SEQ ID NO: 27)
/5AmMC6/AAAAAAAAAACACTGACG+GA+GCCAGC
(SEQ ID NO: 28)
/5AmMC6/AAAAAAAAAAGAGTCTTA+CC+GC+CTTGGC
(SEQ ID NO: 29)
/5AmMC6/AAAAAAAAAAGCCAGC+AA+GT+T+CATTTCC
(SEQ ID NO: 30)
/5AmMC6/AAAAAAAAAAGTACT+TC+C+TT+GGCCGAAAG
(SEQ ID NO: 31)
/5AmMC6/AAAAAAAAAACACT+GA+TG+AA+G+TCAGCG
(SEQ ID NO: 32)
/5AmMC6/AAAAAAAAAAGTACATCA+CCTTGG+CCG

In some embodiments, primers or primer sets are configured to amplify target nucleic acids that are shared by such one or more organisms but have one or more nucleotide differences between such one or more organisms, and thus may serve as target nucleic acids which may be used to distinguish between such one or more organisms in accordance with the methods and devices disclosed herein and throughout. In some embodiments, such organisms comprise pathogenic organisms.

In some embodiments, target nucleic acids are configured to capture amplified target nucleic acids (also referred interchangeable throughout as amplicons and/or distinguishable amplicons) that are shared by such one or more organisms but have one or more nucleotide differences between such one or more organisms, and thus may serve as target nucleic acids which may be used to distinguish between such one or more organisms in accordance with the methods and devices disclosed herein and throughout.

In some embodiments, a genetic variation, such as an allelic variant, is a single-nucleotide polymorphism (SNP). In certain embodiments a genetic variation of interest comprises one or more nucleotide substitutions near (e.g., in a 5′ flanking region, 3′ flanking region or intron) or within (e.g., within an exon or coding region) a gene of interest, non-limiting examples of which include A to C, A to G, A to T, C to A, C to G, C to T, T to A, T to C, T to G, G to A, G to C, G to T, and the like. In some embodiments, a genetic variation, such as an allelic variant, comprises one, two, three, four or more single nucleotide polymorphisms. In some embodiments, a mutation is a single nucleotide deletion, insertion, or substitution. In some embodiments, a genetic variation comprises one or more single nucleotide mutations (e.g., 1, 2, 3, 4 or more single nucleotide mutations) of a target nucleic acid. In some embodiments, a genetic variation (e.g., a mutation) is a variation in a nucleic acid sequence of a target nucleic acid that is not present in a wild-type or reference genome (e.g., reference sequence, reference gene, or portion thereof). In some embodiments, a target sequence of a wild-type or reference genome comprises a nucleic acid sequence that is not associated with a disease or condition (e.g., a cancer). In some embodiments a genetic variation is a somatic mutation that may be present in cells of a tumor or neoplastic tissue, but is not present in normal or non-cancerous cells of the subject. In some embodiments a mutation is an autosomal mutation. In some embodiments, a mutation is an autosomal recessive mutation or an autosomal dominant mutation.

In some embodiments, a genetic variation is a SNP. Accordingly, a method described herein can detect the presence or absence of a predetermined allelic variant of a SNP (e.g., a first allelic variant), where the absence of the allelic variant refers to a target nucleic acid comprising another allelic variant of the SNP (e.g., a second, third or fourth variant). For example, the presence of a predetermined allelic variant or first allelic variant in a target nucleic acid may be a G, where the absence of the first allelic variant refers to the presence of an A, T or C in the same position of the target sequence.

A genetic variation may be presence or absence in a target nucleic acid of one or both chromosomes of a mammalian subject. In some embodiments, a method described herein detects the presence of a genetic variation in one or both alleles of a genome. In some embodiments, a method described herein detects the absence of a genetic variation in both alleles of a genome.

Non-limiting examples of a gene of interest, each of which may comprise a genetic variation of interest, include human genes A2M, AACS, AARSD1, ABCA10, ABCA12, ABCA3, ABCA8, ABCA9, ABCB1, ABCB10, ABCB4, ABCC11, ABCC12, ABCC6, ABCD1, ABCE1, ABCF1, ABCF2, ABT1, ACAA2, ACCSL, ACER2, ACO2, ACOT1, ACOT4, ACOT7, ACP1, ACR, ACRC, ACSBG2, ACSM1, ACSM2A, ACSM2B, ACSM4, ACSM5, ACTA1, ACTA2, ACTB, ACTG1, ACTG2, ACTN1, ACTN4, ACTR1A, ACTR2, ACTR3, ACTR3C, ACTR1, ADAD1, ADAL, ADAM18, ADAM20, ADAM21, ADAM32, ADAMTS7, ADAMTSL2, ADAT2, ADCY5, ADCY6, ADCY7, ADGB, ADH1A, ADH1B, ADH1C, ADH5, ADORA2B, ADRBK2, ADSS, AFF3, AFF4, AFG3L2, AGAP1, AGAP10, AGAP11, AGAP4, AGAP5, AGAP6, AGAP7, AGAP8, AGAP9, AGER, AGGF1, AGK, AGPAT1, AGPAT6, AHCTF1, AHCY, AHNAK2, AHRR, AIDA, AIF1, AIM1L, AIMP2, AK2, AK3, AK4, AKAP13, AKAP17A, AKIP1, AKIRIN1, AKIRIN2, AKR1B1, AKR1B10, AKR1B15, AKR1C1, AKR1C2, AKR1C3, AKR1C4, AKR7A2, AKR7A3, AKTIP, ALDH3B1, ALDH3B2, ALDH7A1, ALDOA, ALG1, ALG10, ALG10B, ALG1L, ALGIL2, ALG3, ALKBH8, ALMS1, ALOX15, ALOX15B, ALOXE3, ALPI, ALPP, ALPPL2, ALYREF, AMD1, AMELX, AMELY, AMMECR1L, AMY1A, AMY1B, AMY1C, AMY2A, AMY2B, AMZ2, ANAPC1, ANAPC10, ANAPC15, ANKRD11, ANKRD18A, ANKRD18B, ANKRD20A1, ANKRD20A19P, ANKRD20A2, ANKRD20A3, ANKRD20A4, ANKRD30A, ANKRD30B, ANKRD36, ANKRD36B, ANKRD49, ANKS1B, ANO10, ANP32A, ANP32B, ANXA2, ANXA2R, ANXA8, ANXA8L1, ANXA8L2, AOC2, AOC3, AP1B1, APIS2, AP2A1, AP2A2, AP2B1, AP2S1, AP3M2, AP3S1, AP4S1, APBA2, APBBIIP, APH1B, API5, APIP, APOBEC3A, APOBEC3B, APOBEC3C, APOBEC3D, APOBEC3F, APOBEC3G, APOC1, APOL1, APOL2, APOL4, APOM, APOOL, AQP10, AQP12A, AQP12B, AQP7, AREG, AREGB, ARF1, ARF4, ARF6, ARGFX, ARHGAP11A, ARHGAP11B, ARHGAP20, ARHGAP21, ARHGAP23, ARHGAP27, ARHGAP42, ARHGAP5, ARHGAP8, ARHGEF35, ARHGEF5, ARID2, ARID3B, ARIH2, ARL14EP, ARL16, ARL17A, ARL17B, ARL2BP, ARL4A, ARL5A, ARL6IP1, ARL6IP6, ARL8B, AMC1, AMC10, ARMC4, ARM8,ARMCX6, ARPC1A, ARPC2, ARPC3, ARPP19, ARSD, ARSE, ARSF, ART3, ASAH2, ASAH2B, ASB9, ASL, ASMT, ASMTL, ASNS, ASS1, ATAD1, ATAD3A, ATAD3B, ATAD3C, ATAT1, ATF4, ATF6B, ATF7IP2, ATG4A, ATM, ATMIN, ATP13A4, ATP13A5, ATP1A2, ATP1A4, ATP1B1, ATP1B3, ATP2B2, ATP2B3, ATP5A1, ATP5C1, ATP5F1, ATP5G1, ATP5G2, ATP5G3, ATP5H, ATP5J, ATP5J2, ATP5J2-PTCD1, ATP5O, ATP6AP2, ATP6V0C, ATP6V1E1, ATP6V1F, ATP6V1G1, ATP6V1G2, ATP7B, ATP8A2, ATP9B, ATXN1L, ATXN2L, ATXN7L3, AURKA, AURKAIP1, AVP, AZGP1, AZI2, B3GALNT1, B3GALT4, B3GAT3, B3GNT2, BAG4, BAG6, BAGE2, BAK1, BANF1, BANP, BCAP31, BCAR1, BCAS2, BCL2A1, BCL2L12, BCL2L2-PABPN1, BCLAF1, BCOR, BCR, BDH2, BDP1, BEND3, BET1, BEX1, BHLHB9, BHLHE22, BHLHE23, BHMT, BHMT2, BIN2, BIRC2, BIRC3, BLOC1S6, BLZF1, BMP2K, BMP8A, BMP8B, BMPR1A, BMS1, BNIP3, BOD1, BOD1L2, BOLA2, BOLA2B, BOLA3, BOP1, BPTF, BPY2, BPY2B, BPY2C, BRAF, BRCA1, BRCC3, BRD2, BRD7, BRDT, BRI3, BRK1, BRPF1, BRPF3, BRWD1, BTBD10, BTBD6, BTBD7, BTF3, BTF3L4, BTG1, BTN2A1, BTN2A2, BTN3A1, BTN3A2, BTN3A3, BTNL2, BTNL3, BTNL8, BUB3, BZW1, C10orf129, C10orf88, C11orf48, C11orf58, C11orf74, C11orf75, C12orf29, C12orf42, C12orf49, C12orf71, C12orf76, C14orf119, C14orf166, C14orf178, C15orf39, C15orf40, C15orf43, C16orf52, C16orf88, C17orf51, C17orf58, C17orf61, C17orf89, C17orf98, C18orf21, C18orf25, C1D, C1GALT1, C1QBP, C1QL1, C1QL4, C1QTNF9, C1QTNF9B, C1QTNF9B-AS1, C1orf100, C1orf106, C1orf114, C2, C22orf42, C22orf43, C2CD4A, C2orf16, C2orf27A, C2orf27B, C2orf69, C2orf78, C2orf81, C4A, C4B, C4BPA, C4orf27, C4orf34, C4orf46, C5orf15, C5orf43, C5orf52, C5orf60, C5orf63, C6orf10, C6orf106, C6orf136, C6orf15, C6orf203, C6orf25, C6orf47, C6orf48, C7orf63, C7orf73, C8orf46, C9orf123, C9orf129, C9orf172, C9orf57, C9orf69, C9orf78, CA14, CA15P3, CA5A, CA5B, CABYR, CACNA1C, CACNA1G, CACNA1H, CACNA1I, CACYBP, CALCA, CALCB, CALM1, CALM2, CAMSAP1, CAP1, CAPN8, CAPZA1, CAPZA2, CARD16, CARD17, CASC4, CASP1, CASP3, CASP4, CASP5, CATSPER2, CBR1, CBR3, CBWD1, CBWD2, CBWD3, CBWD5, CBWD6, CBWD7, CBX1, CBX3, CCDC101, CCDC111, CCDC121, CCDC127, CCDC14, CCDC144A, CCDC144NL, CCDC146, CCDC150, CCDC174, CCDC25, CCDC58, CCDC7, CCDC74A, CCDC74B, CCDC75, CCDC86, CCHCR1, CCL15, CCL23, CCL3, CCL3L1, CCL3L3, CCL4, CCL4L1, CCL4L2, CCNB11P1, CCNB2, CCND2, CCNG1, CCNJ, CCNT2, CCNYL1, CCR2, CCR5, CCRL1, CCRN4L, CCT4, CCT5, CCT6A, CCT7, CCT8, CCT8L2, CCZ1, CCZ1B, CD177, CD1A, CD1B, CD1C, CD1D, CD1E, CD200R1, CD200R1L, CD209, CD276, CD2BP2, CD300A, CD300C, CD300LD, CD300LF, CD33, CD46, CD83, CD8B, CD97, CD99, CDC14B, CDC20, CDC26, CDC27, CDC37, CDC42, CDC42EP3, CDCA4, CDCA7L, CDH12, CDK11A, CDK11B, CDK2AP2, CDK5RAP3, CDK7, CDK8, CDKN2A, CDKN2AIPNL, CDKN2B, CDON, CDPF1, CDRT1, CDRT15, CDRT15L2, CDSN, CDV3, CDY1, CDY2A, CDY2B, CEACAM1, CEACAM18, CEACAM21, CEACAM3, CEACAM4, CEACAM5, CEACAM6, CEACAM7, CEACAM8, CEL, CELA2A, CELA2B, CELA3A, CELA3B, CELSR1, CEND1, CENPC1, CENPI, CENPJ, CENPO, CEP170, CEP19, CEP192, CEP290, CEP57L1, CES1, CES2, CES5A, CFB, CFC1, CFC1B, CFH, CFHR1, CFHR2, CFHR3, CFHR4, CFHR5, CFL1, CFTR, CGB, CGB1, CGB2, CGB5, CGB7, CGB8, CHAF1B, CHCHD10, CHCHD2, CHCHD3, CHCHD4, CHD2, CHEK2, CHIA, CHMP4B, CHMP5, CHORDC1, CHP1, CHRAC1, CHRFAM7A, CHRNA2, CHRNA4, CHRNB2, CHRNB4, CHRNE, CHST5, CHST6, CHSY1, CHTF8, CIAPIN1, CIC, CIDEC, CIR1, CISD1, CISD2, CKAP2, CKMT1A, CKMT1B, CKS2, CLC, CLCN3, CLCNKA, CLCNKB, CLDN22, CLDN24, CLDN3, CLDN4, CLDN6, CLDN7, CLEC17A, CLEC18A, CLEC18B, CLEC18C, CLEC1A, CLEC1B, CLEC4G, CLEC4M, CLIC1, CLIC4, CLK2, CLK3, CLK4, CLNS1A, CMPK1, CMYA5, CNEP1R1, CNN2, CNN3, CNNM3, CNNM4, CNOT6L, CNOT7, CNTNAP3, CNTNAP3B, CNTNAP4, COA5, COBL, COIL, COL11A2, COL12A1, COL19A1, COL25A1, COL28A1, COL4A5, COL6A5, COL6A6, COMMD4, COMMD5, COPRS, COPS5, COPS8, COQ10B, CORO1A, COX10, COX17, COX20, COX5A, COX6A1, COX6B1, COX7B, COX7C, COX8C, CP, CPAMD8, CPD, CPEB1, CPSF6, CR1, CRIL, CRADD, CRB3, CRCP, CREBBP, CRHR1, CRLF2, CRLF3, CRNN, CROCC, CRTC1, CRYBB2, CRYGB, CRYGC, CRYGD, CS, CSAG1, CSAG2, CSAG3, CSDA, CSDE1, CSF2RA, CSF2RB, CSGALNACT2, CSH1, CSH2, CSHL1, CSNK1A1, CSNK1D, CSNK1E, CSNK1G2, CSNK2A1, CSNK2B, CSPG4, CSRP2, CST1, CST2, CST3, CST4, CST5, CST9, CT45A1, CT45A2, CT45A3, CT45A4, CT45A5, CT45A6, CT47A1, CT47A10, CT47A11, CT47A12, CT47A2, CT47A3, CT47A4, CT47A5, CT47A6, CT47A7, CT47A8, CT47A9, CT47B1, CTAG1A, CTAG1B, CTAG2, CTAGE1, CTAGE5, CTAGE6P, CTAGE9, CTBP2, CTDNEP1, CTDSP2, CTDSPL2, CTLA4, CTNNA1, CTNND1, CTRB1, CTRB2, CTSL1, CTU1, CUBN, CUL1, CUL7, CUL9, CUTA, CUX1, CXADR, CXCL1, CXCL17, CXCL2, CXCL3, CXCL5, CXCL6, CXCR1, CXCR2, CXorf40A, CXorf40B, CXorf48, CXorf49, CXorf49B, CXorf56, CXorf61, CYB5A, CYCA, CYP11B1, CYP11B2, CYP1A1, CYP1A2, CYP21A2, CYP2A13, CYP2A6, CYP2A7, CYP2B6, CYP2C18, CYP2C19, CYP2C8, CYP2C9, CYP2D6, CYP2F1, CYP3A4, CYP3A43, CYP3A5, CYP3A7, CYP3A7-CYP3AP1, CYP46A1, CYP4A11, CYP4A22, CYP4F11, CYP4F12, CYP4F2, CYP4F3, CYP4F8, CYP4Z1, CYP51A1, CYorf17, DAP3, DAPK1, DAXX, DAZ1, DAZ2, DAZ3, DAZ4, DAZAP2, DAZL, DBF4, DCAF12L1, DCAF12L2, DCAF13, DCAF4, DCAF4L1, DCAF4L2, DCAF6, DCAF8L1, DCAF8L2, DCLRE1C, DCTN6, DCUN1D1, DCUN1D3, DDA1, DDAH2, DDB2, DDR1, DDT, DDTL, DDX10, DDX11, DDX18, DDX19A, DDX19B, DDX23, DDX26B, DDX39B, DDX3X, DDX3Y, DDX50, DDX55, DDX56, DDX6, DDX60, DDX60L, DEF8, DEFB103A, DEFB103B, DEFB104A, DEFB104B, DEFB105A, DEFB105B, DEFB106A, DEFB106B, DEFB107A, DEFB107B, DEFB108B, DEFB130, DEFB131, DEFB4A, DEFB4B, DENND1C, DENR, DEPDC1, DERL2, DESI2, DEXI, DGCR6, DGCR6L, DGKZ, DHFR, DHFRL1, DHRS2, DHRS4, DHRS4L1, DHRS4L2, DHRSX, DHX16, DHX29, DHX34, DHX40, DICER1, DIMT1, DIS3L2, DKKL1, DLEC1, DLST, DMBT1, DMRTC1, DMRTC1B, DNAH11, DNAJA1, DNAJA2, DNAJB1, DNAJB14, DNAJB3, DNAJB6, DNAJC1, DNAJC19, DNAJC24, DNAJC25-GNG10, DNAJC5, DNAJC7, DNAJC8, DNAJC9, DND1, DOCK1, DOCK11, DOCK9, DOK1, DOM3Z, DONSON, DPCR1, DPEP2, DPEP3, DPF2, DPH3, DPM3, DPP3, DPPA2, DPPA3, DPPA4, DPPA5, DPRX, DPY19L1, DPY19L2, DPY19L3, DPY19L4, DPY30, DRAXIN, DRD5, DRG1, DSC2, DSC3, DSE, DSTN, DTD2, DTWD1, DTWD2, DTX2, DUOX1, DUOX2, DUSP12, DUSP5, DUSP8, DUT, DUXA, DYNC1I2, DYNC1LI1, DYNLT1, DYNLT3, E2F3, EBLN1, EBLN2, EBPL, ECEL1, EDDM3A, EDDM3B, EED, EEF1A1, EEF1B2, EEF1D, EEF1E1, EEF1G, EFCAB3, EFEMP1, EFTUD1, EGFR, EGFL8, EGLNJ, EHD1, EHD3, EHMT2, EI24, EIF1, EIF1AX, EIF2A, EIF2C1, EIF2C3, EIF2S2, EIF2S3, EIF3A, EIF3C, EIF3CL, EIF3E, EIF3F, EIF3J, EIF3L, EIF3M, EIF4A1, EIF4A2, EIF4B, EIF4E, EIF4E2, EIF4EBP1, EIF4EBP2, EIF4H, EIF5, EIF5A, EIF5A2, EIF5AL1, ELF2, ELK1, ELL2, ELMO2, EMB, EMC3, EMR1, EMR2, EMR3, ENAH, ENDOD1, ENO1, ENO3, ENPEP, ENPP7, ENSA, EP300, EP400, EPB41L4B, EPB41L5, EPCAM, EPHA2, EPHB2, EPHB3, EPN2, EPN3, EPPK1, EPX, ERCC3, ERF, ERP29, ERP44, ERVV-1, ERVV-2, ESCO1, ESF1, ESPL1, ESPN, ESRRA, ETF1, ETS2, ETV3, ETV3L, EVA1C, EVPL, EVPLL, EWSR1, EXOC5, EXOC8, EXOG, EXOSC3, EXOSC6, EXTL2, EYS, EZR, F5, F8A1, F8A2, F8A3, FABP3, FABP5, FAF2, FAHD1, FAHD2A, FAHD2B, FAM103A1, FAM104B, FAM108A1, FAM108C1, FAM111B, FAM115A, FAM115C, FAM120A, FAM120B, FAM127A, FAM127B, FAM127C, FAM131C, FAM133B, FAM136A, FAM149B1, FAM151A, FAM153A, FAM153B, FAM154B, FAM156A, FAM156B, FAM157A, FAM157B, FAM163B, FAM165B, FAM175A, FAM177A1, FAM185A, FAM186A, FAM18B1, FAM18B2, FAM190B, FAM192A, FAM197Y1, FAM197Y3, FAM197Y4, FAM197Y6, FAM197Y7, FAM197Y8, FAM197Y9, FAM203A, FAM203B, FAM204A, FAM205A, FAM206A, FAM207A, FAM209A, FAM209B, FAM20B, FAM210B, FAM213A, FAM214B, FAM218A, FAM21A, FAM21B, FAM21C, FAM220A, FAM22A, FAM22D, FAM22F, FAM22G, FAM25A, FAM25B, FAM25C, FAM25G, FAM27E4P, FAM32A, FAM35A, FAM3C, FAM45A, FAM47A, FAM47B, FAM47C, FAM47E-STBD1, FAM58A, FAM60A, FAM64A, FAM72A, FAM72B, FAM72D, FAM76A, FAM83G, FAM86A, FAM86B2, FAM86C1, FAM89B, FAM8A1, FAM90A1, FAM91A1, FAM92A1, FAM96A, FAM98B, FAM9A, FAM9B, FAM9C, FANCD2, FANK1, FAR1, FAR2, FARP1, FARSB, FASN, FASTKD1, FAT1, FAU, FBLIM1, FBP2, FBRSL1, FBXL12, FBXO25, FBXO3, FBXO36, FBXO44, FBXO6, FBXW10, FBXW11, FBXW2, FBXW4, FCF1, FCGBP, FCGR1A, FCGR2A, FCGR2B, FCGR3A, FCGR3B, FCN1, FCN2, FCRL1, FCRL2, FCRL3, FCRL4, FCRL5, FCRL6, FDPS, FDX1, FEM1A, FEN1, FER, FFAR3, FGD5, FGF7, FGFR1OP2, FH, FHL1, FIGLA, FKBP1A, FKBP4, FKBP6, FKBP8, FKBP9, FKBPL, FLG, FLG2, FLI1, FLJ44635, FLNA, FLNB, FLNC, FLOT1, FLT1, FLYWCH1, FMN2, FN3K, FOLH1, FOLH1B, FOLR1, FOLR2, FOLR3, FOSL1, FOXA1, FOXA2, FOXA3, FOXD1, FOXD2, FOXD3, FOXD4L2, FOXD4L3, FOXD4L6, FOXF1, FOXF2, FOXH1, FOXN3, FOXO1, FOXO3, FPR2, FPR3, FRAT2, FREM2, FRG1, FRG2, FRG2B, FRG2C, FRMD6, FRMD7, FRMD8, FRMPD2, FSCN1, FSIP2, FTH1, FTHL17, FTL, FTO, FUNDC1, FUNDC2, FUT2, FUT3, FUT5, FUT6, FXN, FXR1, FZD2, FZD5, FZD8, G2E3, G3BP1, GABARAP, GABARAPL1, GABBR1, GABPA, GABRP, GABRR1, GABRR2, GAGE1, GAGE10, GAGE12C, GAGE12D, GAGE12E, GAGE12F, GAGE12G, GAGE12H, GAGE12I, GAGE12I, GAGE13, GAGE2A, GAGE2B, GAGE2C, GAGE2D, GAGE2E, GAPDH, GAR1, GATS, GATSL1, GATSL2, GBA, GBP1, GBP2, GBP3, GBP4, GBP5, GBP6, GBP7, GCAT, GCDH, GCNT1, GCOMM1, GCSH, GDI2, GEMIN7, GEMIN8, GFRA2, GGCT, GGT1, GGT2, GGT5, GGTLC1, GGTLC2, GH1, GH2, GINS2, GJA1, GJC3, GK, GK2, GLBIL2, GLBIL3, GLDC, GLOD4, GLRA1, GLRA4, GLRX, GLRX3, GLRX5, GLTP, GLTSCR2, GLUD1, GLUL, GLYATL1, GLYATL2, GLYR1, GM2A, GMCL1, GMFB, GMPS, GNA11, GNAQ, GNAT2, GNG10, GNG5, GNGT1, GNL1, GNL3, GNL3L, GNPNAT1, GOLGA2, GOLGA4, GOLGA5, GOLGA6A, GOLGA6B, GOLGA6C, GOLGA6D, GOLGA6L1, GOLGA6L10, GOLGA6L2, GOLGA6L3, GOLGA6L4, GOLGA6L6, GOLGA6L9, GOLGA7, GOLGA8H, GOLGA8J, GOLGA8K, GOLGA8O, GON4L, GOSR1, GOSR2, GOT2, GPAA1, GPANK1, GPAT2, GPATCH8, GPC5, GPCPD1, GPD2, GPHN, GPN1, GPR116, GPR125, GPR143, GPR32, GPR89A, GPR89B, GPR89C, GPS2, GPSM3, GPX1, GPX5, GPX6, GRAP, GRAPL, GRIA2, GRIA3, GRIA4, GRK6, GRM5, GRM8, GRPEL2, GSPT1, GSTA1, GSTA2, GSTA3, GSTA5, GSTM1, GSTM2, GSTM4, GSTM5, GSTO1, GSTT1, GSTT2, GSTT2B, GTF2AIL, GTF2H1, GTF2H2, GTF2H2C, GTF2H4, GTF2I, GTF2IRD1, GTF2IRD2, GTF2IRD2B, GTF3C6, GTPBP6, GUSB, GXYLT1, GYG1, GYG2, GYPA, GYPB, GYPE, GZMB, GZMH, HIFOO, H2AFB1, H2AFB2, H2AFB3, H2AFV, H2AFX, H2AFZ, H2BFM, H2BFWT, H3F3A, H3F3B, H3F3C, HADHA, HADHB, HARS, HARS2, HAS3, HAUS1, HAUS4, HAUS6, HAVCR1, HAX1, HBA1, HBA2, HBB, HBD, HBG1, HBG2, HBSIL, HBZ, HCAR2, HCAR3, HCN2, HCN3, HCN4, HDAC1, HDGF, HDHD1, HEATR7A, HECTD4, HERC2, HIATL1, HIBCH, HIC1, HIC2, HIGD1A, HIGD2A, HINT1, HIST1H1B, HIST1H1C, HIST1H1D, HIST1H2AA, HIST1H2AB, HIST1H2AC, HIST1H2AD, HIST1H2AE, HIST1H2AG, HIST1H2AH, HIST1H2AI, HIST1H2AL, HIST1H2BB, HIST1H2BD, HIST1H2BE, HIST1H2BF, HIST1H2BH, HIST1H2BI, HIST1H2BK, HIST1H2BM, HIST1H2BN, HIST1H2BO, HIST1H3A, HIST1H3B, HIST1H3C, HIST1H3D, HIST1H3E, HIST1H3F, HIST1H3G, HIST1H3H, HIST1H3I, HIST1H3J, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, HIST2H2AA3, HIST2H2AB, HIST2H2AC, HIST2H2BE, HIST2H2BF, HIST2H3A, HIST2H3D, HIST2H4A, HIST2H4B, HIST3H2BB, HIST3H3, HIST4H4, HK2, HLA-A, HLA-B, HLA-C, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB5, HLA-E, HLA-F, HLA-G, HMGA1, HMGB1, HMGB2, HMGB3, HMGCS1, HMGN1, HMGN2, HMGN3, HMGN4, HMX1, HMX3, HNRNPA1, HNRNPA3, HNRNPAB, HNRNPC, HNRNPCL1, HNRNPD, HNRNPF, HNRNPH1, HNRNPH2, HNRNPH3, HNRNPK, HNRNPL, HNRNPM, HNRNPR, HNRNPU, HNRPDL, HOMER2, HORMAD1, HOXA2, HOXA3, HOXA6, HOXA7, HOXB2, HOXB3, HOXB6, HOXB7, HOXD3, HP, HPR, HPS1, HRG, HS3ST3A1, HS3ST3B1, HS6ST1, HSD17B1, HSD17B12, HSD17B4, HSD17B6, HSD17B7, HSD17B8, HSD3B1, HSD3B2, HSF2, HSFX1, HSFX2, HSP90AA1, HSP90AB1, HSP90B1, HSPA14, HSPA1A, HSPA1B, HSPA1L, HSPA2, HSPA5, HSPA6, HSPA8, HSPA9, HSPB1, HSPD1, HSPE1, HSPE1-MOB4, HSPG2, HTN1, HTN3, HTR3C, HTR3D, HTR3E, HTR7, HYDIN, HYPK, IARS, ID2, IDH1, IDI1, IDS, IER3, IFI16, IFIH1, IFIT1, IFIT1B, IFIT2, IFIT3, IFITM3, IFNA1, IFNA10, IFNA14, IFNA16, IFNA17, IFNA2, IFNA21, IFNA4, IFNA5, IFNA6, IFNA7, IFNA8, IFT122, IFT80, IGBP1, IGF2BP2, IGF2BP3, IGFL1, IGFL2, IGFN1, IGLL1, IGLL5, IGLON5, IGSF3, IHH, IK, IKBKG, IL17RE, IL18, IL28A, IL28B, IL29, IL32, IL3RA, IL6ST, IL9R, IMMP1L, IMMT, IMPA1, IMPACT, IMPDH1, ING5, INIP, INTS4, INTS6, IPMK, IPO7, IPPK, IQCB1, IREB2, IRX2, IRX3, IRX4, IRX5, IRX6, ISCA1, ISCA2, ISG20L2, ISL1, ISL2, IST1, ISY1-RAB43, ITFG2, ITGAD, ITGAM, ITGAX, ITGB1, ITGB6, ITIH6, ITLN1, ITLN2, ITSN1, KAL1, KANK1, KANSL1, KARS, KAT7, KATNBL1, KBTBD6, KBTBD7, KCNA1, KCNA5, KCNA6, KCNC1, KCNC2, KCNC3, KCNH2, KCNH6, KCNJ12, KCNJ4, KCNMB3, KCTD1, KCTD5, KCTD9, KDELC1, KDM5C, KDM5D, KDM6A, KHDC1, KHDC1L, KHSRP, KIAA0020, KIAA0146, KIAA0494, KIAA0754, KIAA0895L, KIAA1143, KIAA1191, KIAA1328, KIAA1377, KIAA1462, KIAA1549L, KIAA1551, KIAA1586, KIAA1644, KIAA1671, KIAA2013, KIF1C, KIF27, KIF4A, KIF4B, KIFC1, KIR2DL1, KIR2DL3, KIR2DL4, KIR2DS4, KIR3DL1, KIR3DL2, KIR3DL3, KLF17, KLF3, KLF4, KLF7, KLF8, KLHL12, KLHL13, KLHL15, KLHL2, KLHL5, KLHL9, KLK2, KLK3, KLRC1, KLRC2, KLRC3, KLRC4, KNTC1, KPNA2, KPNA4, KPNA7, KPNB1, KRAS, KRT13, KRT14, KRT15, KRT16, KRT17, KRT18, KRT19, KRT25, KRT27, KRT28, KRT3, KRT31, KRT32, KRT33A, KRT33B, KRT34, KRT35, KRT36, KRT37, KRT38, KRT4, KRT5, KRT6A, KRT6B, KRT6C, KRT71, KRT72, KRT73, KRT74, KRT75, KRT76, KRT8, KRT80, KRT81, KRT82, KRT83, KRT85, KRT86, KRTAP1-1, KRTAP1-3, KRTAP1-5, KRTAP10-10, KRTAP10-11, KRTAP10-12, KRTAP10-2, KRTAP10-3, KRTAP10-4, KRTAP10-7, KRTAP10-9, KRTAP12-1, KRTAP12-2, KRTAP12-3, KRTAP13-1, KRTAP13-2, KRTAP13-3, KRTAP13-4, KRTAP19-1, KRTAP19-5, KRTAP2-1, KRTAP2-2, KRTAP2-3, KRTAP2-4, KRTAP21-1, KRTAP21-2, KRTAP23-1, KRTAP3-2, KRTAP3-3, KRTAP4-12, KRTAP4-4, KRTAP4-6, KRTAP4-7, KRTAP4-9, KRTAP5-1, KRTAP5-10, KRTAP5-3, KRTAP5-4, KRTAP5-6, KRTAP5-8, KRTAP5-9, KRTAP6-1, KRTAP6-2, KRTAP6-3, KRTAP9-2, KRTAP9-3, KRTAP9-6, KRTAP9-8, KRTAP9-9, LITD1, LAGE3, LAIR1, LAIR2, LAMTOR3, LANCL3, LAP3, LAPTM4B, LARP1, LARP1B, LARP4, LARP7, LCE1A, LCE1B, LCE1C, LCE1D, LCE1E, LCE1F, LCE2A, LCE2B, LCE2C, LCE2D, LCE3C, LCE3D, LCE3E, LCMT1, LCN1, LDHA, LDHAL6B, LDHB, LEFTY1, LEFTY2, LETM1, LGALS13, LGALS14, LGALS16, LGALS7, LGALS7B, LGALS9, LGALS9B, LGALS9C, LGMN, LGR6, LHB, LILRA1, LILRA2, LILRA3, LILRA4, LILRA5, LILRA6, LILRB1, LILRB2, LILRB3, LILRB4, LILRB5, LIMK2, LIMS1, LIN28A, LIN28B, LIN54, LLPH, LMLN, LNX1, LOC100129083, LOC100129216, LOC100129307, LOC100129636, LOC100130539, LOC100131107, LOC100131608, LOC100132154, LOC100132202, LOC100132247, LOC100132705, LOC100132858, LOC100132859, LOC100132900, LOC100133251, LOC100133267, LOC100133301, LOC100286914, LOC100287294, LOC100287368, LOC100287633, LOC100287852, LOC100288332, LOC100288646, LOC100288807, LOC100289151, LOC100289375, LOC100289561, LOC100505679, LOC100505767, LOC100505781, LOC100506248, LOC100506533, LOC100506562, LOC100507369, LOC100507607, LOC100652777, LOC100652871, LOC100652953, LOC100996256, LOC100996259, LOC100996274, LOC100996301, LOC100996312, LOC100996318, LOC100996337, LOC100996356, LOC100996369, LOC100996394, LOC100996401, LOC100996413, LOC100996433, LOC100996451, LOC100996470, LOC100996489, LOC100996541, LOC100996547, LOC100996567, LOC100996574, LOC100996594, LOC100996610, LOC100996612, LOC100996625, LOC100996631, LOC100996643, LOC100996644, LOC100996648, LOC100996675, LOC100996689, LOC100996701, LOC100996702, LOC377711, LOC388849, LOC391322, LOC391722, LOC401052, LOC402269, LOC440243, LOC440292, LOC440563, LOC554223, LOC642441, LOC642643, LOC642778, LOC642799, LOC643802, LOC644634, LOC645202, LOC645359, LOC646021, LOC646670, LOC649238, LOC728026, LOC728715, LOC728728, LOC728734, LOC728741, LOC728888, LOC729020, LOC729159, LOC729162, LOC729264, LOC729458, LOC729574, LOC729587, LOC729974, LOC730058, LOC730268, LOC731932, LOC732265, LONRF2, LPA, LPCAT3, LPGAT1, LRPS, LRP5L, LRRC16B, LRRC28, LRRC37A, LRRC37A2, LRRC37A3, LRRC37B, LRRC57, LRRC59, LRRC8B, LRRFIP1, LSM12, LSM14A, LSM2, LSM3, LSP1, LTA, LTB, LUZP6, LY6G5B, LY6G5C, LY6G6C, LY6G6D, LY6G6F, LYPLA1, LYPLA2, LYRM2, LYRM5, LYST, LYZL1, LYZL2, LYZL6, MAD1L1, MAD2L1, MAGEA10-MAGEA5, MAGEA11, MAGEA12, MAGEA2B, MAGEA4, MAGEA5, MAGEA6, MAGEA9, MAGEB2, MAGEB4, MAGEB6, MAGEC1, MAGEC3, MAGED1, MAGED2, MAGED4, MAGED4B, MAGIX, MALL, MAMDC2, MAN1A1, MAN1A2, MANBAL, MANEAL, MAP1LC3B, MAP1LC3B2, MAP2K1, MAP2K2, MAP2K4, MAP3K13, MAP7, MAPK1IP1L, MAPK6, MAPK8IP1, MAPRE1, MAPT, MARC1, MARC2, MAS1L, MASP1, MAST1, MAST2, MAST3, MAT2A, MATR3, MBD3L2, MBD3L3, MBD3L4, MBD3L5, MBLAC2, MCCD1, MCF2L2, MCFD2, MCTS1, MDC1, ME1, ME2, MEAF6, MED13, MED15, MED25, MED27, MED28, MEF2A, MEF2BNB, MEIS3, MEMO1, MEP1A, MESP1, MEST, METAP2, METTL1, METTL15, METTL21A, METTL21D, METTL2A, METTL2B, METTL5, METTL7A, METTL8, MEX3B, MEX3D, MFAP2, MFF, MFN1, MFSD2B, MGAM, MICA, MICB, MINOS1, MIPEP, MKI67, MKI67IP, MKNK1, MKRN1, MLF1IP, MLL3, MLLT10, MLLT6, MMADHC, MMP10, WP23B, MMP3, MOB4, MOCS1, MOCS3, MOG, MORF4L1, MORF4L2, MPEG1, MPHOSPH10, MPHOSPH8, MPO, MPP7, MPPE1, MPRIP, MPV17L, MPZL1, MR1, MRC1, MRE11A, MRFAP1, MRFAP1L1, MRGPRX2, MRGPRX3, MRGPRX4, MRPL10, MRPL11, MRPL19, MRPL3, MRPL32, MRPL35, MRPL36, MRPL45, MRPL48, MRPL50, MRPL51, MRPS10, MRPS16, MRPS17, MRPS18A, MRPS18B, MRPS18C, MRPS21, MRPS24, MRPS31, MRPS33, MRPS36, MRPS5, MRRF, MRS2, MRTO4, MS4A4A, MS4A4E, MS4A6A, MS4A6E, MSANTD2, MSANTD3, MSANTD3-TMEFF1, MSH5, MSL3, MSN, MST1, MSTO1, MSX2, MT1A, MT1B, MT1E, MT1F, MT1G, MT1H, MT1M, MT1X, MT2A, MTAP, MTCH1, MTFR1, MTHFD1, MTHFD1L, MTHFD2, MTIF2, MTIF3, MTMR12, MTMR9, MTRF1L, MTRNR2L1, MTRNR2L5, MTRNR2L6, MTRNR2L8, MTX1, MUC12, MUC16, MUC19, MUC20, MUC21, MUC22, MUC5B, MUC6, MX1, MX2, MXRA5, MXRA7, MYADM, MYEOV2, MYH1, MYH11, MYH13, MYH2, MYH3, MYH4, MYH6, MYH7, MYH8, MYH9, MYL12A, MYL12B, MYL6, MYL6B, MYLK, MYO5B, MZT1, MZT2A, MZT2B, NAA40, NAALAD2, NAB1, NACA, NACA2, NACAD, NACC2, NAGK, NAIP, NAMPT, NANOG, NANOGNB, NANP, NAP1L1, NAP1L4, NAPEPLD, NAPSA, NARG2, NARS, NASP, NAT1, NAT2, NAT8, NAT8B, NBAS, NBEA, NBEAL1, NBPF1, NBPF10, NBPF11, NBPF14, NBPF15, NBPF16, NBPF4, NBPF6, NBPF7, NBPF9, NBR1, NCAPD2, NCF1, NCOA4, NCOA6, NCOR1, NCR3, NDEL1, NDST3, NDST4, NDUFA4, NDUFA5, NDUFA9, NDUFAF2, NDUFAF4, NDUFB1, NDUFB3, NDUFB4, NDUFB6, NDUFB8, NDUFB9, NDUFS5, NDUFV2, NEB, NEDD8, NEDD8-MDP1, NEFH, NEFM, NEIL2, NEK2, NETO2, NEUJ, NEUROD1, NEUROD2, NF1, NFE2L3, NFIC, NFIX, NFKBIL1, NFYB, NFYC, NHLH1, NHLH2, NHP2, NHP2L1, NICN1, NIF3L1, NIP7, NIPA2, NIPAL1, NIPSNAP3A, NIPSNAP3B, NKAP, NKXI-2, NLGN4X, NLGN4Y, NLRP2, NLRP5, NLRP7, NLRP9, NMD3, NME2, NMNAT1, NOB1, NOC2L, NOL11, NOLC1, NOMO1, NOMO2, NOMO3, NONO, NOP10, NOP56, NOS2, NOTCH2, NOTCH2NL, NOTCH4, NOX4, NPAP1, NPEPPS, NPIP, NPIPL3, NPM1, NPSR1, NR2F1, NR2F2, NR3C1, NRBF2, NREP, NRM, NSA2, NSF, NSFL1C, NSMAF, NSRP1, NSUN5, NT5C3, NT5DC1, NTM, NTPCR, NUBP1, NUDC, NUDT10, NUDT11, NUDT15, NUDT16, NUDT19, NUDT4, NUDT5, NUFIP1, NUP210, NUP35, NUP50, NUS1, NUTF2, NXF2, NXF2B, NXF3, NXF5, NXPE1, NXPE2, NXT1, OAT, OBP2A, OBP2B, OBSCN, OCLN, OCM, OCM2, ODC1, OFD1, OGDH, OGDHL, OGFOD1, OGFR, OLA1, ONECUT1, ONECUT2, ONECUT3, OPCML, OPN1LW, OPN1MW, OPN1MW2, OR10A2, OR10A3, OR10A5, OR10A6, OR10C1, OR10G2, OR10G3, OR10G4, OR10G7, OR10G8, OR10G9, OR10H1, OR10H2, OR10H3, OR10H4, OR10H5, OR10J3, OR10J5, OR10K1, OR10K2, OR10Q1, OR11A1, OR11G2, OR11H1, OR11H12, OR11H2, OR12D2, OR12D3, OR13C2, OR13C4, OR13C5, OR13C9, OR13D1, OR14J1, OR1A1, OR1A2, OR1D2, OR1D4, OR1E1, OR1E2, OR1F1, OR1J1, OR1J2, OR1J4, OR1L4, OR1L6, OR1M1, OR1S1, OR1S2, OR2A1, OR2A12, OR2A14, OR2A2, OR2A25, OR2A4, OR2A42, OR2A5, OR2A7, OR2AG1, OR2AG2, OR2B2, OR2B3, OR2B6, OR2F1, OR2F2, OR2H1, OR2H2, OR2J2, OR2J3, OR2L2, OR2L3, OR2L5, OR2L8, OR2M2, OR2M5, OR2M7, OR2S2, OR2T10, OR2T2, OR2T27, OR2T29, OR2T3, OR2T33, OR2T34, OR2T35, OR2T4, OR2T5, OR2T8, OR2V1, OR2V2, OR2W1, OR3A1, OR3A2, OR3A3, OR4A15, OR4A47, OR4C12, OR4C13, OR4C46, OR4D1, OR4D10, OR4D11, OR4D2, OR4D9, OR4F16, OR4F21, OR4F29, OR4F3, OR4K15, OR4M1, OR4M2, OR4N2, OR4N4, OR4N5, OR4P4, OR4Q3, OR51A2, OR51A4, OR52E2, OR52E6, OR52E8, OR52H1, OR52I1, OR52I2, OR52J3, OR52K1, OR52K2, OR52L1, OR56A1, OR56A3, OR56A4, OR56A5, OR56B4, OR5AK2, OR5B2, OR5B3, OR5D16, OR5F1, OR5H14, OR5H2, OR5H6, OR5J2, OR5L1, OR5L2, OR5M1, OR5M10, OR5M3, OR5M8, OR5P3, OR5T1, OR5T2, OR5T3, OR5V1, OR6B2, OR6B3, OR6C6, OR7A10, OR7A5, OR7C1, OR7C2, OR7G3, OR8A1, OR8B12, OR8B2, OR8B3, OR8B8, OR8G2, OR8G5, OR8H1, OR8H2, OR8H3, OR8J1, OR8J3, OR9A2, OR9A4, OR9G1, ORC3, ORM1, ORM2, OSTC, OSTCP2, OTOA, OTOP1, OTUD4, OTUD7A, OTX2, OVOS, OXCT2, OXR1, OXT, P2RX6, P2RX7, P2RY8, PA2G4, PAAF1, PABPC1, PABPC1L2A, PABPC1L2B, PABPC3, PABPC4, PABPN1, PAEP, PAFAH1B1, PAFAH1B2, PAGE1, PAGE2, PAGE2B, PAGE5, PAICS, PAIP1, PAK2, PAM, PANK3, PARG, PARL, PARN, PARP1, PARP4, PARP8, PATL1, PBX1, PBX2, PCBD2, PCBP1, PCBP2, PCDH11X, PCDH11Y, PCDH8, PCDHA1, PCDHA11, PCDHA12, PCDHA13, PCDHA2, PCDHA3, PCDHA5, PCDHA6, PCDHA7, PCDHA8, PCDHA9, PCDHB10, PCDHB11, PCDHB12, PCDHB13, PCDHB15, PCDHB16, PCDHB4, PCDHB8, PCDHGA1, PCDHGA11, PCDHGA12, PCDHGA2, PCDHGA3, PCDHGA4, PCDHGA5, PCDHGA7, PCDHGA8, PCDHGA9, PCDHGB1, PCDHGB2, PCDHGB3, PCDHGB5, PCDHGB7, PCGF6, PCMTD1, PCNA, PCNP, PCNT, PCSK5, PCSK7, PDAP1, PDCD2, PDCD5, PDCD6, PDCD6IP, PDCL2, PDCL3, PDE4DIP, PDIA3, PDLIM1, PDPK1, PDPR, PDSS1, PDXDC1, PDZD11, PDZK1, PEBP1, PEF1, PEPD, PERP, PEX12, PEX2, PF4, PF4V1, PFDN1, PFDN4, PFDN6, PFKFB1, PFN1, PGA3, PGA4, PGA5, PGAM1, PGAM4, PGBD3, PGBD4, PGD, PGGT1B, PGK1, PGK2, PGM5, PHAX, PHB, PHC1, PHF1, PHF10, PHF2, PHF5A, PHKA1, PHLPP2, PHOSPHO1, PI3, PI4K2A, PI4KA, PIEZO2, PIGA, PIGF, PIGH, PIGN, PIGY, PIK3CA, PIK3CD, PILRA, PIN1, PIN4, PIP5K1A, PITPNB, PKD1, PKM, PKP2, PKP4, PLA2G10, PLA2G12A, PLA2G4C, PLAC8, PLAC9, PLAGL2, PLD5, PLEC, PLEKHA3, PLEKHA8, PLEKHM1, PLG, PLGLB1, PLGLB2, PLIN2, PLIN4, PLK1, PLLP, PLSCR1, PLSCR2, PLXNA1, PLXNA2, PLXNA3, PLXNA4, PM20D1, PMCH, PMM2, PMPCA, PMS2, PNKD, PNLIP, PNLIPRP2, PNMA6A, PNMA6B, PNMA6C, PNMA6D, PNO1, PNPLA4, PNPT1, POLD2, POLE3, POLH, POLR2E, POLR2J, POLR2J2, POLR2J3, POLR2M, POLR3D, POLR3G, POLR3K, POLRMT, POM121, POM121C, POMZP3, POTEA, POTEC, POTED, POTEE, POTEF, POTEH, POTEI, POTEJ, POTEM, POU3F1, POU3F2, POU3F3, POU3F4, POU4F2, POU4F3, POU5F1, PPA1, PPAT, PPBP, PPCS, PPEF2, PPFIBP1, PPIA, PPIAL4C, PPIAL4D, PPIAL4E, PPIAL4F, PPIE, PPIG, PPIL1, PPIP5K1, PPIP5K2, PPM1A, PPP1R11, PPP1R12B, PPP1R14B, PPP1R18, PPP1R2, PPP1R26, PPP1R8, PPP2CA, PPP2CB, PPP2R2D, PPP2R3B, PPP2R5C, PPP2R5E, PPP4R2, PPP5C, PPP5D1, PPP6R2, PPP6R3, PPT2, PPY, PRADC1, PRAMEF1, PRAMEF10, PRAMEF11, PRAMEF12, PRAMEF13, PRAMEF14, PRAMEF15, PRAMEF16, PRAMEF17, PRAMEF18, PRAMEF19, PRAMEF20, PRAMEF21, PRAMEF22, PRAMEF23, PRAMEF25, PRAMEF3, PRAMEF4, PRAMEF5, PRAMEF6, PRAMEF7, PRAMEF8, PRAMEF9, PRB1, PRB2, PRB3, PRB4, PRDM7, PRDM9, PRDX1, PRDX2, PRDX3, PRDX6, PRELID1, PRG4, PRH1, PRH2, PRKAR1A, PRKC1, PRKRA, PRKRIR, PRKX, PRMT1, PRMT5, PRODH, PROKR1, PROKR2, PROS1, PRPF3, PRPF38A, PRPF4B, PRPS1, PRR12, PRR13, PRR20A, PRR20B, PRR20C, PRR20D, PRR20E, PRR21, PRR23A, PRR23B, PRR23C, PRR3, PRR5-ARHGAP8, PRRC2A, PRRC2C, PRRT1, PRSS1, PRSS21, PRSS3, PRSS41, PRSS42, PRSS48, PRUNE, PRY, PRY2, PSAT1, PSG1, PSG11, PSG2, PSG3, PSG4, PSG5, PSG6, PSG8, PSG9, PSIP1, PSMA6, PSMB3, PSMB5, PSMB8, PSMB9, PSMC1, PSMC2, PSMC3, PSMC5, PSMC6, PSMD10, PSMD12, PSMD2, PSMD4, PSMD7, PSMD8, PSME2, PSORS1C1, PSORS1C2, PSPH, PTBP1, PTCD2, PTCH1, PTCHD3, PTCHD4, PTEN, PTGES3, PTGES3L-AARSD1, PTGR1, PTMA, PDMS, PTOV1, PTP4A1, PTP4A2, PTPN11, PTPN2, PTPN20A, PTPN20B, PTPRD, PTPRH, PTPRM, PTPRN2, PTPRU, PTTG1, PTTG2, PVRIG, PVRL2, PWWP2A, PYGB, PYGL, PYHIN1, PYROXD1, PYURF, PYY, PZP, QRSL1, R3HDM2, RAB11A, RAB11FIP1, RAB13, RAB18, RAB1A, RAB1B, RAB28, RAB31, RAB40AL, RAB40B, RAB42, RAB43, RAB5A, RAB5C, RAB6A, RAB6C, RAB9A, RABGEF1, RABGGTB, RABL2A, RABL2B, RABL6, RAC1, RACGAP1, RAD1, RAD17, RAD21, RAD23B, RAD51AP1, RAD54L2, RAET1G, RAET1L, RALA, RALBP1, RALGAPA1, RAN, RANBP1, RANBP17, RANBP2, RANBP6, RAP1A, RAP1B, RAP1GDS1, RAP2A, RAP2B, RARS, RASA4, RASA4B, RASGRP2, RBAK, RBAK-LOC389458, RBBP4, RBBP6, RBM14-RBM4, RBM15, RBM17, RBM39, RBM4, RBM43, RBM48, RBM4B, RBM7, RBM8A, RBMS1, RBMS2, RBMX, RBMX2, RBMXL1, RBMXL2, RBMY1A1, RBMY1B, RBMY1D, RBMY1E, RBMY1F, RBMY1J, RBPJ, RCBTB1, RCBTB2, RCC2, RCN1, RCOR2, RDBP, RDH16, RDM1, RDX, RECQL, REG1A, REG1B, REG3A, REG3G, RELA, RERE, RETSAT, REV1, REXO4, RFC3, RFESD, RFK, RFPL1, RFPL2, RFPL3, RFPL4A, RFTN1, RFWD2, RGL2, RGPD1, RGPD2, RGPD3, RGPD4, RGPD5, RGPD6, RGPD8, RGS17, RGS19, RGS9, RHBDF1, RHCE, RHD, RHEB, RHOQ, RHOT1, RHOXF2, RHOXF2B, RHPN2, RIMBP3, RIMBP3B, RIMBP3C, RIMKLB, RING1, RLIM, RLN1, RLN2, RLTPR, RMND1, RMND5A, RNASE2, RNASE3, RNASE7, RNASE8, RNASEH1, RNASET2, RNF11, RNF123, RNF126, RNF13, RNF138, RNF14, RNF141, RNF145, RNF152, RNF181, RNF2, RNF216, RNF39, RNF4, RNF5, RNF6, RNFT1, RNMTL1, RNPC3, RNPS1, ROBO2, ROCK1, ROPN1, ROPN1B, RORA, RP9, RPA2, RPA3, RPAP2, RPE, RPF2, RPGR, RPL10, RPL10A, RPL10L, RPL12, RPL13, RPL14, RPL15, RPL17, RPL17-C180RF32, RPL18A, RPL19, RPL21, RPL22, RPL23, RPL23A, RPL24, RPL26, RPL26L1, RPL27, RPL27A, RPL29, RPL3, RPL30, RPL31, RPL32, RPL35, RPL35A, RPL36, RPL36A, RPL36A-HNRNPH2, RPL36AL, RPL37, RPL37A, RPL39, RPL4, RPL41, RPL5, RPL6, RPL7, RPL7A, RPL7L1, RPL8, RPL9, RPLP0, RPLP1, RPP21, RPS10, RPS10-NUDT3, RPS11, RPS13, RPS14, RPS15, RPS15A, RPS16, RPS17, RPS17L, RPS18, RPS19, RPS2, RPS20, RPS23, RPS24, RPS25, RPS26, RPS27, RPS27A, RPS28, RPS3, RPS3A, RPS4X, RPS4Y1, RPS4Y2, RPS5, RPS6, RPS6KB1, RPS7, RPS8, RPS9, RPSA, RPTN, RRAGA, RRAGB, RRAS2, RRM2, RRN3, RRP7A, RSL24D1, RSPH10B, RSPH10B2, RSPO2, RSRC1, RSU1, RTEL1, RTN3, RTN4IP1, RTN4R, RTP1, RTP2, RUFY3, RUNDC1, RUVBL2, RWDD1, RWDD4, RXRB, RYK, S100A11, S100A7L2, SAA1, SAA2, SAA2-SAA4, SAE1, SAFB, SAFB2, SAGE1, SALL1, SALL4, SAMD12, SAMD9, SAMD9L, SAP18, SAP25, SAP30, SAPCD1, SAPCD2, SAR1A, SATL1, SAV1, SAYSD1, SBDS, SBF1, SCAMP1, SCAND3, SCD, SCGB1D1, SCGBID2, SCGBID4, SCGB2A1, SCGB2A2, SCGB2B2, SCN10A, SCN1A, SCN2A, SCN3A, SCN4A, SCN5A, SCN9A, SCOC, SCXA, SCXB, SCYL2, SDAD1, SDCBP, SDCCAG3, SDHA, SDHB, SDHC, SDHD, SDR42E1, SEC11A, SEC14L1, SEC14L4, SEC14L6, SEC61B, SEC63, SELT, SEMA3E, SEMG1, SEMG2, SEPHS1, SEPHS2, SEPT14, SEPT7, SERBP1, SERF1A, SERF1B, SERF2, SERHL2, SERPINB3, SERPINB4, SERPINH1, SET, SETD8, SF3A2, SF3A3, SF3B14, SF3B4, SFR1, SFRP4, SFTA2, SFTPA1, SFTPA2, SH2D1B, SH3BGRL3, SH3GL1, SHANK2, SHC1, SHCBP1, SHFM1, SHH, SHISA5, SHMT1, SHOX, SHQ1, SHROOM2, SIGLEC10, SIGLEC11, SIGLEC12, SIGLEC14, SIGLEC5, SIGLEC6, SIGLEC7, SIGLEC8, SIGLEC9, SIMC1, SIN3A, SIRPA, SIRPB1, SIRPG, SIX1, SIX2, SKA2, SKIV2L, SKOR2, SKP1, SKP2, SLAIN2, SLAMF6, SLC10A5, SLC16A14, SLC16A6, SLC19A3, SLC22A10, SLC22A11, SLC22A12, SLC22A24, SLC22A25, SLC22A3, SLC22A4, SLC22A5, SLC22A9, SLC25A13, SLC25A14, SLC25A15, SLC25A20, SLC25A29, SLC25A3, SLC25A33, SLC25A38, SLC25A47, SLC25A5, SLC25A52, SLC25A53, SLC25A6, SLC29A4, SLC2A13, SLC2A14, SLC2A3, SLC31A1, SLC33A1, SLC35A4, SLC35E1, SLC35E2, SLC35E2B, SLC35G3, SLC35G4, SLC35G5, SLC35G6, SLC36A1, SLC36A2, SLC39A1, SLC39A7, SLC44A4, SLC41AP, SLC52A1, SLC52A2, SLC5A6, SLC5A8, SLC6A14, SLC6A6, SLC6A8, SLC7A5, SLC8A2, SLC8A3, SLC9A2, SLC9A4, SLC9A7, SLCO1B1, SLCO1B3, SLCO1B7, SLFN11, SLFN12, SLFN12L, SLFN13, SLFN5, SLIRP, SLMO2, SLX1A, SLX1B, SMARCE1, SMC3, SMC5, SMEK2, SMG1, SMN1, SMN2, SMR3A, SMR3B, SMS, SMU1, SMURF2, SNAI1, SNAPC4, SNAPC5, SNF8, SNRNP200, SNRPA1, SNRPB2, SNRPC, SNRPD1, SNRPD2, SNRPE, SNRPG, SNRPN, SNW1, SNX19, SNX25, SNX29, SNX5, SNX6, SOCS5, SOCS6, SOGA1, SOGA2, SON, SOX1, SOX10, SOX14, SOX2, SOX30, SOX5, SOX9, SP100, SP140, SP140L, SP3, SP5, SP8, SP9, SPACA5, SPACA5B, SPACA7, SPAG11A, SPAG11B, SPANXA1, SPANXB1, SPANXD, SPANXN2, SPANXN5, SPATA16, SPATA20, SPATA31A1, SPATA31A2, SPATA31A3, SPATA31A4, SPATA31A5, SPATA31A6, SPATA31A7, SPATA31C1, SPATA31C2, SPATA31D1, SPATA31D3, SPATA31D4, SPATA31E1, SPCS2, SPDYE1, SPDYE2, SPDYE2L, SPDYE3, SPDYE4, SPDYE5, SPDYE6, SPECC1, SPECC1L, SPHAR, SPIC, SPIN1, SPIN2A, SPIN2B, SPOPL, SPPL2A, SPPL2C, SPR, SPRR1A, SPRR1B, SPRR2A, SPRR2B, SPRR2D, SPRR2E, SPRR2F, SPRY3, SPRYD4, SPTLC1, SRD5A1, SRD5A3, SREK1IP1, SRGAP2, SRP14, SRP19, SRP68, SRP72, SRP9, SRPK1, SRPK2, SRRM1, SRSF1, SRSF10, SRSF11, SRSF3, SRSF6, SRSF9, SRXN1, SS18L2, SSB, SSBP2, SSBP3, SSBP4, SSNA1, SSR3, SSX1, SSX2, SSX2B, SSX3, SSX4, SSX4B, SSX5, SSX7, ST13, ST3GAL1, STAG3, STAR, STAT5A, STAT5B, STAU1, STAU2, STBD1, STRAP1, STEAP1B, STH, STIP1, STK19, STK24, STK32A, STMN1, STMN2, STMN3, STRADB, STRAP, STRC, STRN, STS, STUB1, STX18, SUB1, SUCLA2, SUCLG2, SUDS3, SUGP1, SUGT1, SULT1A1, SULT1A2, SULT1A3, SULT1A4, SUMF2, SUMO1, SUMO2, SUPT16H, SUPT4H1, SUSD2, SUZ12, SVIL, SW15, SYCE2, SYNCRIP, SYNGAP1, SYNGR2, SYT14, SYT15, SYT2, SYT3, SZRD1, TAAR6, TAAR8, TACC1, TADA1, TAF1, TAF15, TAF1L, TAF4B, TAF5L, TAF9, TAF9B, TAGLN2, TALDO1, TANC2, TAP1, TAP2, TAPBP, TARBP2, TARDBP, TARP, TAS2R19, TAS2R20, TAS2R30, TAS2R39, TAS2R40, TAS2R43, TAS2R46, TAS2R50, TASP1, TATDN1, TATDN2, TBCID26, TBCID27, TBC1D28, TBC1D29, TBC1D2B, TBC1D3, TBC1D3B, TBC1D3C, TBC1D3F, TBC1D3G, TBC1D3H, TBCA, TBCCD1, TBL1X, TBL1XR1, TBL1Y, TBPL1, TBX20, TC2N, TCEA1, TCEAL2, TCEAL3, TCEAL5, TCEB1, TCEB2, TCEB3B, TCEB3C, TCEB3CL, TCEB3CL2, TCERG1L, TCF19, TCF3, TCHH, TCL1B, TCOF1, TCP1, TCP10, TCP10L, TCP10L2, TDG, TDGF1, TDRD1, TEAD1, TEC, TECR, TEKT4, TERF1, TERF2IP, TET1, TEX13A, TEX13B, TEX28, TF, TFB2M, TFDP3, TFG, TGIF1, TGIF2, TGIF2LX, TGIF2LY, THAP3, THAP5, THEM4, THOC3, THRAP3, THSD1, THUMPD1, TIMM17B, TIMM23B, TIMM8A, TIMM8B, TIMP4, TIPIN, TJAP1, TJP3, TLE1, TLE4, TLK1, TLK2, TLL1, TLR1, TLR6, TMA16, TMA7, TMC6, TMCC1, TMED10, TMED2, TMEM126A, TMEM128, TMEM132B, TMEM132C, TMEM14B, TMEM14C, TMEM161B, TMEM167A, TMEM183A, TMEM183B, TMEM185A, TMEM185B, TMEM189-UBE2V1, TMEM191B, TMEM191C, TMEM230, TMEM231, TMEM236, TMEM242, TMEM251, TMEM254, TMEM30B, TMEM47, TMEM69, TMEM80, TMEM92, TMEM97, TMEM98, TMLHE, TMPRSS11E, TMSB10, TMSB15A, TMSB15B, TMSB4X, TMSB4Y, TMTC1, TMTC4, TMX1, TMX2, TNC, TNF, TNFRSF10A, TNFRSF10B, TNFRSF10C, TNFRSF10D, TNFRSF13B, TNFRSF14, TNIP2, TNN, TNPO1, TNRC18, TNXB, TOB2, TOE1, TOMM20, TOMM40, TOMM6, TOMM7, TOP1, TOP3B, TOR1B, TOR3A, TOX4, TP53TG3, TP53TG3B, TP53TG3C, TPD52L2, TPI1, TPM3, TPM4, TPMT, TPRKB, TPRX1, TPSAB1, TPSB2, TPSD1, TPT1, TPTE, TPTE2, TRA2A, TRAF6, TRAPPC2, TRAPPC2L, TREH, TREML2, TREML4, TRIM10, TRIM15, TRIM16, TRIM16L, TRIM26, TRIM27, TRIM31, TRIM38, TRIM39, TRIM39-RPP21, TRIM40, TRIM43, TRIM43B, TRIM48, TRIM49, TRIM49B, TRIM49C, TRIM49DP, TRIM49L1, TRIM50, TRIM51, TRIM51GP, TRIM60, TRIM61, TRIM64, TRIM64B, TRIM64C, TRIM73, TRIM74, TRIM77P, TRIP11, TRMT1, TRMT11, TRMT112, TRMT2B, TRNT1, TRO, TRPA1, TRPC6, TRPV5, TRPV6, TSC22D3, TSEN15, TSEN2, TSPAN11, TSPY1, TSPY10, TSPY2, TSPY3, TSPY4, TSPY8, TSPYL1, TSPYL6, TSR1, TSSK1B, TSSK2, TTC28, TTC3, TTC30A, TTC30B, TTC4, TTL, TTLL12, TTLL2, TTN, TUBA1A, TUBA1B, TUBA1C, TUBA3C, TUBA3D, TUBA3E, TUBA4A, TUBA8, TUBB, TUBB2A, TUBB2B, TUBB3, TUBB4A, TUBB4B, TUBB6, TUBB8, TUBE1, TUBG1, TUBG2, TUBGCP3, TUBGCP6, TUFM, TWF1, TWIST2, TXLNG, TXN2, TXNDC2, TXNDC9, TYR, TYRO3, TYW1, TYW1B, U2AF1, UAP1, UBA2, UBA5, UBD, UBE2C, UBE2D2, UBE2D3, UBE2D4, UBE2E3, UBE2F, UBE2H, UBE2L3, UBE2M, UBE2N, UBE2Q2, UBE2S, UBE2V1, UBE2V2, UBE2W, UBE3A, UBFD1, UBQLN1, UBQLN4, UBTFL1, UBXN2B, UFD1L, UFM1, UGT1A10, UGT1A3, UGT1A4, UGT1A5, UGT1A7, UGT1A8, UGT1A9, UGT2A1, UGT2A2, UGT2A3, UGT2B10, UGT2B11, UGT2B15, UGT2B17, UGT2B28, UGT2B4, UGT2B7, UGT3A2, UHRF1, UHRF2, ULBP1, ULBP2, ULBP3, ULK4, UNC93A, UNC93B1, UPF3A, UPK3B, UPK3BL, UQCR10, UQCRB, UQCRFS1, UQCRH, UQCRQ, USP10, USP12, USP13, USP17L10, USP17L11, USP17L12, USP17L13, USP17L15, USP17L17, USP17L18, USP17L19, USP17L1P, USP17L2, USP17L20, USP17L21, USP17L22, USP17L24, USP17L25, USP17L26, USP17L27, USP17L28, USP17L29, USP17L3, USP17L30, USP17L4, USP17L5, USP17L7, USP17L8, USP18, USP22, USP32, USP34, USP6, USP8, USP9X, USP9Y, UTP14A, UTP14C, UTP18, UTP6, VAMP5, VAMP7, VAPA, VARS, VARS2, VCX, VCX2, VCX3A, VCX3B, VCY, VCY1B, VDAC1, VDAC2, VDAC3, VENTX, VEZF1, VKORC1, VKORC1L1, VMA21, VN1R4, VNN1, VOPP1, VPS26A, VPS35, VPS37A, VPS51, VPS52, VSIG10, VTCN1, VT11B, VWA5B2, VWA7, VWA8, VWF, WARS, WASF2, WASF3, WASH1, WBP1, WBP11, WBP1L, WBSCR16, WDR12, WDR45, WDR45L, WDR46, WDR49, WDR59, WDR70, WDR82, WDR89, WFDC10A, WFDC10B, WHAMM, WHSC1L1, WIPI2, WIZ, WNT3, WNT3A, WNT5A, WNT5B, WNT9B, WRN, WTAP, WWC2, WWC3, WWP1, XAGE1A, XAGE1B, XAGE1C, XAGE1D, XAGE1E, XAGE2, XAGE3, XAGE5, XBP1, XCL1, XCL2, XG, XIAP, XKR3, XKR8, XKRY, XKRY2, XPO6, XPOT, XRCC6, YAP1, YBX1, YBX2, YES1, YMEIL1, YPEL5, YTHDC1, YTHDF1, YTHDF2, YWHAB, YWHAE, YWHAQ, YWHAZ, YY1, YYIAP1, ZAN, ZBED1, ZBTB10, ZBTB12, ZBTB22, ZBTB44, ZBTB45, ZBTB8OS, ZBTB9, ZC3H11A, ZC3H12A, ZCCHC10, ZCCHC12, ZCCHC17, ZCCHC18, ZCCHC2, ZCCHC7, ZCCHC9, ZCRB1, ZDHHC11, ZDHHC20, ZDHHC3, ZDHHC8, ZEB2, ZFAND5, ZFAND6, ZFP106, ZFP112, ZFP14, ZFP57, ZFP64, ZFP82, ZFR, ZFX, ZFY, ZFYVE1, ZFYVE9, ZIC1, ZIC2, ZIC3, ZIC4, ZIK1, ZKSCAN3, ZKSCAN4, ZMIZ1, ZMIZ2, ZMYM2, ZMYM5, ZNF100, ZNF101, ZNF107, ZNF114, ZNF117, ZNF12, ZNF124, ZNF131, ZNF135, ZNF14, ZNF140, ZNF141, ZNF146, ZNF155, ZNF160, ZNF167, ZNF17, ZNF181, ZNF185, ZNF20, ZNF207, ZNF208, ZNF212, ZNF221, ZNF222, ZNF223, ZNF224, ZNF222, ZNF226, ZNF229, ZNF230, ZNF233, ZNF234, ZNF235, ZNF248, ZNF223, ZNF224, ZNF257, ZNF229, ZNF26, ZNF264, ZNF266, ZNF267, ZNF280A, ZNF280B, ZNF282, ZNF283, ZNF284, ZNF282, ZNF286A, ZNF286B, ZNF300, ZNF302, ZNF311, ZNF317, ZNF320, ZNF322, ZNF323, ZNF324, ZNF324B, ZNF33A, ZNF33B, ZNF341, ZNF347, ZNF35, ZNF350, ZNF354A, ZNF324B, ZNF354C, ZNF366, ZNF37A, ZNF383, ZNF396, ZNF41, ZNF415, ZNF416, ZNF417, ZNF418, ZNF419, ZNF426, ZNF429, ZNF43, ZNF430, ZNF431, ZNF433, ZNF439, ZNF44, ZNF440, ZNF441, ZNF442, ZNF443, ZNF444, ZNF451, ZNF460, ZNF468, ZNF470, ZNF479, ZNF480, ZNF484, ZNF486, ZNF491, ZNF492, ZNF506, ZNF528, ZNF532, ZNF534, ZNF543, ZNF546, ZNF547, ZNF248, ZNF552, ZNF555, ZNF257, ZNF528, ZNF561, ZNF562, ZNF563, ZNF264, ZNF57, ZNF570, ZNF578, ZNF283, ZNF585A, ZNF585B, ZNF586, ZNF587, ZNF587B, ZNF589, ZNF592, ZNF594, ZNF595, ZNF598, ZNF605, ZNF607, ZNF610, ZNF613, ZNF614, ZNF615, ZNF616, ZNF620, ZNF621, ZNF622, ZNF625, ZNF626, ZNF627, ZNF628, ZNF646, ZNF649, ZNF622, ZNF625, ZNF658, ZNF665, ZNF673, ZNF674, ZNF675, ZNF676, ZNF678, ZNF679, ZNF680, ZNF681, ZNF682, ZNF69, ZNF700, ZNF701, ZNF705A, ZNF705B, ZNF705D, ZNF705E, ZNF705G, ZNF706, ZNF708, ZNF709, ZNF710, ZNF714, ZNF716, ZNF717, ZNF718, ZNF720, ZNF721, ZNF726, ZNF727, ZNF728, ZNF729, ZNF732, ZNF735, ZNF736, ZNF737, ZNF746, ZNF747, ZNF749, ZNF75A, ZNF75D, ZNF761, ZNF763, ZNF764, ZNF765, ZNF766, ZNF770, ZNF773, ZNF775, ZNF776, ZNF777, ZNF780A, ZNF780B, ZNF782, ZNF783, ZNF791, ZNF792, ZNF799, ZNF805, ZNF806, ZNF808, ZNF812, ZNF813, ZNF814, ZNF816, ZNF816-ZNF321P, ZNF823, ZNF829, ZNF83, ZNF836, ZNF84, ZNF841, ZNF844, ZNF845, ZNF850, ZNF852, ZNF878, ZNF879, ZNF880, ZNF90, ZNF91, ZNF92, ZNF93, ZNF98, ZNF99, ZNRD1, ZNRF2, ZP3, ZRSR2, ZSCAN5A, ZSCAN5B, ZSCAN5D, ZSWIM5, ZXDA, ZXDB, and ZXDC.

In some embodiments, a method described herein detects the presence or absence of a mutation in an EGFR gene. In some embodiments a genetic variation of interest comprises the presence of a c.2573T>G (T becomes a G) substitution in exon 21 of EGFR. In some embodiments, a method described herein detects the presence of, or absence of a c.2573T>G (T becomes a G) substitution in exon 21 of EGFR.

In some embodiments, a method described herein detects the presence or absence of a mutation in an KRAS gene. In some embodiments a genetic variation of interest comprises the presence of a G to a T or a G to an A at position 35 of the KRAS gene (i.e., the codon of the KRAS gene that codes for amino acid 12 and gives rise to the G12D and G12V mutation, respectively. In some embodiments a KRAS genetic variation of interest includes a polymorphism or mutation that produces a G12D, G12V, G13D, G12C, G12A, G12S, G12R, or G13C amino acid mutation.

In some embodiments, a method described herein detects the presence or absence of a mutation in an KRAS gene by employing at least one of the following primers and blocking oligonucleotides in the method:

Forward primer:
(SEQ ID NO: 7)
/5Biosg/ATTGTTGGATCATATTCGTCCAC
Reverse primer:
(SEQ ID NO: 8)
/5Phos/AGGCCTGCTGAAAATGACTG
Blocking oligonucleotide:
(SEQ ID NO: 9)
5′-C+T+G+G+T+G+G+C+G+T+A-3′,
where “+” indicates locked nucleic acid.

In some embodiments, a method described herein detects the presence or absence of a mutation in an KRAS gene by employing the following primers and blocking oligonucleotides in the method:

Forward primer:
(SEQ ID NO: 7)
/5Biosg/ATTGTTGGATCATATTCGTCCAC
Reverse primer:
(SEQ ID NO: 8)
/5Phos/AGGCCTGCTGAAAATGACTG
Blocking oligonucleotide:
(SEQ ID NO: 9)
5′-C+T+G+G+T+G+G+C+G+T+A-3′,
where “+” indicates locked nucleic acid.

In some embodiments, a method described herein detects the presence or absence of a mutation in an KRAS gene by employing at least one of the following capture nucleic acids:

KRAS G12D Probe:
(SEQ ID NO: 10)
/5AmMC6/AAAAAAAAAAGTTGGAG+CTG+ATG+GCGTAG,
KRAS G12V probe:
(SEQ ID NO: 11)
/5AmMC6/AAAAAAAAAAGTTGGAG+CTG+TT+GGC+GTAG
KRAS G12C probe:
(SEQ ID NO: 12)
/5AmMC6/AAAAAAAAAAGTTGGAG+CT+TGT+GGC+GTAG
KRAS G12A probe:
(SEQ ID NO: 13)
/5AmMC6/AAAAAAAAAAGTTGGAGCTG+CTGGCGTAG
KRAS G12S probe:
(SEQ ID NO: 14)
/5AmMC6/AAAAAAAAAAGTTGGAG+CT+AGT+GGC+GTAG

In some embodiments, a method described herein detects the presence or absence of one or more mutations in an KRAS gene by employing employing at least one of the following capture nucleic acids:

KRAS G12D Probe:
(SEQ ID NO: 10)
/5AmMC6/AAAAAAAAAAGTTGGAG+CTG+ATG+GCGTAG,
KRAS G12V probe:
(SEQ ID NO: 11)
/5AmMC6/AAAAAAAAAAGTTGGAG+CTG+TT+GGC+GTAG
KRAS G12C probe:
(SEQ ID NO: 12)
/5AmMC6/AAAAAAAAAAGTTGGAG+CT+TGT+GGC+GTAG
KRAS G12A probe:
(SEQ ID NO: 13)
/5AmMC6/AAAAAAAAAAGTTGGAGCTG+CTGGCGTAG
KRAS G12S probe:
(SEQ ID NO: 14)
/5AmMC6/AAAAAAAAAAGTTGGAG+CT+AGT+GGC+GTAG

In some embodiments, a method described herein detects the presence or absence of one or more mutations in an KRAS gene by employing the following capture nucleic acids the following primers and blocking oligonucleotides and capture nucleic acids in the method:

Forward primer:
(SEQ ID NO: 7)
/5Biosg/ATTGTTGGATCATATTCGTCCAC
Reverse primer:
(SEQ ID NO: 8)
/5Phos/AGGCCTGCTGAAAATGACTG
Blocking oligonucleotide:
(SEQ ID NO: 9)
5′-C+T+G+G+T+G+G+C+G+T+A-3′,
where “+” indicates locked nucleic acid
Capture nucleic acids:
KRAS G12D Probe:
(SEQ ID NO: 10)
/5AmMC6/AAAAAAAAAAGTTGGAG+CTG+ATG+GCGTAG,
KRAS G12V probe:
(SEQ ID NO: 11)
/5AmMC6/AAAAAAAAAAGTTGGAG+CTG+TT+GGC+GTAG
KRAS G12C probe:
(SEQ ID NO: 12)
/5AmMC6/AAAAAAAAAAGTTGGAG+CT+TGT+GGC+GTAG
KRAS G12A probe:
(SEQ ID NO: 13)
/5AmMC6/AAAAAAAAAAGTTGGAGCTG+CTGGCGTAG
KRAS G12S probe:
(SEQ ID NO: 14)
/5AmMC6/AAAAAAAAAAGTTGGAG+CT+AGT+GGC+GTAG

In some embodiments, a method described herein detects the presence or absence of an organism in a sample. In some embodiments, a method herein detects one or more organism in a sample, or suspected of being present in a a sample, by detecting one or more genetic variants that characterizes and distinguishes such one or more organisms from another organism (or class of organisms). In some embodiments, primers, or sets of primers, that are configured to amplify one or more target nucleic acids that are useful in distinguishing such genetic variants are employed in accordance with the disclosed methods. In some embodiments, target nucleic acids, or sets of target nucleic acids, are identified and targeted (by, for example, designing primer or primer sets that amplify such target nucleic acids) to detection in accordance with the disclosed methods so that one or more organisms are detected and distinguished. In some embodiments, capture nucleic acids, or sets of capture nucleic acids are configured capture amplicons (also referred to throughout as distinguishable amplicons), that are generated according to the disclosed methods and which may be detected and used to distinguish the presence of one or more organisms from at least one other organism in a sample.

In some embodiments, a method described herein determines that a subject, has or is at risk of developing a disease or condition non-limiting examples of which include cancer. One potential practical application of the technology is to identify mutations that are present in a cancer so that a patient can be administered an appropriate effective treatment for that cancer. Certain non-limiting examples of such cancers are shown in the table below, with their associated recommended treatments.

In some embodiments, a method described herein determines that a subject, has or is at risk of developing a disease or condition non-limiting examples of which include cancer. One potential practical application of the technology is to identify mutations that are present in a cancer so that a patient can be administered an appropriate effective treatment for that cancer. Certain non-limiting examples of such cancers are shown in Table 2 below, with their associated recommended treatments.

TABLE 2

| | Mutations that can be | |
|---|---|---|
| Cancer | detected | Targeted drug therapy |
| Lung | EGFR: exon 19 deletions, | Afatinib, gefitinib, |
| Cancer | L858R mutation, T790M | erlotinib, osimertinib, |
| | mutation, exon 20 mutations, | alectinib, crizotinib, |
| | ALK rearrangements, | or ceritinib |
| | BRAF V600E | |
| Melanoma | BRAF V600E or V600K | Dabrafenib or vemurafenib |
| Breast | PIK3CA mutations, or | PIQRAY, trastuzumab, ado- |
| Cancer | ERBB2 (HER2) amplification | trastuzumabemtansine, |
| | | pertuzumab |
| Colorectal | KRAS mutations or NRAS | Cetuximab or panitumumab |
| cancer | mutations | for non-mutant patients |
| Ovarian | BRCA1/2 mutations | rucaparib |
| Cancer | | |

### Methods

Presented herein, in certain embodiments, is a method of detecting the presence or absence of a genetic variation or an allelic variant in a sample. In certain embodiments, a method comprises detecting the presence or absence of a genetic variation or an allelic variant in a target nucleic acid. In some such embodiments, a method comprises detecting the presence or absence of a cancer in a subject. In some embodiments, a method or detecting process comprises detecting a presence, absence, amount, or change thereof, of magnetic particles at, on, near or associated with a surface of a magnetic sensor. In some embodiments, a presence, absence, amount, or change thereof, of magnetic particles bound to a surface of a magnetic sensor is detected. In certain embodiments, a detection process or detection step comprises detecting a change in an amount of magnetic particles at, near, or on the surface of a magnetic sensor over a period of time.

In some embodiments, a detection process comprises a dynamic detection process. In certain embodiments, a dynamic detection process comprises detecting a presence, absence, amount, or change in an amount of magnetic particles at, near, or on the surface of a magnetic sensor over time, while conditions at, near or on the surface of a magnetic sensor are changed. Non-limiting examples of conditions that can be changed during a dynamic detection process include temperature, salt concentration, cation concentration, ion concentration, pH, detergent concentration, chaotropic agent concentration, ionic kosmotrope concentration, the like or combinations thereof. Often, conditions are changed during a dynamic detection process to increase stringency of protein-protein interactions or protein-DNA interactions at, on or near a surface of a magnetic sensor.

In some embodiments, a dynamic detection process comprises detecting a change in an amount of magnetic particles at, near, or on a surface of a magnetic sensor over time, while temperature is increased over a period of time. In some embodiments, a dynamic detection process comprises detecting a change in an amount of magnetic particles at, near, or on the surface of a magnetic sensor over a period of time, while a concentration of cations (e.g., Na, Ca, Mg, Zn and the like) is increases or decreased. In some embodiments, a dynamic detection process comprises detecting a change in an amount of magnetic particles at, near, or on a surface of a magnetic sensor over a period of time, while temperature is increased and/or while a concentration of salt is increased or decreased.

In some embodiments, a method comprises detecting or determining a magnetoresistance, current, voltage potential, or change thereof on, near or at the surface of a magnetic sensor. In some embodiments, a magnetoresistance, current, voltage potential, or change thereof, on, near or at the surface of a magnetic sensor is determined or detected once, continuously (e.g., during a predetermined period of time), or periodically (e.g., two or more times) before, during and/or after a magnetic sensor is contacted with magnetic particles as described herein. In some embodiments, a magnetoresistance, current, voltage potential, or change thereof, on, near or at the surface of a magnetic sensor is determined or detected continuously (e.g., during a predetermined period of time), or periodically (at two or more times) while a temperature is increased at the surface of a magnetic sensor.

In some embodiments, some or all aspects of a method and/or some or all steps of a method described herein are performed in a microfluidic device described herein.

In some embodiments, a method comprises extracting, isolating or purifying nucleic acids from a sample. In some embodiments, nucleic acids are extracted, isolated or purified from a sample by contacting the sample with a suitable cell lysis solution. A cell lysis solution is often configured to lyse whole cells, and/or separate nucleic acids from contaminants (e.g., proteins, carbohydrates and fatty acids). A cell lysis solution may comprise one or more lysis reagents, non-limiting examples of which include detergents, hypotonic solutions, high salt solutions, alkaline solutions, organic solvents (e.g., phenol, chloroform), chaotropic salts, enzymes, the like, and combination thereof.

In some embodiments, nucleic acids are extracted, isolated or purified from a sample by contacting the sample with a membrane (e.g., a membrane of a microfluidic device described herein), optionally after contacting a sample with a cell lysis solution. In some embodiments, a device described herein performs a process of extracting, isolating or purifying nucleic acids from a sample which process comprises contacting a sample with a cell lysis solution and/or a membrane. In some embodiments, a silica membrane is employed as part of the extraction process. In some embodiments, a method comprising selectively amplifying a target nucleic such that one or more amplicons (e.g., copies) of the target nucleic acid are produced.

Nucleic acid may be provided for conducting methods described herein without processing of a sample containing the nucleic acid, in certain embodiments. In some embodiments, nucleic acid is provided for conducting methods described herein after processing of a sample containing the nucleic acid. For example, a nucleic acid can be extracted, isolated, purified, partially purified or amplified from a sample prior to, during or after a method described herein.

In some embodiments, a target nucleic acid is amplified using a suitable method. In some embodiments, an amplification process comprises a process where one or both strands of a nucleic acid are enzymatically replicated such that amplicons (e.g., copies or complimentary copies) of a target nucleic acid are generated. A nucleic acid amplification process can linearly or exponentially generate amplicons having the same or substantially the same nucleotide sequence as a template or target nucleic acid, or segment thereof. In some embodiments, a target nucleic acid is amplified by a suitable amplification process non-limiting examples of which include polymerase chain reaction (PCR), nested (n) PCR, quantitative (q) PCR, real-time PCR, reverse transcription (RT) PCR, isothermal amplification (e.g., loop mediated isothermal amplification (LAMP)), quantitative nucleic acid sequence-based amplification (QT-NASBA), the like, variations thereof, and combinations thereof. In some embodiments, an amplification process comprises a polymerase chain reaction. In some embodiments, an amplification process comprises performing at least 30, at least 40, at least 45 or at least 50 cycles of a polymerase chain reaction. A cycle of a polymerase chain reaction comprises at least one denaturation step and an optional annealing step, followed by at least one extension step. In some embodiments, a target nucleic acid is amplified using a suitable heat-stable polymerase. In some embodiments, an amplification process comprises an isothermal amplification process.

In some embodiments, an amplification process comprises contacting a target nucleic acid comprising a genetic variation of interest (e.g., an allelic variant of interest) with (i) a first primer, (ii) a second primer comprising a first member of a binding pair, (iii) a suitable polymerase and (iv) a blocking oligonucleotide, wherein the blocking oligonucleotide comprises a sequence complementary to a second allelic variant of the target nucleic acid, and the first and second primers are configured for amplification of the target nucleic acid. In some embodiments, the first primer is attached to a solid substrate or to a surface (e.g., a surface of an amplification chamber. In some embodiments, a first primer comprises a free 5′ hydroxyl group. Any suitable member of a binding pair can be used. In certain embodiments, a first member of a binding pair comprises biotin. In certain embodiments, the first and second primers are configured to amplify the target sequence, or portion thereof.

In some embodiments, the blocking oligonucleotide comprises a locked nucleic acid. In some embodiments, the blocking oligonucleotide comprises one or more locked nucleotides (e.g., at least 1, at least 2, at least 3, at least 4 or at least 5 locked nucleotides).

A blocking oligonucleotide is often configured to anneal or hybridize specifically to a target sequence that does not comprise the genetic variation of interest. For example, where the genetic variation of interest is a single nucleotide variant (e.g., a Guanine (G)), at a specific position within the sequence of the target nucleic acid, alternative variants may include a cytosine (C), adenine (A) or thymine (T) at the specified position. Accordingly, in this example, a blocking oligonucleotide can be configured to specifically hybridize to a target sequence comprising one of the alternative variants such that the blocking oligonucleotide includes a C, A or T at the specified position. Further, in this example, up to three blocking oligonucleotides may be required to block amplification of each of the three alternative variants that may be present in a sample. Often, where a genetic variation of interest is a known single nucleotide substitution (e.g., a single nucleotide mutation associated with a cancer), a blocking oligonucleotide is configured to hybridize to the wild-type variant (i.e., the variant that is not associated with a cancer, e.g., the variant that is found in a healthy subject). The presence of locking nucleotides in a blocking oligonucleotide allows the blocking oligonucleotide to specifically hybridize to its target sequence with a higher melting temperature than each of the primers used for the amplification reaction, thereby substantially blocking amplification of a target nucleic acid that includes an alternative or wild-type variant, if present. In some embodiments, a blocking oligonucleotide comprises a higher melting temperature one or both of the primers used in an amplification reaction. In some embodiments, a blocking oligonucleotide, when hybridized to its complementary sequence, comprises a melting temperature that is at least 10° C., at least ° 20 or at least 25° C. higher than a melting temperature of one or both of the primers used in an amplification reaction. In some embodiments, an amplification reaction is performed in an amplification chamber of a microfluidic device described herein.

In some embodiments, amplicons produced by an amplification reaction are contacted with a suitable exonuclease (e.g., a suitable 5′-3′ exonuclease) such that amplicons comprising a free 5′ hydroxyl group are selectively degraded and/or digested. In certain embodiments, a suitable 5′-3′ exonuclease does not degrade or digest an amplicon comprising a member of a binding pair (e.g., a biotin) that is conjugated to the 5′ hydroxyl group of the amplicon. In some embodiments, amplicons are transported through a microfluidic channel from an amplification chamber of a device described herein to a chamber (e.g., 218, 216 or 210 of FIG. 24) comprising a suitable exonuclease, wherein the amplicons are contacted with the exonuclease.

In some embodiment, amplicons are contacted with a sensor described herein. In some embodiment, amplicons are contacted with a capture nucleic acid, wherein the capture nucleic acid is attached to a surface of a magnetic magnetoresistance sensor. In some embodiments, amplicons are transported through a microfluidic channel, from an amplification chamber of a device described herein to a sensor of a device described herein, such that the amplicons contact a capture nucleic acid that is attached to a surface of the sensor. In certain embodiments, a capture nucleic acid hybridizes specifically to a target nucleic acid, or amplicon thereof, that comprises a genetic variation of interest. In certain embodiments, a capture nucleic acid comprises one or more locked nucleotides. In certain embodiments, a capture nucleic acid comprises a sequence that is at least 80%, at least 90% or 100% identical to a target nucleic acid, or complement thereof. In certain embodiments, a capture nucleic acid comprises a sequence that is at least 80%, at least 90% or 100% identical to a portion of a target nucleic acid that include a genetic variation of interest, or complement thereof. In some embodiments, a capture nucleic acids comprises a sequence complementary to a first allelic variant of a target sequence, where the first allelic variant comprises a genetic variation of interest. Once amplicons are contacted with, and or hybridized to a capture nucleic acid, the surface of the magnetic comprises captured nucleic acids (e.g., captured amplicons). In some embodiments, the captured amplicons are amplicons comprising a member of a binding pair (e.g., biotin). In some embodiments, amplicons comprising a first member of a binding pair (e.g., biotin) are contacted with magnetic particles comprising the second member of the binding pair (e.g., streptavidin) such that the first and second members of the binding pair bind to each other. Amplicons may be contacted with the magnetic particles bearing a member of a binding pair before, during or after the amplicons are captured at the surface of the sensor. Amplicons that are captured on a sensor can be washed one or more times by contacting the surface of the sensor with one or more wash solutions/wash buffers thereby removing unbound and/or non-specifically bound nucleotides and/or magnetic particles.

In some embodiments, captured amplicons are contacted with positively charged ions. In some embodiments, a solution comprising one or more salts or positive ions is introduced into a microfluidic channel such that a concentration of the positively charged ions in fluid contact with the captured amplicons is increased or decreased. For example, in some embodiments, a solution comprising a water, or a diluted buffer comprising a low amount of salts or positive ions, is introduced into a microfluidic channel such that a concentration of positively charged ions in fluid contact with captured amplicons is decreased to 50 mM or less, 30 mM or less, 15 mM or less, 10 mM or less, 5 mM or less, or to 1 mM or less. In some embodiments, a solution or buffer is introduced into a microfluidic channel such that a concentration of positively charged ions in fluid contact with captured amplicons is decreased to a range of about 50 mM to 0.1 mM, about 20 mM to 1 mM, about 10 mM to 1 mM, or intervening ranges thereof In some embodiments, a solution or buffer is introduced into a microfluidic channel such that a concentration of the positively charged ions in fluid contact with the captured amplicons is decreased by about 20%, by about 50%, by about 100%, by about 200% or by about 400%. A concentration of positive ions in contact with a sensor can be decreased prior to, during or after capture of the amplicons to the surface of the sensor.

In some embodiments, a temperature of a fluid in contact with the surface of a sensor and/or amplicons (e.g., captured amplicons) is increased by at least 10° C., by at least 15° C., by at least 20° C., by at least 25° C., by at least 30° C., by at least 40° C., by at least 60° C., or by at least 80° C. over a period of 1 second to 30 minutes, 1 second to 10 minutes, 1 second to 5 minutes, 1 second to 1 minute, or intervening ranges thereof. In some embodiments, a temperature of a fluid in contact with the surface of a sensor and/or amplicons (e.g., captured amplicons) is increased from about 10° C. to about 120° C., from about 10° C. to about 80° C., from about 10° C. to about 70° C., from about 10° C. to about 65° C., from about 10° C. to about 60° C., from about 20° C. to about 120° C., from about 20° C. to about 80° C., from about 20° C. to about 70° C., from about 20° C. to about 65° C., from about 20° C. to about 60° C., from about 25° C. to about 80° C., from about 25° C. to about 70° C., from about 25° C. to about 65° C., from about 25° C. to about 60° C., or intervening ranges thereof, over a period of 1 second to 30 minutes, 1 second to 10 minutes, 1 second to 5 minutes, 1 second to 1 minute, or intervening ranges thereof.

In some embodiments, a method comprises increasing a temperature at the surface of a sensor (e.g., a magnetic sensor comprising captured amplicons and associated magnetic particles) by at least 10° C., by at least 15° C., by at least 20° C., by at least 25° C., by at least 30° C., by at least 40° C., by at least 60° C., or by at least 80° C. over a period of 1 second to 30 minutes, 1 second to 10 minutes, 1 second to 5 minutes, 1 second to 1 minute, or intervening ranges thereof. In some embodiments, a method comprises increasing a temperature at the surface of a sensor (e.g., a magnetic sensor comprising captured amplicons and associated magnetic particles) from about 10° C. to about 120° C., from about 10° C. to about 80° C., from about 10° C. to about 70° C., from about 10° C. to about 65° C., from about 10° C. to about 60° C., from about 20° C. to about 120° C., from about 20° C. to about 80° C., from about 20° C. to about 70° C., from about 20° C. to about 65° C., from about 20° C. to about 60° C., from about 25° C. to about 80° C., from about 25° C. to about 70° C., from about 25° C. to about 65° C., from about 25° C. to about 60° C., or intervening ranges thereof, over a period of 1 second to 30 minutes, 1 second to 10 minutes, 1 second to 5 minutes, 1 second to 1 minute, or intervening ranges thereof.

In some embodiments, a method or detecting process comprises detecting a presence, absence, or amount thereof, of a detectable label. In certain embodiments, a presence, absence, or amount thereof, of a detectable label is detected by a sensor. In certain embodiments, a presence, absence, or amount thereof, of a detectable label is detected at or near a surface of a sensor. In some embodiments, a presence, absence, or amount, of a detectable label bound to a surface of a sensor is detected. In certain embodiments, a detection process or detection step comprises detecting a change in an amount of a detectable label at, near, or on the surface of a sensor over time.

In some embodiments, a detection process comprises a dynamic detection process. In certain embodiments, a dynamic detection process comprises detecting a presence, absence, amount, or change in an amount of a detectable label at, near, or on the surface of a sensor over time, while conditions at, near or on the surface of a sensor are changed. Non-limiting examples of conditions that can be changed during a dynamic detection process include temperature, salt concentration, cation concentration, ion concentration, pH, detergent concentration, chaotropic agent concentration, ionic kosmotrope concentration, the like or combinations thereof. Often, conditions are changed during a dynamic detection process to increase stringency of hybridization conditions at, on or near a surface of a sensor where capture nucleic acids are present. The dynamic detection process allows discrimination between hybridized nucleic acid duplexes having an exact complementary match with the capture nucleic acid, and non-specific nucleic acid duplexes having one or more mismatches with a capture nucleic acid. This is because duplexes that have mismatches with the capture nucleic acid will typically melt, dissociate or denature under less stringent hybridization conditions than a duplex having an exact match with a capture nucleic acid.

In some embodiments, a dynamic detection process comprises detecting a change in an amount of a detectable label at, near, or on a surface of a sensor over time, while temperature is increased over a period of time. In some embodiments, a dynamic detection process comprises detecting a change in an amount of a detectable label at, near, or on the surface of a sensor over a period of time, while a concentration of cations (e.g., Na, Ca, Mg, Zn and the like) is decreased. In some embodiments, a dynamic detection process comprises detecting a change in an amount of a detectable label at, near, or on a surface of a sensor over a period of time, while temperature is increased and/or while a concentration of cations (e.g., Na, Ca, Mg, Zn and the like) is decreased.

In some embodiments, a method comprises detecting or determining a magnetoresistance, current, voltage potential, or change thereof on, near or at the surface of a magnetic sensor. In some embodiments, a magnetoresistance, current, voltage potential, or change thereof, on, near or at the surface of a magnetic sensor is determined or detected once, continuously (e.g., during a predetermined period of time), or periodically (at two or more times) after captured amplicons are contacted with magnetic particles as described herein. In some embodiments, a magnetoresistance, current, voltage potential, or change thereof, on, near or at the surface of a magnetic sensor is determined or detected continuously (e.g., during a predetermined period of time), or periodically (at two or more times) while a temperature is increased at the surface of a magnetic sensor.

In some embodiments a method described herein determines the presence, absence or amount of a genetic variation in a genome of a subject, and/or in a sample comprising nucleic acids obtained from a subject. In some embodiments, the presence, absence or amount of a genetic variation of interest is determined according to a magnetoresistance, current, voltage potential, or change thereof, that is detected or measured on, near or at the surface of a magnetic sensor when performing a method described herein.

In some embodiments, a method described herein does not include a sequencing step wherein one or more nucleic acids are subjected to DNA sequencing. In some embodiments, a method described herein excludes a nucleic acid sequencing process. Accordingly, in some embodiments, a method described herein does not include determining a sequence of a nucleic acid.

Further, in some embodiments, a method described herein does not include a ligation step. In some embodiments, a method described herein excludes a ligation process. Accordingly, in some embodiments, a method described herein does not include the use of a ligase or contacting a nucleic acid with a ligase.

Further, in some embodiments, a method or device described herein does not include a microarray, or use of a microarray.

### EXAMPLES

### Example 1

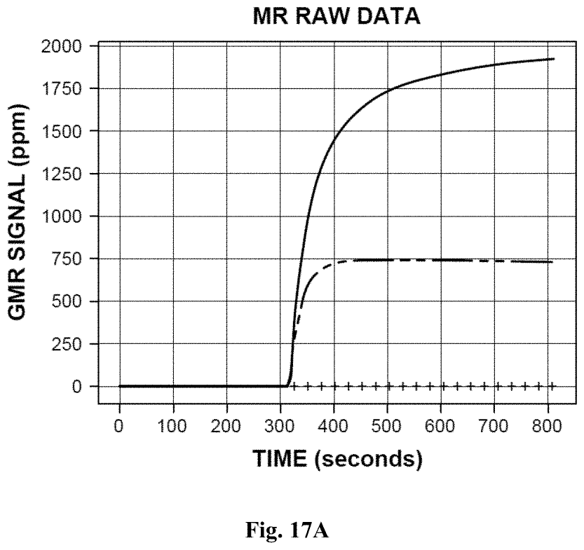

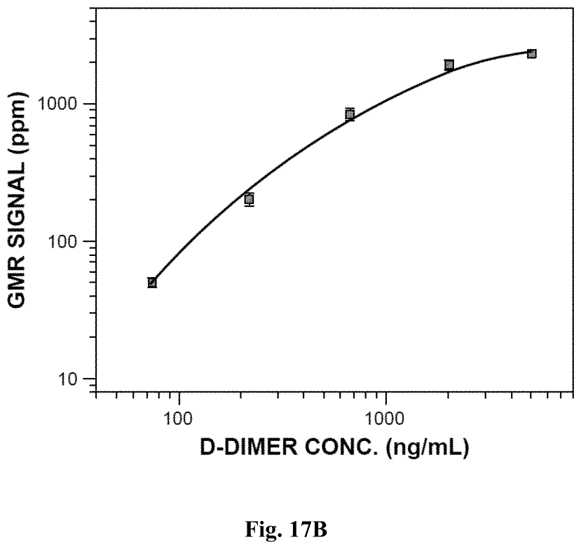

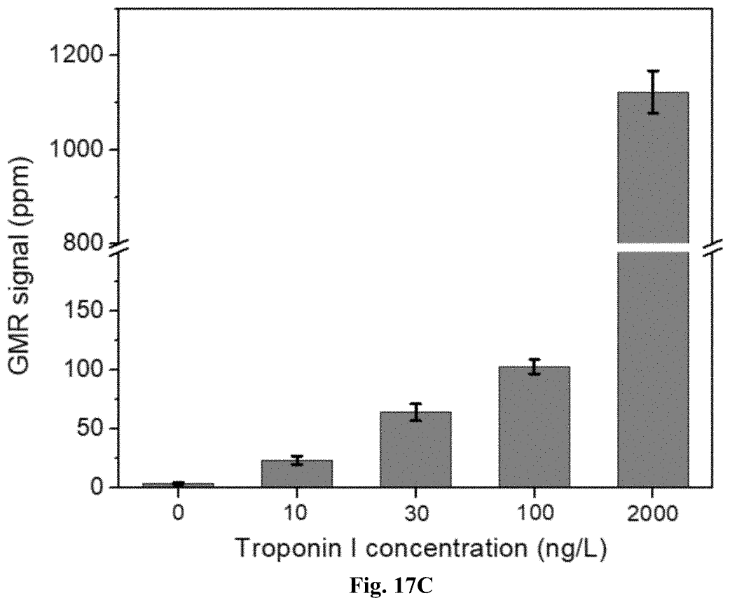

To demonstrate detection of an exemplary biomarker, the schemes of FIGS. 16A and 16B were employed with cardiac biomarkers. Results are are shown in FIGS. 17A-C. FIG. 17A shows a plot of GMR signal (in ppm) over time (in seconds) in a test run designed to detect cardiac biomarker D-dimer. To generate this data, a biosurface was prepared on sensors by functionalizing the surface of the sensors (via crosslinking of the biotin moiety to a polymer composition on the sensor, as described above) and printing a D-dimer capture antibody using 2 nL of a 1 mg/mL of D-dimer antibody in PBS buffer with 0.05% sodium azide. For testing potential cross reactivity, the biosurface was also functionalized with troponin I capture antibody by printing two combined capture antibodies using 2 nL of a solution of 1 mg/mL troponin I antibody in PBS buffer with 0.05% sodium azide. Additionally, two other controls were printed on the biosurface. The first is a negative control prepared by printing 2 nL of a solution of 0.5% BSA in PBS buffer with 0.05% sodium azide and the second is a positive control prepared by printing 2 nL of lmg/mL of biotin conjugated to mouse IgG in PBS buffer with 0.05% sodium azide. The printed sensors were incorporated into a cardiac test cartridge and is configured to use the “sandwich” assay described above in FIGS. 16A and 16B.

In the sample test 120 microliters of plasma or whole blood was loaded into a sample well in the cartridge. A membrane filter serves to remove blood cells as the sample is pulled into the flow channel from the sample well. 40 microliters of plasma (or plasma portion of whole blood) is flowed into a metering channel and deposited powder including antibody/biotin conjugates, blockers, and mouse IgG in the channel dissolve into the sample solution. While flowing over the sensor area, the analytes, antibody/biotin conjugates and antibodies immobilized on the sensor surface formed a sandwich of antibody-analyte-biotinylated antibody. Flow rates were modulated depending on the test. For troponin I, the sample was flowed over the sensor for 20 minutes at a flow rate of 1 microliter/minute. For D-dimer, the sample was flowed for 5 minutes at a flow rate of 4 microliters/minute. Following flow of the sample streptavidin-coated magnetic beads were introduced which allow binding to the sensor surface wherever there wasa biotinylated antibody bound. The GMR sensor measure bound magnetic beads, which was proportional to the concentration of analytes with the sample. The bead solution was flowed over the sensor for 5 minutes at a flow rate of 4 to 10 microliters/minute. The signals were read from the peak value within 300 seconds after beads started to bind.

As indicated in the plot of FIG. 17A, a negative control with just printed BSA did not bind D-Dimer and thus, the signal remained near baseline as expected. The positive control with biotinylated mouse IgG showed competent bead binding, as expected. A plot of the actual sample of 666.6 ng/mL of human D-dimer appeared with a peak detection signal of about 750 ppm indicating successful detection of the D-dimer in an actual sample. There was virtually no cross reactivity with the two bound troponin I capture antibodies (not shown for clarity because these lines were very close to the line with the negative control).

FIG. 17B shows a calibration curve (GMR signal in ppm vs. D-dimer concentration) for D-dimer by running samples with varied, fixed concentrations of D-dimer. The calibration curve allows concentrations to be computed for a future unknown sample containing the D-dimer as the query analyte. A similar plot in FIG. 17C is provided for the cardiac biomarker troponin I. Together, these results establish the viability of detecting D-dimer and troponin I in, blood or plasma samples of a subject.

### Example 2

To demonstrate amplification of GMR signal for analyte detection a sandwich immunoassay format as depicted, for example, in FIG. 16A, was performed. Biotinylated-troponin I capture antibodies were flowed over independent GMR sensors to create a series of troponin I biosurface-attached sensors (via crosslinking of the biotin moiety to a polymer composition on the sensor) in order to test varying concentrations of Troponin I. Each of the query samples containing the different concentrations of Troponin I as indicated in Table 3, below, was flowed over a Troponin I capture antibody-printed sensor. Other biotinylated-anti-troponin I antibodies where then flowed over the sensors. Subsequently, straptavidin-coated magnetic nanoparticles were then flowed over each sensor surface and bound to the biotinylated-anti-troponin I antibodies that were bound to the surface via biotin-streptavidin interaction. Sensor signal readings (change in magnetoresistance) were recorded as indicated in Table 3 (“Primary signal”).

Subsequently, biotin-coated magnetic nanoparticles were flowed over the sensors; these biotin-coated magnetic nanoparticles then bound to the free strepatavidin groups on the streptavidin -coated magnetic nanoparticles. Sensor signal readings (change in magnetoresistance) were recorded as indicated in Table 3 (“1^{st} enhanced signal signal”).

Subsequently, another sample of streptavidin-coated magnetic nanoparticles were flowed over the sensors; these steptavidin-coated magnetic nanoparticles then bound to the free biotin groups on the biotin-coated magnetic nanoparticles. Sensor signal readings (change in magnetoresistance) were recorded as indicated in Table 3 (“2^{st} enhanced signal signal”).

TABLE 3
Human troponin I test data without and with signal enhancement

| Troponin I | Primary signal | 1^{st} enhanced | 2^{nd} enhanced |
|---|---|---|---|
| (ng/L) | (ppm) | signal (ppm) | signal (ppm) |
| 0 | 0.8 | 2.4 | 4.4 |
| 7.8 | 4.8 | 60 | 94.7 |
| 31.5 | 11.7 | 178 | 373.9 |
| 125 | 39.5 | 266.2 | 554.9 |

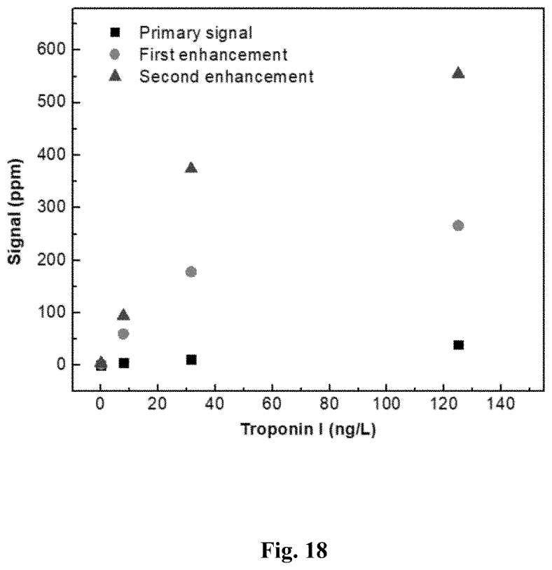

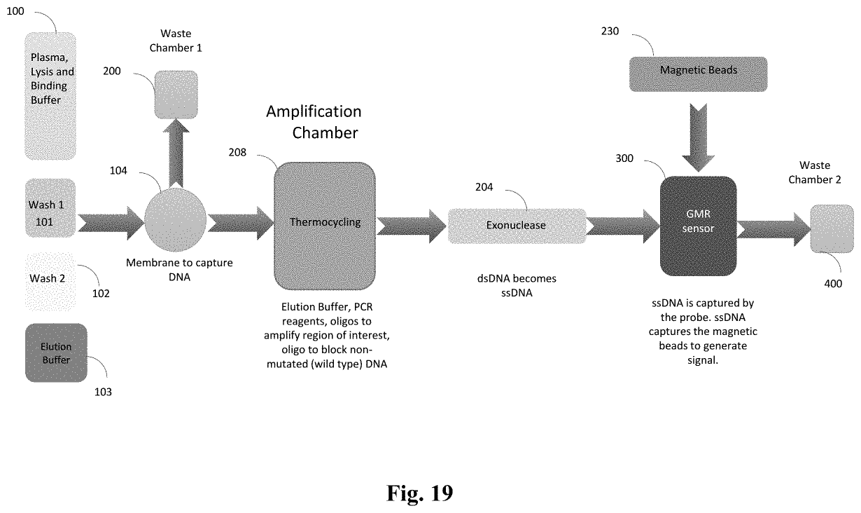

The results, shown in Table 3 and in FIG. 18, demonstrate that for low levels (0-125 ng/L) of tropinin I tests, all signals were significantly increased after 1st and 2nd signal enhancement processes. For blank sample (0 ng/L troponin I), the signal modestly increased (from 0.8 ppm to 2.4 and 4.4) after each enhancement, indicating that assay “noise” was slightly increase. However, for 7.8 ng/L, the SNR (signal to noise ratio) increased from 6 to 25 and 21.5 after each subsequent enhancement. Significant signal enhancements were also achieved at the 31.5 and 125 nh/mL Troponin I concentrations, as well.

The magnetic (GMR) sensor measures bound magnetic beads which are proportional to the concentration of analytes in the sample. In situations where the amount of bound beads is very low, the GMR sensor signal to noise ratio may be lower than desired. The results described herein demonstrate that the signal to noise ratio can be markedly enhanced in such by flowing magnetic beads coated with biotin (MB-Biotin) which was captured by the initial magnetic beads coated with straptavidin (MB-SA) that was captured on the surface of sensors that had been previously exposed to samples containing troponin I on surface. Then MB-SA flowed again over sensor surface and additional signal enhancement was generated due to MB-SA subsequent binding by MB-Biotin on the sensor. The altering of MB-Biotin and MB-SA can be repeated for multiple rounds of enhancement to further increase the GMR signals.

Signal amplification as described above may be employed for methods of detecting biomarkers, as well as genetic variants, and/or allelic variants, and/or for distinguishing between possible genetic and/or allelic variants that are present, or are suspected of being present, in one or more samples.

### Example 3

EGFR is a gene that encodes the Epidermal Growth Factor Receptor, which is a transmembrane glycoprotein receptor for members of the epidermal growth factor family. A single nucleotide mutation c.2573T>G (T becomes a G) in exon 21 of EGFR results in an amino acid substitution of leucine (L) at position 858 by an arginine (R) (L858R), which is causative and predictive of lung cancer. An Epidermal Growth Factor Receptor having the L858R mutation is constantly activated causing uncontrolled cell growth and cell proliferation.

The c.2573T>G mutation was detected with high sensitivity in a plasma sample containing cell free DNA (cfDNA) obtained from a subject. The process was non-invasive, as a tissue biopsy was not required. Also, the assay method only required the presence of cfDNA, and does not require purification and lysis of lymphocytes obtained from a buffy coat fraction. However, DNA from blood cells can also be analyzed using this method by implementing an optional lysis buffer step as demonstrated in this example. All of the following processes were performed on a microfluidic device described herein (e.g., see FIGS. 1-15 and 24-26).

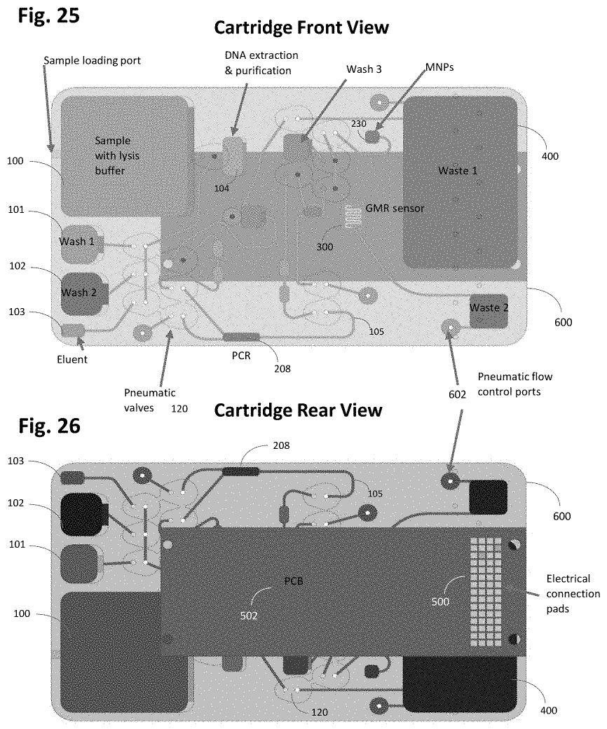

Briefly, and referring to FIGS. 25 and 26, a plasma sample was introduced into the sample loading port 605 where it was contacted with a cell lysis buffer containing Guanidine Hydrochloride (GuHCl, Sigma: G3272), Tris-HCl, pH 8.0, Triton X-100 and Isopropanol. The sample was transported through valve V1 and microfluidic channel 105 to a silica fiber membrane (e.g., 104), where nucleic acids in the sample were bound to the silica fiber membrane. The membrane was washed by introducing a wash buffer from chamber 101 and/or chamber 102 through valves V2 and/or V3 into the microfluidic channel 105. The wash buffer was passed through the membrane, proceeded through the microfluidic channel to valve V5 and to the extraction waste chamber 200 by applying a negative pressure using Diaphragm pump 1. After washing, valves V1, V2 and V3 were switched inline with V4, valve V4 was opened, V5 was closed and V6 was opened. Nucleic acids that were bound to the membrane were eluted and passed to the elution collection chamber 201 by directing the elution buffer stored in chamber 103 through the microfluidic channel to the membrane 104 and subsequently to the elution collection chamber 201 by applying a negative pressure using Diaphragm Pump 1 to chamber 201.

After the DNA was eluted, the elution product was contacted with lyophilized amplification reagents stored in chamber 204, and the mixture was moved into mixing chamber 206 and through valve V7 to the amplification chamber 208 (referring to FIG. 6). The amplification reagents included an amplification buffer, dNTPs, a biotinylated forward primer, a reverse primer comprising a free 5′-hydroxyl, a blocking oligonucleotide and a heat stable polymerase (KLEN TAQ®).

The mutant target nucleic acid of the EGFR gene is below with the genetic variation of interest (mutation) shown underlined and bolded.

(SEQ ID NO: 1)
CAGCCAGGAACGTACTGGTGAAAACACCGCAGCATGTCAAG
ATCACAGATTTTGGGCGGGCCAAACTGCTGGGTGCGGAAGA
GAAAGAATACCATGCAGAAGGAGGCAAAGT

The wild type non-mutated target sequence is shown below.

(SEQ ID NO: 2)
CAGCCAGGAACGTACTGGTGAAAACACCGCAGCATGTCAA
GATCACAGATTTTGGGCTGGCCAAACTGCTGGGTGCGGAA
GAGAAAGAATACCATGCAGAAGGAGGCAAAGT

The underlined portion of SEQ ID NO:2 (CAGCCAGGAACGTACTGGTGAAAACACCGCAGCATGTCAAGATCACAGATTTTGGG CTGGCCAAACTGCTGGGTGCGGAAGAGAAAGAATACCATGCAGAAGGAGGCAAAG T) is complementary to the sequence of the blocking oligonucleotide of SEQ ID NO:5 (5′-TTTGGCCAGC). The forward primer and reverse primer are also shown below. The forward primer contained a 5′-conjugated biotin moiety. The reverse primer included a 5′-phosphate group. The blocking oligonucleotide is a locked nucleic acid (LNA) and all nucleotides of the blocking oligonucleotide are locked nucleotides. The locked nucleotides comprised an extra methylene bridge fixed to the ribose moiety either in the C3′-endo (beta-D-LNA) or C2′-endo (alpha-L-LNA) conformation.

Forward Primer:
(SEQ ID NO: 3)
/5′-Biosg/CAGCCAGGAACGTACTGGTG
Reverse Primer:
(SEQ ID NO: 4)
/5′-Phos/ACTTTGCCTCCTTCTGCATG
Blocking oligonucleotide:
(SEQ ID NO: 5)
5′-TTTGGCCAGC

Valves V7, V8 and V9 were closed and the nucleic acids and reagents in the amplification chamber were subjected to thermal cycling for >40 cycles with a denaturation (melting) step at 95° C. and an annealing/extension step at 58° C. The amplification chamber/module is a serpentine shaped thin plastic PCR micro reactor. The thermocycling temperature was achieved by a Peltier cooling module.

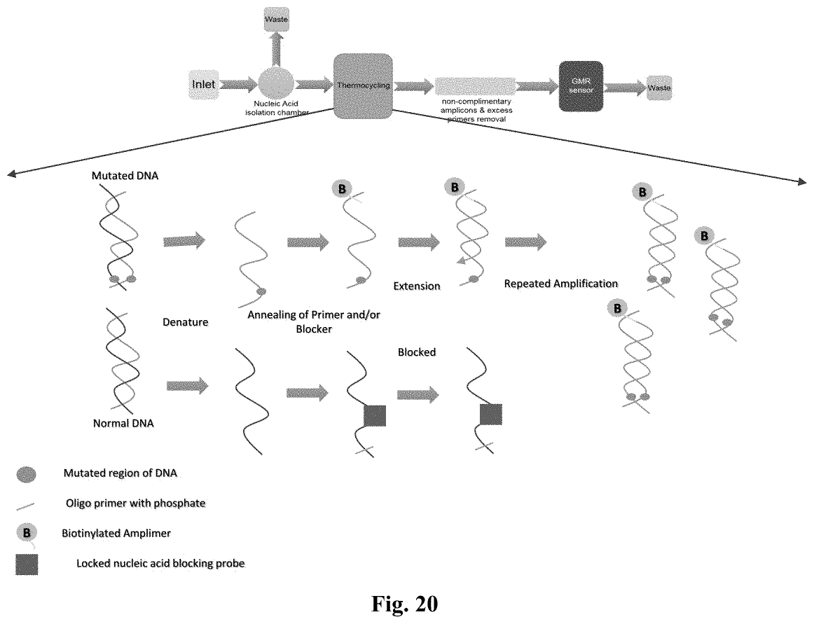

The blocking oligonucleotide was designed in the same orientation as the reverse primer, Accordingly, only one strand was blocked during PCR. After amplification, the amplification chamber was expected to include both double-stranded amplicons and single-stranded DNA (e.g., see FIG. 20).

The PCR products (amplicons) were moved to chamber 218 which contained a dried 5′-3′ exonuclease, the exonuclease was rehydrated, mixed with the amplicons in mixing chamber 216 and moved to chamber 210 where they were contacted with an exonuclease that digested the double-stranded. DNA into single-stranded DNA by digesting only amplicons having a 5′ phosphate (e.g., see FIG. 21).

The resulting single stranded, biotinylated amplicons were then moved to the GMR sensor 300 by opening valve V12.

The surface of the GMR sensor included a plurality of surface-bound capture nucleic acids. The sequence of the capture nucleic acid is shown below (i.e., SEQ ID NO:6).

Probe:
(SEQ ID NO: 6)
/5AmMC6/AAAAAAAAAAAAAAAGTTTGG+CC+CGCCC+AAA

The nucleotide bases preceded by a “+” symbol are locked nucleotides. The capture nucleic acid also included a C6 5′ amino modifier, which allowed the capture nucleic acid to be printed on the surface of the GMR. The capture nucleic acids were configured to bind specifically to the biotinylated amplicons comprising the target mutation (shown bold and underlined) as they flowed over the sensor. The capture nucleic acid was designed in the same orientation as the blocking oligonucleotide and the reverse primer. Accordingly, the blocking oligonucleotide could not hybridize to the capture nucleic acid.

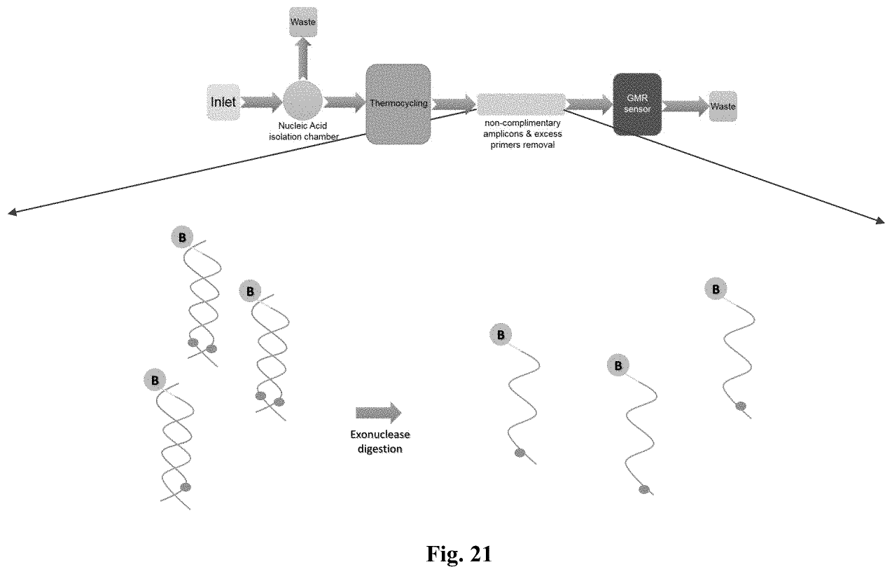

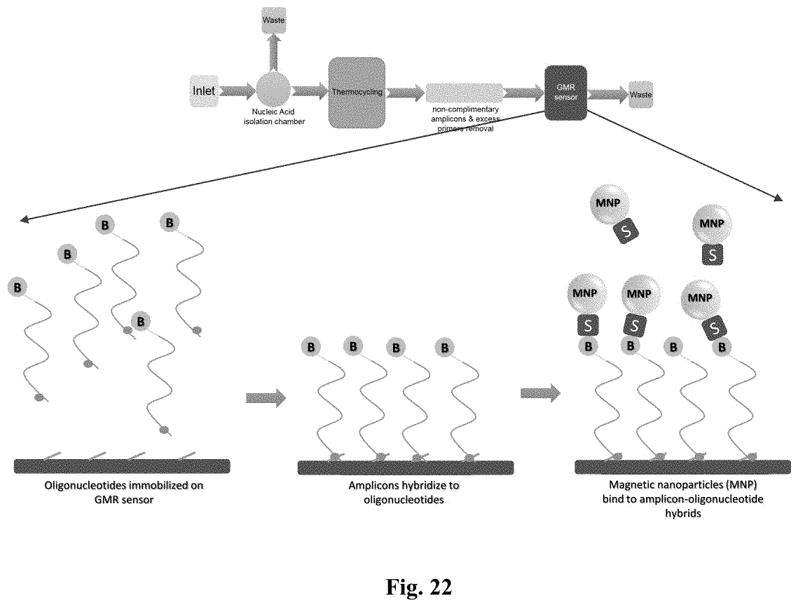

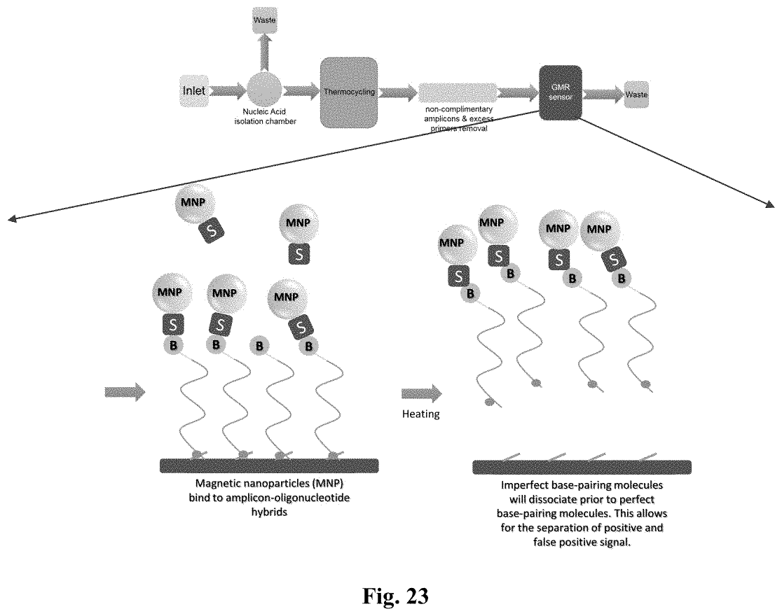

Magnetic beads stored in chamber 230 were moved to the GMR sensor by opening valve V13. The magnetic beads were streptavidin conjugated and bound tightly to the biotinylated amplicons captured on the surface of the GMR sensor (e.g., see FIG. 22). The binding of, or later release of, the magnetic beads to and from the sensor causes a change in magnetoresistance at the surface of the sensor which was detected and quantitated.

After binding of the biotinylated amplicons and subsequent binding of the magnetic streptavidin beads, the GMR sensor was washed by opening valve V14, which allowed the wash buffer in chamber 250 to flow over the surface of the GMR sensor. The wash buffer also decreased the sodium ion concentration from 50 mM to 10 mM, which resulted in an increase in the stringency of hybridization conditions. In this case, the melting temperature difference between the wild type and mutated target seqeunces to the capture nucleic acid of SEQ ID NO:6 (/5AmMC6/AAAAAAAAAAAAAAAGTTTGG+CC+CGCCC+AAA) increased. Accordingly, after addition of the wash buffer, the difference in melting temperature between the wild type and mutated sequences was 15° C.

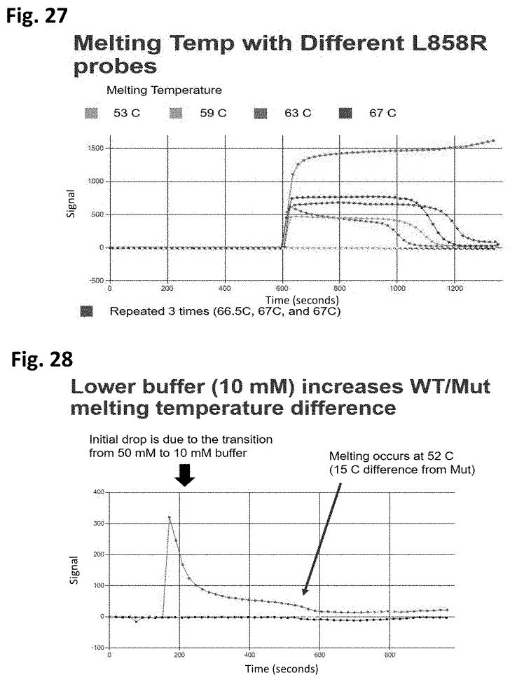

After washing, the temperature of the surface of the GMR sensor was slowly heated to increase the temperature from 45° C. to 85° C. over a period of 5 to 20 minutes while the magnetoresistance at the surface of the GMR sensor was simultaneously detected and recorded (e.g., see FIG. 27). Due to a 15 degree difference in the melting temperature of the capture nucleic acid with the wild type EGFR target sequence compared to the mutated EGFR target sequence, mutated EGFR target sequence leaves the surface at a later time. Accordingly, the presence of a the target mutation (c.2573T>G mutation) in the EGFR gene can be discriminated from the presence of a wild type sequence that may be non-specifically bound to the capture nucleic acid.

Different capture nucleic acids were generated and tested, each comprising a different number of locked nucleic acids, length and/or locked nucleotides in different positions. Each of the capture nucleic acids had a different melting temperature when hybridized to the mutated target nucleic acid and the binding/melting of each capture nucleic acid from the target could be diffentiated using the GMR sensor. These results (e.g., see FIG. 27) showed that a variety of capture probes can be designed to detect a variety of different genomic mutations which will allow multiplex detection of several different genomic mutations in a single run.

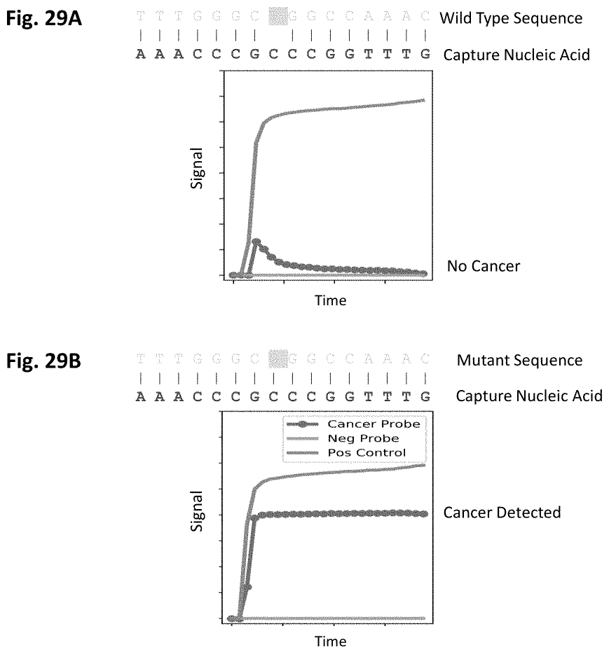

In a second experiment, the blocker oligonucleotide was excluded from the amplification chamber. Therefore the PCR reaction was conducted in the absence of a blocking oligonucleotide. After capturing the amplicons on the surface of the GMR (300), the Na⁺ concentration in the buffer flowing across the magnetic sensor was decreased from 50 mM to 10 mM. The results (FIG. 28) showed that false-positive signals representing captured wild-type DNA could be distinguished from true-positive signals (i.e., mutated target sequence, data not shown) where wild-type sequences denatured and dissociated from the surface of the magnetic sensor at a lower temperature and time (see arrow), while mutated target sequence was not denatured until the temperature hits about 67° C. (FIG. 27). Therefore, specificity and sensitivity of the assay was increased by dropping the positive ion concentration at the surface of the magnetic sensor and by increasing the temperature.

### Example 4

Using the microfluidic device and assay described for Example 3, cfDNA samples obtained from the plasma of a healthy patient (FIG. 29A) and from a cancer patient having a c.2573T>G mutation in the EGFR gene (FIG. 29B) were tested using a dynamic detection process. Briefly, samples were introduced into a loading chamber of the device, the sample was exposed to a lysis buffer to lyse any whole cells that may have been present, and the nucleic acids were purified using a silica membrane. Eluted nucleic acids were amplified using the primers of SEQ ID NO:3 (/5′-Biosg/CAGCCAGGAACGTACTGGTG) and SEQ ID NO:4 (/5′-Phos/ACTTTGCCTCCTTCTGCATG) in the presence of the blocking nucleotide of SEQ ID NO:5 (5′-TTTGGCCAGC). Fifty cycles of amplification were performed and the amplicons were digested with a 5-3′ exonuclease. The remaining biotinylated amplicons were captured on the surface of a GMR sensor using the capture nucleic acid of SEQ ID NO:6 (/5AmMC6/AAAAAAAAAAAAAAAGTTTGG+CC+CGCCC+AAA). The captured amplicons were contacted with streptavidin coated magnetic beads while dropping the sodium ion concentration to 10 mM and magnetoresistance at the sensor surface was measured while increasing the temperature from 45° C. to 80° C. The signal generated in FIG. 29A (blue line) indicated the absence of cancer in the subject. The signal generated in FIG. 29B (blue line) showed the presence of cancer the subject. The detection sensitivity in this assay was about 15 copies of mutated target sequence per mL of plasma. The sensitivity of the assay can be as low as 1 copy or less of mutated target sequence per mL of plasma, depending on the amount of cfDNA in a patient's plasma sample.

### Example 5

The device described in Example 3 was adapted such that the GMR sensor is replaced with a digital camera for the detection of fluorescent signal and a UV light source. Also, the streptavidin-magnetic beads was replaced with streptavidin coated quantum dots that emit fluorescent light upon excitation with a UV light source. The exonuclease chamber and exonuclease was omitted and the primer of SEQ ID NO:4 (5′-Phos/ACTTTGCCTCCTTCTGCATG) was directly coated on the surface of the PCR chamber, such that amplicons derived from SEQ ID NO:4 (5′-Phos/ACTTTGCCTCCTTCTGCATG) were permanently affixed to the PCR chamber. The dynamic detection process was essentially the same as that of Example 1 and 2 except that fluorescent light intensity (i.e., the signal) was detected by means of the digital camera at the sensor surface instead of resistance.

### Example 6

This example demonstrated multiple replicates from samples obtained from patients showing detection of a KRAS G12D mutation from samples with G12D mutation as low as 0.1%. This example also demonstrates that the same blocker and primers can be used to detect multiple different mutations within a single region.

Cell-free DNA was purchased from Horizon (HD780). Microfluidic device configuration, sensor surface functionalization, and assay method as described in Example 3 was employed to detect the KRAS G12D mutation. KRAS primers KRAS blocking oligonucleotide, were as follows:

Fonvard primer:
(SEQ ID NO: 7)
/5Biosg/ATTGTTGGATCATATTCGTCCAC
Reverse primer:
(SEQ ID NO: 8)
/5Phos/AGGCCTGCTGAAAATGACTG
Blocking oligonucleotide:
(SEQ ID NO: 9)
5′-C+T+G+G+T+G+G+C+G+T+A-3′.
Where “+” indicates locked nucleic acid.

The surface of the GMR sensor included a plurality of surface-bound capture nucleic acids. The sequence of the capture nucleic acid is shown below:

KRAS G12D Probe.
(SEQ ID NO: 10)
/5AmMC6/AAAAAAAAAAGTTGGAG+CTG+ATG+GCGTAG,
where nucleic acids preceded by “+”
are locked nucleic acids.

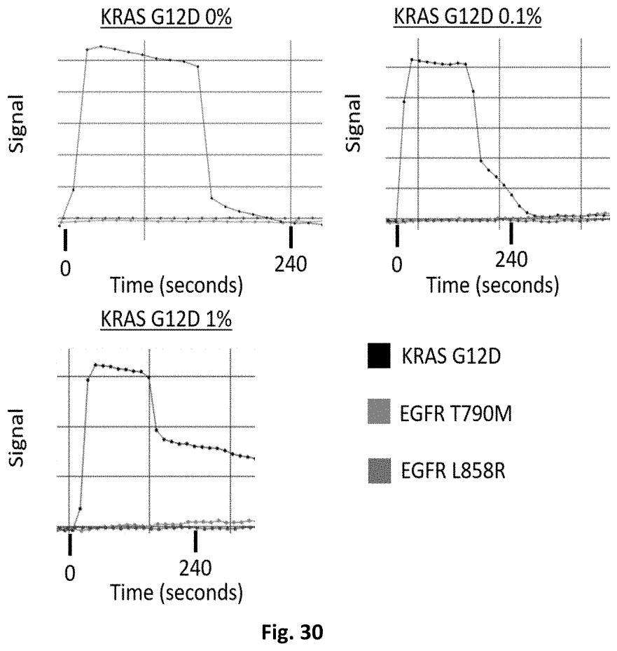

Signal readings were taken 240 seconds after magnetic beads were added. A student t-test was used to compare the mutant value to the wild type value. As shown in FIG. 30, both the 0.1% mutant and the 1.0% mutant had a p-value <0.0001, showing the strong specificity of the assay to distinguish the difference between mutant and wild type (non-mutant) with strong statistical significance. Parallel assays using probes for the EGFR T790M and EGFR L858 mutations as negative controls are also shown.

Table 4 below provides the signal strength of replicates 240 seconds after beads flowed over the sample.

TABLE 4

| KRAS G12D (0%) | KRAS G12D (0.1%) | KRAS G12D (1%) |
|---|---|---|
| 1.99 | 43.85 | 87.87 |
| 5.36 | 53.82 | 71.72 |
| −0.48 | 34.88 | 83.41 |
| 1.46 | 31.54 | 86.21 |
| −6.86 | 34.76 | 76.10 |
| 6.96 | 60.37 | 65.62 |
| 6.92 | 38.74 | 84.20 |
| −5.08 | 35.39 | 62.54 |
| Average 1.28 | Average: 41.67 | Average 77.21 |
| (−5.08-6.92) | (31.54-60.37) | (62.54-87.87) |

To demonstrate multiplex capability and better clinical utility, the same KRAS blocking oligonucleotide (5′-C+T+G+G+T+G+G+C+G+T+A-3′(SEQ ID NO:9)) and KRAS forward and reverse primers were used, but the capture nucleic acids (i.e., probes) provided below, were employed in order to detect the KRAS mutations outlined in Table 5:

KRAS G12V probe:
(SEQ ID NO: 11)
/5AmMC6/AAAAAAAAAAGTTGGAG+CTG+TT+GGC+GTAG
KRAS G12C probe:
(SEQ ID NO: 12)
/5AmMC6/AAAAAAAAAAGTTGGAG+CT+TGT+GGC+GTAG
KRAS G12A probe:
(SEQ ID NO: 13)
/5AmMC6/AAAAAAAAAAGTTGGAGCTG+CTGGCGTAG
KRAS G12S probe:
(SEQ ID NO: 14)
/5AmMC6/AAAAAAAAAAGTTGGAG+CT+AGT+GGC+GTAG
KRAS G12R probe:
(SEQ ID NO: 15)
/5AmMC6/AAAAAAAAAAGTTGGAG+CT+CGTGGCGTAG
KRAS G13D probe:
(SEQ ID NO: 16)
/5AmMC6/AAAAAAAAAAGAGCTG+GTG+AC+GTAGGCAA

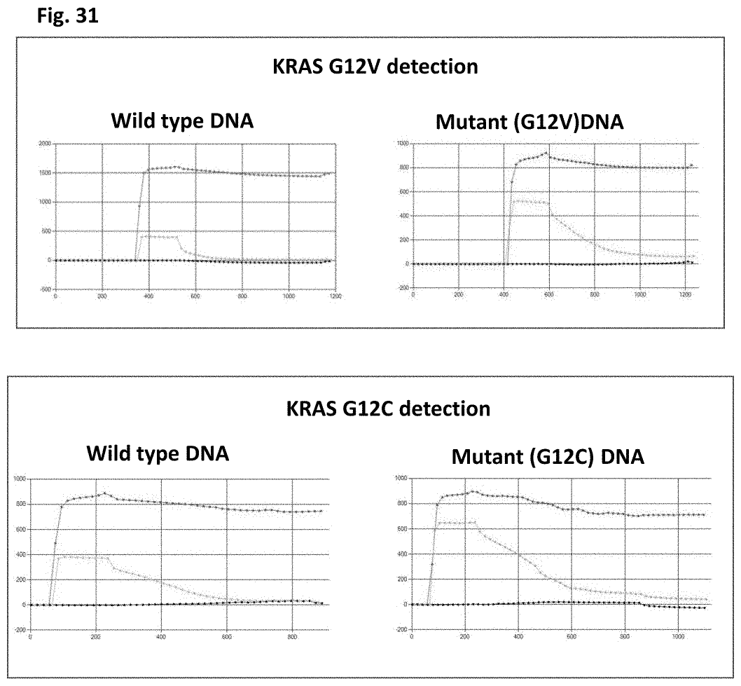

As depicted in FIG. 31, the same blocker and primers were demonstrated to be able to detect different mutations at position 35 of the KRAS gene. KRAS G12V is a change from a G to a T instead of a G to an A (which produces the KRAS G12D mutant protein). The results also demonstrate that at 500 seconds after bead flow the signal was still strong for the mutation and produced a distinguishably greater signal than that observed with wild-type DNA. The blocker and probe was also able to be used to detect a nearby nucleotide mutation, KRAS G12C, which is a mutation at position 34 instead of position 35. Similar reslts were obtained using probes to detect amplicons of the G13D and G13C mutations.

TABLE 5

| Mutation nucleotide | | Amino acid |
|---|---|---|
| location | Cancer ID | change |
| 35 G > A | COSM521 | G12D |
| 35 G > T | COSM520 | G12V |
| 38 G > A | COSM532 | G13D |
| 34 G > T | COSM516 | G12C |
| 35 G > C | COSM522 | G12A |
| 34 G > A | COSM517 | G12S |
| 34 G > C | COSM518 | G12R |
| 37 G > T | COSM527 | G13C |

### Example 7

To demonstrate the ability to detect genetic variants that can be used to detect and identify one or more species of organisms in one or more samples, a plurality of probes and primers were developed for use in detecting one or more fungal genera. In such methods, a blocking primer is not necessary and thus is not utilized in the assay. The plurality of probes was used in tandem to identify which fungal genera was present in each sample. from having a single probe looking for a single mutation.

To identify primers and probes to determine genus or species of target genera of fungi detected in samples, sequences from the target genera in the curated 18S fungal gene collection on NCBI were downloaded (BioProject PRJNA39195). These sequences were aligned by using muscle (v2.27.1; Edgar et al 2004), and a consensus sequence was constructed from the alignment. Then, all genome sequences available on NCBI for the target genera were downloaded, and the consensus sequence was used as the query in blast searches to identify the 18S locus in each genome (blastn from the NCBI BLAST+ package [v2.9.0; Camacho et al. 2009] with dc-megablast settings). For each genome the top hit was chosen using custom python scripts, and the entire set of sequences was aligned by using the linsi program from the MAFFT package (v7.407; Katoh & Standley 2014). The alignment was manually edited to remove sequences that appeared to be large outliers or that were unusually short. Then, genus-specific variable and conserved regions were identified with a custom python script.

A total of ten probes and 6 primers were used to identify and distinguish between fungi from 10 different categories encompassing 9 genera, and Candida auris in tested samples. These ten probes and 6 primers allowed for the identification of at least 25 species of fungus and categorizing them into 10 groups. Nine groups are based on genus and the last group for the species Candida auris. The ten groups are as follows:
1. Candida auris, Candida albicans, Candida tropicalis, Candida parapsilosis, Candida glabrata, Candida krusei, Candida haemulonis
2. Aspergillus fumigatus, Aspergillus flavus, Aspergillus niger, Aspergillus terreus
3. Cryptococcus neoformans, Cryptococcus gattii
4. Coccidioides immitis, Coccidioides posadasii
5. Fusarium solani, Fusarium oxysporum, Fusarium verticillioidis, and Fusarium moniliforme
6. Pneumocystis jirovecii
7. Blastomyces dermatitidis
8. Histoplasma capsulatum
9. Rhizopus oryzae, Rhizopus microspores
10. Candida auris

Probes and primers that were used to distinguish between and identify the presence and/or absence of fungi from these ten groups in tested samples were as follows.

Primers:
Reverse Primer:
(SEQ ID NO: 17)
/5Phos/GGAGTGATTTGTCTGCTTAATTGC
Forward Primer:
(SEQ ID NO: 18)
/5Biosg/GGCTTGAGCCGATAGTCCC
Forward Primer:
(SEQ ID NO: 19)
/5Biosg/GCCTCAAACTTCCATCGACTTC
Reverse Primer:
(SEQ ID NO: 20)
/5Phos/CGATAACGAACGAGACCTTAACC
Reverse Primer:
(SEQ ID NO: 21)
/5Phos/CAGGTCTGTGATGCCCTTAG
(SEQ ID NO: 22)
CAATGCTCTATCCCCAGCAC

The following primer, Forward Primer: 5Biosg/CATCGGCTTGAGCCGATAGTC (SEQ ID NO: 33) was used in lieu of Forward Primer: 5Biosg/GGCTTGAGCCGATAGTCCC (SEQ ID NO: 18) in independent experiments. Both Forward Primers, 5Biosg/CATCGGCTTGAGCCGATAGTC (SEQ ID NO: 33) and 5Biosg/GGCTTGAGCCGATAGTCCC (SEQ ID NO: 18), were found to successfully distinguish and identify fungi in tested samples.

Probes:
(SEQ ID NO: 23)
/5AmMC6/AAAAAAAAAAGTGCTGCCAGCGCGCCTCTTG
(SEQ ID NO: 24)
/5AmMC6/AAAAAAAAAACCGACCC+ACGT+TTG+TGG
(SEQ ID NO: 25)
/5AmMC6/AAAAAAAAAACGA+CCCGCGT+CTG+CG
(SEQ ID NO: 26)
/5AmMC6/AAAAAAAAAACGAGACCT+CG+GCCCTTAA
(SEQ ID NO: 27)
/5AmMC6/AAAAAAAAAACACTGACG+GA+GCCAGC
(SEQ ID NO: 28)
/5AmMC6/AAAAAAAAAAGAGTCTTA+CC+GC+CTTGGC
(SEQ ID NO: 29)
/5AmMC6/AAAAAAAAAAGCCAGC+AA+GT+T+CATTTCC
(SEQ ID NO: 30)
/5AmMC6/AAAAAAAAAAGTACT+TC+C+TT+GGCCGAAAG
(SEQ ID NO: 31)
/5AmMC6/AAAAAAAAAACACT+GA+TG+AA+G+TCAGCG
(SEQ ID NO: 32)
/5AmMC6/AAAAAAAAAAGTACATCA+CCTTGG+CCG

The 6 primers (SEQ ID Nos: 17-22) were used together in a single PCR reaction. DNA from 5 different fungi were amplified. Human cell-free DNA was used as a negative control. The 10 probes used for fungal classification (SEQ ID Nos: 23-32) and a positive and negative control were printed on GMR sensors, as described above, in triplicate.

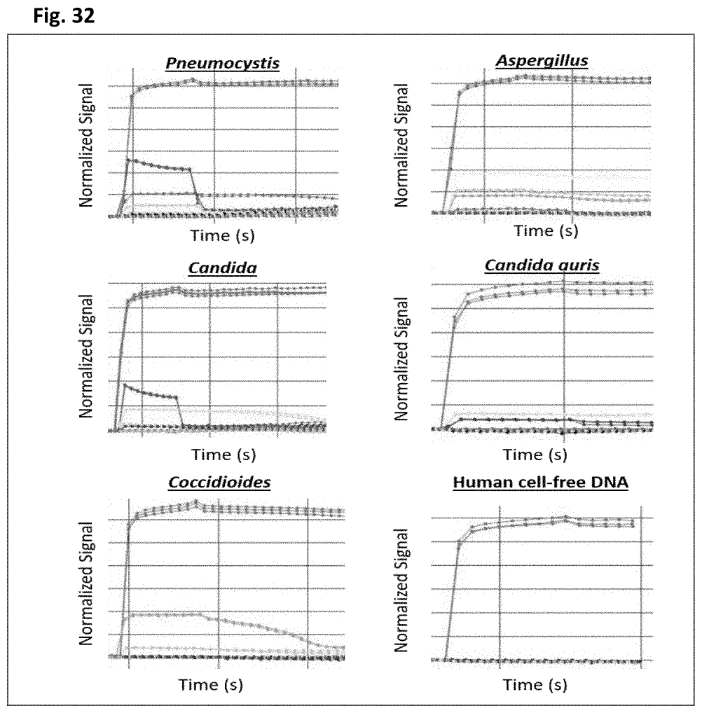

As depicted in FIG. 32, the assays were used to distinguish between and detect the indicated fungi in patent samples. The red trace indicates the measurements from the positive control and black trace indicates the measurements from negative control. The different probes when analyzed in combination correctly identified which fungal genera was present in the sample. Positive and negative external control samples were used as quality control samples.

### REFERENCES

Camacho, C., Coulouris, G., Avagyan, V., Ma, N., Papadopoulos, J., Bealer, K. and Madden, T. L., 2009. BLAST+: architecture and applications, BMC bioinformatics, 10(1), p. 421.

Edgar, R. C., 2004. MUSCLE: multiple sequence alignment with high accuracy and high throughput. Nucleic acids research, 32(5), pp. 1792-1797.

Katoh, K. and Standley, D. M., 2014. MAFFT: iterative refinement and additional methods. In Multiple sequence alignment methods (pp. 131-146). Humana Press, Totowa, N.J.

In some embodiments, all aspects of a method and/or all steps of a method described herein are performed in a microfluidic device described herein.

It will be understood that all embodiments disclosed herein may be combined in any manner to carry out a method of detecting an analyte and that such methods may be carried out using any combination of embodiments disclosed herein describing the various system components.

While the principles of the disclosure have been made clear in the illustrative embodiments set forth above, it will be apparent to those skilled in the art that various modifications may be made to the structure, arrangement, proportion, elements, materials, and components used in the practice of the disclosure.

It will thus be seen that the features of this disclosure have been fully and effectively accomplished. It will be realized, however, that the foregoing preferred specific embodiments have been shown and described for the purpose of illustrating the functional and structural principles of this disclosure and are subject to change without departure from such principles. Therefore, this disclosure includes all modifications encompassed within the spirit and scope of the following claims.

1. A method of detecting the presence of a first genetic variant in a target nucleic acid comprising:
(a) contacting the target nucleic acid with (i) a first primer, (ii) a second primer comprising a first member of a binding pair, (iii) a polymerase and (iv) a blocking oligonucleotide, wherein the blocking oligonucleotide comprises a sequence complementary to a second genetic variant of the target nucleic acid, and the first and second primers are configured for amplification of the target nucleic acid;
(b) amplifying the target nucleic acid thereby providing amplicons of the target nucleic acid;
(c) contacting the amplicons with a capture nucleic acid, wherein the capture nucleic acid comprises a sequence complementary to the first genetic variant of the target sequence, thereby providing captured amplicons comprising the first member of the binding pair;
(d) contacting the captured amplicons with a first detectable label comprising a second member of the binding pair; and
(e) detecting a presence, absence, amount, or change thereof, of the first detectable label.

2. The method of claim 1, wherein the capture nucleic acid is attached to a surface of a sensor.

3. The method of claim wherein the detecting of (e) comprises detecting the presence, absence, amount, or change thereof, of the first detectable label at the surface of the sensor.

4. The method of claim 2, wherein the detecting of (e) comprises a dynamic detection process.

5. The method of claim 4, wherein the dynamic detection process comprises increasing a temperature at or near the sensor, or at the surface of the sensor; changing a salt or cation concentration at or near the sensor, or at the surface of the sensor; or flowing a fluid across the surface of the sensor, during the detecting of (e).

6-7. (canceled)

8. The method of claim 1, wherein the detecting of (e) comprises detecting binding of one or more amplicons that bind to the capture nucleic acid.

9. The method of claim 2, wherein the detecting of (e) comprises detecting a change in an amount of amplicons that are bound to the surface of the sensor.

10. The method of claim 2, wherein the sensor comprises a magnetic sensor, the first detectable label comprises a magnetic particle, and the detecting of (e) comprises detecting a presence, absence, amount, or change of magnetoresistance at or near the surface of the magnetic sensor.

11. The method of claim 10, wherein the detecting of (e) comprises detecting a change in magnetoresistance at the surface of the sensor.

12-14. (canceled)

15. The method of claim 11, wherein the detecting the change in magnetoresistance comprises increasing the temperature of the surface by at least 5° C. while detecting the magnetoresistance at the surface of the sensor prior to, during and/or after increasing the temperature.

16. The method of claim 1, wherein the blocking oligonucleotide is selected from the group consisting of:
a blocking oligonucleotide which, when hybridized to the second genetic variant,
substantially prevents amplification of the target nucleic acid;
a blocking oligonucleotide with a melting temperature of at least 75° C.;
a blocking oligonucleotide with a length of from 9 to 20 oligonucleotides, and
a blocking oligonucleotide comprising at least 3 locked nucleotides.

17-20. (canceled)

21. The method of claim 1, wherein the capture nucleic acid is selected from the group consisting of:
a capture nucleic acid with a length of from 9 to 30 oligonucleotides;
a capture nucleic acid with a melting temperature of at least 50° C., and
a capture nucleic acid comprising at least 3 locked nucleotides.

22-23. (canceled)

24. The method of claim 1, wherein the presence of the first genetic variant in the target nucleic acid is determined according to a change of magnetoresistance detected in (e).

25. The method of claim 2, wherein the detecting of (e) comprises distinguishing the presence, absence, or amount of the first genetic variant at the surface of the sensor compared to a presence, absence, or amount of the second genetic variant or another nucleic acid at the surface of the sensor.

26. (canceled)

27. The method of claim 1, wherein the first member of the binding pair comprises biotin and the second member of the binding pair comprises streptavidin.

28. The method of claim 1, wherein the amplifying of (b) comprises a polymerase chain reaction.

29. (canceled)

30. The method of claim 2, wherein the method is conducted on a sample obtained from a subject, wherein the sample comprises the target nucleic acid.

31-32. (canceled)

33. The method of claim 30, wherein prior to (a) the sample is contacted with a microfluid channel, wherein the microfluidic channel is operably and/or fluidically connected to the sensor.

34. The method of claim 33, wherein prior to (a) the sample is contacted with a membrane configured to reversibly and/or non-specifically bind to nucleic acids in the sample, thereby providing bound nucleic acids, wherein the membrane is operably and/or fluidically connected to the microfluidic channel and to the sensor.

35. (canceled)

36. The method of claim 35, wherein prior to (a), the method comprises (i) contacting the sample with (i) a cell lysis solution, (ii) the membrane, (iii) optionally a wash solution, and (iv) an elution buffer, wherein after the contacting of (iv) bound nucleic acids are released from the membrane.

37. (canceled)

38. The method of claim 2, wherein the sensor comprises a giant magnetomagnetoresistance (GMR) sensor.

39. The method of claim 1, wherein the first genetic variant comprises at least one single nucleotide polymorphism (SNP); at least one single nucleotide mutation; or at least one single nucleotide deletion or insertion.

40-41. (canceled)

42. The method of claim 1, wherein the captured amplicons are in fluid contact with a buffer and prior to, or during, the detecting of (e), a concentration of positively charged cations in the buffer is decreased by at least 50%.

43-45. (canceled)

46. The method of claim 1, wherein the method is performed in a mircrofluidic device.

47. A mircrofluidic device for carrying out the method of claim 1, the device comprising:
(a) a microfluidic channel;
(b) a first chamber comprising a membrane;
(c) an amplification chamber;
(d) 3 or more miniature solenoid valves; and
(d) a sensor comprising a surface comprising a plurality of capture nucleic acids;
wherein the microfluidic channel is operably connected and/or fluidically connected with the first chamber, the amplification chamber, the 3 or more valves and the sensor.

48-103. (canceled)

104. The method of claim 1, further comprising, amplifying a detection signal measured by performing a detecting step, comprising, prior to performing the detecting step:
(a) contacting the captured amplicons with a second detectable label comprising magnetic particles and the second member of the binding pair, wherein the first detectable label associates with the second detectable label through an interaction between the first and second binding pairs of the first and second detectable labels;
thereby amplifying the detection signal that is measured upon performing the detecting step.

105. The method of claim 1, wherein the first genetic variant and the second genetic variant each comprise an allelic variant.

106-107. (canceled)

108. The method of claim 30, wherein each genetic variant that is detected distinguishes the presence of one organism from another organism in the sample.

109. The method of claim 1, wherein the first genetic variant comprises at least two single nucleotide polymorphisms (SNP); at least two single nucleotide mutations; or at least two single nucleotide deletions or insertions.

110-114. (canceled)

115. The method of claim 1, wherein the first primer comprises at least one of:
/5Phos/AGGCCTGCTGAAAATGACTG (SEQ ID NO:8),
/5Phos/GGAGTGATTTGTCTGCTTAATTGC (SEQ ID NO: 17),
/5Phos/CGATAACGAACGAGACCTTAACC (SEQ ID NO: 20), and
/5Phos/CAGGTCTGTGATGCCCTTAG (SEQ ID NO: 21);
wherein the second primer comprises at least one of:
5Biosg/ATTGTTGGATCATATTCGTCCAC (SEQ ID NO: 7),
5Biosg/GGCTTGAGCCGATAGTCCC (SEQ ID NO: 18),
5Biosg/GCCTCAAACTTCCATCGACTTC (SEQ ID NO: 19), and
5Biosg/CAATGCTCTATCCCCAGCAC (SEQ ID NO: 22); and
wherein the capture nucleic acid comprises at least one of:
/5AmMC6/AAAAAAAAAAGTTGGAG+CTG+ATG+GCGTAG (SEQ ID NO: 10),
/5AmMC6/AAAAAAAAAAGTTGGAG+CTG+TT+GGC+GTAG (SEQ ID NO: 11),
/5AmMC6/AAAAAAAAAAGTTGGAG+CT+TGT+GGC+GTAG (SEQ ID NO: 12),
/5AmMC6/AAAAAAAAAAGTTGGAGCTG+CTGGCGTAG (SEQ ID NO: 13),
/5AmMC6/AAAAAAAAAAGTTGGAG+CT+AGT+GGC+GTAG (SEQ ID NO: 14),
/5AmMC6/AAAAAAAAAAGTGCTGCCAGCGCGCCTCTTG (SEQ ID NO: 23),
/5AmMC6/AAAAAAAAAACCGACCC+ACGT+TTG+TGG (SEQ ID NO: 24),
/5AmMC6/AAAAAAAAAACGA+CCCGCGT+CTG+CG (SEQ ID NO: 25),
/5AmMC6/AAAAAAAAAACGAGACCT+CG+GCCCTTAA (SEQ ID NO: 26),
/5AmMC6/AAAAAAAAAACACTGACG+GA+GCCAGC (SEQ ID NO: 27),
/5AmMC6/AAAAAAAAAAGAGTCTTA+CC+GC+CTTGGC (SEQ ID NO: 28),
/5AmMC6/AAAAAAAAAAGCCAGC+AA+GT+T+CATTTCC (SEQ ID NO: 29),
/5AmMC6/AAAAAAAAAAGTACT+TC+C+TT+GGCCGAAAG (SEQ ID NO: 30)
/5AmMC6/AAAAAAAAAACACT+GA+TG+AA+G+TCAGCG (SEQ ID NO: 31), and
/5AmMC6/AAAAAAAAAAGTACATCA+CCTTGG+CCG (SEQ ID NO: 32).

116. (canceled)

117. The method of claim 1, wherein the blocking oligonuceloetide comprises 5′-C+T+G+G+T+G+G+C+G+T+A-3′ (SEQ ID NO:9).

118-126. (canceled)
